SEQUENCE LISTING (85 sequences)

SEQ ID NO: 1 - DNA, 2211 bp, Artificial Sequence
  adeno-associated virus hu68
  misc_feature (1)..(2208): vp1
  misc_feature (412)..(2208): vp2
  misc_feature (604)..(2208): vp3
atggctgccgatggttatcttccagattggctcgaggacaacctcagtgaaggcattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgaagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgtgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcaaagactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctaacgcttaatgatggaagccaagccgtgggtcgttcgtccttttactgcctggaatatttcccgtcgcaaatgctaagaacgggtaacaacttccagttcagctacgagtttgagaacgtacctttccatagcagctatgctcacagccaaagcctggaccgactcatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactaccaacccagtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtttattctgaaccccgccccattggcaccagatacctgactcgtaatctgtaa

SEQ ID NO: 2 - protein, 736 aa, Artificial Sequence
  adeno-associated virus hu68
  MISC_FEATURE (1)..(736): vp1
  MISC_FEATURE (138)..(736): vp2
  MISC_FEATURE (202)..(736): vp3
MAADGYLPDWLEDNLSEGIREWWALKPGAPQPKANQQHQDNARGLVLPGYKYLGPGNGLDKGEPVNEADAAALEHDKAYDQQLKAGDNPYLKYNHADAEFQERLKEDTSFGGNLGRAVFQAKKRLLEPLGLVEEAAKTAPGKKRPVEQSPQEPDSSVGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 3 - DNA, 2208 bp, Artificial Sequence
  adeno-associated virus hu71/74
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaagagcgtcttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaagcgggccagcagcctgcaagaaaaagattgaattttggtcagactggagacgcagactccgtacctgacccccagcctctcggacagccaccagcagccccctctggtttgggatctactacaatggctacaggcagtggcgcaccagtggcagacaataacgagggtgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagcctcaaacgacaaccactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcgggtcggcgcatcaaggatgcctcccgccgtttccagcggacgtcttcatggtcccacagtatggatacctcaccctgaacaacgggagtcaagcggtaggacgctcttcattttactgcctggagtactttccttctcagatgcttcgtaccggaaacaactttcagttcagctacacctttgaagacgtgcctttccacagcagctacgctcacagccagagtctggatcggctgatgaatcctctgatagaccagtacctgtattatctgaacaagacacaatcaaatagtggaactcttcagcagtctcggctactgtttagccaagctggacccaccaacatgtctcttcaagctaaaaactggctgcctggaccttgctacagacagcagcgtttgtcaaagcaggcaaacgacaacaacaacagcaactttccctggactgcagctacaaagtatcatctaaatggccgggactcgttggttaatccaggaccagcaatggccagccacaaagacgatgaagaaaagtttttccccatgcatggaaccctgatatttggtaaacaaggaacaaatgctaacgacgcggatttggacaatgtcatgattacagatgaagaagaaatccgcaccaccaatccagtggctactgagcagtacggatatgtgtcaaataatttgcaaaactcaaatactggtccaactactggaactgtcaatcaccaaggagcgttacctggtatggtgtggcaggatcgagatgtgtacctgcagggacccatttgggccaagattcctcacaccgatggacactttcatccttctccactgatgggaggttttggactcaaacacccgcctcctcagatcatgatcaaaaacactcccgttccagccaatccccccacaaacttcagttctgccaagtttgcttccttcatcacacagtattccacggggcaggtcagcgtggagatcgagtgggagctgcagaaggagaacagtaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 4 - protein, 735 aa, Artificial Sequence
  adeno-associated virus hu71/74
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTNMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 5 - DNA, 2208 bp, Artificial Sequence
  adeno-associated virus hu79
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcacaaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggcaacctcggacgagcagtcttccaggcaaaaaagagggttcttgaacctctgggcctggttgaggaacctgttaagacggctccgggaaaaaagaggccggtagagcactctcctgcggagccagattcctcctccggaactggaaagtcgggcaaccagcctgcaagaaagagattgaatttcggtcagactggagacgcagactccgtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagccagcaacgacaaccactactttggctacagcaccccctgggggtattttgacttcaacagattccactgccacttttcaccacgtgactggcaaagactcatcaacaacaattggggattccggcccaaaagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgcttcgtaccggaaacaactttaccttcagctacacctttgaggacgttcctttccacagcagctacgctcacagccagagtttggaccgtctcatgaatcctctcatcgaccagtacctgtattacttgagcaaaacaaacacgccgagcggaaccaccacgcagtccaggcttcagttttctcaggccggagcgagtgacattcgggaccagtctaggaactggcttcctggaccctgttaccgccagcagcgagtatcaaagacagctgcggacaacaacaatagtgattactcgtggactggagctaccaagtaccacctcaatggaagagactctttggtgaatccgggaccggccatggccagccacaaggacgatgaagaaaagtattttcctcagagcggggttctcatctttggaaaacaagactcgggaaaaactaatgtggacattgaaaaggtcatgattacagacgaagaggaaatcaggaccaccaatcccgtggctacggagcagtatggtgctgtatctaccaacctccagagcggccacacacaagcagctaccgcagatgtcaacacacaaggcgttcttccaggcatggtctggcaggacagagacgtgtacctgcaggggcccatctgggcaaagattccacacacggacggacattttcacccctctcccctcatgggcggattcggacttaaacacccgcctcctcagattctcatcaagaacaccccggtacctgcgaatccttcgaccaccttcagtgcggcaaagtttgcttccttcatcacacagtactccacagggcaggtcagtgtggagatcgagtgggagctgcagaaggagaacagcaaacgctggaatcccgagatccagtacacttccaactacaacaagtctgttaatgtggactttactgtggacactaatggcgtgtattcagagcctcgccccattggcaccagatacctgactcgtaatctgtaa

SEQ ID NO: 6 - protein, 735 aa, Artificial Sequence
  adeno-associated virus hu79
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKSGNQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNTPSGTTTQSRLQFSQAGASDIRDQSRNWLPGPCYRQQRVSKTAADNNNSDYSWTGATKYHLNGRDSLVNPGPAMASHKDDEEKYFPQSGVLIFGKQDSGKTNVDIEKVMITDEEEIRTTNPVATEQYGAVSTNLQSGHTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 7 - DNA, 2208 bp, Artificial Sequence
  adeno-associated virus hu80
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggcaacctcggacgagcagtcttccaggccaaaaagaggattcttgaacctctgggcctggttgaggaacctgttaaaacggctccgggaaaaaagaggccggtagagcactctcctgcggagccagactcctcctcgggaaccggaaaagcgggccagcagcctgcaagaaaaagattgaatttcggtcagactggagacgcagactccgtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggctctgcccacctacaacaaccatctgtacaagcagatttccagccagtctggagccagcaacgacaaccactactttggctacagcaccccttgggggtattttgacttcaacagattccactgccacttttcaccacgtgactggcaaagactcatcaataacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgtttccagcggacgtcttcatggtcccacagtatggatacctcaccctgaacaacgggagtcaggcagtaggacgctcttcattttactgcctggagtactttccttctcagatgctgcgtaccggaaacaactttaccttcagctacacctttgaggacgttcctttccacagcagctacgctcacagccagagtttggaccgtctcatgaatcctctcatcgaccagtacctgtattacttgagcagaacaaacactccaagcggaaccaccacgcagtccaggcttcagttttctcaggccggagcgagtgacattcgggaccagtctaggaactggcttcctggaccctgttatcgccagcagcgagtatcaaagacatctgcggataacaacaacagtgaatactcgtggactggagctaccaagtaccacctcaatggcagagactctctggtgaatccgggcccggccatggccagtcacaaggacgatgaagaaaagttttttcctcagagcggggttctcatctttgggaagcaaggctcagagaaaacaaatgtggacattgaaaaggtcatgattacagacgaagaggaaatcaggaccaccaatcccgtggctacggagcagtatggttctgtatctaccaacctccagagcggcaacacacaagcagctaccgcagatgtcaacacacaaggcgttcttccaggcatggtctggcaggacagagacgtgtacctgcaggggcccatctgggcaaagattccacacacggacggacattttcacccctctcccctcatgggcggatttggactgaaacaccctcctccacagattctcattaagaataccccggtacctgcgaatccttctaccactttcagcgcggcaaagtttgcttccttcatcacacagtattccacggggcaggtcagcgtggagatcgagtgggagctgcagaaagagaacagcaaacgctggaatcccgagattcagtacacttccaactacaacaaatctgttaatgtggactttactgtggacactaatggtgtgtattcagagcctcgccccattggcaccagatacctgactcgtaatctgtaa

SEQ ID NO: 8 - protein, 735 aa, Artificial Sequence
  adeno-associated virus hu80
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPAEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 9 - DNA, 2208 bp, Artificial Sequence
  adeno-associated virus hu83
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcaaaaaagagggttcttgaacctctgggcctggttgaggaacctgttaagacggctccgggaaaaaagaggccggtagagcactctcctgcggagccagattcctcctccggaactggaaagtcgggcaaccagcctgcaagaaagagattgaatttcggtcagactggagactcagactccgtacctgacccccagcctctcggacagccaccagcagccccctctggtctgggaactaatacgatggcttcaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccatctgtacaagcaaatatccagccagtctggagccagcaacgacaatcactactttggctacagcaccccctgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacctttgaggacgtgcctttccacagcagctacgctcacagccagagtttggaccgtctcatgaatcctctcatcgaccagtacctgtattacttgagcagaacaaacactccaagcggaaccaccacgcagtccaggcttcagttttctcaggccggagcgagtgacattcgggaccagtctaggaactggcttcctggaccctgttatcgccagcagcgagtatcaaagacatctgcggacaacaacaacagtgaatactcgtggactggagctaccaagtaccacctcaatggcagagactctctggtgaatccgggcccggccatggccagtcacaaggacgatgaagaaaagttttttcctcagagcggggttctcatctttgggaagcaaggctcagagaaaacaaatgtggacattgaaaaggtcatgattacagacgaagaggaaatcagaaccaccaatcccgtggccacggagcagtatggttctgtatctaccaacctccagagcggcaacacacaagcagctactgcagatgtcaacacacaaggcgttcttccaggcatggtctggcaggacagagacgtgtacctgcaggggcctatctgggcaaaaattccacacacggacggacattttcacccctctcccctcatgggcggattcggacttaaacaccctcccccgcagattctcatcaagaacacccctgtacctgcgaatccttcgaccaccttcagtgcggcaaagtttgcttccttcattacacagtattccacgggacaggtcagcgtggagatcgagtgggagctgcagaaggaaaacagcaaacgttggaatcccgagattcagtacacttccaactacaacaagtctgttaatgtggactttactgtggacactaatggtgtgtattcagagcctcgccccattggcaccagatacctgactcgtaatctgtaa

SEQ ID NO: 10 - protein, 735 aa, Artificial Sequence
  adeno-associated virus hu83
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKSGNQPARKRLNFGQTGDSDSVPDPQPLGQPPAAPSGLGTNTMASGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQSGNTQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 11 - DNA, 2208 bp, Artificial Sequence
  adeno-associated virus hu74/71
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggccaaaaagagggttcttgaacctctgggcctggttgaggaacctgttaagacggctccgggaaaaaagaggccagtagagcactctcctgtggagccagactcctcctcgggaaccggaaaagcgggccagcagcctgcaagaaagagattgaattttggtcagactggagacgcagactccgtacctgacccccagcctctcggacaaccaccagcagccccctctggtctgggatctactacaatggctacaggcagtggcgcaccagtggcagacaataacgagggtgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagccagcaacgacaaccactactttggctacagcaccccctgggggtattttgacttcaacagattccactgccacttttcaccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgctgcgtaccggaaacaactttcagttcagctacacctttgaagacgttcctttccatagcagctacgctcacagccagagtctggatcggctgatgaatcctctgatcgaccagtacctgtattatctgaacaagacacaatcaaatagtggaactcttcagcagtctcggctactgtttagccaagctggacctaccaacatgtctcttcaagctaaaaactggctgcctggaccttgctacagacagcagcgtctgtcaaagcaggcaaacgacaacaacaacagcaactttccctggactgcggctacaaagtatcatctaaatggccgggactcgttggttaatccaggaccagctatggccagccacaaagacgatgaagaaaagtttttccccatgcatggaaccctgatatttggtaaacaaggaacaaatgctaacgacgcggatttggacaatgtcatgattacagatgaagaagaaatccgcaccaccaatcccgtggctacggagcagtacggatatgtgtcaaataatttgcaaaactcaaatactggtccaactactggaactgtcaatcaccaaggagcgttacctggtatggtgtggcaggatcgagacgtgtacctgcagggacccatttgggccaagattcctcacaccgatggacactttcacccttctccactgatgggaggttttggactcaaacacccgcctcctcaaatcatgatcaaaaacactcccgttccagccaatcctcccacaaacttcagttctgccaagtttgcttctttcatcacacagtattccacggggcaggtcagcgtggagattgagtgggagctgcagaaggagaacagcaagcgctggaaccccgagattcagtacacttccaactacaacaagtctgttaatgtggactttactgtggacactaatggtgtgtattcagagcctcgccccattggcaccagatacctgactcgtaatctgtaa

SEQ ID NO: 12 - protein, 735 aa, Artificial Sequence; identical to SEQ ID NO: 4
  adeno-associated virus hu74/71
  MISC_FEATURE (1)..(735): vp1
  MISC_FEATURE (138)..(735): vp2
  MISC_FEATURE (203)..(735): vp3

SEQ ID NO: 13 - DNA, 2208 bp, Artificial Sequence
  adeno-associated virus hu77
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaagcgggccagcagcctgcaagaaaaagattgaattttggtcagactggagacgcagactccgtacctgacccccagcctctcggacagccaccagcagccccctctggtttgggatctactacaatggctacaggcagtggcgcaccagtggcagacaataacgagggtgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagcctcaaacgacaaccactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtatcagctcccgtacgtcctcggctcggcgcatcaaggatgcctcccgccgttcccagcggacgtcttcatggtcccacagtatggatacctcaccctgaacaacgggagtcaggcggtaggacgctcttccttttactgcctggagtactttccttctcagatgctgcgtactggaaacaactttcagttcagctacacttttgaagacgttcctttccacagcagctacgctcacagccagagtctggatcggctgatgaatcctctgatcgaccagtacctgtattatctgaacaaaacacaatcaaatagtggaactcttcagcagtctcggctactgtttagtcaagctggacccaccagcatgtctcttcaagctaaaaactggttacctggaccttgctacagacagcagcgtctgtcaaagcaggcaaacgacaacaacaacagcaactttccctggactgcggctacaaagtatcacctaaatggccgggactcgttggttaatccaggaccagctatggccagccacaaagacgatgaagaaaagtttttccctatgcatggaaccctgatatttggtaaacaaggaacaaatgctaacgacgcggatttggacaatgtcatgattacagatgaagaagaaatccgcaccaccaatcccgtggctacggagcagtacggatatgtgtcaaataatttgcaaaactcaaatactggtccaactactggaactgtcaatcaccaaggagcgttacctggcatggtgtggcaggatcgagacgtgtacctgcagggacccatttgggccaagattcctcacacagatggacactttcatccttctccactgatgggcggattcggactcaaacacccgcctcctcagatcatgatcaaaaacactcccgttccagccaatcctcctacaaactttagctctgccaagtttgcttccttcatcacacagtattccacggggcaggtcagcgtggagatcgagtgggagctgcagaaggagaacagcaaacgctggaatcccgagatccagtacacttccaactacaacaagtctgttaatgtggactttactgtggacaccaatggcgtgtattcagagcctcgccccattggcaccagatacctgactcgtaatctgtaa

SEQ ID NO: 14 - protein, 735 aa, Artificial Sequence
  adeno-associated virus hu77
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 15 - DNA, 2208 bp, Artificial Sequence
  adeno-associated virus hu78/88
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagaaccggtcaacgaggcagacgccgcggccctcgagcacgacaaggcctacgaccggcagctcgacagcggagacaacccgtacctcaagtacaaccacgccgacgcggagtttcaggaacgtcttaaagaagatacgtcttttgggggcaacctcggacgagcagtcttccaggcaaaaaagagggttcttgaacctctgggcctggttgaggagcctgttaagacggctccgggaaaaaagaggccggtagagcactctcctgcggagccagactcctcctcgggaaccggaaaggcgggccagcagcctgctagaaaaagactgaatttcggtcagactggagacgcagactccgtaccagaccctcaacctctcggagaaccaccagcagcccccacaagtttgggatctactacaatggcttcaggcggtggcgcaccagtggcagacaataacgagggtgccgatggagtgggtaattcctcgggaaattggcattgcgattcccaatggctgggcgacagagtcattaccaccagcacccgaacctgggccctgcccacctacaacaaccatctgtacaagcaaatttccagccaatcaggagccagcaacgacaaccactactttggctacagcaccccctgggggtattttgacttcaacagattccactgccacttttcaccacgtgactggcaaagacttatcaacaacaactggggattccggcccaagagactcaacttcaagctctttaacattcaagtcaaagaggtcacgcagaatgacggtacgacaacgattgccaataaccttaccagcacggttcaggtgtttactgactcggagtaccagctcccgtacgtcctcggctcggcgcatcaaggatgcctcccgccgttcccagcggacgtcttcatggtcccacagtatggatacctcaccctgaacaacgggagtcaggcggtaggacgctcttccttttactgcctggagtactttccttctcagatgctgcgtacaggaaacaactttcagttcagctacacctttgaagacgtgcctttccacagcagctacgctcacagccagagtctggatcggctgatgaatcctctgatcgaccagtacctgtattatctgaataagacacaaaactctagtggaactgttcaacagtctcggctactgtttagtcaagctggacccaccagtatgtctcttcaagctaaaaactggctgcctggaccttgctacagacagcagcgtctgtcaaagcaggcaagcgacaacaacaacagcaactttccctggactgcggccacaaagtatcatctaaatggccgggactcattggttaatccaggaccagctatggccagtcacaaggatgacgaagaaaagtttttccccatgcatggaaccctgatatttggtaaagaaggaacaactgctaacaacgcggatttggaacatgtcatgattacagatgaagaagaaatcagaaccaccaatcccgtggctacggagcagtacggaaatgtgtcaaataatttgcaaaactcaaatactggtccaactactgaaaatgtcaataaccaaggagcgttacctggtatggtgtggcaggatcgagacgtgtacctgcagggacccatttgggccaagattcctcacaccgatggacactttcatccttctccactgatgggaggctttggactcaaacacccgcctcctcagatcatgatcaaaaacactcccgttccagccaatcctcccacaaacttcagtgcggcaaagtttgcttccttcatcacacagtattccacagggcaggtcagcgtggagatcgagtgggagctgcagaaggagaacagcaaacgctggaaccccgaaatccagtacacttccaactataacaaatctgttaatgtggactttactgtggacactaatggtgtgtattcagagcctcgccccattggcaccagatacctgactcgtaatctgtaa

SEQ ID NO: 16 - protein, 735 aa, Artificial Sequence
  adeno-associated virus hu78/88
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGEPPAAPTSLGSTTMASGGGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQNSSGTVQQSRLLFSQAGPTSMSLQAKNWLPGPCYRQQRLSKQASDNNNSNFPWTAATKYHLNGRDSLVNPGPAMASHKDDEEKFFPMHGTLIFGKEGTTANNADLEHVMITDEEEIRTTNPVATEQYGNVSNNLQNSNTGPTTENVNNQGALPGMVWQDRDVYLQGPIWAKIPHTDGHFHPSPLMGGFGLKHPPPQIMIKNTPVPANPPTNFSAAKFASFITQYSTGQVSVEIEWELQKENSKRWNPEIQYTSNYNKSVNVDFTVDTNGVYSEPRPIGTRYLTRNL

SEQ ID NO: 17 - DNA, 2208 bp, Artificial Sequence
  adeno-associated virus hu70
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaaaggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcaaaaaagagggttcttgaacctctgggcctggttgaggaacctgttaagacggctccgggaaaaaagaggccggtagagcactctcctgcggagccagactcctcctcgggaaccggaaaagcgggccagcagcctgcaagaaaaagattgaattttggtcagactggagacgcagactccgtacctgacccccagcctctcggacagccaccagcagccccctctggtctgggatctactacaatggctacaggcagtggcgcaccaatggcagacaataacgagggtgccgatggagtgggtaattcctcaggaaattggcattgcgattcccaatggctgggcgacagagtcatcaccaccagcacccgaacttgggccctgcccacctacaacaaccacctctacaagcaaatctccagccaatcaggagcctcaaacgacaaccactactttggctacagcaccccttgggggtattttgacttcaacagattccactgccacttttcaccacgtgactggcaaagactcatcaacaacaactggggattccggcccaaaagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtcccacagtatggatacctcaccctgaacaacgggagtcaggcggtaggacgctcttccttttactgcctggagtactttccttctcagatgctgcgtactggaaacaactttcagttcagctacacctttgaagacgtgcctttccacagcagctacgctcacagtcagagtctggatcggctgatgaatcctctcatcgaccagtacctgtattatctgaacaagacacaaacaaatagtggaactcttcagcagtctcggctactgtttagccaagctggacccaccaacatgtctcttcaagctaaaaactggctgcctggaccttgctacagacagcagcgtttgtcaaagcaggcaaacgacaacaacaacagcaactttccctggactgcggctacaaagtatcatctaaatggccgggactcattggttaatccaggaccagctatggccagtcacaaggatgacgaagaaaagtttttccccatgcatggaaccctgatatttggtaaacaaggaacaaatgccaacgacgcggatttggaaaatgtcatgattacagatgaagaagaaatcagaaccaccaatcccgtggctacggagcagtacggatatgtgtcaaataatttgcaaaactcaaacactggtccaactactggaactgtcaatcaccaaggagcgttacctggtatggtgtggcaggatcgagacgtgtacctgcagggacccatttgggccaaaattcctcacaccgacggacactttcatccttctccactgatgggaggttttggactcaaacacccacctcctcagatcatgattaaaaacactcccgttccagccaatcctcccacaaacttcagttctgccaagtttgcttctttcatcacacagtattccacgggacaggtcagcgtggagatcgagtgggagctgcagaaggagaacagcaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 18 - protein, 735 aa, Artificial Sequence
  adeno-associated virus hu70
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQTNSGTLQQSRLLFSQAGPTNMSLQAKNWLPGPCYRQQRLSKQANDNNNSNFPWTAATKYHLNGRDSLVNPGPAMASHKDDEEKFFPMHGTLIFGKQGTNANDADLENVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 19 - DNA, 2208 bp, Artificial Sequence
  adeno-associated virus hu72
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaagacggctccgggaaaaaagaggccggtagagcactctcctgcggagccagactcctcctcgggaaccggaaaagcgggcaaccagcctgcaagaaagagattgaatttcggtcagactggagacgcagactccgtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccacctctacaagcaaatctccagccaatcaggagcctcaaacgacaaccactactttggctacagcaccccttgggggtattttgacttcaacagattccactgccacttttcgccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaataacgggagtcaggcggtaggacgctcttcattttactgcctggagtactttccttctcagatgcttcgtaccggaaacaactttaccttcagctacacctttgaggacgttcctttccacagcagctacgctcacagccagagtttggaccgtctcatgaatcctctcatcgaccagtacctgtattacttgagcagaacaaacactccaagcggaaccaccacgcagtccaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctttggtgaatccgggcccggccatggccagccacaaggacgatgaagaaaagttttttcctcagagcggggttctcatctttgggaagcaaggctcagagaaaacaaatgtggacattgaaaaggtcatgattacagacgaagaggaaatcaggaccaccaatcccgtggctacggagcagtatggtgctgtatctaccaacctccagagcggcaacacacaagcagctacctcagatgtcaatacacaaggcgttcttccaggcatggtctggcaggacagagacgtgtacctgcaggggcccatctgggcaaagattccacacacggacggacattttcacccctctcccctcatgggtggattcggacttaaacaccctcccccgcagattctcatcaagaacaccccggtacctgcgaatccttcgaccaccttcagtgcggcaaagtttgcttccttcatcacacagtactccacggggcaggtcagcgtggagatcgagtgggagctgcagaaggagaacagcaaacgctggaatcccgaaattcagtacacttccaactacaacaaatctattaatgtggactttactgtggacactaatggtgtgtattcagagcctcgccccattggcaccagatatctgactcgtaatctgtaa

SEQ ID NO: 20 - protein, 735 aa, Artificial Sequence
  adeno-associated virus hu72
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGNQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGAVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSINVDFTVDTNGVYSEPRPIGTRYLTRNL

SEQ ID NO: 21 - DNA, 2208 bp, Artificial Sequence
  adeno-associated virus hu75
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaagacggctccgggaaaaaagaggccggtagagcactctcctgtggagccagattcctcctccggaactggaaaagcgggcaaccagcctgcaagaaagagattgaatttcggtcagactggagacgcagactccgtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatatccagccagtctggagccagcaacgacaaccactactttggctacagcaccccttgggggtattttgacttcaacagattccactgccacttttcaccacgtgactggcaaagacttatcaacaacaactggggattccggcccaaaagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttcattttactgcctggagtacttcccttctcagatgctgcgtaccggaaacaactttaccttcagctacacctttgaggacgtgcctttccacagcagctacgctcacagccaaagtctggaccgtctcatgaatcctctcatcgaccagtacctgtattacttgagcagaacaaacactccaagcggaaccaccacgcagtccaggcttcagttttctcaggccggagcgagtgacattcgggaccagtctaggaactggcttcctggaccctgttatcgccagcagcgagtatcaaagacagctgcggataacaacaacagtgaatactcgtggactggagctaccaagtaccacctcaatggcagagactctctggtaaatccgggcccggcaatggccagccacaaggacgatgaagaaaagttttttcctcagagcggggttctcatctttggaaaacaagactcgggaaaaactaatgtggacattgaaaaggtcatgattacagacgaagaggaaatcaggaccaccaatcccgtggctacggagcagtatggtgctgtatctaccaatctccagagcggcaacacacaagcagctacctcagatgtcaacacacaaggcgttcttccaggcatggtctggcaggacagagacgtgtacctgcaggggcccatctgggcaaagattccacacacggacggacattttcacccctctcccctcatgggcggattcggacttaaacaccctcctccacagattctcatcaagaacaccccggtacctgcgaatccttcgaccaccttcagtgcggcaaagtttgcttccttcattacacagtactccacggggcaggtcagcgtggagatcgagtgggagctgcagaaggagaacagcaaacgctggaatcccgagattcagtacacttccaactacaacaaatctgttaatgtggactttactgtggacactaatggtgtgtattcagagccccgccccattggcaccagatacctgactcgtaatctgtaa

SEQ ID NO: 22 - protein, 735 aa, Artificial Sequence
  adeno-associated virus hu75
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGNQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTAADNNNSEYSWTGATKYHLNGRDSLVNPGPAMASHKDDEEKFFPQSGVLIFGKQDSGKTNVDIEKVMITDEEEIRTTNPVATEQYGAVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 23 - DNA, 2205 bp, Artificial Sequence
  adeno-associated virus hu76
  misc_feature (1)..(2202): vp1
  misc_feature (412)..(2202): vp2
  misc_feature (607)..(2202): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgcggagccagactcctccttgggaaccggaaaagcgggccagcagcctgcaagaaaaagactgaatttcggtcagactggagacgcagactccgtacctgacccccagcctctcggacagccaccagcagcccccacaagtttgggatctactacaatggctacaggcagtggcgcaccaatggcagacaataacgagggtgccgatggagtgggtaattcctcaggaaattggcattgcgattcccaatggctgggcgacagagtcatcaccaccagcacccgaacctgggccctgcccacctacaacaaccacctttacaagcaaatctccagccaatcaggagcctcaaacgacaaccactactttggctacagcaccccctgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgtttccagcggacgtcttcatggtcccacagtatggatacctcaccctgaacaacgggagtcaggcggtaggacgctcttccttttactgcctggagtactttccttctcagatgctgcgtactggaaacaactttcagttcagctacacttttgaagacgtgcctttccacagcagctacgctcacagccagagtttggatcggctgatgaatcctctgatcgaccagtacctgtattatctaaacagaacacaaacagctagtggaactcagcagtctcggctactgtttagccaagctggacccacaagcatgtctcttcaagctaaaaactggctgcctggaccgtgttatcgccagcagcgtttgtcaaagcaggcaaacgacaacaacaacagcaactttccctggactggagctaccaagtaccacctcaatggcagagactctttggtgaacccgggcccggccatggccagccacaaggacgatgaagaaaagtttttccccatgcatggaaccctaatatttggtaaagaaggaacaaatgctaccaacgcggaattggaaaatgtcatgattacagatgaagaggaaatcaggaccaccaatcccgtggctacagagcagtacggatatgtgtcaaataatttgcaaaactcaaatactgctgcaagtactgaaactgtgaatcaccaaggagcattacctggtatggtgtggcaggatcgagacgtgtacctgcagggacccatttgggccaagattcctcacaccgatggacactttcatccttctccactgatgggaggttttggactcaaacacccgcctcctcagattatgatcaaaaacactcccgttccagccaatcctcccacaaacttcagttctgccaagtttgcttccttcatcacacagtattccacgggacaggtcagcgtggagatcgagtgggagctgcagaaggagaacagcaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 24 - protein, 734 aa, Artificial Sequence
  adeno-associated virus hu76
  MISC_FEATURE (1)..(734): vp1
  MISC_FEATURE (138)..(734): vp2
  MISC_FEATURE (203)..(734): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSLGTGKAGQQPARKRLNFGQTGDADSVPDPQPLGQPPAAPTSLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTGATKYHLNGRDSLVNPGPAMASHKDDEEKFFPMHGTLIFGKEGTNATNAELENVMITDEEEIRTTNPVATEQYGYVSNNLQNSNTAASTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 25 - DNA, 2208 bp, Artificial Sequence
  adeno-associated virus hu81
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaaaggagagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggcaacctcggacgagcagtcttccaggccaaaaagaggattcttgaacctctgggcctggttgaggagcctgttaagacggctccgggaaaaaagaggccggtagagcactctcctgtggagccagactcctcctcgggaaccggaaaagcgggccagcagcctgcaagaaaaagattgaatttcggtcagactggagacgcagactccgtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagcctcaaacgacaaccactactttggctacagcaccccttgggggtattttgacttcaacagattccactgtcacttctctccacgtgactggcaaagactcatcaacaacaactggggattccggcccaaaagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacctttgaggacgttcctttccacagcagctacgctcacagccagagtttggaccgtctcatgaatcctctcatcgaccagtacctgtattacttgagcagaacaaacactccaagcggaaccaccacgcagtccaggcttcagttttctcaggccggagcgagtgacattcgggaccagtctaggaactggcttcctggaccctgttaccgccagcagcgagtatcaaagacatctgcggataacaacaacagtgattactcgtggactggagctaccaagtaccacctcaatggcagagactctctagtgaatccgggcccggccatggccagccacaaggacgatgaagaaaaatattttcctcagagcggggttctcatctttggaaaacaaggttcaaataaaactaatgtggacattgaaaaggtcatgattacagacgaagaagaaatcaggaccaccaatcccgtggctacggagcagtatggtgctgtatctaccaacctccagagcggcaacacacaagcagctacctcagatgtcaacacacaaggcgttcttccaggcatggtctggcaggacagagacgtgtacctgcaggggcccatctgggcaaagattccacacacggacggacattttcacccctctcccctcatgggcggattcggacttaaacaccctcccccgcagattctcatcaagaacaccccggtacctgcgaatccttcgaccaccttcagtgcggcaaagtttgcttccttcattacacagtattcaacggggcaggtcagcgtggaaatcgagtgggagctgcagaaagagaacagcaaacgctggaaccccgagatccagtacacttccaactacaacaagtctgttaatgtggactttactgtggacactaatggcgtgtattcagagcctcgccccattggcaccagatacctgactcgtaatctgtaa

SEQ ID NO: 26 - protein, 735 aa, Artificial Sequence
  adeno-associated virus hu81
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRILEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSDYSWTGATKYHLNGRDSLVNPGPAMASHKDDEEKYFPQSGVLIFGKQGSNKTNVDIEKVMITDEEEIRTTNPVATEQYGAVSTNLQSGNTQAATSDVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 27 - DNA, 2208 bp, Artificial Sequence
  adeno-associated virus hu82
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgcagcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcaaaaaagagggttcttgaacctctgggcctggttgaggagcctgttaagacggctccgggaaaaaagaggccggtagagcactctcctgcggagccagactcctcctccggaactggaaagtcgggcaaccagcctgcaagaaagagattaaacttcggtcagactggagactcagactccgtacctgacccccagcctctcggacaaccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagcctcaaacgacaaccactactttggctacagcaccccctgggggtattttgacttcaacagattccactgccacttttcaccacgtgactggcaaagactcatcaacaacaattggggattccggcccaaaagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcagatgcttcgtaccggaaacaactttaccttcagctacacctttgaggacgttcctttccacagcagctacgctcacagccagagtttggaccgtctcatgaatcctctcatcgaccagtacctgtattacttgagcagaacaaacactccaagcggaaccaccacgcagtccaggcttcagttttctcaggccggagcaagtgacattcgggaccagtctaggaattggcttcctggaccctgttaccgccagcagcgagtatcaaagacacctgcggataataacaacagtgaatactcgtggactggagctaccaagtaccacctcaatggcagagactctctggtgaatccgggcccggccatggccagccacaaggacgatgaagaaaagtttttccctcagagcggggttctcatctttgggaagcaaggctcagagaaaacaaatgtggacattgaaaaggtcatgattacagacgaagaggaaatcaggaccaccaatcctgtggctacggagcagtatggtactgtatctactaaccttcagagcagcaacacacaagcagctacctcagatgtcaacacacaaggcgttcttccaggcatggtctggcaggacagagacgtgtacctgcaggggcctatctgggcaaagattccacacacggacggacattttcacccctctcccctcatgggcggattcggacttagacaccctcctcctcagattctcatcaagaacaccccggtacctgcaaatccttcgaccaccttcagtgcggcaaagtttgcttccttcattacacagtactccacggggcaggtcagcgtggagatcgagtgggagctgcagaaggagaacagcaaacgctggaatcccgagattcagtacacttccaactataacaagtctgttaatgtggactttactgtggacactaatggcgtatattcagagcctcgccccattggcaccagatacctgactcgtaatctgtaa

SEQ ID NO: 28 - protein, 735 aa, Artificial Sequence
  adeno-associated virus hu82
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKSGNQPARKRLNFGQTGDSDSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTPADNNNSEYSWTGATKYHLNGRDSLVNPGPAMASHKDDEEKFFPQSGVLIFGKQGSEKTNVDIEKVMITDEEEIRTTNPVATEQYGTVSTNLQSSNTQAATSDVNTQGVLPGMVWQDRDVYLQGPIWAKIPHTDGHFHPSPLMGGFGLRHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 29 - DNA, 2208 bp, Artificial Sequence
  adeno-associated virus hu84
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaaaggagagccggtcaacgaggcagacgccgcggccctcgagcacgacaaggcctacgaccggcagctcgacagcggagacaacccgtacctcaagtacaaccacgccgacgcggagttccaggagcgtcttaaagaagatacgtcttttgggggcaacctcggacgagcagtcttccaggcgaaaaagagggttcttgaacctctgggcctggttgaggagcctgttaaaacggctccgggaaaaaagaggccggtagagcactctcctgcggagccagactcctcctcgggaaccggaaaagcgggccagcagcctgcaagaaaaagattgaattttggtcagactggagacgcagactccgtacctgacccccagcctctcggacagccaccagcagccccctctggtctgggatctactacaatggctacaggcagtggcgcaccaatggcagacaataacgagggtgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagcctcaaacgacaaccactactttggctacagcaccccttgggggtattttgacttcaacagattccactgccacttttcaccacgtgactggcaaagactcatcaacaacaactggggattccgacccaaaagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcggacgtcttcatggtcccacagtatggatacctcaccctgaacaacgggagtcaggcggtaggacgctcttccttttactgcctggagtactttccttctcagatgctgcgtactggaaacaactttcagttcagctacacttttgaagacgtgcctttccacagcagctacgctcacagccagagtctggatcggctgatgaatcctctgatcgaccagtacctgtattatctgaacaagacacaaacaaatagtggaactcttcagcagtctcgactactgtttagccaagctggacccaccaacatgtctcttcaagctaaaaactggctgcctggaccttgctacagacagcagcgtttgtcaaagcaggcaaacgacaacaacaacagcaactttccctggactgcagctacaaagtatcatctaaatggccgggactcgttggttaatccaggaccagctatggccagtcacaaggatgacgaagaaaagtttttccccatgcatggaaccttgatatttggtaaacaaggaacaaatgccaacgacgcggatttggaaaatgtcatgattacagatgaagaagaaataaggaccaccaatcccgtggctacggagcagtacgggactgtgtcaaataatttgcaaaactcaaacactggtccaactactggaactgtcaatcaccaaggagcgttacctggtatggtgtggcaggatcgagacgtgtacctgcagggacccatttgggccaagattcctcacaccgatggacactttcatccttctccactgatgggaggttttggactcaaacacccacctcctcagatcatgatcaaaaacactcccgttccagcaaatcctcccacaaacttcagttctgccaaatttgcttccttcatcacacagtattccacggggcaggtcagcgtggagatcgagtgggagctgcagaaggagaacagcaagcgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 30 - protein, 735 aa, Artificial Sequence
  adeno-associated virus hu84
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQTNSGTLQQSRLLFSQAGPTNMSLQAKNWLPGPCYRQQRLSKQANDNNNSNFPWTAATKYHLNGRDSLVNPGPAMASHKDDEEKFFPMHGTLIFGKQGTNANDADLENVMITDEEEIRTTNPVATEQYGTVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 31 - DNA, 2208 bp, Artificial Sequence
  adeno-associated virus hu86
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaaaggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgcggagccagattcctcctccggaactggaaagtcgggcaaccagcctgcaagaaagagattgaatttcggtcagactggagacgcagactccgtacctgacccccagcctctcggacagccaccagcagccccctctagtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgcacctgggctctgcccacctacaacaaccatctgtacaagcagatttccagccaatcaggagcctcaaacgacaaccactactttggctacagcaccccctgggggtattttgacttcaacagattccactgccacttttcaccacgtgactggcaaagactcatcaacaacaattggggattccggcccaaaagactcaacttcaagctctttaacattcaagtcaaggaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcggtaggacgctcttccttttactgcctggagtactttccttctcaaatgcttcgtaccggaaacaactttaccttcagctacacctttgaggacgtgcctttccacagcagctacgctcacagccagagtttggaccgtctcatgaatcctctcatcgaccagtacctgtattacttgagcaaaacaaacacgccgagcggaaccaccacgcagtccaggcttcagttttctcaggccggagcgagtgacattcgggaccagtctaggaactggcttcctggaccctgttaccgccagcagcgagtatcaaaaacagctgcggacaacaacaatagtgattactcgtggactggagctaccaagtaccacctcaatggaagagactctctggtgaatccgggaccggccatggccagccacaaggatgatgaagaaaagtattttcctcagagcggggttctcatctttggaaagcaaggctcagagaaaacaaatgtggacattgaaaaggtcatgattacagacgaagaggaaatcaggaccaccaatcccgtggctacggagcagtatggtgctgtatctaccaacctccagagcggccacacacaagcaactaccgcagatgtcaacacacaaggcgttcttccaggcatggtctggcaggacagagacgtgtacctgcaggggcctatctgggcaaagattccccacacggatggacactttcacccctctcccctcatgggcggattcggacttaaacaccctcctccacaaattctcatcaagaacaccccggtacctgcgaatccttcgaccaccttcagtgcggcaaagtttgcttccttcatcacacagtattccacgggacaggttagcgtggagattgagtgggagctgcagaaggagaacagcaaacgctggaatcccgagatccagtacacttccaactataacaagtctgttaatgtggactttactgtggacactaatggcgtgtattcagagcctcgccccattggcaccagatacctgactcgtaatctgtaa

SEQ ID NO: 32 - protein, 735 aa, Artificial Sequence
  adeno-associated virus hu86
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKSGNQPARKRLNFGQTGDADSVPDPQPLGQPPAAPSSLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSKTNTPSGTTTQSRLQFSQAGASDIRDQSRNWLPGPCYRQQRVSKTAADNNNSDYSWTGATKYHLNGRDSLVNPGPAMASHKDDEEKYFPQSGVLIFGKQGSEKTNVDIEKVMITDEEEIRTTNPVATEQYGAVSTNLQSGHTQATTADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 33 - DNA, 2205 bp, Artificial Sequence
  adeno-associated virus hu87
  misc_feature (1)..(2202): vp1
  misc_feature (412)..(2202): vp2
  misc_feature (607)..(2202): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgccttaaagaagatacgtcttttgggggcaaccttggacgagcagtcttccaggcgaaaaagagggttcttgaacctctgggcctggttgaggaacctgttaaaacggctccgggaaaaaagaggccggtagagcactctcctgtggagccagactcctcctcgggaaccggaaaagcgggccagcagcctgcaagaaagagattgaattttggtcagactggagacgcagactccgtacctgacccccagcctctcggacagccaccagcagccccctctggtctgggatctactacaatggctacaggcagtggcgcaccaatggcagacaataacgagggtgccgatggagtgggtaattcctcaggaaattggcattgcgattcccaatggctgggcgacagagtcatcaccaccagcacccgcacctgggccctgcccacctacaacaaccacctctacaagcaaatctccagccaatcaggagcctcaaacgacaaccactactttggctacagcaccccctgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgtttccagcggacgtcttcatggtcccacagtatggatacctcaccctgaacaacgggagtcaggcggtaggacgctcttccttttactgcctggagtactttccttctcagatgctgcgtactggaaacaactttcagttcagctacacttttgaagacgtgcctttccacagcagctacgctcacagccagagtctggatcggctgatgaatcctctgatcgaccagtacctgtactatctgaacaggacacaaacagccagtggaactcagcagtctcggctactgtttagtcaagctggacccaccagtatgtctcttcaagctaaaaactggctgcctggaccttgctacagacagcagcgtctgtcaaagcaggcaaacgacaacaacaacagcaactttccctggactgcggctacaaagtaccacctcaatggtagagactcgttggtgaacccgggccctgctatggccagccacaaagacgatgaagaaaagtttttccccatgcatggaaccctgatatttggtaaagaaggaacaaatgctgccaacgcggatttggacaatgtcatgattacagatgaagaagaaatccgcactaccaatcctgtagctacggagcagtatggatatgtgtcaaataatttgcaaaactcaaatactgctgcaactactgaaactgtcaatcaccaaggagcgttacctggtatggtgtggcaggatcgagacgtgtacttgcagggacccatttgggccaaaattcctcacaccgatggacactttcatccttctccacttatgggaggttttggactcaaacacccacctcctcagatcatgatcaaaaacactcccgttccagccaatcctcccacaaacttcagttctgccaagtttgcttccttcatcacacagtattccacggggcaggtcagcgtggagatcgagtgggaattgcagaaggagaacagcaaacgctggaatcccgaaattcagtacacttccaactacaacaaatctgttaatgtggactttactgtggacactaatggtgtgtattcagagcctcgccccattggcaccagatacctgactcgtaatctgtaa

SEQ ID NO: 34 - protein, 734 aa, Artificial Sequence
  adeno-associated virus hu87
  MISC_FEATURE (1)..(734): vp1
  MISC_FEATURE (138)..(734): vp2
  MISC_FEATURE (203)..(734): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQTASGTQQSRLLFSQAGPTSMSLQAKNWLPGPCYRQQRLSKQANDNNNSNFPWTAATKYHLNGRDSLVNPGPAMASHKDDEEKFFPMHGTLIFGKEGTNAANADLDNVMITDEEEIRTTNPVATEQYGYVSNNLQNSNTAATTETVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 35 - DNA, 2208 bp, Artificial Sequence
  adeno-associated virus hu88/78
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaagacggctccgggaaaaaagaggccggtagagcactctcctgcggagccagattcctcctcgggaaccggaaaggcgggccagcagcctgctagaaaaagactgaatttcggtcagactggagacgcagactccgtaccagaccctcaacctctcggagaaccaccagcagcccccacaagtttgggatctactacaatggcttcaggcggtggcgcaccagtggcagacaataacgagggtgccgatggagtgggtaattcctcgggaaattggcattgcgattcccaatggctgggcgacagagtcattaccaccagcacccgaacctgggccctgcccacttacaacaaccatctgtacaaacaaatatccagccaatcaggagccagcaacgacaaccactactttggctacagcaccccctgggggtattttgacttcaacagattccactgccacttttcaccacgtgactggcaaagacttatcaacaacaactggggattccggcccaagagactcaacttcaagctctttaacattcaagtcaaagaggtcacgcagaatgacggtacgacgacgattgccaataaccttaccagcacggttcaggtgtttactgactcggaataccagctcccgtacgtcctcggctcggcgcatcaaggatgcctcccgccgttcccagcagacgtcttcatggtcccacagtatggatacctcaccctgaacaacgggagtcaggcggtaggacgctcttccttttactgcctggagtactttccttctcagatgctgcgtactggaaataactttcagttcagctacacctttgaagacgtgcctttccacagcagctacgctcacagccagagtctggatcggctgatgaatcctctgatcgaccagtacctgtattatctgaataagacacaaaactccagtggaactgttcaacagtctcggctactgtttagtcaagctggacccaccagtatgtctcttcaagctaaaaactggctgcctggaccttgctacagacagcagcgtctgtcaaagcaggcaagcgacaacaacaacagcaactttccctggactgcggccacaaagtatcatctaaatggccgggactcattggttaatccaggaccagctatggccagtcataaggatgacgaagaaaagtttttccccatgcatggaaccctaatatttggtaaagaaggaacaactgctaacaacgcggatttggaacatgtcatgattacagatgaagaagaaatcaggaccaccaatccagtggctacggagcagtacggaaatgtgtcaaataatttgcaaaactcaaatactggtccaactaccgaaaatgtcaataaccaaggagcgttacctggtatggtgtggcaggatcgagacgtgtacctgcagggacccatttgggccaaaattccacacacggacggacactttcatccttctccactgatgggaggttttggactcaaacacccacctcctcagatcatgatcaaaaacactcccgttccagccaatcctcctacaaacttcagtgcggcaaagtttgcttctttcattacacagtactccacggggcaggtcagcgtggagatcgagtgggagctgcagaaggagaacagcaaacgctggaatcccgagattcagtacacttccaactacaacaaatctgttaatgtggactttactgtggacactaatggtgtatattcagagccccgccccattggcaccagatacctgactcgtaatctgtaa

SEQ ID NO: 36 - protein, 735 aa, Artificial Sequence; identical to SEQ ID NO: 16
  adeno-associated virus hu88/78
  MISC_FEATURE (1)..(735): vp1
  MISC_FEATURE (138)..(735): vp2
  MISC_FEATURE (203)..(735): vp3

SEQ ID NO: 37 - DNA, 2208 bp, Artificial Sequence
  adeno-associated virus hu69
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaggcctacgaccggcagctcgaaagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggagcctgttaaaacggctccgggaaaaaagaggccggtagagcactctcctgtggagccagactcctcctcgggaaccggaaaagcgggccagcagcctgcaagaaaaagattgaattttggtcagactggagacgcagactccgtacctgacccccagcctctcggacagccaccagcagccccctctggtctgggatctactacaatggcttcaggcagtggcgcaccagtggcagacaataacgagggtgccgatggagtgggtaattcctcaggaaattggcattgcgattcccaatggctgggcgacagagtcatcaccaccagcacccgaacctgggccctgcccacctacaacaaccacctctacaagcaaatctccagccaatcaggagcctcaaacgacaaccactactttggctacagcaccccctgggggtattttgacttcaacagattccactgccacttttcaccacgtgactggcaaagactcatcaacaacaactggggattccgacccaaaagactcaacttcaagctctttaacattcaagtcaaagaggtcacgcagaatgacggtacgacgacgattgccaataaccttaccagcacggttcaagtgtttactgactcggagtaccagctcccgtacgtcctcggctcggcgcatcaaggatgcctcccgccgttcccagcagacgtctttatggtcccacagtatggatacctcaccctgaacaacgggagtcaggcggtaggacgctcttccttttactgcctggagtactttccttctcagatgctgcgtactggaaacaactttcagttcagctacacctttgaagacgttcccttccacagcagctacgctcacagccagagtctggatcggctgatgaatcctctgatcgaccagtacctatattatctgaacaagacacaatcaaatagtggaactcttcagcagtctcggctactgtttagccaagctggacccaccagcatgtctcttcaagctaaaaactggctgcctggaccttgctacagacagcagcgtctgtcaaagcaggcaaacgacaacaacaacagcaactttccctggactgcggctacaaagtatcatctaaatggccgggactcgttggttaatccaggaccagctatggccagccacaaagacgatgaagaaaagtttttccccatgcatggaaccctgatatttggtaaacaaggaacaaatgctaacgacgcggatttggacaatgtcatgattacagatgaagaagaaatccgcaccaccaatcccgtggctacggagcagtacggatatgtgtcaaataatttgcaaaactcaaatactggtccaactactggaactgtcaatcaccaaggagcgttacctggtatggtgtggcaggatcgagacgtgtacctgcagggacccatttgggccaagattcctcacacggacggacactttcatccttctccactaatgggaggttttgggctcaaacacccgcctcctcagatcatgatcaaaaacactcccgttccagccaatcctcctacaaacttcagttcttccaagtttgcttctttcatcacacagtattccacggggcaggtcagcgtggagatcgagtgggagctgcagaaggagaacagcaaacgctggaatcccgaaattcagtatacttccaactacaacaaatctgttaatgtggactttactgtggacactaatggtgtgtattcagagcctcgccccattggcaccagatacctgactcgtaatctgtaa

SEQ ID NO: 38 - protein, 735 aa, Artificial Sequence
  adeno-associated virus hu69
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLESGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGSTTMASGSGAPVADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSSKFASFITQYSTGQVSVEIEWELQKENSKRWNPEIQYTSNYNKSVNVDFTVDTNGVYSEPRPIGTRYLTRNL

SEQ ID NO: 39 - DNA, 2211 bp, Artificial Sequence
  adeno-associated virus rh75
  misc_feature (1)..(2208): vp1
  misc_feature (412)..(2208): vp2
  misc_feature (607)..(2208): vp3
atggctgctgacggttatcttccagattggctcgaggacaacctctctgagggcattcgcgagtggtggaacctgaaacctggagcccccaagcccaaggccaaccagcaaaagcaggacgacggccggggtctggtgcttcctggctacaagtacctcggaccctttaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaaaacggctcctggaaagaagagaccagtagagcagtcaccccaagaaccagactcctcgtcgggcatcggcaagacaggccagcagcccgctaaaaagagactcaattttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcaccagaacctgggccctgcccacctacaacaaccacctgtacaagcaaatctcaaacggcacatcgggaggaagcaccaacgacaacacctactttggctacagcaccccctgggggtattttgacttcaacagattccactgccacttttcaccacgtgactggcagcgactcatcaacaacaactggggattccggccaaagcgactcaacttcaagctgttcaatatccaggtcaaggaagttacgacgaacgaaggcaccaagaccatcgccaataatctcaccagcaccgtgcaggtctttacggactcggagtaccagctaccgtacgtgttaggctctgcccatcaaggatgcctgcctccctttcctgcggacgtgttcatggttcctcagtacggctacctgactctcaacaatggcagtcaagccttgggacgttcttctttctactgcctggagtatttcccttctcaaatgctgagaacgggcaacaactttcagttcagctacacttttgaggatgtgcctttccacagcagctacgcgcacagccagagcttggacagactgatgaatcccctgattgaccagtatttgtattacctggtcagaacacagacaaccggaacaggggggacccagacgctggcattcagccaagcaggtcccagctcaatggccaaccaggctagaaactgggtacctgggccttgctatcggcagcagcgtgtgtccacaactacaaaccaaaacaacaacagcaattttgcctggactggagcagctaagtttaagctgaatggccgagactccctgatgaatcctggcgtggctatggcttctcacaaggacgacgatgaccgctttttcccatcgagtggcgttctgatatttggcaagcaaggagccgggaacgatggagttgactacagccaggtgctaatcacagatgaagaagaaatcaaggccacaaaccctgtggctacagaagaatatggagcagtagccatcaataaccaggcagctaacacgcaggcgcagactggactggtgcacaaccagggagttattcctggtatggtctggcagaatcgggacgtgtacctgcagggtcctatttgggccaaaattcctcatacggatggcaactttcatccgtctccactgatgggaggctttggactcaagcatccgcctcctcagatcctcatcaaaaacactccggtaccggcagaccctcctcttaccttcaaccaggccaagttgaattctttcatcacgcagtacagcaccggacaagtcagcgtggagatcgagtgggagctacagaaggagaacagcaaacgctggaacccagagattcagtacacctccaactactacaaatctacaaatgtggactttgctgtcaataccgagggtgtttattctgagcctcgcccaattggaactcgttacctcacccgtaatctgtaa

SEQ ID NO: 40 - protein, 736 aa, Artificial Sequence
  adeno-associated virus rh75
  MISC_FEATURE (1)..(736): vp1
  MISC_FEATURE (138)..(736): vp2
  MISC_FEATURE (203)..(736): vp3
MAADGYLPDWLEDNLSEGIREWWNLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 41 - DNA, 2214 bp, Artificial Sequence
  adeno-associated virus rh76
  misc_feature (1)..(2211): vp1
  misc_feature (412)..(2211): vp2
  misc_feature (610)..(2211): vp3
atggctgctgacggttatcttccagattggctcgaggacaacctctctgagggcattcgcgagtggtggaacctgaaacctggagcccccaagcccaaggccaaccagcaaaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaaaaagagaccggtagaaccgtcacctcagcgctccccagactcctccacgggcatcggcaagaaaggccagcagcccgcgagaaagagactgaactttgggcagactggcgactcagagtcagtccccgaccctcaaccaatcggagaaccaccagcaggcccctctggtctgggatctggtacaatggctgcaggcggtggcgcaccaatggctgacaataacgagggcgccgacggagtgggtaatgcctcaggaaattggcattgcgattccacatggctgggcgacagagtcatcaccaccagcacccgaacctgggccctgcccacctacaacaaccacctctacaagcaaatctccagtcagtcagcaggtagcaccaacgacaacgtctacttcggctacagcaccccctgggggtattttgacttcaacagattccattgccacttctcaccacgtgactggcagcgactcatcaacaacaattggggattccggcccaagaagctcaacttcaagctcttcaacatccaagtcaaagaagtcacgacgaatgatggcgtcacaaccatcgctaataaccttaccagcacggttcaggtcttttcggactcggaataccagctgccgtacgtcctcgggtccgcgcaccagggctgcctgcctccgttcccggcggatgtcttcatgattcctcagtacggctacctgacactgaacaatggcagccaatcggtgggccgttcctccttctactgcctggaatattttccatctcagatgctaaggactggaaacaacttcaccttcagctacaccttcgaggacgtgccattccacagcagctacgctcacagccagagcctggaccggctgatgaatcctctcattgaccagtacctgtactacctggccagaacacagagcaacgcaggaggcacagctggcaatcgggaactgcagttttaccagggcggacctaccaccatggccgaacaagccaagaactggctgcctggaccttgcttccggcaacaaagagtctccaagacgctggatcaaaacaataacagcaactttgcttggactggtgccaccaaatatcatctaaatggaagaaattcattggttaaccccggtgtcgccatggcaacccacaaggacgatgaggagcgcttcttcccttcgagtggagtcctgatttttggcaaaaccggagcagctaacaaaactacattggaaaacgtgctaatgacaaatgaagaagaaattcgtcctaccaacccggtggccacggaggaatatgggactgtcagtagcaatctgcaggcggctaacactgcagcccagacccagactgtcaacaaccagggagccttacctggcatggtctggcagaaccgggacgtgtacctgcagggtcccatctgggccaagattcctcacacggacggcaacttccacccttcaccactgatgggaggctttgggctgaagcatccacctcctcagatcctgatcaagaacactcctgttcctgctaatcctccggaggtgtttacgcctgccaagtttgcttctttcatcacgcagtacagcaccggccaggtcagcgtggaaatcgagtgggagctgcagaaggagaacagcaagcgctggaacccagagattcagtatacctccaattttgacaaacagactggtgtggactttgccgttgacagccagggtgtttattctgagcctcgccccattggtactcgttatctgacacgtaatctgtaa

SEQ ID NO: 42 - protein, 737 aa, Artificial Sequence
  adeno-associated virus rh76
  MISC_FEATURE (1)..(737): vp1
  MISC_FEATURE (138)..(737): vp2
  MISC_FEATURE (204)..(737): vp3
MAADGYLPDWLEDNLSEGIREWWNLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAGPSGLGSGTMAAGGGAPMADNNEGADGVGNASGNWHCDSTWLGDRVITTSTRTWALPTYNNHLYKQISSQSAGSTNDNVYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNAGGTAGNRELQFYQGGPTTMAEQAKNWLPGPCFRQQRVSKTLDQNNNSNFAWTGATKYHLNGRNSLVNPGVAMATHKDDEERFFPSSGVLIFGKTGAANKTTLENVLMTNEEEIRPTNPVATEEYGTVSSNLQAANTAAQTQTVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFDKQTGVDFAVDSQGVYSEPRPIGTRYLTRNL

SEQ ID NO: 43 - DNA, 2202 bp, Artificial Sequence
  adeno-associated virus rh77
  misc_feature (1)..(2199): vp1
  misc_feature (412)..(2199): vp2
  misc_feature (589)..(2199): vp3
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagttttccaggccaagaagagggtactcgaacctctgggcctggttgaagaaggtgctaagacggctcctggaaagaagagaccgttagagtcaccacaagagcccgactcctcctcaggaatcggcaaaaaaggcaaacaaccagccaaaaagagactcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcggctcggaacaacatcaagcagcaacacctacaacggattctccaccccctggggatactttgactttaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagagggaagtctgcctcctttccccaatgacgtcttcatggtgcctcaatatggctactgtggcattgtgactggcgaaaatcagaaccagacggacagaaatgctttctactgcctggagtattttccttcacaaatgctgagaactggcaataactttgaaatggcttacaactttgagaaggtgccgttccactcaatgtatgctcacagccagagcctggacagactgatgaatcccctcctggaccagtacctgtggcacttacagtcgaccacctctggagagactctgaatcaaggcaatgcagcaaccacatttggaaaaatcaggagtggagactttgccttttacagaaagaactggctgcctgggccttgtgttaaacagcagagattctcaaagactgccagtcaaaattacaagattcctgccagcgggggcaacgctctgttaaagtatgacacccactataccttaaacaaccgctggagcaacatcgcgcccggacctccaatggccacagccggaccttcggatggggacttcagtaacgcccagctcatcttccctggaccatcagtcaccggaaacacaacaacttcagccaacaatctgttgtttacatcagaagaagaaattgctgccaccaacccaagagacacggacatgtttggccagattgctgacaataatcagaatgctacaactgctcccataaccggcaacgtgactgctatgggagtgctgcctggcatggtgtggcaaaacagagacatttactaccaagggccaatttgggccaagatcccacacgcggacggacattttcatccttcaccgctgattggtgggtttggactgaaacacccgcctccccagatattcatcaagaacactcccgtacctgccaatcctgcgacaaccttcactgcagccagagtggactctttcattacacaatacagcaccggccaggtcgctgttcagattgaatgggaaattgaaaaggaacgctccaaacgctggaatcctgaagtgcagtttacttcaaactatgggaaccagtcttctatgttgtgggctcctgatacaactgggaagtatacagagccgcgggttattggctctcgttatttgactaatcatttgtaa

SEQ ID NO: 44 - protein, 733 aa, Artificial Sequence
  adeno-associated virus rh77
  MISC_FEATURE (1)..(733): vp1
  MISC_FEATURE (138)..(733): vp2
  MISC_FEATURE (197)..(733): vp3
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 45 - DNA, 2202 bp, Artificial Sequence
  adeno-associated virus rh78
  misc_feature (1)..(2199): vp1
  misc_feature (412)..(2199): vp2
  misc_feature (589)..(2199): vp3
atggctgctgacggttatcttccagattggctcgaggacaacctctctgagggcattcgcgagtggtgggacctgaaacctggagcccccaagcccaaggccaaccagcaaaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagagggtactcgaacctctgggcctggttgaagaaggtgctaagacggctcctggaaagaagagaccgttagagtcgccacaagaacccgactcctcctcgggaatcggcaaaaaaggcaaacaaccagccaaaaaaaggctcaactttgaagaggacactggagccggagacggaccacctgaaggatcagataccagcgccatgtcttcagacattgaaatgcgtgcagcaccgggcggaaatgctgtcgatgcgggacaaggttccgatggagtgggtaatgcctcaggtgattggcattgcgattccacctggtctgagggcaaggtcacaacaacctcgaccagaacctgggtcttgcccacctacaacaaccacttgtacctgcggctcggaacaacatcaagcagcaacacctacaacggattctccaccccctggggatactttgactttaacagattccactgtcacttctcaccacgtgactggcaaaggctcatcaacaacaactggggactacggccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctgggcaagaggggagtctgcctccgttccccaatgacgtctttatggtgcctcaatatggctactgtggcatcgttactggtgaaaatcagaaccagacggacagaaatgccttctattgcctggagtattttccttcacaaatgctgagaactggtaacaattttgaaatggcttacaactttgagaaggtgccgttccactccatgtatgctcacagccagagtctggacagactgatgaatcccctcctggaccagtacctgtggcacttgcagtcaaccacctctggagagactctgaatcaaggcaacgcagcaaccacatttggaaagatcagaagtggagactttgccttttacagaaagaactggctgcctgggccttgtgtcaaacagcagagattttcaaaaactgctagccaaaattacaagattcctgccagtgggggcaacgctctattaaagtatgatacccactataccttgaacaaccgatggagcaacattgcgcccggacctccaatggccacggctgcaccttcagatggggacttcagcaacgcgcagctcatctttcctggaccatctgtcaccggaaacacaacaacttcagccaacaacctgttgtttacatcagaagaagaaattgctgccaccaaccctagagacacagacatgtttggtcagattgctgacaataatcaaaatgctacaacggctcccataaccggcaacgtgacagctatgggagtgctgcctggtatggtctggcaaaacagagacatttactaccaggggcctatttgggccaagatcccacacacggacggacactttcatccatcgccgctgattggtgggtttggactcaaacacccacctccccagatcttcattaagaacacccccgtacctgccaatcctgcgacaaccttcactgcagccagagtggactctttcatcacacaatacagcactggccaagtcgctgttcaaatcgagtgggagattgaaaaggaacgttccaaacgctggaatcctgaagtgcagtttacctcaaactatgggaatcaatcttctatgttgtgggctcctgatacaaatgggaagtatacagagccgcgggttattggctctcgttatttgactaatcacttgtaa

SEQ ID NO: 46 - protein, 733 aa, Artificial Sequence
  adeno-associated virus rh78
  MISC_FEATURE (1)..(733): vp1
  MISC_FEATURE (138)..(733): vp2
  MISC_FEATURE (197)..(733): vp3
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAAPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHTDGHFHPSPLIGGFGLKHPPPQIFIKNTPVPANPATTFTAARVDSFITQYSTGQVAVQIEWEIEKERSKRWNPEVQFTSNYGNQSSMLWAPDTNGKYTEPRVIGSRYLTNHL

SEQ ID NO: 47 - DNA, 2217 bp, Artificial Sequence
  adeno-associated virus rh79
  misc_feature (1)..(2214): vp1
  misc_feature (412)..(2214): vp2
  misc_feature (610)..(2214): vp3
atggctgctgacggttatcttccagattggctcgaggacaacctctctgagggcattcgcgagtggtgggacctgaaacctggagcccccaagcccaaggccaaccagcagaagcaggacgacggccggggtctggtgcttcctggctacaagtacctcggacccttcaacggactcgacaagggggagcccgtcaacgaggcggacgccgcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaagctgctaagacggctcctggaaagaagagaccggtagaaccgtcacctcagcgatcccccgactcctccacgggcatcggcaaaaaaggccagcagcccgcgagaaagagactgaactttgggcagactggcgactcagagtcagtccccgaccctcaaccaatcggagaaccaccagcaggcccctctggtctgggatctggtacaatggctgcaggcggtggcgctccaatggcagacaataacgaaggcgccgacggagtgggtagttcctcaggaaattggcattgcgattccacatggctgggcgacagagtcatcaccaccagcacccgaacctgggccctgcccacctacaacaaccacctctacaagcaaatctccaatgggacatcgggaggaagcaccaacgacaacacctacttcggctacagcaccccctgggggtattttgacttcaacagattccactgtcacttctcaccacgtgactggcagagactcatcaacaacaactggggattccggcccaagagactcagcttcaagctcttcaacatccaggttaaggaggtcacgcagaatgaaggcaccaagaccatcgccaataaccttaccagcacgattcaggtatttacggactcggaataccagctgccgtacgtcctcggctccgcgcaccagggctgcctgcctccgttcccggcggatgtcttcatgattccccagtacggctacctgacactgaacaacggaagtcaagccgtaggccgttcctcattctactgcctggaatattttccatctcaaatgctgcggactggaaacaactttgaatttagctacacctttgaggacgtgcccttccacagcagctacgcacacagccagagcctggaccggctgatgaaccctctcatcgaccagtacctgtattacctatccagaactcagtccacaggaggaactcaaggtacacagcaattgttattttctcaagccgggcctgcaaatatgtcggctcaggccaagaactggctacctggaccttgctaccggcagcagcgagtctccacgacactgtcgcaaaacaacaacagcaactttgcttggactggtgccacgaaatatcatctgaacggaagagactctttggtgaatcccggtgttgctatggcaacgcataaggacgacgaggaacgtttctttccatcgagcggagtcctgatgtttggaaaacagggtgctggaagagacaatgtggactatagcagcgttatgctaaccagcgaggaagaaatcaagaccactaaccctgtagccactgaacaatacggcgtggtggctgataacttgcagcaaaccaatacaggacctatcgtgggaaatgtcaacagccaaggagccttacctggcatggtctggcagaaccgagacgtgtacctgcagggtcccatttgggccaagattcctcacacggacggcaactttcacccgtctcctctgatgggcggctttggactgaaacacccgcctcctcaaatcctgatcaagaacactcccgttcctgcggatcctccaacgacgttcagccaggcgaaattggcttccttcatcacgcagtatagtaccggccaggtcagcgtggagatcgagtgggagctgcagaaggagaacagcaagcgctggaacccagaaattcagtatacttccaactactacaaatctacaaatgtggactttgctgtcaataccgagggtacatattcagagcctcgccccattggaactcgttacctcacccgtaatctgtaa

SEQ ID NO: 48 - protein, 738 aa, Artificial Sequence
  adeno-associated virus rh79
  MISC_FEATURE (1)..(738): vp1
  MISC_FEATURE (138)..(738): vp2
  MISC_FEATURE (204)..(738): vp3
MAADGYLPDWLEDNLSEGIREWWDLKPGAPKPKANQQKQDDGRGLVLPGYKYLGPFNGLDKGEPVNEADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPARKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYTFEDVPFHSSYAHSQSLDRLMNPLIDQYLYYLSRTQSTGGTQGTQQLLFSQAGPANMSAQAKNWLPGPCYRQQRVSTTLSQNNNSNFAWTGATKYHLNGRDSLVNPGVAMATHKDDEERFFPSSGVLMFGKQGAGRDNVDYSSVMLTSEEEIKTTNPVATEQYGVVADNLQQTNTGPIVGNVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTEGTYSEPRPIGTRYLTRNL

SEQ ID NO: 49 - DNA, 2220 bp, Artificial Sequence
  adeno-associated virus rh81
  misc_feature (1)..(2217): vp1
  misc_feature (412)..(2217): vp2
  misc_feature (619)..(2217): vp3
atggctgctgatggttatcttccagattggctcgaggacaacctctctgagggcattcgcgagtggctgaagctcaaagctggagcaccgccgcccaagcccaaccagcagaaacaggacaatagccggggtcttgtgcttcctggatacaagtacctcggacccttcaacggactcgacaagggacagccggtcaacgaggcagacgccgcggccctcgagcacgacaaggcctacgacaagcagctcgagcagggggacaacccgtacctcaagtacaaccacgcggacgccgagtttcaggagcgcctggaaaaagatacgtcttttgggggcaaccttgggaaggcagtcttccaggccaaaaagcgcattctcgaaccttttggcttggttgaggctcctgttaagacggctccgggaaagaagaggccgctagaaaagactcccaaccagcccacggactctgaggccgcaggtcaggccccagccaaaaaaaagcagcacggagagcagtcgggcgactccgccaagcgccgactcagcttcagcgaggaggccccagagcagccacccgcacccaacccagccaaccccccaagtgtgggacctgttacaatggcttcaggcggtggggcaccagtggcagacaataacgagggcgccgacggagtgggtaattcctcgggaaattggcattgcgattccacatggctgggcgacagagtcatcaccaccagcacccgaacctgggccctgcccacctacaacaaccacctctacaagcaaatctccagccagaacggagccacaaacgacaaccactacttcggctacagcaccccctggggatattttgatttcaacagattccactgccacttctcaccacgtgactggcagcgactcatcaacaacaactggggattccggcccaagcgactcagcttcaagctcttcaacatccaggtcaaggaggtcacgcagacggatggcaccacgaccatcgccaataaccttaccagcacggttcaggtgtttacggactcggagtaccagctcccgtacgtgctcgggtcggcccaccagggctgcctcccgccgttcccggccgacgtcttcatgatcccgcagtacgggtacctgactctgaataacggcagccaggccgtgggacgctcttccttctactgcctggagtactttccatcccagatgctgaggactggaaacaacttttcgttcagctacgtgtttgaggacgtgcccttccacagcagctacgcgcacagccagagcctggaccggctgatgaatcctctcatcgaccagtacctctactacctgagcaaaacacaagggaccaacgccaccgttcagggcgccaagctgcagttttcccaggccgggcccgagaacatgcgcgaccaggccagaaactggatgcccggtcccatgtatcgccagcagcgggtgtctaagaccgccggagacaacaacaacagcgagtatgcctggacgggggccactaaataccacctgaacggtagagactctctggtgaaccccgggcccgccatggccagccacaaggacgacgaggaaaagtttttccccatgaatggcgtcctggtctttggcagacagggcaccggcaaatccaacgtggacattgagaacgtcatgatcaccgacgaggaggagatccggaccaccaaccccgtgtctaccgagcagtacggggtggtctcggacaatctgcagagcagcaactcccggccggtaacaggggacgtggacagtcagggcgtcctacctggcatggtttggcaggaccgcgacgtgtacctgcagggtcccatctgggccaagattcctcacacggacggacactttcacccctctcctctcatgggcggctttggactgaagcatcctcctccccagatcatgattaaaaacacgcccgttccggcgaatcccgcaaccacgttctcggcggataagtttgcctctttcatcacccagtacagcaccgggcaggtcagcgtagagatcgagtgggagctgcagaaggaaaacagcaagcgctggaacccggagatccagtacacctccaactacaacaagtctgtaaatgtggactttaccgtgaacgctgacggtgtttattccgaaccccgccccatcggcactcgttacctcacccgtaatttgtaa

SEQ ID NO: 50 - protein, 739 aa, Artificial Sequence
  adeno-associated virus rh81
  MISC_FEATURE (1)..(739): vp1
  MISC_FEATURE (138)..(739): vp2
  MISC_FEATURE (207)..(739): vp3
MAADGYLPDWLEDNLSEGIREWLKLKAGAPPPKPNQQKQDNSRGLVLPGYKYLGPFNGLDKGQPVNEADAAALEHDKAYDKQLEQGDNPYLKYNHADAEFQERLEKDTSFGGNLGKAVFQAKKRILEPFGLVEAPVKTAPGKKRPLEKTPNQPTDSEAAGQAPAKKKQHGEQSGDSAKRRLSFSEEAPEQPPAPNPANPPSVGPVTMASGGGAPVADNNEGADGVGNSSGNWHCDSTWLGDRVITTSTRTWALPTYNNHLYKQISSQNGATNDNHYFGYSTPWGYFDFNRFHCHFSPRDWQRLINNNWGFRPKRLSFKLFNIQVKEVTQTDGTTTIANNLTSTVQVFTDSEYQLPYVLGSAHQGCLPPFPADVFMIPQYGYLTLNNGSQAVGRSSFYCLEYFPSQMLRTGNNFSFSYVFEDVPFHSSYAHSQSLDRLMNPLIDQYLYYLSKTQGTNATVQGAKLQFSQAGPENMRDQARNWMPGPMYRQQRVSKTAGDNNNSEYAWTGATKYHLNGRDSLVNPGPAMASHKDDEEKFFPMNGVLVFGRQGTGKSNVDIENVMITDEEEIRTTNPVSTEQYGVVSDNLQSSNSRPVTGDVDSQGVLPGMVWQDRDVYLQGPIWAKIPHTDGHFHPSPLMGGFGLKHPPPQIMIKNTPVPANPATTFSADKFASFITQYSTGQVSVEIEWELQKENSKRWNPEIQYTSNYNKSVNVDFTVNADGVYSEPRPIGTRYLTRNL

SEQ ID NO: 51 - DNA, 2187 bp, Artificial Sequence
  adeno-associated virus rh89
  misc_feature (1)..(2184): vp1
  misc_feature (412)..(2184): vp2
  misc_feature (595)..(2184): vp3
atggctgccgatggttatcttccagattggctcgaggacaatctctctgagggcattcgcgagtggtgggacctgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagacccatagactctccagactcctccacgggcatcggcaagaaaggccagcagcccgctaaaaagaaactcaactttgggcagactggcgactcagagtcagtccccgaccctcaacctctctcagaacctccagcagcgcccactggtgtgggatctggtacagtggctgcaggcggtggcgcaccaatggcagacaataacgaaggtgccgacggagtgggtaatgcctcaggaaattggcattgcgattccacatggttgggcgacagagtcatcaccaccagcacccgaacatgggctttgcccacctacaacaaccacctctacaagcaaatctccagtcagagcggggctaccaacgacaaccacttcttcggctacagcaccccctgggggtattttgacttcaacagattccactgccacttctcaccacgtgactggcagcgactcatcaacaacaactggggattccggcccaagaagctgcggttcaagctcttcaacatccaggtcaaggaggtcacgacgaatgacggcgtcacgaccatcgctaataaccttaccagcacaattcaggtcttctcggactcggagtaccagctgccgtacgtcctcggctctgcgcaccagggctgcctccctccgttcccggcggacgtcttcatgattcctcagtacggctacctaacgctgaacaatggcagtcaagccgtgggccgttcatccttctactgcctggagtatttcccctctcagatgctgagaacgggtaacaactttgaattcagctacacctttgaggacgtgcctttccacagcagctacgcgcacagccagagcctggaccgggtgatgaatcctctgatcgaccagtacctgtactacctggcccggacccagagcaccacgggttctaccagagagctgcagtttcatcaggctgggcccaacactatggccgagcaatcaaagaactggttacctggtccttgcttccggcaacaacgcgtttccaaggtgctggaccagaacgccaacagcaactttgcctggactgctgccactaaatatcacctaaatgggcgtaactctctgaccaatccgggagttcccatggcaacacacaaggacgacgaggacaggttttttcccatcaacggggtgctggtttttggcaagacaggagccgccaacaaaacaacgctggaaaatgtcctgatgacaaacgaagaagagatcaaaactaccaacccggtggctacagaagaatatggcgtggtttccagcaatttgcaggctggaactacaaacccacagacactgactgttaacaaccagggggccttacctggcatggtctggcagaaccgggacgtgtatctccagggtcccatctgggccaagattcctcaaacggacggcaactttcacccgtctcctttgatgggcggctttggactcaaacatccgcctccacagatcctgattaaaaacacacctgttcctgctaatcctccggaggtgtttactcctgccaagtttgcttcgttcatcacgcagtacagcaccggacaagtcagcgtggaaatcgaatgggagctgcagaaagaaaacagcaagcgctggaacccagagattcagtacacttccaattttgataaatctaataatgtggactttgctgttaacaatgaaggtgtttactctgagcctcgccccattggcactcgttacctcacccgtaatctgtaa

SEQ ID NO: 52 - protein, 728 aa, Artificial Sequence
  adeno-associated virus rh89
  MISC_FEATURE (1)..(728): vp1
  MISC_FEATURE (138)..(728): vp2
  MISC_FEATURE (199)..(728): vp3
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPIDSPDSSTGIGKKGQQPAKKKLNFGQTGDSESVPDPQPLSEPPAAPTGVGSGTVAAGGGAPMADNNEGADGVGNASGNWHCDSTWLGDRVITTSTRTWALPTYNNHLYKQISSQSGATNDNHFFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQAVGRSSFYCLEYFPSQMLRTGNNFEFSYTFEDVPFHSSYAHSQSLDRVMNPLIDQYLYYLARTQSTTGSTRELQFHQAGPNTMAEQSKNWLPGPCFRQQRVSKVLDQNANSNFAWTAATKYHLNGRNSLTNPGVPMATHKDDEDRFFPINGVLVFGKTGAANKTTLENVLMTNEEEIKTTNPVATEEYGVVSSNLQAGTTNPQTLTVNNQGALPGMVWQNRDVYLQGPIWAKIPQTDGNFHPSPLMGGFGLKHPPPQILIKNTPVPANPPEVFTPAKFASFITQYSTGQVSVEIEWELQKENSKRWNPEIQYTSNFDKSNNVDFAVNNEGVYSEPRPIGTRYLTRNL

SEQ ID NO: 53 - DNA, 2202 bp, Artificial Sequence
  adeno-associated virus rh82
  misc_feature (1)..(2199): vp1
  misc_feature (412)..(2199): vp2
  misc_feature (589)..(2199): vp3
atggctgctgacggttatcttccagattggctcgaggacaacctctctgagggcattcgcgagtggtgggacctgaaacctggagcccccaagcccaaggctaaccagcagaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagttttccaggccaagaagagggtactcgaacctctgggcctggttgaagaaggtgctaagacggctcctggaaagaagagaccgttagagtcaccacaagagcccgactcctcctcaggaatcggcaaaaaaggcaaacaaccagccaaaaagagactcaactttgaagaggacactggagccggagacggaccccctgaaggatcagataccagcgccatgtcttcagacattgaaatgcgtgcagcaccgggcggaaatgctgtcgatgcgggacaaggttccgatggagtgggtaatgcctcgggtgattggcattgcgattccacctggtctgagggcaaggtcacaacaacctcgaccagaacctgggtcttgcccacctacaacaaccacttgtacctgcggctcggaacaacatcaagcagcaacacctacaacggattctccaccccctggggatactttgactttaacagattccactgtcacttctcaccacgtgactggcaaagactcatcaacaacaactggggactacgaccaaaagccatgcgcgttaaaatcttcaatatccaagttaaggaggtcacaacgtcgaacggcgagactacggtcgctaataaccttaccagcacggttcagatatttgcggactcgtcgtatgagctcccgtacgtgatggacgctggacaagagggaagtctgcctcctttccccaatgacgtcttcatggtgcctcaatatggctactgtggcattgtgactggcgaaaatcagaaccagacggacagaaatgctttctactgcctggagtattttccttcacaaatgctgagaactggcaataactttgaaatggcttacaactttgagaaggtgccgttccactcaatgtatgctcacagccagagcctggacagactgatgaatcccctcctggaccagtacctgtggcacttacagtcgaccacctctggagagactctgaatcaaggcaatgcagcaaccacatttggaaaaatcaggagtggagactttgccttttacagaaagaactggctgcctgggccttgtgttaaacagcagagattctcaaaaactgccagtcaaaattacaagattcctgccagcgggggcaacgctctgttaaagtatgacacccactataccttaaacaaccgctggagcaacatagcgcctggacctccaatggcaacagctggaccttcagatggggacttcagcaacgcccagctcatcttccctggaccatcagtcaccggaaacacaacaacctcagcaaacaatctgttgtttacatcagaagaagaaattgctgccaccaacccaagagacacggacatgtttggtcagattgctgacaataatcagaatgctacaactgctcccataaccggcaacgtgactgctatgggagtgcttcctggcatggtgtggcaaaacagagacatttactaccaagggccaatttgggccaagatcccacacgcggacggacattttcatccttcaccgctgattggcgggtttggactgaaacacccgcctccccagatatttatcaaaaacacccccgtacctgccaatcctgcgacaaccttcactgcagccagagtggactctttcatcacacaatacagcaccggccaggtcgctgttcagattgaatgggaaatcgaaaaggaacgctccaaacgctggaatcctgaagtgcagtttacttcaaactatgggaaccagtcttctatgttgtgggctcccgatacaactgggaagtatacagagccgcgggttattggctctcgttatttgactaatcatttgtaa

SEQ ID NO: 54 - protein, 733 aa, Artificial Sequence
  adeno-associated virus rh82
  MISC_FEATURE (1)..(733): vp1
  MISC_FEATURE (138)..(733): vp2
  MISC_FEATURE (197)..(733): vp3
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 55 - DNA, 2214 bp, Artificial Sequence
  adeno-associated virus rh83
  misc_feature (1)..(2211): vp1
  misc_feature (412)..(2211): vp2
  misc_feature (610)..(2211): vp3
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaaaacggctcctggaaagaagagaccggtagagccgtcaccgcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtgaaactgcaggtagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgacgaatgacggcgtcacgaccatcgctaataaccttaccagcacggttcaggtattctcggactcggagtaccagctgccgtacgtcctcggctctgcgcaccagggctgcctgcctccgttcccggcggacgtcttcatgattcctcagtacggctacctgactctcaacaatggcagccaatcggtgggacgttcatccttctactgcctggaatacttcccttctcagatgctgagaacgggtaacaacttcaccttcagctacacctttgaggacgtgcctttccacagcagctacgcgcacagccagagcctggaccggctgatgaatcccctcatcgaccagtacctgtactacctgtcaagaacccagtctacgggaggcacagcgggaacccagcagttgctgttttctcaggccgggcctagcaacatgtcggctcaggccagaaactggctgcctggaccctgctacagacagcagcgcgtctccacgacactgtcgcaaaacaacaacagcaactttgcctggactggtgccaccaagtatcatctgaacggcagagactctctggtgaatccgggcgtcgccatggcaaccaacaaggacgacgaggaccgcttcttcccatccagcggcatcctcatgtttggcaagcagggagctggaaaagacaacgtggactatagcaacgtgatgctaaccagcgaggaagaaatcaagaccaccaaccccgtggccacagaacagtatggcgtggtggctgataacctacagcagcaaaacaccgctcctattgtgggggccgtcaacagccagggagccttacctggcatggtctggcagaaccgggacgtgtacctgcagggtcctatttgggccaagattcctcacacagatggcaactttcacccgtctcctttaatgggcggctttggacttaaacatccgcctcctcagatcctcatcaaaaacactcctgttcctgcggatcctccaacagcgttcaaccaggccaagctgaattctttcatcacgcagtacagcaccggacaagtcagcgtggagatcgagtgggagctgcagaaggagaacagcaagcgctggaacccagagattcagtatacttccaactactacaaatctacaaatgtggactttgctgttaatactgagggtgtttactctgagcctcgccccattggcactcgttacctcacccgtaatttgtaa

SEQ ID NO: 56 - protein, 737 aa, Artificial Sequence
  adeno-associated virus rh83
  MISC_FEATURE (1)..(737): vp1
  MISC_FEATURE (138)..(737): vp2
  MISC_FEATURE (204)..(737): vp3
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 57 - DNA, 2214 bp, Artificial Sequence
  adeno-associated virus rh84
  misc_feature (1)..(2211): vp1
  misc_feature (412)..(2211): vp2
  misc_feature (610)..(2211): vp3
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaaaacggctcctggaaagaagagaccggtagagccgtcaccgcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtgaaactgcaggtagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgacgaatgacggcgtcacgaccatcgctaataaccttaccagcacggttcaggtattctcggactcggagtaccagctgccgtacgtcctcggctctgcgcaccagggctgcctgcctccgttcccggcggacgtcttcatgattcctcagtacggctacctgactctcaacaatggcagccaatcggtgggacgttcatccttctactgcctggaatacttcccttctcagatgctgagaacgggtaacaacttcaccttcagctacacctttgaggacgtgcctttccacagcagctacgcgcacagccagagcctggaccggctgatgaatcccctcatcgaccagtacctgtactacctgtcaagaacccagtctacgggaggcacagcgggaacccagcagttgctgttttctcaggccgggcctagcaacatgtcggctcaggccagaaactggctgcctggaccctgctacagacagcagcgcgtctccacgacactgtcgcaaaacaacaacagcaactttgcctggactggtgccaccaagtatcatctgaacggcagagactctctggtgaatccgggcgtcgccatggcaaccaacaaggacgacgaggaccgcttcttcccatccagcggcatcctcatgtttggcaagcagggagctggaaaagacaacgtggactatagcaacgtgatgctaaccagcgaggaagaaatcaagaccaccaaccccgtggccacagaacagtatggcgtggtggctgataacctacagcagcaaaacaccgctcctattgtgggggccgtcaacagccagggagccttacctggcatggtctggcagaaccgggacgtgtacctgcagggtcctatttgggccaagattcctcacacagatggcaactttcacccgtctcctttaatgggcggctttggacttaaacatccgcctcctcagattctcattaagaacactcccgttcctgctaatcctccggaggtgtttactcctgccaagtttgcttcgttcatcacgcagtacagcaccggacaagtcagcgtggagatcgagtgggagctgcagaaagaaaacagcaagcgttggaacccagaaattcagtatacctccaactttgaaaaacagactggtgtggactttgccgttgacagccagggtatttactctgagcctcgccccattggcactcgttacctcacccgtaatctgtaa

SEQ ID NO: 58 - protein, 737 aa, Artificial Sequence
  adeno-associated virus rh84
  MISC_FEATURE (1)..(737): vp1
  MISC_FEATURE (138)..(737): vp2
  MISC_FEATURE (204)..(737): vp3
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLSRTQSTGGTAGTQQLLFSQAGPSNMSAQARNWLPGPCYRQQRVSTTLSQNNNSNFAWTGATKYHLNGRDSLVNPGVAMATNKDDEDRFFPSSGILMFGKQGAGKDNVDYSNVMLTSEEEIKTTNPVATEQYGVVADNLQQQNTAPIVGAVNSQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGIYSEPRPIGTRYLTRNL

SEQ ID NO: 59 - DNA, 2214 bp, Artificial Sequence
  adeno-associated virus rh85
  misc_feature (1)..(2211): vp1
  misc_feature (412)..(2211): vp2
  misc_feature (610)..(2211): vp3
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaaaacggctcctggaaagaagagaccggtagagccgtcaccgcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtgaaactgcaggtagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgacgaatgacggcgtcacgaccatcgctaataaccttaccagcacggttcaggtattctcggactcggagtaccagctgccgtacgtcctcggctctgcgcaccagggctgcctgcctccgttcccggcggacgtcttcatgattcctcagtacggctacctgactctcaacaatggcagccaatcggtgggacgttcatccttctactgcctggaatacttcccttctcagatgctgagaacgggtaacaacttcaccttcagctacacctttgaggacgtgcctttccacagcagctacgcgcacagccagagcctggaccggctgatgaatcccctcatcgaccagtacctgtactacctggctagaacacagagtaacccaggaggcacatctggcaatcgggaactgcagttttaccagggcggcccttctactatggccgaacaagccaagaactggttacctggaccttgcttccggcaacagagagtgtccaaaacgctggatcaaaacaacaacagcaactttgcttggactggtgccactaaatatcacctgaacggcagaaactcattggttaatcctggtgttgccatggcaactcacaaggacgacgaggaccgctttttcccatccagcggagtcctgatttttgggaaaactggagcaaccaacaaaactacattggaaaacgtgttaatgacaaatgaagaagaaattcgtcctactaaccctgtagccacggaagaatacggaatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagattctcattaagaacactcccgttcctgctaatcctccggaggtgtttactcctgccaagtttgcttcgttcatcacgcagtacagcaccggacaagtcagcgtggagatcgagtgggagctgcagaaagaaaacagcaagcgttggaacccagaaattcagtatacctccaactttgaaaaacagactggtgtggactttgccgttgacagccagggtatttactctgagcctcgccccattggcactcgttacctcacccgtaatctgtaa

SEQ ID NO: 60 - protein, 737 aa, Artificial Sequence
  adeno-associated virus rh85
  MISC_FEATURE (1)..(737): vp1
  MISC_FEATURE (138)..(737): vp2
  MISC_FEATURE (204)..(737): vp3
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNPGGTSGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGIYSEPRPIGTRYLTRNL

SEQ ID NO: 61 - DNA, 2214 bp, Artificial Sequence
  adeno-associated virus rh87
  misc_feature (1)..(2211): vp1
  misc_feature (412)..(2211): vp2
  misc_feature (610)..(2211): vp3
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctccaagcgggtgacaatccgtacctgcggtataatcacgccgacgccgagtttcaggagcgtctgcaagaagatacgtcatttgggggcaacctcgggcgagcagtcttccaggccaagaagcgggttctcgaacctctcggtctggttgaggaaggcgctaaaacggctcctggaaagaagagaccggtagagccgtcaccgcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacaatggctgcaggcggtggcgcaccaatggcagacaataacgaaggtgccgacggagtgggtaatgcctcaggaaattggcattgcgattccacatggctgggcgacagagtcatcaccaccagcacccgaacctgggccctgcccacctacaacaaccacctctacaagcaaatctccagtgaaactgcaggtagcaccaacgacaacacctacttcggctacagcaccccctgggggtattttgactttaacagattccactgccacttctcaccacgtgactggcagcgactcatcaacaacaactggggattccggcccaagaaactcaacttcaagctcttcaacatccaggtcaaggaggtcacgacgaatgacggcgtcacgaccatcgctaataaccttaccagcacggttcaggtattctcggactcggagtaccagctgccgtacgtcctcggctctgcgcaccagggctgcctgcctccgttcccggcggacgtcttcatgattcctcagtacggctacctgactctcaacaatggcagccaatcggtgggacgttcatccttctactgcctggaatacttcccttctcagatgctgagaacgggtaacaacttcaccttcagctacacctttgaggacgtgcctttccacagcagctacgcgcacagccagagcctggaccggctgatgaatcccctcatcgaccagtacctgtactacctggctagaacacagagtaacccaggaggcacatctggcaatcgggaactgcagttttaccagggcggcccttctactatggccgaacaagccaagaactggttacctggaccttgcttccggcaacagagagtgtccaaaacgctggatcaaaacaacaacagcaactttgcttggactggtgccactaaatatcacctgaacggcagaaactcattggttaatcctggtgttgccatggcaactcacaaggacgacgaggaccgctttttcccatccagcggagtcctgatttttgggaaaactggagcaaccaacaaaactacattggaaaacgtgttaatgacaaatgaagaagaaattcgtcctactaaccctgtagccacggaagaatacggaatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagattctcattaagaacactcctgttcctgcggatcctccaacagcgttcaaccaggccaagctgaattctttcatcacgcagtacagcaccggacaagtcagcgtggagatcgagtgggagctgcagaaggagaacagcaagcgctggaacccagagattcagtatacttccaactactacaaatctacaaatgtggactttgctgttaatactgagggtgtttactctgagcctcgccccattggcactcgttacctcacccgtaatttgtaa

SEQ ID NO: 62 - protein, 737 aa, Artificial Sequence
  adeno-associated virus rh87
  MISC_FEATURE (1)..(737): vp1
  MISC_FEATURE (138)..(737): vp2
  MISC_FEATURE (204)..(737): vp3
MAADGYLPDWLEDNLSEGIREWWDLKPGAPKPKANQQKQDNGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSSVGSGTMAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLNFKLFNIQVKEVTTNDGVTTIANNLTSTVQVFSDSEYQLPYVLGSAHQGCLPPFPADVFMIPQYGYLTLNNGSQSVGRSSFYCLEYFPSQMLRTGNNFTFSYTFEDVPFHSSYAHSQSLDRLMNPLIDQYLYYLARTQSNPGGTSGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPADPPTAFNQAKLNSFITQYSTGQVSVEIEWELQKENSKRWNPEIQYTSNYYKSTNVDFAVNTEGVYSEPRPIGTRYLTRNL

SEQ ID NO: 63 - DNA, 26 bp, Artificial Sequence
  primer sequence
gctgcgtcaactggaccaatgagaac

SEQ ID NO: 64 - DNA, 28 bp, Artificial Sequence
  primer sequence
cgcagagaccaaagttcaactgaaacga

SEQ ID NO: 65 - DNA, 34 bp, Artificial Sequence
  primer sequence
atcgatactagtccatcgacgtcagacgcggaag

SEQ ID NO: 66 - DNA, 40 bp, Artificial Sequence
  primer sequence
atcgatgcggccgcagttcaactgaaacgaattaaacggt

SEQ ID NO: 67 - DNA, 2211 bp, Artificial Sequence
  adeno-associated virus 9
  misc_feature (1)..(2208): vp1
  misc_feature (412)..(2208): vp2
  misc_feature (604)..(2208): vp3
atggctgccgatggttatcttccagattggctcgaggacaaccttagtgaaggaattcgcgagtggtgggctttgaaacctggagcccctcaacccaaggcaaatcaacaacatcaagacaacgctcgaggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 68 - protein, 736 aa, Artificial Sequence
  adeno-associated virus 9
  MISC_FEATURE (1)..(736): vp1
  MISC_FEATURE (138)..(736): vp2
  MISC_FEATURE (202)..(736): vp3
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 69 - DNA, 2211 bp, Artificial Sequence
  adeno-associated virus hu32
  misc_feature (1)..(2208): vp1
  misc_feature (412)..(2208): vp2
  misc_feature (604)..(2208): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatgggagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagcgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 70 - protein, 736 aa, Artificial Sequence
  adeno-associated virus hu32
  MISC_FEATURE (1)..(736): vp1
  MISC_FEATURE (138)..(736): vp2
  MISC_FEATURE (202)..(736): vp3
MAADGYLPDWLEDTLSEGIRQWWKLKPGPPPPKPAERHKDDSRGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGSQPAKKKLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 71 - DNA, 2279 bp, Artificial Sequence
  adeno-associated virus rh8
atggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaagcctacgaccagcagctcaaagcgggtgacaatccgtacctgcggtataatcacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcgccacaagagccagactcctcctcgggcatcggcaagacaggccagcagcccgctaaaaagagactcaattttggtcagactggcgactcagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctattttggctacagcaccccctgggggtattttgacttcaacagattccactgtcacttttcaccacgtgactggcaacgactcatcaacaacaattggggattccggcccaaaagactcaacttcaagctgttcaacatccaggtcaaggaagtcacgacgaacgaaggcaccaagaccatcgccaataatctcaccagcaccgtgcaggtctttacggactcggagtaccagttaccgtacgtgctaggatccgctcaccagggatgtctgcctccgttcccggcggacgtcttcatggttcctcagtacggctatttaactttaaacaatggaagccaagccctgggacgttcctccttctactgtctggagtatttcccatcgcagatgctgagaaccggcaacaactttcagttcagctacaccttcgaggacgtgcctttccacagcagctacgcgcacagccagagcctggacaggctgatgaatcccctcatcgaccagtacctgtactacctggtcagaacgcaaacgactggaactggagggacgcagactctggcattcagccaagcgggtcctagctcaatggccaaccaggctagaaattgggtgcccggaccttgctaccggcagcagcgcgtctccacgacaaccaaccagaacaacaacagcaactttgcctggacgggagctgccaagtttaagctgaacggccgagactctctaatgaatccgggcgtggcaatggcttcccacaaggatgacgacgaccgcttcttcccttcgagcggggtcctgatttttggcaagcaaggagccgggaacgatggagtggattacagccaagtgctgattacagatgaggaagaaatcaaggctaccaaccccgtggccacagaagaatatggagcagtggccatcaacaaccaggccgccaatacgcaggcgcagaccggactcgtgcacaaccagggggtgattcccggcatggtgtggcagaatagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcaatacacttccaactactacaaatctacaaatgtggactttgctgtcaacacggagggggtttatagcgagcctcgccccattggcacccgttacctcacccgcaacctgtaattacatgttaatcaataaaccggttaattcgtttcagttgaactttggtctctgcgaagggcgaattc

SEQ ID NO: 72 - protein, 736 aa, Artificial Sequence
  adeno-associated virus rh8
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDDDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 73 - DNA, 2208 bp, Artificial Sequence
  adeno-associated virus hu73
  misc_feature (1)..(2205): vp1
  misc_feature (412)..(2205): vp2
  misc_feature (607)..(2205): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggggagccggtcaacgaggcagacgccgcggccctcgaacacgacaaggcctacgaccggcagctcgacagcggagacaacccgtacctcaagtacaaccacgccgacgcggagtttcaggagcgtcttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgcggagccagactcctcctcgggaaccggaaaagcgggccagcagcctgcaagaaaaagattgaattttggtcagactggagacgcagactccgtacctgacccccagcctctcggacagccaccagcagccccctctggtctgggatctactacaatggctacaggcagtggcgcaccaatggcagacaataacgagggtgccgatggagtgggtaattcctcaggaaattggcattgcgattcccaatggttgggcgacagagtcatcaccaccagcacccgaacctgggccctgcccacctacaacaaccacctctacaagcaaatctccagccaatcaggagccagcaacgacaaccactactttggctacagcaccccctgggggtattttgacttcaacagattccactgccacttttccccacgtgactggcaaagactcatcaacaacaactggggattccggcccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtcccacagtatggatacctcaccctgaacaacgggagtcaggcggtaggacgctcttcattttactgcctggagtactttccttctcagatgctgcgtaccggaaacaactttaccttcagctacacctttgaggacgttcctttccacagcagctacgctcacagtcaaagtctggaccggctgatgaatcctcttatcgaccagtacctgtattatctaaacaagacacaatcaaatagtggaactcttcagcagtctcggctactgtttagtcaagctggacccaccagcatgtctcttcaagctaaaaactggctgcctggaccttgctacagacaacagcgtctgtcaaagcaggcaaacgacaacaacaacagcaactttccctggactgcggctacaaagtaccatctaaatggccgggactcgttggttaatccaggaccagctatggccagccacaaagacgatgaagaaaagtttttccccatgcatggaaccctgatatttggtaaacaaggaacaaatgctaacgacgcggatttggacaatgtcatgattacagatgaagaagaaatccgcaccaccaatcccgtggctacggagcagtacggatatgtgtcaaataatttgcaaaactcaaatactggtccaactactggaactgtcaatcaccaaggagcgttacctggtatggtgtggcaggatcgagacgtgtacctgcagggacccatttgggccaagattcctcacaccgacggacactttcatccttctccactgatgggaggttttggactcaaacacccgcctcctcaaatcatgatcaaaaacactcccgttccagccaatcctcctacaaacttcagttctgccaagtttgcttctttcatcacacagtattccacgggacaggtcagcgtggagattgagtgggagctgcagaaggagaacagcaaacgctggaaccccgagatccagtacacttccaactacaacaaatctgttaatgtggactttactgtggacactaatggtgtgtattcagagcctcgccccattggcaccagatacctgactcgtaatttgtaa

SEQ ID NO: 74 - protein, 735 aa, Artificial Sequence
  adeno-associated virus hu73
  MISC_FEATURE (1)..(735): vp1
  MISC_FEATURE (138)..(735): vp2
  MISC_FEATURE (203)..(735): vp3
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPAEPDSSSGTGKAGQQPARKRLNFGQTGDADSVPDPQPLGQPPAAPSGLGSTTMATGSGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLNKTQSNSGTLQQSRLLFSQAGPTSMSLQAKNWLPGPCYRQQRLSKQANDNNNSNFPWTAATKYHLNGRDSLVNPGPAMASHKDDEEKFFPMHGTLIFGKQGTNANDADLDNVMITDEEEIRTTNPVATEQYGYVSNNLQNSNTGPTTGTVNHQGALPGMVWQDRDVYLQGPIWAKIPHTDGHFHPSPLMGGFGLKHPPPQIMIKNTPVPANPPTNFSSAKFASFITQYSTGQVSVEIEWELQKENSKRWNPEIQYTSNYNKSVNVDFTVDTNGVYSEPRPIGTRYLTRNL

SEQ ID NO: 75 - protein, 733 aa, Artificial Sequence
  adeno-associated virus rh.32.33
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPAKKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSNSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 76 - DNA, 2211 bp, Artificial Sequence
  adeno-associated virus 9 isolated nucleic acid sequence
  misc_feature (1)..(2208): vp1
  misc_feature (412)..(2208): vp2
  misc_feature (604)..(2208): vp3
atggctgccgatggttatcttccagattggctcgaggacaaccttagtgaaggaattcgcgagtggtgggctttgaaacctggagcccctcaacccaaggcaaatcaacaacatcaagacaacgctcggggtcttgtgcttccgggttacaaataccttggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggtgcacagcccgctaaaaagagactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgctaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaagccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgtctcatgaatccactcatcgaccaatacttgtactatctctcaaagactattaacggttctggacagaatcaacaaacgctaaaattcagtgtggccggacccagcaacatggcagtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcttatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggctttcaacaaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 77 - DNA, 2211 bp, Artificial Sequence
  adeno-associated virus hu32 isolated nucleic acid sequence
  misc_feature (1)..(2208): vp1
  misc_feature (412)..(2208): vp2
  misc_feature (604)..(2208): vp3
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccggcaacggactcgacaagggggagccggtcaacgcagcagacgcggcggccctcgagcacgacaaggcctacgaccagcagctcaaggccggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcagtctcctcaggaaccggactcctccgcgggtattggcaaatcgggttcacagcccgctaaaaagaaactcaatttcggtcagactggcgacacagagtcagtccccgaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctaatgggagggtttggaatgaagcacccgcctcctcagatcctcatcaaaaacacacctgtacctgcggatcctccaacggctttcaataaggacaagctgaactctttcatcacccagtattctactggccaagtcagcgtggagattgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctgtaa

SEQ ID NO: 78 - DNA, 22 bp, Artificial Sequence
  synthetic construct
agtgaattctaccagtgccata

SEQ ID NO: 79 - DNA, 23 bp, Artificial Sequence
  synthetic construct
agcaaaaatgtgctagtgccaaa

SEQ ID NO: 80 - DNA, 24 bp, Artificial Sequence
  synthetic construct
agtgtgagttctaccattgccaaa

SEQ ID NO: 81 - DNA, 23 bp, Artificial Sequence
  synthetic construct
agggattcctgggaaaactggac

SEQ ID NO: 82 - DNA, 2217 bp, adeno-associated virus 8
atggctgccgatggttatcttccagattggctcgaggacaacctctctgagggcattcgcgagtggtgggcgctgaaacctggagccccgaagcccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctgcaggcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaa

SEQ ID NO: 83 - protein, 738 aa, adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 84 - DNA, 2214 bp, adeno-associated virus 7
atggctgccgatggttatcttccagattggctcgaggacaacctctctgagggcattcgcgagtggtgggacctgaaacctggagccccgaaacccaaagccaaccagcaaaagcaggacaacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcatttgggggcaacctcgggcgagcagtcttccaggccaagaagcgggttctcgaacctctcggtctggttgaggaaggcgctaagacggctcctgcaaagaagagaccggtagagccgtcacctcagcgttcccccgactcctccacgggcatcggcaagaaaggccagcagcccgccagaaagagactcaatttcggtcagactggcgactcagagtcagtccccgaccctcaacctctcggagaacctccagcagcgccctctagtgtgggatctggtacagtggctgcaggcggtggcgcaccaatggcagacaataacgaaggtgccgacggagtgggtaatgcctcaggaaattggcattgcgattccacatggctgggcgacagagtcattaccaccagcacccgaacctgggccctgcccacctacaacaaccacctctacaagcaaatctccagtgaaactgcaggtagtaccaacgacaacacctacttcggctacagcaccccctgggggtattttgactttaacagattccactgccacttctcaccacgtgactggcagcgactcatcaacaacaactggggattccggcccaagaagctgcggttcaagctcttcaacatccaggtcaaggaggtcacgacgaatgacggcgttacgaccatcgctaataaccttaccagcacgattcaggtattctcggactcggaataccagctgccgtacgtcctcggctctgcgcaccagggctgcctgcctccgttcccggcggacgtcttcatgattcctcagtacggctacctgactctcaacaatggcagtcagtctgtgggacgttcctccttctactgcctggagtacttcccctctcagatgctgagaacgggcaacaactttgagttcagctacagcttcgaggacgtgcctttccacagcagctacgcacacagccagagcctggaccggctgatgaatcccctcatcgaccagtacttgtactacctggccagaacacagagtaacccaggaggcacagctggcaatcgggaactgcagttttaccagggcgggccttcaactatggccgaacaagccaagaattggttacctggaccttgcttccggcaacaaagagtctccaaaacgctggatcaaaacaacaacagcaactttgcttggactggtgccaccaaatatcacctgaacggcagaaactcgttggttaatcccggcgtcgccatggcaactcacaaggacgacgaggaccgctttttcccatccagcggagtcctgatttttggaaaaactggagcaactaacaaaactacattggaaaatgtgttaatgacaaatgaagaagaaattcgtcctactaatcctgtagccacggaagaatacgggatagtcagcagcaacttacaagcggctaatactgcagcccagacacaagttgtcaacaaccagggagccttacctggcatggtctggcagaaccgggacgtgtacctgcagggtcccatctgggccaagattcctcacacggatggcaactttcacccgtctcctttgatgggcggctttggacttaaacatccgcctcctcagatcctgatcaagaacactcccgttcccgctaatcctccggaggtgtttactcctgccaagtttgcttcgttcatcacacagtacagcaccggacaagtcagcgtggaaatcgagtgggagctgcagaaggaaaacagcaagcgctggaacccggagattcagtacacctccaactttgaaaagcagactggtgtggactttgccgttgacagccagggtgtttactctgagcctcgccctattggcactcgttacctcacccgtaatctgtaa

SEQ ID NO: 85 - protein, 737 aa, adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

(19) United States
(12) Patent Application Publication
(10) Pub. No.: US 2023/0407333 A1
(43) Pub. Date: Dec. 21, 2023

(54) AAV CAPSIDS AND COMPOSITIONS CONTAINING SAME

(71) Applicant: The Trustees of the University of Pennsylvania, Philadelphia, PA (US)

(72) Inventors: James M. Wilson, Philadelphia, PA (US); Kalyani Nambiar, New York, NY (US); Qiang Wang, Philadelphia, PA (US)

(73) Assignee: The Trustees of the University of Pennsylvania, Philadelphia, PA (US)

(21) Appl. No.: 18/249,842

(22) PCT Filed: Oct. 29, 2021
(86) PCT No.: PCT/US2021/057201

§ 371 Date: Apr. 20, 2023

Related U.S. Application Data
(60) Provisional application No. 63/214,530, filed on Jun. 24, 2021.
(60) Provisional application No. 63/107,030, filed on Oct. 29, 2020.

(51) Int. Cl. C12N 15/86; C07K 14/005

(52) U.S. Cl. CPC C12N 15/86; C07K 14/005; C12N 2750/14143; C12N 2750/14122

(57) ABSTRACT

Novel AAV capsids and recombinant AAV vectors comprising the same are provided.

### BACKGROUND OF THE INVENTION

Adeno-associated virus (AAV) vectors hold great promise in human gene therapy and have been widely used to target liver, muscle, heart, brain, eye, kidney, and other tissues in various studies due to their ability to provide long-term gene expression and lack of pathogenicity. AAV belongs to the parvovirus family and contains a single-stranded DNA genome flanked by two inverted terminal repeats. Dozens of naturally occurring AAV capsids have been reported; their unique capsid structures enable them to recognize and transduce different cell types and organs.

Since the first trial which started in 1981, there has not been any vector-related toxicity reported in clinical trials of AAV vector-based gene therapy. The ever-accumulating safety records of AAV vector in clinical trials, combined with demonstrated efficacy, show that AAV is an attractive platform. In particular, AAV is easily manipulated as the virus has a single-stranded DNA virus with a relatively small genome (˜4.7 kb) and simple genetic components-inverted terminal repeats (ITR), the Rep and Cap genes. Only the ITRs and AAV capsid protein are required in AAV vectors, with the ITRs serving as replication and packaging signals for vector production and the capsid proteins playing a central role by forming capsids to accommodate vector genome DNA and determining tissue tropism.

AAVs are among the most effective vector candidates for gene therapy due to their low immunogenicity and non-pathogenic nature. However, despite allowing for efficient gene transfer, the AAV vectors currently used in the clinic can be hindered by preexisting immunity to the virus and restricted tissue tropism. Thus, additional AAV vectors are needed.

### SUMMARY OF THE INVENTION

In one aspect, provided herein is a recombinant adeno-associated virus (rAAV) comprising a capsid and a vector genome comprising an AAV 5′ inverted terminal repeat (ITR), an expression cassette comprising a nucleic acid sequence encoding a gene product operably linked to expression control sequences, and an AAV 3′ ITR, wherein the capsid is: (a) an AAVrh75 capsid consisting of (i) a capsid produced from a nucleic acid sequence encoding SEQ ID NO: 40 or a sequence at least 99% identical thereto having an Asn (N) amino acid residue at position 24 based on the numbering of SEQ ID NO: 40; (ii) a capsid produced from a nucleic acid sequence of SEQ ID NO: 39 of a sequence or a sequence at least 95% identical thereto encoding SEQ ID NO: 40; or (iii) a capsid which is heterogeneous mixture of AAVrh75 vp1, vp2 and vp3 proteins which are 95% to 100% deamidated in at least position N57, N262, N384, and/or N512 of SEQ ID NO: 40, and optionally deamidated in other positions; (b) an AAVhu71/74 capsid consisting of (i) a capsid produced from a nucleic acid sequence encoding SEQ ID NO: 4; (ii) a capsid produced from a nucleic acid sequence of SEQ ID NO: 3 of a sequence or a sequence at least 95% identical thereto encoding SEQ ID NO: 4; or (iii) a capsid which is a heterogeneous mixture of AAVrh71/74 vp1, vp2, and vp3 proteins which are 95% to 100% deamidated in at least 4 positions of SEQ ID NO: 4, and optionally deamidated in other positions; (c) an AAVhu79 capsid consisting of (i) a capsid produced from a nucleic acid sequence encoding SEQ ID NO: 6; (ii) a capsid produced from a nucleic acid sequence of SEQ ID NO: 5 of a sequence or a sequence at least 95% identical thereto encoding SEQ ID NO: 6; or (iii) a capsid which is a heterogeneous mixture of AAVhu79 vp1, vp2, and vp3 proteins which are 95% to 100% deamidated in at least four positions of SEQ ID NO: 6, and optionally deamidated in other positions; (d) an AAVhu80 capsid consisting of (i) a capsid produced from a nucleic acid sequence encoding SEQ ID NO: 8; (ii) a capsid produced from a nucleic acid sequence of SEQ ID NO: 7 of a sequence or a sequence at least 95% identical thereto encoding SEQ ID NO: 8; or (iii) a capsid which is a heterogeneous mixture of AAVhu80 vp1, vp2, and vp3 proteins which are 95% to 100% deamidated in at least four positions of SEQ ID NO: 8, and optionally deamidated in other positions; (e) an AAVhu83 capsid consisting of (i) a capsid produced from a nucleic acid sequence encoding SEQ ID NO: 10; (i) a capsid produced from a nucleic acid sequence of SEQ ID NO: 9 of a sequence or a sequence at least 95% identical thereto encoding SEQ ID NO: 10; or (iii) a capsid which is a heterogeneous mixture of AAVhu83 vp1, vp2, and vp3 proteins which are 95% to 100% deamidated in at least four positions of SEQ ID NO: 10, and optionally deamidated in other positions; (f) an AAVhu74/71 capsid consisting of (i) a capsid produced from a nucleic acid sequence encoding SEQ ID NO: 12; (ii) a capsid produced from a nucleic acid sequence of SEQ ID NO: 11 of a sequence or a sequence at least 95% identical thereto encoding SEQ ID NO: 12; or (iii) a capsid which is a heterogeneous mixture of AAVhu74/71 vp1, vp2, and vp3 proteins which are 95% to 100% deamidated in at least four positions of SEQ ID NO: 12, and optionally deamidated in other positions; (g) an AAVhu77 capsid, consisting of (i) a capsid produced from a nucleic acid sequence encoding SEQ ID NO: 14; (ii) a capsid produced from a nucleic acid sequence of SEQ ID NO: 13 of a sequence or a sequence at least 95% identical thereto encoding SEQ ID NO: 14; or (iii) a capsid which is a heterogeneous mixture of AAVhu77 vp1, vp2, and vp3 proteins which are 95% to 100% deamidated in at least four positions of SEQ ID NO: 14, and optionally deamidated in other positions; (h) an AAVhu78/88 capsid consisting of (i) a capsid produced from a nucleic acid sequence encoding SEQ ID NO: 16; (ii) a capsid produced from a nucleic acid sequence of SEQ ID NO: 15 of a sequence or a sequence at least 95% identical thereto encoding SEQ ID NO: 16; or (iii) a capsid which is a heterogeneous mixture of AAVhu78/88 vp1, vp2, and vp3 proteins which are 95% to 100% deamidated in at least four positions of SEQ ID NO: 16, and optionally deamidated in other positions; (i) an AAVhu70 capsid consisting of (i) a capsid produced from a nucleic acid sequence encoding SEQ ID NO: 18; (ii) a capsid produced from a nucleic acid sequence of SEQ ID NO: 17 of a sequence or a sequence at least 95% identical thereto encoding SEQ ID NO: 18; or (iii) a capsid which is a heterogeneous mixture of AAVhu70 vp1, vp2, and vp3 proteins which are 95% to 100% deamidated in at least four positions of SEQ ID NO: 18, and optionally deamidated in other positions; (j) an AAVhu72 capsid consisting of (i) a capsid produced from a nucleic acid sequence encoding SEQ ID NO: 20; (ii) a capsid produced from a nucleic acid sequence of SEQ ID NO: 19 of a sequence or a sequence at least 95% identical thereto encoding SEQ ID NO: 20; or (iii) a capsid which is a heterogeneous mixture of AAVhu72 vp1, vp2, and vp3 proteins which are 95% to 100% deamidated in at least four positions of SEQ ID NO: 20, and optionally deamidated in other positions; (k) an AAVhu75 capsid consisting of (i) a capsid produced from a nucleic acid sequence encoding SEQ ID NO: 22; (ii) a capsid produced from a nucleic acid sequence of SEQ ID NO: 21 of a sequence or a sequence at least 95% identical thereto encoding SEQ ID NO: 22; or (iii) a capsid which is a heterogeneous mixture of AAVhu75 vp1, vp2, and vp3 proteins which are 95% to 100% deamidated in at least four positions of SEQ ID NO: 22, and optionally deamidated in other positions; (1) an AAVhu76 capsid consisting of (i) a capsid produced from a nucleic acid sequence encoding SEQ ID NO: 24; (ii) a capsid produced from a nucleic acid sequence of SEQ ID NO: 23 of a sequence or a sequence at least 95% identical thereto encoding SEQ ID NO: 24; or (iii) a capsid which is a heterogeneous mixture of AAVhu76 vp1, vp2, and vp3 proteins which are 95% to 100% deamidated in at least four positions of SEQ ID NO: 24, and optionally deamidated in other positions; (m) an AAVhu81 capsid consisting of (i) a capsid produced from a nucleic acid sequence encoding SEQ ID NO: 26; (ii) a capsid produced from a nucleic acid sequence of SEQ ID NO: 25 of a sequence or a sequence at least 95% identical thereto encoding SEQ ID NO: 26; or (iii) a capsid which is a heterogeneous mixture of AAVhu81 vp1, vp2, and vp3 proteins which are 95% to 100% deamidated in at least four positions of SEQ ID NO: 26, and optionally deamidated in other positions; (n) an AAVhu82 capsid consisting of (i) a capsid produced from a nucleic acid sequence encoding SEQ ID NO: 28; (ii) a capsid produced from a nucleic acid sequence of SEQ ID NO: 27 of a sequence or a sequence at least 95% identical thereto encoding SEQ ID NO: 28; or (iii) a capsid which is a heterogeneous mixture of AAVhu82 vp1, vp2, and vp3 proteins which are 95% to 100% deamidated in at least four positions of SEQ ID NO: 28, and optionally deamidated in other positions; (o) an AAVhu84 capsid consisting of (i) a capsid produced from a nucleic acid sequence encoding SEQ ID NO: 30; (ii) a capsid produced from a nucleic acid sequence of SEQ ID NO: 29 of a sequence or a sequence at least 95% identical thereto encoding SEQ ID NO: 30; or (iii) a capsid which is a heterogeneous mixture of AAVhu84 vp1, vp2, and vp3 proteins which are 95% to 100% deamidated in at least four positions of SEQ ID NO: 30, and optionally deamidated in other positions; (p) an AAVhu86 capsid consisting of (i) a capsid produced from a nucleic acid sequence encoding SEQ ID NO: 32; (ii) a capsid produced from a nucleic acid sequence of SEQ ID NO: 31 of a sequence or a sequence at least 95% identical thereto encoding SEQ ID NO: 32; or (iii) a capsid which is a heterogeneous mixture of AAVhu86 vp1, vp2, and vp3 proteins which are 95% to 100% deamidated in at least four positions of SEQ ID NO: 32, and optionally deamidated in other positions; (q) an AAVhu87 capsid consisting of (i) a capsid produced from a nucleic acid sequence encoding SEQ ID NO: 34; (ii) a capsid produced from a nucleic acid sequence of SEQ ID NO: 33 of a sequence or a sequence at least 95% identical thereto encoding SEQ ID NO: 34; or (iii) a capsid which is a heterogeneous mixture of AAVhu87 vp1, vp2, and vp3 proteins which are 95% to 100% deamidated in at least four positions of SEQ ID NO: 34, and optionally deamidated in other positions; (r) an AAVhu88/78 capsid consisting of (i) a capsid produced from a nucleic acid sequence encoding SEQ ID NO: 36; (ii) a capsid produced from a nucleic acid sequence of SEQ ID NO: 35 of a sequence or a sequence at least 95% identical thereto encoding SEQ ID NO: 36; or (iii) a capsid which is a heterogeneous mixture of AAVhu88/78 vp1, vp2, and vp3 proteins which are 95% to 100% deamidated in at least four positions of SEQ ID NO: 36, and optionally deamidated in other positions; (s) an AAVhu69 capsid consisting of (i) a capsid produced from a nucleic acid sequence encoding SEQ ID NO: 38; (ii) a capsid produced from a nucleic acid sequence of SEQ ID NO: 37 of a sequence or a sequence at least 95% identical thereto encoding SEQ ID NO: 38; or (iii) a capsid which is a heterogeneous mixture of AAVhu69 vp1, vp2, and vp3 proteins which are 95% to 100% deamidated in at least four positions of SEQ ID NO: 38, and optionally deamidated in other positions; (t) an AAVrh76 capsid consisting of (i) a capsid produced from a nucleic acid sequence encoding SEQ ID NO: 42; (ii) a capsid produced from a nucleic acid sequence of SEQ ID NO: 41 of a sequence or a sequence at least 95% identical thereto encoding SEQ ID NO: 42; or (iii) a capsid which is a heterogeneous mixture of AAVhu69 vp1, vp2, and vp3 proteins which are 95% to 100% deamidated in at least four positions of SEQ ID NO: 42, and optionally deamidated in other positions; (u) an AAVrh77 capsid consisting of (i) a capsid produced from a nucleic acid sequence encoding SEQ ID NO: 44; (ii) a capsid produced from a nucleic acid sequence of SEQ ID NO: 43 of a sequence or a sequence at least 95% identical thereto encoding SEQ ID NO: 44; or (iii) a capsid which is a heterogeneous mixture of AAVrh71 vp1, vp2, and vp3 proteins which are 95% to 100% deamidated in at least four positions of SEQ ID NO: 44, and optionally deamidated in other positions; (v) an AAVrh78 capsid consisting of (i) a capsid produced from a nucleic acid sequence encoding SEQ ID NO: 46; (ii) a capsid produced from a nucleic acid sequence of SEQ ID NO: 45 of a sequence or a sequence at least 95% identical thereto encoding SEQ ID NO: 46; or (iii) a capsid which is a heterogeneous mixture of AAVrh78 vp1, vp2, and vp3 proteins which are 95% to 100% deamidated in at least four positions of SEQ ID NO: 46, and optionally deamidated in other positions; (w) an AAVrh81 capsid consisting of (i) a capsid produced from a nucleic acid sequence encoding SEQ ID NO: 50; (ii) a capsid produced from a nucleic acid sequence of SEQ ID NO: 49 of a sequence or a sequence at least 95% identical thereto encoding SEQ ID NO: 50; or (iii) a capsid which is a heterogeneous mixture of AAVrh81 vp1, vp2, and vp3 proteins which are 95% to 100% deamidated in at least four positions of SEQ ID NO: 50, and optionally deamidated in other positions; (x) an AAVrh89 capsid consisting of (i) a capsid produced from a nucleic acid sequence encoding SEQ ID NO: 52; (ii) a capsid produced from a nucleic acid sequence of SEQ ID NO: 51 of a sequence or a sequence at least 95% identical thereto encoding SEQ ID NO: 52; or (iii) a capsid which is a heterogeneous mixture of AAVrh89 vp1, vp2, and vp3 proteins which are 95% to 100% deamidated in at least four positions of SEQ ID NO: 52, and optionally deamidated in other positions; (y) an AAVrh82 capsid consisting of (i) a capsid produced from a nucleic acid sequence encoding SEQ ID NO: 54; (ii) a capsid produced from a nucleic acid sequence of SEQ ID NO: 53 of a sequence or a sequence at least 95% identical thereto encoding SEQ ID NO: 54; or (iii) a capsid which is a heterogeneous mixture of AAVrh82 vp1, vp2, and vp3 proteins which are 95% to 100% deamidated in at least four positions of SEQ ID NO: 54, and optionally deamidated in other positions; (z) an AAVrh83 capsid consisting of (i) a capsid produced from a nucleic acid sequence encoding SEQ ID NO: 56; (ii) a capsid produced from a nucleic acid sequence of SEQ ID NO: 55 of a sequence or a sequence at least 95% identical thereto encoding SEQ ID NO: 56; or (iii) a capsid which is a heterogeneous mixture of AAVrh83 vp1, vp2, and vp3 proteins which are 95% to 100% deamidated in at least four positions of SEQ ID NO: 56, and optionally deamidated in other positions; (aa) an AAVrh84 capsid consisting of (i) a capsid produced from a nucleic acid sequence encoding SEQ ID NO: 58; (ii) a capsid produced from a nucleic acid sequence of SEQ ID NO: 57 of a sequence or a sequence at least 95% identical thereto encoding SEQ ID NO: 58; or (iii) a capsid which is a heterogeneous mixture of AAVrh84 vp1, vp2, and vp3 proteins which are 95% to 100% deamidated in at least four positions of SEQ ID NO: 58, and optionally deamidated in other positions; (bb) an AAVrh85 capsid consisting of (i) a capsid produced from a nucleic acid sequence encoding SEQ ID NO: 60; (ii) a capsid produced from a nucleic acid sequence of SEQ ID NO: 59 of a sequence or a sequence at least 95% identical thereto encoding SEQ ID NO: 60; or (iii) a capsid which is a heterogeneous mixture of AAVrh85 vp1, vp2, and vp3 proteins which are 95% to 100% deamidated in at least four positions of SEQ ID NO: 60, and optionally deamidated in other positions; (cc) an AAVrh87 capsid consisting of (i) a capsid produced from a nucleic acid sequence encoding SEQ ID NO: 62; (ii) a capsid produced from a nucleic acid sequence of SEQ ID NO: 61 of a sequence or a sequence at least 95% identical thereto encoding SEQ ID NO: 62; or (iii) a capsid which is a heterogeneous mixture of AAVrh87 vp1, vp2, and vp3 proteins which are 95% to 100% deamidated in at least four positions of SEQ ID NO: 62, and optionally deamidated in other positions; (dd) an AAVhu73 capsid consisting of (i) a capsid produced from a nucleic acid sequence encoding SEQ ID NO: 74; (ii) a capsid produced from a nucleic acid sequence of SEQ ID NO: 73 of a sequence or a sequence at least 95% identical thereto encoding SEQ ID NO: 74; or (iii) a capsid which is a heterogeneous mixture of AAVrh73 vp1, vp2, and vp3 proteins which are 95% to 100% deamidated in at least four positions of SEQ ID NO: 74, and optionally deamidated in other positions.

In one aspect, provided herein is a pharmaceutical composition comprising a rAAV, and a physiologically compatible carrier, buffer, adjuvant, and/or diluent.

In one aspect, provided herein is a method of delivering a transgene to a cell, said method comprising the step of contacting the cell with the rAAV according to any one of claims 1 to 5, wherein said rAAV comprises the transgene.

In one aspect, provided herein is a method of generating a recombinant adeno-associated virus (rAAV) comprising an AAV capsid, the method comprising culturing a host cell containing: (a) a molecule encoding an AAV vp1, vp2, and/or vp3 capsid protein of AAVrh75 (SEQ ID NO: 40), AAVhu71/74 (SEQ ID NO: 4), AAVhu79 (SEQ ID NO: 6), AAVhu80 (SEQ ID NO: 8), AAVhu83 (SEQ ID NO: 10), AAVhu74/71 (SEQ ID NO: 12), AAVhu77 (SEQ ID NO: 14), AAVhu78/88 (SEQ ID NO: 16), AAVhu70 (SEQ ID NO: 18), AAVhu72 (SEQ ID NO: 20), AAVhu75 (SEQ ID NO: 22), AAVhu76 (SEQ ID NO: 24), AAVhu81 (SEQ ID NO: 26), AAVhu82 (SEQ ID NO: 28), AAVhu84 (SEQ ID NO: 30), AAVhu86 (SEQ ID NO: 32), AAVhu87 (SEQ ID NO: 34), AAVhu88/78 (SEQ ID NO: 36), AAVhu69 (SEQ ID NO: 38), AAVrh76 (SEQ ID NO: 42), AAVrh77 (SEQ ID NO: 44), AAVrh78 (SEQ ID NO: 46), AAVrh81 (SEQ ID NO: 50), AAVrh89 (SEQ ID NO: 52), AAVrh82 (SEQ ID NO: 54), AAVrh83 (SEQ ID NO: 56), AAVrh84 (SEQ ID NO: 58), AAVrh85 (SEQ ID NO: 60), AAVrh87 (SEQ ID NO: 62), or AAVhu73 (SEQ ID NO: 74), or an AAV vp1, vp2, and/or vp3 capsid protein sharing at least 99% identity with any of SEQ ID NOs: 40, 4, 6, 8, 10, 12, 14, 16, 18, 20, 22, 24, 26, 28, 30, 32, 34, 36, 38, 42, 44, 46, 50, 52, 54, 56, 58, 60, 62, or 74, (b) a functional rep gene; (c) a vector genome comprising AAV inverted terminal repeats (ITRs) and a transgene; and (d) sufficient helper functions to permit packaging of the vector genome into the AAV capsid protein.

In one aspect, provided herein is a plasmid comprising a vp1, vp2, and/or vp3 sequence of AAVrh75 (SEQ ID NO: 39), AAVhu71/74 (SEQ ID NO: 3), AAVhu79 (SEQ ID NO: 5), AAVhu80 (SEQ ID NO: 7), AAVhu83 (SEQ ID NO: 9), AAVhu74/71 (SEQ ID NO: 11), AAVhu77 (SEQ ID NO: 13), AAVhu78/88 (SEQ ID NO: 15), AAVhu70 (SEQ ID NO: 17), AAVhu72 (SEQ ID NO: 19), AAVhu75 (SEQ ID NO: 21), AAVhu76 (SEQ ID NO: 23), AAVhu81 (SEQ ID NO: 25), AAVhu82 (SEQ ID NO: 27), AAVhu84 (SEQ ID NO: 29), AAVhu86 (SEQ ID NO: 31), AAVhu87 (SEQ ID NO: 33), AAVhu88/78 (SEQ ID NO: 35), AAVhu69 (SEQ ID NO: 37), AAVrh76 (SEQ ID NO: 41), AAVrh77 (SEQ ID NO: 43), AAVrh78 (SEQ ID NO: 45), AAVrh81 (SEQ ID NO: 49), AAVrh89 (SEQ ID NO: 51), AAVrh82 (SEQ ID NO: 53), AAVrh83 (SEQ ID NO: 55), AAVrh84 (SEQ ID NO: 57), AAVrh85 (SEQ ID NO: 59), AAVrh87 (SEQ ID NO: 61), or AAVhu73 (SEQ ID NO: 73), or vp1, vp2, and/or vp3 sequence sharing at least 95% identity with any of SEQ ID NO: 39, 3, 5, 7, 9, 11, 13, 15, 17, 19, 21, 23, 25, 27, 29, 31, 33, 35, 37, 41, 43, 45, 49, 51, 53, 55, 57, 59, 61, or 73. In a further embodiment, a cultured host cell containing such a plasmid is provided.

Other aspects and advantages of these compositions and methods are described further in the following detailed description.

### BRIEF DESCRIPTION OF THE DRAWINGS

FIG. 1 shows a diagram for AAV-Single Genome Amplification (AAV-SGA). Bulk mammalian genomic DNA samples were screened by PCR using AAV-specific primers that amplify a 3.1 kb region of the AAV genome encompassing the terminal third of the Rep gene and the complete Cap gene sequence. A sample that yields positive results for AAV detection PCR is endpoint-diluted in a 96-well plate format and used as the template for 3.1 kb amplicon AAV-specific PCR. The dilution of gDNA that results in less than a 30% positive PCR rate contains one amplifiable AAV genome in each reaction. Each positive amplicon is size selected and sequenced using the Illumina MiSeq platform. Reads originating from single genomes are de novo assembled to recover full-length AAV contigs containing the VP1 capsid gene.

FIG. 2A-FIG. 2D show an analysis of variable fidelity of DNA polymerases and bioactivity of PCR mutants. (FIG. 2A) Comparison of PCR errors induced by HiFi and Q5 DNA polymerases on circular and linearized plasmid template. PCR products were cloned and sequenced. Each dot represents an individual plasmid clone. HiFi circular, n=19; HiFi Linear, n=20; Q5 Circular, n=24; Q5 Linear, n=20 plasmid clones. (FIG. 2B) Vector production titers of AAV9-mutant PCR isolates generated by HiFi PCR. Mutant capsids were packaged with the CB7.ffluciferase.rBG transgene. We measured genome copy titers by qPCR of the total HEK293 triple-transfection cell lysates. (FIG. 2C) Huh7 infectious titers of PCR mutants, as measured by luciferase luminescence. “n/a”: “not available” because luminescence values were below the limit of detection. For B and C, The AAV9 controls were set to 100%; values are shown as the mean and standard deviation (SD). Statistical significance was assessed with the Wilcoxon rank sum test (FIG. 2A) and Student's t-test (FIG. 2B and FIG. 2C); not significant (NS): p>=0.05, *p<0.05, **p<0.01 and ***p<0.001. (FIG. 2D) Schematic of aligned PCR mutant AAV Cap DNA sequences. Each nucleotide mismatch to AAV9 is shown as a black line. Sequence information for the mismatches in these experiments are detailed in Table 1.

FIG. 3A-FIG. 3C show phylogenetic analyses of positive selection of AAV VP1 genes Neighbor-joining phylogenies of AAV VP1 DNA sequences from human isolates (FIG. 3A), rhesus macaque isolates (FIG. 3B), and previously reported human AAV HSC (FIG. 3C). Branches where BUSTED detected evidence of positive selection are colored in red. Circled branch nodes represent bootstrap support values >70.

FIG. 4 shows a phylogenetic analysis of HiFi PCR mutant AAV VP1 genes. Neighbor-joining phylogeny of AAV VP1 DNA sequences of HiFi PCR mutants.

FIG. 5A-FIG. 5C show an alignment of amino acid sequences for AAVhu72 (SEQ ID NO: 20), AAVhu75 (SEQ ID NO: 22), AAVhu79 (SEQ ID NO: 6), AAVhu80 (SEQ ID NO: 81), AAVhu81 (SEQ ID NO: 26), AAVhu82 (SEQ ID NO: 28), AAVhu83 (SEQ ID NO: 10), and AAVhu86 (SEQ ID NO: 32).

FIG. 6A-FIG. 6G show an alignment of nucleotide sequences for AAVhu72 (SEQ ID NO: 19), AAVhu75 (SEQ ID NO: 21), AAVhu79 (SEQ ID NO: 5), AAVhu80 (SEQ ID NO: 7), AAVhu81 (SEQ ID NO: 25), AAVhu82 (SEQ ID NO: 27), AAVhu83 (SEQ ID NO: 9), and AAVhu86 (SEQ ID NO: 31).

FIG. 7A-FIG. 7D show an alignment of amino acid sequences for AAVhu69 (SEQ ID NO: 38), AAVhu70 (SEQ ID NO: 18), AAVhu71.74 (SEQ ID NO: 4), AAVhu73 (SEQ ID NO: 74), AAVhu74.71 (SEQ ID NO: 12), AAVhu76 (SEQ ID NO: 24), AAVhu77 (SEQ ID NO: 14), AAVhu78.88 (SEQ ID NO: 16), AAVhu84 (SEQ ID NO: 30), AAVhu87 (SEQ ID NO: 34), AAVhu88.78 (SEQ ID NO: 36), and AAVrh81 (SEQ ID NO: 50).

FIG. 8A-FIG. 8J show an alignment of nucleotide sequences for AAVhu69 (SEQ ID NO: 37), AAVhu70 (SEQ ID NO: 17), AAVhu71.74 (SEQ ID NO: 3), AAVhu73 (SEQ ID NO: 73), AAVhu74.71 (SEQ ID NO: 11), AAVhu76 (SEQ ID NO: 23), AAVhu77 (SEQ ID NO: 13), AAVhu78.88 (SEQ ID NO: 15), AAVhu84 (SEQ ID NO: 29), AAVhu87 (SEQ ID NO: 33), AAVhu88.78 (SEQ ID NO: 25), and AAVrh81 (SEQ ID NO: 49).

FIG. 9A-FIG. 9B show an alignment of amino acid sequences for, AAVrh76 (SEQ ID NO: 42), AAVrh85 (SEQ ID NO: 60), AAVrh87 (SEQ ID NO: 62), AAVrh89 (SEQ ID NO: 52), and AAV7 (SEQ ID NO: 85).

FIG. 10A-FIG. 10E show an alignment of nucleotide sequences for AAVrh75 (SEQ ID NO: 39), AAVrh76 (SEQ ID NO: 41), AAVrh85 (SEQ ID NO: 59), AAVrh87 (SEQ ID NO: 61), AAVrh89 (SEQ ID NO: 51), and AAV7 (SEQ ID NO: 84).

FIG. 11A-FIG. 11B show an alignment of amino acid sequences for AAVrh75 (SEQ ID NO: 40), AAVrh79 (SEQ ID NO: 48), AAVrh83 (SEQ ID NO: 56), AAVrh84 (SEQ ID NO: 58), and AAV8 (SEQ ID NO: 83).

FIG. 12A-FIG. 12E show an alignment of nucleotide sequences for AAVrh79 (SEQ ID NO: 47), AAVrh83 (SEQ ID NO: 55), AAVrh84 (SEQ ID NO: 57), and AAV8 (SEQ ID NO: 82).

FIG. 13 shows an alignment of amino acid sequences for AAVrh77 (SEQ ID NO: 44), AAVrh78 (SEQ ID NO: 46), and AAVrh82 (SEQ ID NO: 54).

FIG. 14A-FIG. 14C show an alignment of nucleotide sequences for AAVrh77 (SEQ ID NO: 43), AAVrh78 (SEQ ID NO: 45), and AAVrh82 (SEQ ID NO: 53).

FIG. 15 shows AAV vector yields. Cis plasmids containing the capsid genes for the indicated isolates were used to package a vector genome containing the TBG promoter and an eGFP transgene. The vectors were manufactured with triple-transfection (one CellStack each), purified with a iodixanol gradient, and titrated using qPCR. “E+#” refers to the exponent which follows the E+ in numerical value, e.g., E+13 refers to “×10¹³” “,GC” refers vector genome copies.

FIG. 16 shows infectious titers for AAVrh75 and AAVrh81 vector preparations. Vectors (carrying a reporter transgene cassette) with AAVrh75 and AAVrh81 capsids were prepared at the plate scale, with AAV8 as the control. Crude lysates were then used to transduce a human and a mouse cell line. The infectious titers for AAVrh75 and AAVrh81 are presented as the transduction relative to AAV8 control.

FIG. 17 shows liver transduction for an AAVrh81 vector. C57BL/6J mice were dosed with AAVrh91.LSP.hF9 or AAV8.LSP.hF9 at 1×10¹⁰ gc/animal intravenously and plasma was collected 28 days after dosing for human F9 (hF9) measurement.

FIG. 18 shows liver transduction for AAVrh83 and AAVrh84 vectors. C57BL/6J mice were dosed with AAVrh83.TBG.eGFP or AAVrh84.TBG.eGFP at a dose of 1×10¹¹ gc/animal intravenously. Livers were harvested 14 days later for GFP imaging. Representative images from each animal are shown.

FIG. 19 shows liver transduction for novel AAV isolates. C57BL/6J mice were dosed with AAVrh78.TBG.eGFP, AAVrh78.TBG.eGFP, AAVrh78.TBG.eGFP, or AAVrh78.TBG.eGFP, or AAV8.TBG.eGFP at a dose of 1×10¹¹ gc/animal (AAVrh87 was 6.4×10¹⁰ gc/animal due to low prep titer) intravenously. Livers were harvested 14 days later and genomic DNA was extracted for vector genome copy measurement by qPCR. The liver transduction levels for AAVrh78, AAVrh85, AAVrh87, and AAVrh89 were ˜49%, 72%, 16% and 22% of AAV8, respectively. The p values (t-test, compared to the AAV8 group) are shown.

### DETAILED DESCRIPTION OF THE INVENTION

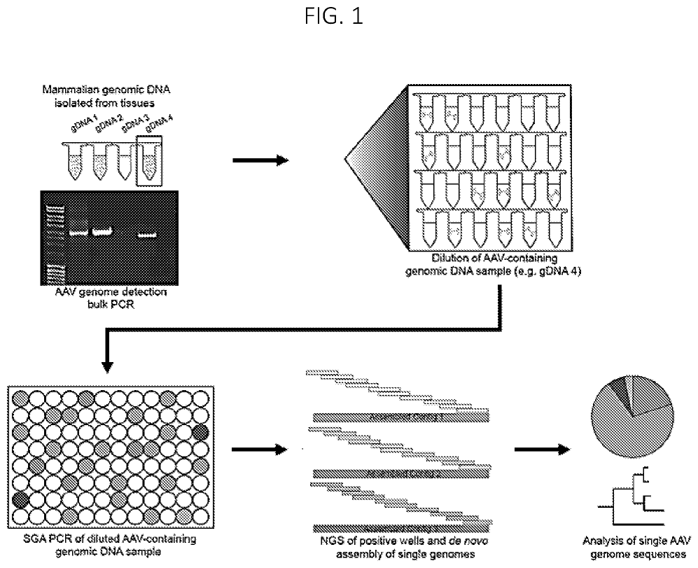

The genetic variation of AAVs in their natural mammalian hosts was explored by using AAV single genome amplification, a technique used to accurately isolate individual AAV genomes from within a viral population (FIG. 1). Described herein is the isolation of novel AAV sequences from rhesus macaque tissues and human tissues that can be categorized in various clades. The 12 novel AAV isolates from rhesus macaque tissues can be categorized in clades D, E, and the primate clade outgroup that contains AAVrh32.33. Additionally, the 20 novel AAV isolates from human tissues can be categorized in clades B and C, or similar to AAV2 and AAV2-AAV3 hybrids, respectively.

Unless defined otherwise, technical and scientific terms used herein have the same meaning as commonly understood by one of ordinary skill in the art to which this invention belongs and by reference to published texts, which provide one skilled in the art with a general guide to many of the terms used in the present application. The following definitions are provided for clarity only and are not intended to limit the claimed invention.

The term “substantial homology” or “substantial similarity,” when referring to a nucleic acid, or fragment thereof, indicates that, when optimally aligned with appropriate nucleotide insertions or deletions with another nucleic acid (or its complementary strand), there is nucleotide sequence identity in at least about 95 to 99% of the aligned sequences. Preferably, the homology is over full-length sequence, or an open reading frame thereof, or another suitable fragment which is at least 15 nucleotides in length. Examples of suitable fragments are described herein.

The terms “sequence identity” “percent sequence identity” or “percent identical” in the context of nucleic acid sequences refers to the residues in the two sequences which are the same when aligned for maximum correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired. Similarly, “percent sequence identity” may be readily determined for amino acid sequences, over the full-length of a protein, or a fragment thereof. Suitably, a fragment is at least about 8 amino acids in length and may be up to about 700 amino acids. Examples of suitable fragments are described herein.

The term “substantial homology” or “substantial similarity,” when referring to amino acids or fragments thereof, indicates that, when optimally aligned with appropriate amino acid insertions or deletions with another amino acid (or its complementary strand), there is amino acid sequence identity in at least about 95 to 99% of the aligned sequences. Preferably, the homology is over full-length sequence, or a protein thereof, e.g., a cap protein, a rep protein, or a fragment thereof which is at least 8 amino acids, or more desirably, at least 15 amino acids in length. Examples of suitable fragments are described herein.

By the term “highly conserved” is meant at least 80% identity, preferably at least 90% identity, and more preferably, over 97% identity. Identity is readily determined by one of skill in the art by resort to algorithms and computer programs known by those of skill in the art.

Generally, when referring to “identity”, “homology”, or “similarity” between two different adeno-associated viruses, “identity”, “homology” or “similarity” is determined in reference to “aligned” sequences. “Aligned” sequences or “alignments” refer to multiple nucleic acid sequences or protein (amino acids) sequences, often containing corrections for missing or additional bases or amino acids as compared to a reference sequence. In the examples, AAV alignments are performed using the published AAV9 sequences as a reference point. Alignments are performed using any of a variety of publicly or commercially available Multiple Sequence Alignment Programs. Examples of such programs include, “Clustal Omega”, “Clustal W”, “CAP Sequence Assembly”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOPAM factor for the scoring matrix) as provided in GCG Version 6.1, herein incorporated by reference. Multiple sequence alignment programs are also available for amino acid sequences, e.g., the “Clustal Omega”, “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

The term “AAV intermediate” or “AAV vector intermediate” refers to an assembled rAAV capsid which lacks the desired genomic sequences packaged therein. These may also be termed an “empty” capsid. Such a capsid may contain no detectable genomic sequences of an expression cassette, or only partially packaged genomic sequences which are insufficient to achieve expression of the gene product.

A “genetic element” includes any nucleic acid molecule, e.g., naked DNA, a plasmid, phage, transposon, cosmid, episome, virus, etc., which transfers the sequences carried thereon. Optionally, such a genetic element may utilize a lipid-based carrier. Unless otherwise specified, the genetic element may be delivered by any suitable method, including transfection, electroporation, liposome delivery, membrane fusion techniques, high velocity DNA-coated pellets, viral infection and protoplast fusion.

A “stable host cell” for rAAV production is a host cell with had been engineered to contain one or more of the required rAAV production elements (e.g., minigene, rep sequences, the AAVhu68 engineered cap sequences as defined herein, and/or helper functions) and its progeny. A stable host cell may contain the required component(s) under the control of an inducible promoter. Alternatively, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from HEK293 cells (which contain E1 helper functions under the control of a constitutive promoter), Huh7 cells, Vero cells, engineered to contain helper functions under the control of a suitable promoter, which optionally further contains the rep and/or cap proteins under the control of inducible promoters. Still other stable host cells may be generated by one of skill in the art.

As used herein, an “expression cassette” refers to a nucleic acid molecule which comprises a biologically useful nucleic acid sequence (e.g., a gene cDNA encoding a protein, enzyme or other useful gene product, mRNA, etc.) and regulatory sequences operably linked thereto which direct or modulate transcription, translation, and/or expression of the nucleic acid sequence and its gene product.

The abbreviation “sc” refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

As used herein, the term “operably linked” refers to both expression control sequences that are contiguous with the gene of interest and expression control sequences that act in trans or at a distance to control the gene of interest.

The term “heterologous” when used with reference to a protein or a nucleic acid indicates that the protein or the nucleic acid comprises two or more sequences or subsequences which are not found in the same relationship to each other in nature. For instance, the nucleic acid is typically recombinantly produced, having two or more sequences from unrelated genes arranged to make a new functional nucleic acid. For example, in one embodiment, the nucleic acid has a promoter from one gene arranged to direct the expression of a coding sequence from a different gene. Thus, with reference to the coding sequence, the promoter is heterologous.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In one embodiment, the genome of the viral vector does not include genes encoding the enzymes required to replicate (the genome can be engineered to be “gutless”—containing only the gene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication.

In many instances, rAAV particles are referred to as DNase resistant. However, in addition to this endonuclease (DNase), other endo- and exo-nucleases may also be used in the purification steps described herein, to remove contaminating nucleic acids. Such nucleases may be selected to degrade single stranded DNA and/or double-stranded DNA, and RNA. Such steps may contain a single nuclease, or mixtures of nucleases directed to different targets, and may be endonucleases or exonucleases.

The term “nuclease-resistant” indicates that the AAV capsid has fully assembled around the expression cassette which is designed to deliver a gene to a host cell and protects these packaged genomic sequences from degradation (digestion) during nuclease incubation steps designed to remove contaminating nucleic acids which may be present from the production process.

As used herein, an “effective amount” refers to the amount of the rAAV composition which delivers and expresses in the target cells an amount of the gene product from the vector genome. An effective amount may be determined based on an animal model, rather than a human patient. Examples of a suitable murine model are described herein.

The term “translation” in the context of the present invention relates to a process at the ribosome, wherein an mRNA strand controls the assembly of an amino acid sequence to generate a protein or a peptide.

As used herein, the terms “a” or “an”, refers to one or more, for example, “an expression cassette” is understood to represent one or more expression cassettes. As such, the terms “a” (or “an”), “one or more,” and “at least one” are used interchangeably herein.

As used herein, the term “about” means a variability of 10% from the reference given, unless otherwise specified.

While various embodiments in the specification are presented using “comprising” language, under other circumstances, a related embodiment is also intended to be interpreted and described using “consisting of” or “consisting essentially of” language.

With regard to the following description, it is intended that each of the compositions herein described, is useful, in another embodiment, in the methods of the invention. In addition, it is also intended that each of the compositions described as useful in the methods, is, in another embodiment, itself an embodiment of the invention.

### A. The AAV Capsid

Nucleic acids encoding AAV capsids include three overlapping coding sequences, which vary in length due to alternative start codon usage. The translated proteins are referred to as VP1, VP2 and VP3, with VP1 being the longest and VP3 being the shortest. The AAV particle consists of all three capsid proteins at a ratio of ˜1:1:10 (VP1:VP2:VP3). VP3, which is comprised in VP1 and VP2 at the N-terminus, is the main structural component that builds the particle. The capsid protein can be referred to using several different numbering systems. For convenience, as used herein, the AAV sequences are referred to using VP1 numbering, which starts with aa 1 for the first residue of VP1. However, the capsid proteins described herein include VP1, VP2, and VP3 (used interchangeably herein with vp1, vp2, and vp3).

### Clade B

Provided herein are novel AAV capsid proteins having vp1 sequences set forth in the sequence listing: AAVhu72 (SEQ ID NO: 20), AAVhu75 (SEQ ID NO: 22), AAVhu79 (SEQ ID NO: 6), AAVhu80 (SEQ ID NO: 8), AAVhu81 (SEQ ID NO: 26), AAVhu82 (SEQ ID NO: 28), AAVhu83 (SEQ ID NO: 10), or AAVhu86 (SEQ ID NO: 32). The numbering of the nucleotides and amino acids corresponding to the vp1, vp2, and vp3 are as follows:

Nucleotides (nt)
AAVhu72: vp1—nt 1 to 2205; vp2—nt 412 to 2205; vp3—nt 607 to 2205 of SEQ ID NO: 19;
AAVhu75: vp1—nt 1 to 2205; vp2—nt 412 to 2205; vp3—nt 607 to 2205 of SEQ ID NO: 21;
AAVhu79: vp1—nt 1 to 2205; vp2—nt 412 to 2205; vp3—nt 607 to 2205 of SEQ ID NO: 5;
AAVhu80: vp1—nt 1 to 2205; vp2—nt 412 to 2205; vp3—nt 607 to 2205 of SEQ ID NO: 7;
AAVhu81: vp1—nt 1 to 2205; vp2—nt 412 to 2205; vp3—nt 607 to 2205 of SEQ ID NO: 25;
AAVhu82: vp1—nt 1 to 2205; vp2—nt 412 to 2205; vp3—nt 607 to 2205 of SEQ ID NO: 27;
AAVhu83: vp1—nt 1 to 2205; vp2—nt 412 to 2205; vp3—nt 607 to 2205 of SEQ ID NO: 9;
AAVhu86: vp1—nt 1 to 2205; vp2—nt 412 to 2205; vp3—nt 607 to 2205 of SEQ ID NO: 31.

Amino Acids (aa)
AAVhu72: aa vp1—1 to 735; vp2—aa 138 to 735; vp3—aa 203 to 735 of SEQ ID NO: 20;
AAVhu75: aa vp1—1 to 735; vp2—aa 138 to 735; vp3—aa 203 to 735 of SEQ ID NO: 22;
AAVhu79: aa vp1—1 to 735; vp2—aa 138 to 735; vp3—aa 203 to 735 of SEQ ID NO: 6;
AAVhu80: aa vp1—1 to 735; vp2—aa 138 to 735; vp3—aa 203 to 735 of SEQ ID NO: 8;
AAVhu81: aa vp1—1 to 735; vp2—aa 138 to 735; vp3—aa 203 to 735 of SEQ ID NO: 26;
AAVhu82: aa vp1—1 to 735; vp2—aa 138 to 735; vp3—aa 203 to 735 of SEQ ID NO: 28;
AAVhu83: aa vp1—1 to 735; vp2—aa 138 to 735; vp3—aa 203 to 735 of SEQ ID NO: 10;
AAVhu86: aa vp1—1 to 735; vp2—aa 138 to 735; vp3—aa 203 to 735 of SEQ ID NO: 32.

In certain embodiments, provided herein are rAAV comprising at least one of the vp1, vp2, and vp3 of any of AAVhu72 (SEQ ID NO: 20), AAVhu75 (SEQ ID NO: 22), AAVhu79 (SEQ ID NO: 6), AAVhu80 (SEQ ID NO: 8), AAVhu81 (SEQ ID NO: 26), AAVhu82 (SEQ ID NO: 28), AAVhu83 (SEQ ID NO: 10), or AAVhu86 (SEQ ID NO: 32). In certain embodiments, rAAV having a capsid protein comprising a vp1, vp2, and/or vp3 sequence at least 95%, at least 96%, at least 97%, at least 98%, or at least 99% identical to AAVhu72 (SEQ ID NO: 20), AAVhu75 (SEQ ID NO: 22), AAVhu79 (SEQ ID NO: 6), AAVhu80 (SEQ ID NO: 8), AAVhu81 (SEQ ID NO: 26), AAVhu82 (SEQ ID NO: 28), AAVhu83 (SEQ ID NO: 10), or AAVhu86 (SEQ ID NO: 32) are provided. In certain embodiments, the vp1, vp2, and/or vp3 has up to 1, up to 2, up to 3, up to 4, up to 5, up to 6, up to 7, up to 8, up to 9, or up to 10 amino acid differences relative to the vp1, vp2, and/or vp3 of AAVhu72 (SEQ ID NO: 20), AAVhu75 (SEQ ID NO: 22), AAVhu79 (SEQ ID NO: 6), AAVhu80 (SEQ ID NO: 8), AAVhu81 (SEQ ID NO: 26), AAVhu82 (SEQ ID NO: 28), AAVhu83 (SEQ ID NO: 10), or AAVhu86 (SEQ ID NO: 32). Also provided herein are rAAV comprising AAV capsids encoded by at least one of the vp1, vp2, vp3 sequence of AAVhu72 (SEQ ID NO: 19), AAVhu75 (SEQ ID NO: 21), AAVhu79 (SEQ ID NO: 5), AAVhu80 (SEQ ID NO: 7), AAVhu81 (SEQ ID NO: 25), AAVhu82 (SEQ ID NO: 27), AAVhu83 (SEQ ID NO: 9), or AAVhu86 (SEQ ID NO: 31), or a sequence at least 95%, at least 96%, at least 97%, at least 98%, or at least 99% identical to SEQ ID NO: 19, 21, 5, 7, 25, 27, 9, or 31. In certain embodiments, the sequence encodes a full-length vp1, vp2 and/or vp3 of AAVhu72 (SEQ ID NO: 20), AAVhu75 (SEQ ID NO: 22), AAVhu79 (SEQ ID NO: 6), AAVhu80 (SEQ ID NO: 8), AAVhu81 (SEQ ID NO: 26), AAVhu82 (SEQ ID NO: 28), AAVhu83 (SEQ ID NO: 10), or AAVhu86 (SEQ ID NO: 32). In other embodiments, the vp1, vp2 and/or vp3 has an N-terminal and/or a C-terminal truncation (e.g. truncation(s) of about 1 to about 10 amino acids).

### Clade C

Provided herein are novel AAV capsid proteins having vp1 sequences set forth in the sequence listing: AAVrh81 (SEQ ID NO: 50), AAVhu71.74 (SEQ ID NO: 4), AAVhu73 (SEQ ID NO: 74), AAVhu74.71 (SEQ ID NO: 12), AAVhu77 (SEQ ID NO: 14), AAVhu78.88 (SEQ ID NO: 16), AAVhu70 (SEQ ID NO: 18), AAVhu76 (SEQ ID NO: 24), AAVhu84 (SEQ ID NO: 30), hu87 (SEQ ID NO: 34), AAVhu88.78 (SEQ ID NO: 36), or AAVhu69 (SEQ ID NO: 38). The numbering of the nucleotides and amino acids corresponding to the vp1, vp2, and vp3 are as follows:

Nucleotides (nt)
AAVrh81: vp1—nt 1 to 2217; vp2—nt 412 to 2217; vp3—nt 619 to 2217 of SEQ ID NO: 49;
AAVhu71.74: vp1—nt 1 to 2205; vp2—nt 412 to 2205; vp3—nt 607 to 2205 of SEQ ID NO: 3;
AAVhu73: vp1—nt 1 to 2205; vp2—nt 412 to 2205; vp3—nt 607 to 2205 of SEQ ID NO: 73;
AAVhu74.71: vp1—nt 1 to 2205; vp2—nt 412 to 2205; vp3—nt 607 to 2205 of SEQ ID NO: 11;
AAVhu77: vp1—nt 1 to 2205; vp2—nt 412 to 2205; vp3—nt 607 to 2205 of SEQ ID NO: 13;
AAVhu78.88: vp1—nt 1 to 2205; vp2—nt 412 to 2205; vp3—nt 607 to 2205 of SEQ ID NO: 15;
AAVhu70: vp1—nt 1 to 2205; vp2—nt 412 to 2205; vp3—nt 607 to 2205 of SEQ ID NO: 17;
AAVhu76: vp1—nt 1 to 2202; vp2—nt 412 to 2202; vp3—nt 607 to 2202 of SEQ ID NO: 23;
AAVhu84: vp1—nt 1 to 2205; vp2—nt 412 to 2205; vp3—nt 607 to 2205 of SEQ ID NO: 29;
AAVhu87: vp1—nt 1 to 2202; vp2—nt 412 to 2202; vp3—nt 607 to 2202 of SEQ ID NO: 33;
AAVhu88.78: vp1—nt 1 to 2205; vp2—nt 412 to 2205; vp3—nt 607 to 2205 of SEQ ID NO: 35;
AAVhu69: vp1—nt 1 to 2205; vp2—nt 412 to 2205; vp3—nt 607 to 2205 of SEQ ID NO: 37.

Amino Acids (aa)
AAVrh81: aa vp1—1 to 735; vp2—aa 138 to 735; vp3—aa 207 to 739 of SEQ ID NO: 50;
AAVhu71.74: aa vp1—1 to 735; vp2—aa 138 to 735; vp3—aa 203 to 735 of SEQ ID NO: 4;
AAVhu73: aa vp1—1 to 735; vp2—aa 138 to 735; vp3—aa 203 to 735 of SEQ ID NO: 74;
AAVhu74.71: aa vp1—1 to 735; vp2—aa 138 to 735; vp3—aa 203 to 735 of SEQ ID NO: 12;
AAVhu77: aa vp1—1 to 735; vp2—aa 138 to 735; vp3—aa 203 to 735 of SEQ ID NO: 14;
AAVhu78.88: aa vp1—1 to 735; vp2—aa 138 to 735; vp3—aa 203 to 735 of SEQ ID NO: 16;
AAVhu70: aa vp1—1 to 735; vp2—aa 138 to 735; vp3—aa 203 to 735 of SEQ ID NO: 18;
AAVhu76: aa vp1—1 to 735; vp2—aa 138 to 735; vp3—aa 203 to 734 of SEQ ID NO: 24;
AAVhu84: aa vp1—1 to 735; vp2—aa 138 to 735; vp3—aa 203 to 735 of SEQ ID NO: 30;
AAVhu87: aa vp1—1 to 735; vp2—aa 138 to 735; vp3—aa 203 to 734 of SEQ ID NO: 34;
AAVhu88.78: aa vp1—1 to 735; vp2—aa 138 to 735; vp3—aa 203 to 735 of SEQ ID NO: 36;
AAVhu69: aa vp1—1 to 735; vp2—aa 138 to 735; vp3—aa 203 to 735 of SEQ ID NO: 38.

In certain embodiments, provided herein are rAAV comprising at least one of the vp1, vp2, and vp3 of any of AAVrh81 (SEQ ID NO: 50), AAVhu71.74 (SEQ ID NO: 4), AAVhu73 (SEQ ID NO: 74), AAVhu74.71 (SEQ ID NO: 12), AAVhu77 (SEQ ID NO: 14), AAVhu78.88 (SEQ ID NO: 16), AAVhu70 (SEQ ID NO: 18), AAVhu76 (SEQ ID NO: 24), AAVhu84 (SEQ ID NO: 30), hu87 (SEQ ID NO: 34), AAVhu88.78 (SEQ ID NO: 36), or AAVhu69 (SEQ ID NO: 38). In certain embodiments, rAAV having a capsid protein comprising a vp1, vp2, and/or vp3 sequence at least 95%, at least 96%, at least 97%, at least 98%, or at least 99% identical to AAVrh81 (SEQ ID NO: 50), AAVhu71.74 (SEQ ID NO: 4), AAVhu73 (SEQ ID NO: 74), AAVhu74.71 (SEQ ID NO: 12), AAVhu77 (SEQ ID NO: 14), AAVhu78.88 (SEQ ID NO: 16), AAVhu70 (SEQ ID NO: 18), AAVhu76 (SEQ ID NO: 24), AAVhu84 (SEQ ID NO: 30), hu87 (SEQ ID NO: 34), AAVhu88.78 (SEQ ID NO: 36), or AAVhu69 (SEQ ID NO: 38) are provided. In certain embodiments, the vp1, vp2, and/or vp3 has up to 1, up to 2, up to 3, up to 4, up to 5, up to 6, up to 7, up to 8, up to 9, or up to 10 amino acid differences relative to the vp1, vp2, and/or vp3 of AAVrh81 (SEQ ID NO: 50), AAVhu71.74 (SEQ ID NO: 4), AAVhu73 (SEQ ID NO: 74), AAVhu74.71 (SEQ ID NO: 12), AAVhu77 (SEQ ID NO: 14), AAVhu78.88 (SEQ ID NO: 16), AAVhu70 (SEQ ID NO: 18), AAVhu76 (SEQ ID NO: 24), AAVhu84 (SEQ ID NO: 30), hu87 (SEQ ID NO: 34), AAVhu88.78 (SEQ ID NO: 36), or AAVhu69 (SEQ ID NO: 38). Also provided herein are rAAV comprising AAV capsids encoded by at least one of the vp1, vp2 and the vp3 sequence of AAVrh81 (SEQ ID NO: 49), AAVhu71.74 (SEQ ID NO: 3), AAVhu73 (SEQ ID NO: 73), AAVhu74.71 (SEQ ID NO: 11), AAVhu77 (SEQ ID NO: 13), AAVhu78.88 (SEQ ID NO: 15), AAVhu70 (SEQ ID NO: 17), AAVhu76 (SEQ ID NO: 23), AAVhu84 (SEQ ID NO: 29), hu87 (SEQ ID NO: 33), AAVhu88.78 (SEQ ID NO: 35), or AAVhu69 (SEQ ID NO: 37) or a sequence at least 95%, at least 96%, at least 97%, at least 98%, or at least 99% identical to SEQ ID NO: 49, 3, 73, 11, 13, 15, 17, 23, 29, 33, 35, or 37. In certain embodiments, the sequence encodes a full-length vp1, vp2 and/or vp3 of AAVrh81 (SEQ ID NO: 50), AAVhu71.74 (SEQ ID NO: 4), AAVhu73 (SEQ ID NO: 74), AAVhu74.71 (SEQ ID NO: 12), AAVhu77 (SEQ ID NO: 14), AAVhu78.88 (SEQ ID NO: 16), AAVhu70 (SEQ ID NO: 18), AAVhu76 (SEQ ID NO: 24), AAVhu84 (SEQ ID NO: 30), hu87 (SEQ ID NO: 34), AAVhu88.78 (SEQ ID NO: 36), or AAVhu69 (SEQ ID NO: 38). In other embodiments, the vp1, vp2 and/or vp3 has an N-terminal and/or a C-terminal truncation (e.g. truncation(s) of about 1 to about 10 amino acids).

### Clade D

Provided herein are novel AAV capsid proteins having vp1 sequences set forth in the sequence listing: AAVrh76 (SEQ ID NO: 42), AAVrh89 (SEQ ID NO: 52), AAVrh85 (SEQ ID NO: 60), or AAVrh87 (SEQ ID NO: 62). The numbering of the nucleotides and amino acids corresponding to the vp1, vp2, and vp3 are as follows:

Nucleotides (nt)
AAVrh76: vp1—nt 1 to 2211; vp2—nt 412 to 2211; vp3—nt 610 to 2211 of SEQ ID NO: 41;
AAVrh89: vp1—nt 1 to 2184; vp2—nt 412 to 2184; vp3—nt 595 to 2184 of SEQ ID NO: 51;
AAVrh85: vp1—nt 1 to 2211; vp2—nt 412 to 2211; vp3—nt 610 to 2211 of SEQ ID NO: 59;
AAVrh87: vp1—nt 1 to 2211; vp2—nt 412 to 2211; vp3—nt 610 to 2211 of SEQ ID NO: 61.

Amino Acids (aa)
AAVrh76: aa vp1—1 to 737; vp2—aa 138 to 737; vp3—aa 204 to 737 of SEQ ID NO: 42;
AAVrh89: aa vp1—1 to 728; vp2—aa 138 to 728; vp3—aa 199 to 728 of SEQ ID NO: 52;
AAVrh85: aa vp1—1 to 737; vp2—aa 138 to 737; vp3—aa 204 to 737 of SEQ ID NO: 60;
AAVrh87: aa vp1—1 to 737; vp2—aa 138 to 737; vp3—aa 204 to 737 of SEQ ID NO: 62.

In certain embodiments, provided herein are rAAV comprising at least one of the vp1, vp2, and vp3 of any of AAVrh76 (SEQ ID NO: 42), AAVrh89 (SEQ ID NO: 52), AAVrh85 (SEQ ID NO: 60), or AAVrh87 (SEQ ID NO: 62). In certain embodiments, rAAV having a capsid protein comprising a vp1, vp2, and/or vp3 sequence at least 95%, at least 96%, at least 97%, at least 98%, or at least 99% identical to AAVrh75 (SEQ ID NO: 40), AAVrh76 (SEQ ID NO: 42), AAVrh89 (SEQ ID NO: 52), AAVrh85 (SEQ ID NO: 60), or AAVrh87 (SEQ ID NO: 62) are provided. In certain embodiments, the vp1, vp2, and/or has up to 1, up to 2, up to 3, up to 4, up to 5, up to 6, up to 7, up to 8, up to 9, or up to 10 amino acid differences relative to the vp1, vp2, and/or vp3 of AAVrh76 (SEQ ID NO: 42), AAVrh89 (SEQ ID NO: 52), AAVrh85 (SEQ ID NO: 60), or AAVrh87 (SEQ ID NO: 62). Also provided herein are rAAV comprising AAV capsids encoded by at least one of the vp1, vp2, and the vp3 sequence of any of AAVrh75 (SEQ ID NO: 39), AAVrh76 (SEQ ID NO: 41), AAVrh89 (SEQ ID NO: 51), AAVrh85 (SEQ ID NO: 59), or AAVrh87 (SEQ ID NO: 61) or a sequence at least 95%, at least 96%, at least 97%, at least 98%, or at least 99% identical to SEQ ID NO: 39, 41, 51, 59, or 61. In certain embodiments, the sequence encodes a full-length vp1, vp2 and/or vp3 of AAVrh75 (SEQ ID NO: 40), AAVrh76 (SEQ ID NO: 42), AAVrh89 (SEQ ID NO: 52), AAVrh85 (SEQ ID NO: 60), or AAVrh87 (SEQ ID NO: 62). In other embodiments, the vp1, vp2 and/or vp3 has an N-terminal and/or a C-terminal truncation (e.g. truncation(s) of about 1 to about 10 amino acids).

### Clade E

Provided herein are novel AAV capsid proteins having vp1 sequences set forth in the sequence listing: AAVrh75 (SEQ ID NO: 40), AAVrh79 (SEQ ID NO: 48), AAVrh83 (SEQ ID NO: 56), or AAVrh84 (SEQ ID NO: 58). The numbering of the nucleotides and amino acids corresponding to the vp1, vp2, and vp3 are as follows:

Nucleotides (nt)
AAVrh75: vp1—nt 1 to 2208; vp2—nt 412 to 2208; vp3—nt 607 to 2208 of SEQ ID NO: 39;
AAVrh79: vp1—nt 1 to 2214; vp2—nt 412 to 2214; vp3—nt 610 to 2214 of SEQ ID NO: 47;
AAVrh83: vp1—nt 1 to 2211; vp2—nt 412 to 2211; vp3—nt 610 to 2211 of SEQ ID NO: 55;
AAVrh84: vp1—nt 1 to 2211; vp2—nt 412 to 2211; vp3—nt 610 to 2211 of SEQ ID NO: 57.

Amino Acids (aa)
AAVrh75: aa vp1—1 to 736; vp2—aa 138 to 736; vp3—aa 203 to 736 of SEQ ID NO: 40;
AAVrh79: aa vp1—1 to 738; vp2—aa 138 to 738; vp3—aa 204 to 738 of SEQ ID NO: 48;
AAVrh83: aa vp1—1 to 737; vp2—aa 138 to 737; vp3—aa 204 to 737 of SEQ ID NO: 56;
AAVrh84: aa vp1—1 to 737; vp2—aa 138 to 737; vp3—aa 204 to 737 of SEQ ID NO: 58.

In certain embodiments, provided herein are rAAV comprising at least one of the vp1, vp2 and the vp3 of any of AAVrh75 (SEQ ID NO: 40), AAVrh79 (SEQ ID NO: 48), AAVrh83 (SEQ ID NO: 56), or AAVrh84 (SEQ ID NO: 58). In certain embodiments, rAAV having a capsid protein comprising a vp1, vp2, and/or vp3 sequence at least 95%, at least 96%, at least 97%, at least 98%, or at least 99% identical to AAVrh75 (SEQ ID NO: 40), AAVrh79 (SEQ ID NO: 48), AAVrh83 (SEQ ID NO: 56), or AAVrh84 (SEQ ID NO: 58) are provided. In certain embodiments, the vp1, vp2, and/or vp3 has up to 1, up to 2, up to 3, up to 4, up to 5, up to 6, up to 7, up to 8, up to 9, or up to 10 amino acid differences relative to the vp1, vp2, and/or vp3 of AAVrh79 (SEQ ID NO: 48), AAVrh83 (SEQ ID NO: 56), or AAVrh84 (SEQ ID NO: 58). Also provided herein are rAAV comprising AAV capsids encoded by at least one of the vp1, vp2, and vp3 of AAVrh75 (SEQ ID NO: 40), AAVrh79 (SEQ ID NO: 47), AAVrh83 (SEQ ID NO: 55), or AAVrh84 (SEQ ID NO: 57), or a sequence at least 95%, at least 96%, at least 97%, at least 98%, or at least 99% identical to a SEQ ID NOs: 47, 55, or 57. In certain embodiments, the sequence encodes a full-length vp1, vp2 and/or vp3 of AAVrh79 (SEQ ID NO: 48), AAVrh83 (SEQ ID NO: 56), or AAVrh84 (SEQ ID NO: 58). In other embodiments, the vp1, vp2 and/or vp3 has an N-terminal and/or a C-terminal truncation (e.g. truncation(s) of about 1 to about 10 amino acids).

### “Fringe Clade” Outgroup

Provided herein are novel AAV capsid proteins having vp1 sequences set forth in the sequence listing: AAVrh77 (SEQ ID NO: 44), AAVrh78 (SEQ ID NO: 46), or AAVrh82 (SEQ ID NO: 54). The numbering of the nucleotides and amino acids corresponding to the vp1, vp2, and vp3 are as follows:

Nucleotides (nt)
AAVrh77: vp1—nt 1 to 2199; vp2—nt 412 to 2199; vp3—nt 589 to 2199 of SEQ ID NO: 43;
AAVrh78: vp1—nt 1 to 2199; vp2—nt 412 to 2199; vp3—nt 589 to 2199 of SEQ ID NO: 45;
AAVrh82: vp1—nt 1 to 2199; vp2—nt 412 to 2199; vp3—nt 589 to 2199 of SEQ ID NO: 53.

Amino Acids (aa)
AAVrh77: aa vp1—1 to 733; vp2—aa 138 to 733; vp3—aa 197 to 733 of SEQ ID NO: 44;
AAVrh78: aa vp1—1 to 733; vp2—aa 138 to 733; vp3—aa 197 to 733 of SEQ ID NO: 46;
AAVrh82: aa vp1—1 to 733; vp2—aa 138 to 733; vp3—aa 197 to 733 of SEQ ID NO: 82.

In certain embodiments, provided herein are rAAV comprising at least one of the vp1, vp2, and vp3 of any of AAVrh77 (SEQ ID NO: 44), AAVrh78 (SEQ ID NO: 46), or AAVrh82 (SEQ ID NO: 54). In certain embodiments, rAAV having a capsid protein comprising a vp1, vp2, and/or vp3 sequence at least 95%, at least 96%, at least 97%, at least 98%, or at least 99% identical to AAVrh77 (SEQ ID NO: 44), AAVrh78 (SEQ ID NO: 46), or AAVrh82 (SEQ ID NO: 54) are provided. In certain embodiments, the vp1, vp2, and/or vp3 has up to 1, up to 2, up to 3, up to 4, up to 5, up to 6, up to 7, up to 8, up to 9, or up to 10 amino acid differences relative to the vp1, vp2, and/or vp3 AAVrh77 (SEQ ID NO: 44), AAVrh78 (SEQ ID NO: 46), or AAVrh82 (SEQ ID NO: 54). Also provided herein are rAAV comprising AAV capsids encoded by at least one of the vp1, vp2, and vp3 of AAVrh77 (SEQ ID NO: 43), AAVrh78 (SEQ ID NO: 45), or AAVrh82 (SEQ ID NO: 53), or a sequence at least 95%, at least 96%, at least 97%, at least 98%, or at least 99% identical to SEQ ID NO: 43, 45, 53. In certain embodiments, the vp1, vp2 and/or vp3 is the full-length capsid protein of AAVrh77 (SEQ ID NO: 44), AAVrh78 (SEQ ID NO: 46), or AAVrh82 (SEQ ID NO: 54). In other embodiments, the vp1, vp2 and/or vp3 has an N-terminal and/or a C-terminal truncation (e.g. truncation(s) of about 1 to about 10 amino acids).

A “recombinant AAV” or “rAAV” is a DNAse-resistant viral particle containing two elements, an AAV capsid and a vector genome containing at least a non-AAV coding sequence packaged within the AAV capsid. Unless otherwise specified, this term may be used interchangeably with the phrase “rAAV vector”. The rAAV is a “replication-defective virus” or “viral vector”, as it lacks any functional AAV rep gene or functional AAV cap gene and cannot generate progeny. In certain embodiments, the only AAV sequences are the AAV inverted terminal repeat sequences (ITRs), typically located at the extreme 5′ and 3′ ends of the vector genome in order to allow the gene and regulatory sequences located between the ITRs to be packaged within the AAV capsid.

As used herein, a “vector genome” refers to the nucleic acid sequence packaged inside the rAAV capsid which forms a viral particle. Such a nucleic acid sequence contains AAV inverted terminal repeat sequences (ITRs). In the examples herein, a vector genome contains, at a minimum, from 5′ to 3′, an AAV 5′ ITR, coding sequence(s), and an AAV 3′ ITR. ITRs from AAV2, a different source AAV than the capsid, or other than full-length ITRs may be selected. In certain embodiments, the ITRs are from the same AAV source as the AAV which provides the rep function during production or a transcomplementing AAV. Further, other ITRs may be used. Further, the vector genome contains regulatory sequences which direct expression of the gene products. Suitable components of a vector genome are discussed in more detail herein. The vector genome is sometimes referred to herein as the “minigene”.

A rAAV is composed of an AAV capsid and a vector genome. An AAV capsid is an assembly of a heterogeneous population of vp1, a heterogeneous population of vp2, and a heterogeneous population of vp3 proteins. As used herein when used to refer to vp capsid proteins, the term “heterogeneous” or any grammatical variation thereof, refers to a population consisting of elements that are not the same, for example, having vp1, vp2 or vp3 monomers (proteins) with different modified amino acid sequences.

As used herein, the term “heterogeneous population” as used in connection with vp1, vp2 and vp3 proteins (alternatively termed isoforms), refers to differences in the amino acid sequence of the vp1, vp2 and vp3 proteins within a capsid. The AAV capsid contains subpopulations within the vp1 proteins, within the vp2 proteins and within the vp3 proteins which have modifications from the predicted amino acid residues. These subpopulations include, at a minimum, certain deamidated asparagine (N or Asn) residues. For example, certain subpopulations comprise at least one, two, three or four highly deamidated asparagines (N) positions in asparagine-glycine pairs and optionally further comprising other deamidated amino acids, wherein the deamidation results in an amino acid change and other optional modifications.

As used herein, a “subpopulation” of vp proteins refers to a group of vp proteins which has at least one defined characteristic in common and which consists of at least one group member to less than all members of the reference group, unless otherwise specified. For example, a “subpopulation” of vp1 proteins may be at least one (1) vp1 protein and less than all vp1 proteins in an assembled AAV capsid, unless otherwise specified. A “subpopulation” of vp3 proteins may be one (1) vp3 protein to less than all vp3 proteins in an assembled AAV capsid, unless otherwise specified. For example, vp1 proteins may be a subpopulation of vp proteins; vp2 proteins may be a separate subpopulation of vp proteins, and vp3 are yet a further subpopulation of vp proteins in an assembled AAV capsid. In another example, vp1, vp2 and vp3 proteins may contain subpopulations having different modifications, e.g., at least one, two, three or four highly deamidated asparagines, e.g., at asparagine-glycine pairs.

Unless otherwise specified, highly deamidated refers to at least 45% deamidated, at least 50% deamidated, at least 60% deamidated, at least 65% deamidated, at least 70%, at least 75%, at least 80%, at least 85%, at least 90%, at least 95%, at least 97%, at least 99%, or up to about 100% deamidated at a referenced amino acid position, as compared to the predicted amino acid sequence at the reference amino acid position. Such percentages may be determined using 2D-gel, mass spectrometry techniques, or other suitable techniques.

Without wishing to be bound by theory, the deamidation of at least highly deamidated residues in the vp proteins in the AAV capsid is believed to be primarily non-enzymatic in nature, being caused by functional groups within the capsid protein which deamidate selected asparagines, and to a lesser extent, glutamine residues. Efficient capsid assembly of the majority of deamidation vp1 proteins indicates that either these events occur following capsid assembly or that deamidation in individual monomers (vp1, vp2 or vp3) is well-tolerated structurally and largely does not affect assembly dynamics. Extensive deamidation in the VP1-unique (VP1-u) region (˜aa 1-137), generally considered to be located internally prior to cellular entry, suggests that VP deamidation may occur prior to capsid assembly.

Without wishing to be bound by theory, the deamidation of N may occur through its C-terminus residue's backbone nitrogen atom conducts a nucleophilic attack to the Asn side chain amide group carbon atom. An intermediate ring-closed succinimide residue is believed to form. The succinimide residue then conducts fast hydrolysis to lead to the final product aspartic acid (Asp) or iso aspartic acid (IsoAsp). Therefore, in certain embodiments, the deamidation of asparagine (N or Asn) leads to an Asp or IsoAsp, which may interconvert through the succinimide intermediate e.g., as illustrated below.

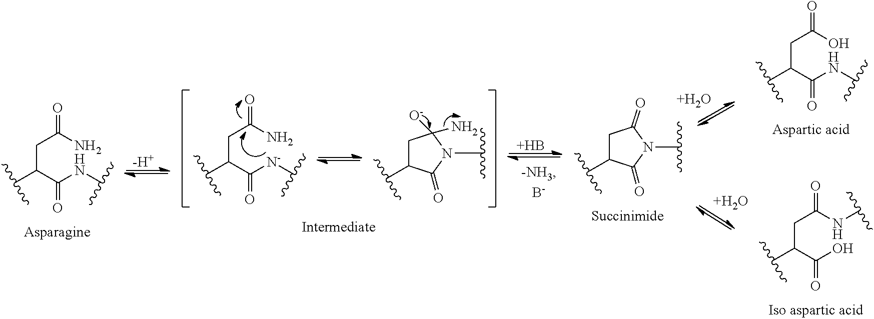

As provided herein, each deamidated N in the VP1, VP2 or VP3 may independently be aspartic acid (Asp), isoaspartic acid (isoAsp), aspartate, and/or an interconverting blend of Asp and isoAsp, or combinations thereof. Any suitable ratio of α- and isoaspartic acid may be present. For example, in certain embodiments, the ratio may be from 10:1 to 1:10 aspartic to isoaspartic, about 50:50 aspartic:isoaspartic, or about 1:3 aspartic:isoaspartic, or another selected ratio.

In certain embodiments, one or more glutamine (Q) may deamidates to glutamic acid (Glu), i.e., α-glutamic acid, γ-glutamic acid (Glu), or a blend of α- and γ-glutamic acid, which may interconvert through a common glutarinimide intermediate. Any suitable ratio of α- and γ-glutamic acid may be present. For example, in certain embodiments, the ratio may be from 10:1 to 1:10 α to γ, about 50:50 α:γ, or about 1:3 α:γ, or another selected ratio.

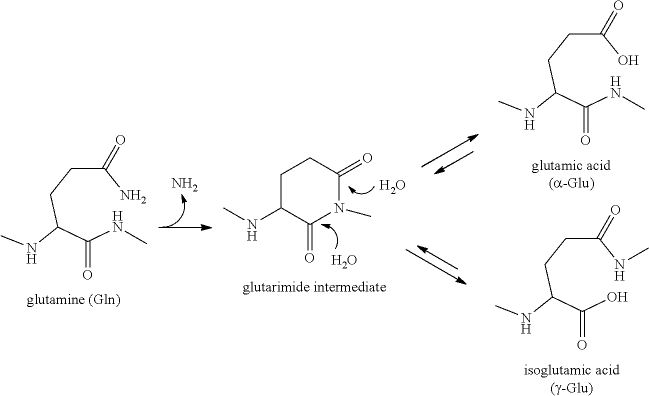

Thus, an rAAV includes subpopulations within the rAAV capsid of vp1, vp2 and/or vp3 proteins with deamidated amino acids, including at a minimum, at least one subpopulation comprising at least one highly deamidated asparagine. In addition, other modifications may include isomerization, particularly at selected aspartic acid (D or Asp) residue positions. In still other embodiments, modifications may include an amidation at an Asp position.

In certain embodiments, an AAV capsid contains subpopulations of vp1, vp2 and vp3 having at least 1, at least 2, at least 3, at least 4, at least 5 to at least about 25 deamidated amino acid residue positions, of which at least 1 to 10%, at least 10 to 25%, at least 25 to 50%, at least 50 to 70%, at least 70 to 100%, at least 75 to 100%, at least 80-100%, or at least 90-100% are deamidated as compared to the encoded amino acid sequence of the vp proteins. The majority of these may be N residues. However, Q residues may also be deamidated.

As used herein, “encoded amino acid sequence” refers to the amino acid which is predicted based on the translation of a known DNA codon of a referenced nucleic acid sequence being translated to an amino acid. The following table illustrates DNA codons and twenty common amino acids, showing both the single letter code (SLC) and three letter code (3LC).

| Amino Acid | SLC | 3 LC | DNA codons |
|---|---|---|---|
| Isoleucine | I | Ile | ATT, ATC, ATA |
| Leucine | L | Leu | CTT, CTC, CTA, CTG, TTA, TTG |
| Valine | V | Val | GTT, GTC, GTA, GTG |
| Phenylalanine | F | Phe | TTT, TTC |
| Methionine | M | Met | ATG |
| Cysteine | C | Cys | TGT, TGC |
| Alanine | A | Ala | GCT, GCC, GCA, GCG |
| Glycine | G | Gly | GGT, GGC, GGA, GGG |
| Proline | P | Pro | CCT, CCC, CCA, CCG |
| Threonine | T | Thr | ACT, ACC, ACA, ACG |
| Serine | S | Ser | TCT, TCC, TCA, TCG, AGT, AGC |
| Tyrosine | Y | Tyr | TAT, TAC |
| Tryptophan | W | Trp | TGG |
| Glutamine | Q | Gln | CAA, CAG |
| Asparagine | N | Asn | AAT, AAC |
| Histidine | H | His | CAT, CAC |
| Glutamic acid | E | Glu | GAA, GAG |
| Aspartic acid | D | Asp | GAT, GAC |
| Lysine | K | Lys | AAA, AAG |
| Arginine | R | Arg | CGT, CGC, CGA, CGG, AGA, AGG |
| Stop codons | Stop | | TAA, TAG, TGA |

In certain embodiments, a rAAV has an AAV capsid having vp1, vp2 and vp3 proteins having subpopulations comprising combinations of two, three, four, five or more deamidated residues at the positions set forth in the tables provided herein and incorporated herein by reference.

Deamidation in the rAAV may be determined using 2D gel electrophoresis, and/or mass spectrometry, and/or protein modelling techniques. Online chromatography may be performed with an Acclaim PepMap column and a Thermo UltiMate 3000 RSLC system (Thermo Fisher Scientific) coupled to a Q Exactive HF with a NanoFlex source (Thermo Fisher Scientific). MS data is acquired using a data-dependent top-20 method for the Q Exactive HF, dynamically choosing the most abundant not-yet-sequenced precursor ions from the survey scans (200-2000 m/z). Sequencing is performed via higher energy collisional dissociation fragmentation with a target value of 1e5 ions determined with predictive automatic gain control and an isolation of precursors was performed with a window of 4 m/z. Survey scans were acquired at a resolution of 120,000 at m/z 200. Resolution for HCD spectra may be set to 30,000 at m/z200 with a maximum ion injection time of 50 ms and a normalized collision energy of 30. The S-lens RF level may be set at 50, to give optimal transmission of the m/z region occupied by the peptides from the digest. Precursor ions may be excluded with single, unassigned, or six and higher charge states from fragmentation selection. BioPharma Finder 1.0 software (Thermo Fischer Scientific) may be used for analysis of the data acquired. For peptide mapping, searches are performed using a single-entry protein FASTA database with carbamidomethylation set as a fixed modification; and oxidation, deamidation, and phosphorylation set as variable modifications, a 10-ppm mass accuracy, a high protease specificity, and a confidence level of 0.8 for MS/MS spectra. Examples of suitable proteases may include, e.g., trypsin or chymotrypsin. Mass spectrometric identification of deamidated peptides is relatively straightforward, as deamidation adds to the mass of intact molecule+0.984 Da (the mass difference between —OH and —NH₂ groups). The percent deamidation of a particular peptide is determined by mass area of the deamidated peptide divided by the sum of the area of the deamidated and native peptides. Considering the number of possible deamidation sites, isobaric species which are deamidated at different sites may co-migrate in a single peak. Consequently, fragment ions originating from peptides with multiple potential deamidation sites can be used to locate or differentiate multiple sites of deamidation. In these cases, the relative intensities within the observed isotope patterns can be used to specifically determine the relative abundance of the different deamidated peptide isomers. This method assumes that the fragmentation efficiency for all isomeric species is the same and independent on the site of deamidation. It will be understood by one of skill in the art that a number of variations on these illustrative methods can be used. For example, suitable mass spectrometers may include, e.g, a quadrupole time of flight mass spectrometer (QTOF), such as a Waters Xevo or Agilent 6530 or an orbitrap instrument, such as the Orbitrap Fusion or Orbitrap Velos (Thermo Fisher). Suitably liquid chromatography systems include, e.g., Acquity UPLC system from Waters or Agilent systems (1100 or 1200 series). Suitable data analysis software may include, e.g., MassLynx (Waters), Pinpoint and Pepfinder (Thermo Fischer Scientific), Mascot (Matrix Science), Peaks DB (Bioinformatics Solutions). Still other techniques may be described, e.g., in X. Jin et al, Hu Gene Therapy Methods, Vol. 28, No. 5, pp. 255-267, published online Jun. 16, 2017.

In addition to deamidations, other modifications may occur do not result in conversion of one amino acid to a different amino acid residue. Such modifications may include acetylated residues, isomerizations, phosphorylations, or oxidations. Modulation of Deamidation: In certain embodiments, the AAV is modified to change the glycine in an asparagine-glycine pair, to reduce deamidation. In other embodiments, the asparagine is altered to a different amino acid, e.g., a glutamine which deamidates at a slower rate; or to an amino acid which lacks amide groups (e.g., glutamine and asparagine contain amide groups); and/or to an amino acid which lacks amine groups (e.g., lysine, arginine and histidine contain amine groups). As used herein, amino acids lacking amide or amine side groups refer to, e.g., glycine, alanine, valine, leucine, isoleucine, serine, threonine, cystine, phenylalanine, tyrosine, or tryptophan, and/or proline. Modifications such as described may be in one, two, or three of the asparagine-glycine pairs found in the encoded AAV amino acid sequence. In certain embodiments, such modifications are not made in all four of the asparagine-glycine pairs. Thus, a method for reducing deamidation of AAV and/or engineered AAV variants having lower deamidation rates. Additionally, or alternatively one or more other amide amino acids may be changed to a non-amide amino acid to reduce deamidation of the AAV. In certain embodiments, a mutant AAV capsid as described herein contains a mutation in an asparagine-glycine pair, such that the glycine is changed to an alanine or a serine. A mutant AAV capsid may contain one, two or three mutants where the reference AAV natively contains four NG pairs. In certain embodiments, an AAV capsid may contain one, two, three or four such mutants where the reference AAV natively contains five NG pairs. In certain embodiments, a mutant AAV capsid contains only a single mutation in an NG pair. In certain embodiments, a mutant AAV capsid contains mutations in two different NG pairs. In certain embodiments, a mutant AAV capsid contains mutation is two different NG pairs which are located in structurally separate location in the AAV capsid. In certain embodiments, the mutation is not in the VP1-unique region. In certain embodiments, one of the mutations is in the VP1-unique region. Optionally, a mutant AAV capsid contains no modifications in the NG pairs, but contains mutations to minimize or eliminate deamidation in one or more asparagines, or a glutamine, located outside of an NG pair.

In certain embodiments, a method of increasing the potency of a rAAV vector is provided which comprises engineering an AAV capsid which eliminating one or more of the NGs in the wild-type AAV capsid. In certain embodiments, the coding sequence for the “G” of the “NG” is engineered to encode another amino acid. In certain examples below, an “S” or an “A” is substituted. However, other suitable amino acid coding sequences may be selected.

Amino acid modifications may be made by conventional genetic engineering techniques. For example, a nucleic acid sequence containing modified AAV vp codons may be generated in which one to three of the codons encoding glycine in asparagine-glycine pairs are modified to encode an amino acid other than glycine. In certain embodiments, a nucleic acid sequence containing modified asparagine codons may be engineered at one to three of the asparagine-glycine pairs, such that the modified codon encodes an amino acid other than asparagine. Each modified codon may encode a different amino acid. Alternatively, one or more of the altered codons may encode the same amino acid. In certain embodiments, these modified nucleic acid sequences may be used to generate a mutant rAAV having a capsid with lower deamidation than the native AAV3B variant capsid. Such mutant rAAV may have reduced immunogenicity and/or increase stability on storage, particularly storage in suspension form.

Also provided herein are nucleic acid sequences encoding the AAV capsids having reduced deamidation. It is within the skill in the art to design nucleic acid sequences encoding this AAV capsid, including DNA (genomic or cDNA), or RNA (e.g., mRNA). Such nucleic acid sequences may be codon-optimized for expression in a selected system (i.e., cell type) and can be designed by various methods. This optimization may be performed using methods which are available on-line (e.g., GeneArt), published methods, or a company which provides codon optimizing services, e.g., DNA2.0 (Menlo Park, CA). One codon optimizing method is described, e.g., in International Patent Publication No. WO 2015/012924, which is incorporated by reference herein in its entirety. See also, e.g., US Patent Publication No. 2014/0032186 and US Patent Publication No. 2006/0136184. Suitably, the entire length of the open reading frame (ORF) for the product is modified. However, in some embodiments, only a fragment of the ORF may be altered. By using one of these methods, one can apply the frequencies to any given polypeptide sequence and produce a nucleic acid fragment of a codon-optimized coding region which encodes the polypeptide. A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional methods would be immediately apparent to the skilled artisan. In addition, gene synthesis is readily available commercially.

In certain embodiments, AAV capsids are provided which have a heterogeneous population of AAV capsid isoforms (i.e., VP1, VP2, VP3) which contain multiple highly deamidated “NG” positions. In certain embodiments, the highly deamidated positions are in the locations identified below, with reference to the predicted full-length VP1 amino acid sequence. In other embodiments, the capsid gene is modified such that the referenced “NG” is ablated and a mutant “NG” is engineered into another position.

B. rAAV Vectors and Compositions

In one aspect, provided herein are molecules which utilize the AAV capsid sequences described herein, including fragments thereof, for production of viral vectors useful in delivery of a heterologous gene or other nucleic acid sequences to a target cell. In certain embodiments, the rAAV provided have a capsid as described herein, and have packaged in the capsid a vector genome comprising a non-AAV nucleic acid sequence. In certain embodiments, the vectors useful in compositions and methods described herein contain, at a minimum, sequences encoding a selected AAV capsid as described herein, e.g., an AAVhu71/74 (SEQ ID NO: 4), AAVhu79 (SEQ ID NO: 6), AAVhu80 (SEQ ID NO: 8), AAVhu83 (SEQ ID NO: 10), AAVhu74/71 (SEQ ID NO: 12), AAVhu77 (SEQ ID NO: 14), AAVhu78/88 (SEQ ID NO: 16), AAVhu70 (SEQ ID NO: 18), AAVhu72 (SEQ ID NO: 20), AAVhu75 (SEQ ID NO: 22), AAVhu76 (SEQ ID NO: 24), AAVhu81 (SEQ ID NO: 26), AAVhu82 (SEQ ID NO: 28), AAVhu84 (SEQ ID NO: 30), AAVhu86 (SEQ ID NO: 32), AAVhu87 (SEQ ID NO: 34), AAVhu88/78 (SEQ ID NO: 36), AAVhu69 (SEQ ID NO: 38), AAVrh75 (SEQ ID NO: 40), AAVrh76 (SEQ ID NO: 42), AAVrh77 (SEQ ID NO: 44), AAVrh78 (SEQ ID NO: 46), AAVrh79 (SEQ ID NO: 48), AAVrh81 (SEQ ID NO: 50), AAVrh89 (SEQ ID NO: 52), AAVrh82 (SEQ ID NO: 54), AAVrh83 (SEQ ID NO: 56), AAVrh84 (SEQ ID NO: 58), AAVrh85 (SEQ ID NO: 60), AAVrh87 (SEQ ID NO: 62), or AAVhu73 (SEQ ID NO: 74) capsid, or a fragment thereof, including the vp1, vp2, or vp3 capsid protein. In certain embodiments, useful vectors contain, at a minimum, sequences encoding a selected AAV serotype rep protein, or a fragment thereof. Optionally, such vectors may contain both AAV cap and rep proteins. In vectors in which both AAV rep and cap are provided, the AAV rep and AAV cap sequences can both be of one serotype origin, e.g., all AAVhu71/74, AAVhu79, AAVhu80, AAVhu83, AAVhu74/71, AAVhu77, AAVhu78/88, AAVhu70, AAVhu72, AAVhu75, AAVhu76, AAVhu81, AAVhu82, AAVhu84, AAVhu86, AAVhu87, AAVhu88/78, AAVhu69, AAVrh75, AAVrh76, AAVrh77, AAVrh78, AAVrh79, AAVrh81, AAVrh89, AAVrh82, AAVrh83, AAVrh84, AAVrh85, AAVrh87, or AAVhu73 origin. Alternatively, vectors may be used in which the rep sequences are from an AAV which differs from the wild type AAV providing the cap sequences, e.g., the same AAV providing the ITRs and rep.

In one embodiment, the rep and cap sequences are expressed from separate sources (e.g., separate vectors, or a host cell and a vector). In another embodiment, these rep sequences are fused in frame to cap sequences of a different AAV serotype to form a chimeric AAV vector, such as AAV2/8 described in U.S. Pat. No. 7,282,199, which is incorporated by reference herein. Optionally, the vectors further contain a minigene comprising a selected transgene which is flanked by AAV 5′ ITR and AAV 3′ ITR. In another embodiment, the AAV is a self-complementary AAV (sc-AAV) (See, US 2012/0141422 which is incorporated herein by reference). Self-complementary vectors package an inverted repeat genome that can fold into dsDNA without the requirement for DNA synthesis or base-pairing between multiple vector genomes. Because scAAV have no need to convert the single-stranded DNA (ssDNA) genome into double-stranded DNA (dsDNA) prior to expression, they are more efficient vectors. However, the trade-off for this efficiency is the loss of half the coding capacity of the vector, ScAAV are useful for small protein-coding genes (up to ˜55 kd) and any currently available RNA-based therapy.

Pseudotyped vectors, wherein the capsid of one AAV is replaced with a heterologous capsid protein, are useful herein. For example, AAV vectors utilizing an AAVhu71/74, AAVhu79, AAVhu80, AAVhu83, AAVhu74/71, AAVhu77, AAVhu78/88, AAVhu70, AAVhu72, AAVhu75, AAVhu76, AAVhu81, AAVhu82, AAVhu84, AAVhu86, AAVhu87, AAVhu88/78, AAVhu69, AAVrh75, AAVrh76, AAVrh77, AAVrh78, AAVrh79, AAVrh81, AAVrh89, AAVrh82, AAVrh83, AAVrh84, AAVrh85, AAVrh87, or AAVhu73 capsid as described herein, have AAV2 ITRs. See, Mussolini et al. Unless otherwise specified, the AAV ITRs, and other selected AAV components described herein, may be individually selected from among any AAV serotype, including, without limitation, AAV1, AAV2, AAV3, AAV4, AAV5, AAV6, AAV7, AAV8, AAV9 or other known and unknown AAV serotypes. In one desirable embodiment, the ITRs of AAV serotype 2 are used. However, ITRs from other suitable serotypes may be selected. These ITRs or other AAV components may be readily isolated using techniques available to those of skill in the art from an AAV serotype. Such AAV may be isolated or obtained from academic, commercial, or public sources (e.g., the American Type Culture Collection, Manassas, VA). Alternatively, the AAV sequences may be obtained through synthetic or other suitable means by reference to published sequences such as are available in the literature or in databases such as, e.g., GenBank, PubMed, or the like.

The rAAV provided herein comprise a vector genome. The vector genome is composed of, at a minimum, a non-AAV or heterologous nucleic acid sequence (e.g., a transgene), as described below, regulatory sequences, and 5′ and 3′ AAV inverted terminal repeats (ITRs). It is this minigene which is packaged into a capsid protein and delivered to a selected target cell or target tissue.

The transgene is a nucleic acid sequence, heterologous to the vector sequences flanking the transgene, which encodes a polypeptide, protein, or other product, of interest. The nucleic acid coding sequence is operatively linked to regulatory components in a manner which permits transgene transcription, translation, and/or expression in a target cell. The heterologous nucleic acid sequence (transgene) can be derived from any organism. The AAV may comprise one or more transgenes.

As used herein, the terms “target cell” and “target tissue” can refer to any cell or tissue which is intended to be transduced by the subject AAV vector. The term may refer to any one or more of muscle, liver, lung, airway epithelium, central nervous system, neurons, eye (ocular cells), or heart. In one embodiment, the target tissue is liver. In another embodiment, the target tissue is the heart. In another embodiment, the target tissue is brain. In another embodiment, the target tissue is muscle.

As used herein, the term “mammalian subject” or “subject” includes any mammal in need of the methods of treatment described herein or prophylaxis, including particularly humans. Other mammals in need of such treatment or prophylaxis include dogs, cats, or other domesticated animals, horses, livestock, laboratory animals, including non-human primates, etc. The subject may be male or female.

As used herein, the term “host cell” may refer to the packaging cell line in which the rAAV is produced from the plasmid. In the alternative, the term “host cell” may refer to a target cell in which expression of the transgene is desired.

Therapeutic Transgenes

Useful products encoded by the transgene include a variety of gene products which replace a defective or deficient gene, inactivate or “knock-out”, or “knock-down” or reduce the expression of a gene which is expressing at an undesirably high level, or delivering a gene product which has a desired therapeutic effect. In most embodiments, the therapy will be “somatic gene therapy”, i.e., transfer of genes to a cell of the body which does not produce sperm or eggs. In certain embodiments, the transgenes express proteins have the sequence of native human sequences. However, in other embodiments, synthetic proteins are expressed. Such proteins may be intended for treatment of humans, or in other embodiments, designed for treatment of animals, including companion animals such as canine or feline populations, or for treatment of livestock or other animals which come into contact with human populations.

Examples of suitable gene products may include those associated with familial hypercholesterolemia, muscular dystrophy, cystic fibrosis, and rare or orphan diseases. Examples of such rare disease may include spinal muscular atrophy (SMA), Huntingdon's Disease, Rett Syndrome (e.g., methyl-CpG-binding protein 2 (MeCP2); UniProtKB-P51608), Amyotrophic Lateral Sclerosis (ALS), Duchenne Type Muscular dystrophy, Friedrichs Ataxia (e.g., frataxin), ATXN2 associated with spinocerebellar ataxia type 2 (SCA2)/ALS; TDP-43 associated with ALS, progranulin (PRGN) (associated with non-Alzheimer's cerebral degenerations, including, frontotemporal dementia (FTD), progressive non-fluent aphasia (PNFA) and semantic dementia), among others. See, e.g., orpha.net/consor/cgi-bin/Disease_Search_List.php; rarediseases.info.nih.gov/diseases. In one embodiment, the transgene is not human low-density lipoprotein receptor (hLDLR). In another embodiment, the transgene is not an engineered human low-density lipoprotein receptor (hLDLR) variant, such as those described in WO 2015/164778.

Examples of suitable genes may include, e.g., hormones and growth and differentiation factors including, without limitation, insulin, glucagon, glucagon-like peptide-1 (GLP1), growth hormone (GH), parathyroid hormone (PTH), growth hormone releasing factor (GRF), follicle stimulating hormone (FSH), luteinizing hormone (LH), human chorionic gonadotropin (hCG), vascular endothelial growth factor (VEGF), angiopoietins, angiostatin, granulocyte colony stimulating factor (GCSF), erythropoietin (EPO) (including, e.g., human, canine or feline epo), connective tissue growth factor (CTGF), neutrophic factors including, e.g., basic fibroblast growth factor (bFGF), acidic fibroblast growth factor (aFGF), epidermal growth factor (EGF), platelet-derived growth factor (PDGF), insulin growth factors I and II (IGF-I and IGF-II), any one of the transforming growth factor α superfamily, including TGFα, activins, inhibins, or any of the bone morphogenic proteins (BMP) BMPs 1-15, any one of the heregluin/neuregulin/ARIA/neu differentiation factor (NDF) family of growth factors, nerve growth factor (NGF), brain-derived neurotrophic factor (BDNF), neurotrophins NT-3 and NT-4/5, ciliary neurotrophic factor (CNTF), glial cell line derived neurotrophic factor (GDNF), neurturin, agrin, any one of the family of semaphorins/collapsins, netrin-1 and netrin-2, hepatocyte growth factor (HGF), ephrins, noggin, sonic hedgehog and tyrosine hydroxylase.

Other useful transgene products include proteins that regulate the immune system including, without limitation, cytokines and lymphokines such as thrombopoietin (TPO), interleukins (IL) IL-1 through IL-36 (including, e.g., human interleukins IL-1, IL-1α, IL-1β, IL-2, IL-3, IL-4, IL-6, IL-8, IL-12, IL-11, IL-12, IL-13, IL-18, IL-31, IL-35), monocyte chemoattractant protein, leukemia inhibitory factor, granulocyte-macrophage colony stimulating factor, Fas ligand, tumor necrosis factors α and β, interferons α, β, and γ, stem cell factor, flk-2/flt3 ligand. Gene products produced by the immune system are also useful in the invention. These include, without limitations, immunoglobulins IgG, IgM, IgA, IgD and IgE, chimeric immunoglobulins, humanized antibodies, single chain antibodies, T cell receptors, chimeric T cell receptors, single chain T cell receptors, class I and class II MHC molecules, as well as engineered immunoglobulins and MHC molecules. For example, in certain embodiments, the rAAV antibodies may be designed to delivery canine or feline antibodies, e.g., such as anti-IgE, anti-IL31, anti-IL33, anti-CD20, anti-NGF, anti-GnRH Useful gene products also include complement regulatory proteins such as complement regulatory proteins, membrane cofactor protein (MCP), decay accelerating factor (DAF), CR1, CF2, CD59, and C1 esterase inhibitor (C1-INH).

Still other useful gene products include any one of the receptors for the hormones, growth factors, cytokines, lymphokines, regulatory proteins and immune system proteins. The invention encompasses receptors for cholesterol regulation and/or lipid modulation, including the low-density lipoprotein (LDL) receptor, high density lipoprotein (HDL) receptor, the very low density lipoprotein (VLDL) receptor, and scavenger receptors. The invention also encompasses gene products such as members of the steroid hormone receptor superfamily including glucocorticoid receptors and estrogen receptors, Vitamin D receptors and other nuclear receptors. In addition, useful gene products include transcription factors such as jun, fos, max, mad, serum response factor (SRF), AP-1, AP2, myb, MyoD and myogenin, ETS-box containing proteins, TFE3, E2F, ATF1, ATF2, ATF3, ATF4, ZF5, NFAT, CREB, HNF-4, C/EBP, SP1, CCAAT-box binding proteins, interferon regulation factor (IRF-1), Wilms tumor protein, ETS-binding protein, STAT, GATA-box binding proteins, e.g., GATA-3, and the forkhead family of winged helix proteins.

Other useful gene products include hydroxymethylbilane synthase (HMBS), carbamoyl synthetase I, omithine transcarbamylase (OTC), arginosuccinate synthetase, arginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase, glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase E3A (UBE3A). Still useful gene products include UDP Glucuronosyltransferase Family 1 Member A1 (UGT1A1).

In certain embodiments, the rAAV may be used in gene editing systems, which system may involve one rAAV or co-administration of multiple rAAV stocks. For example, the rAAV may be engineered to deliver SpCas9, SaCas9, ARCUS, Cpf1 (also known as Cas12a), CjCas9, and other suitable gene editing constructs.

Still other useful gene products include those used for treatment of hemophilia, including hemophilia B (including Factor IX) and hemophilia A (including Factor VIII and its variants, such as the light chain and heavy chain of the heterodimer and the B-deleted domain; U.S. Pat. Nos. 6,200,560 and 6,221,349). In some embodiments, the minigene comprises first 57 base pairs of the Factor VIII heavy chain which encodes the 10 amino acid signal sequence, as well as the human growth hormone (hGH) polyadenylation sequence. In alternative embodiments, the minigene further comprises the A1 and A2 domains, as well as 5 amino acids from the N-terminus of the B domain, and/or 85 amino acids of the C-terminus of the B domain, as well as the A3, C1 and C2 domains. In yet other embodiments, the nucleic acids encoding Factor VIII heavy chain and light chain are provided in a single minigene separated by 42 nucleic acids coding for 14 amino acids of the B domain [U.S. Pat. No. 6,200,560].

Other useful gene products include non-naturally occurring polypeptides, such as chimeric or hybrid polypeptides having a non-naturally occurring amino acid sequence containing insertions, deletions, or amino acid substitutions. For example, single-chain engineered immunoglobulins could be useful in certain immunocompromised patients. Other types of non-naturally occurring gene sequences include antisense molecules and catalytic nucleic acids, such as ribozymes, which could be used to reduce overexpression of a target.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce “self”-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Further illustrative genes which may be delivered via the rAAV provided herein for treatment of, for example, liver indications include, without limitation, glucose-6-phosphatase, associated with glycogen storage disease or deficiency type TA (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with omithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; α-fucosidase associated with fucosidosis; α-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

Additional genes and diseases of interest include, e.g., dystonin gene related diseases such as Hereditary Sensory and Autonomic Neuropathy Type VI (the DST gene encodes dystonin; dual AAV vectors may be required due to the size of the protein (˜7570 aa); SCN9A related diseases, in which loss of function mutants cause inability to feel pain and gain of function mutants cause pain conditions, such as erythromelagia. Another condition is Charcot-Marie-Tooth (CMT) type 1F and 2E due to mutations in the NEFL gene (neurofilament light chain) characterized by a progressive peripheral motor and sensory neuropathy with variable clinical and electrophysiologic expression. Other gene products associated with CMT include mitofusin 2 (MFN2).

In certain embodiments, the rAAV described herein may be used in treatment of mucopolysaccaridoses (MPS) disorders. Such rAAV may contain carry a nucleic acid sequence encoding α-L-iduronidase (IDUA) for treating MPS I (Hurler, Hurler-Scheie and Scheie syndromes); a nucleic acid sequence encoding iduronate-2-sulfatase (IDS) for treating MPS II (Hunter syndrome); a nucleic acid sequence encoding sulfamidase (SGSH) for treating MPSIII A, B, C, and D (Sanfilippo syndrome); a nucleic acid sequence encoding N-acetylgalactosamine-6-sulfate sulfatase (GALNS) for treating MPS IV A and B (Morquio syndrome); a nucleic acid sequence encoding arylsulfatase B (ARSB) for treating MPS VI (Maroteaux-Lamy syndrome); a nucleic acid sequence encoding hyaluronidase for treating MPSI IX (hyaluronidase deficiency) and a nucleic acid sequence encoding beta-glucuronidase for treating MPS VII (Sly syndrome).

In some embodiments, an rAAV vector comprising a nucleic acid encoding a gene product associated with cancer (e.g., tumor suppressors) may be used to treat the cancer, by administering a rAAV harboring the rAAV vector to a subject having the cancer. In some embodiments, an rAAV vector comprising a nucleic acid encoding a small interfering nucleic acid (e.g., shRNAs, miRNAs) that inhibits the expression of a gene product associated with cancer (e.g., oncogenes) may be used to treat the cancer, by administering a rAAV harboring the rAAV vector to a subject having the cancer. In some embodiments, an rAAV vector comprising a nucleic acid encoding a gene product associated with cancer (or a functional RNA that inhibits the expression of a gene associated with cancer) may be used for research purposes, e.g., to study the cancer or to identify therapeutics that treat the cancer. The following is a non-limiting list of exemplary genes known to be associated with the development of cancer (e.g., oncogenes and tumor suppressors): AARS, ABCB1, ABCC4, ABI2, ABL1, ABL2, ACK1, ACP2, ACY1, ADSL, AK1, AKR1C2, AKT1, ALB, ANPEP, ANXA5, ANXA7, AP2M1, APC, ARHGAP5, ARHGEF5, ARID4A, ASNS, ATF4, ATM, ATP5B, ATP50, AXL, BARD1, BAX, BCL2, BHLHB2, BLMH, BRAF, BRCA1, BRCA2, BTK, CANX, CAP1, CAPN1, CAPNS1, CAV1, CBFB, CBLB, CCL2, CCND1, CCND2, CCND3, CCNE1, CCT5, CCYR61, CD24, CD44, CD59, CDC20, CDC25, CDC25A, CDC25B, CDC2L5, CDK10, CDK4, CDK5, CDK9, CDKL1, CDKN1A, CDKN1B, CDKN1C, CDKN2A, CDKN2B, CDKN2D, CEBPG, CENPC1, CGRRF1, CHAF1A, CIB1, CKMT1, CLK1, CLK2, CLK3, CLNS1A, CLTC, COL1A1, COL6A3, COX6C, COX7A2, CRAT, CRHR1, CSF1R, CSK, CSNK1G2, CTNNA1, CTNNB1, CTPS, CTSC, CTSD, CUL1, CYR61, DCC, DCN, DDX10, DEK, DHCR7, DHRS2, DHX8, DLG3, DVL1, DVL3, E2F1, E2F3, E2F5, EGFR, EGR1, EIF5, EPHA2, ERBB2, ERBB3, ERBB4, ERCC3, ETV1, ETV3, ETV6, F2R, FASTK, FBN1, FBN2, FES, FGFR1, FGR, FKBP8, FN1, FOS, FOSL1, FOSL2, FOXG1A, FOXO1A, FRAP1, FRZB, FTL, FZD2, FZD5, FZD9, G22P1, GAS6, GCN5L2, GDF15, GNA13, GNAS, GNB2, GNB2L1, GPR39, GRB2, GSK3A, GSPT1, GTF2I, HDAC1, HDGF, HMMR, HPRT1, HRB, HSPA4, HSPA5, HSPA8, HSPB1, HSPH1, HYAL1, HYOU1, ICAM1, ID1, ID2, IDUA, IER3, IFITM1, IGF1R, IGF2R, IGFBP3, IGFBP4, IGFBP5, IL1B, ILK, ING1, IRF3, ITGA3, ITGA6, ITGB4, JAKI, JARID1A, JUN, JUNB, JUND, K-ALPHA-1, KIT, KITLG, KLK10, KPNA2, KRAS2, KRT18, KRT2A, KRT9, LAMB1, LAMP2, LCK, LCN2, LEP, LITAF, LRPAP1, LTF, LYN, LZTR1, MADH1, MAP2K2, MAP3K8, MAPK12, MAPK13, MAPKAPK3, MAPRE1, MARS, MAS1, MCC, MCM2, MCM4, MDM2, MDM4, MET, MGST1, MICB, MLLT3, MME, MMP1, MMP14, MMP17, MMP2, MNDA, MSH2, MSH6, MT3, MYB, MYBL1, MYBL2, MYC, MYCL1, MYCN, MYD88, MYL9, MYLK, NEO1, NF1, NF2, NFKB1, NFKB2, NFSF7, NID, NINE, NMBR, NME1, NME2, NME3, NOTCH1, NOTCH2, NOTCH4, NPM1, NQO1, NR1D1, NR2F1, NR2F6, NRAS, NRG1, NSEP1, OSM, PA2G4, PABPC1, PCNA, PCTK1, PCTK2, PCTK3, PDGFA, PDGFB, PDGFRA, PDPK1, PEA15, PFDN4, PFDN5, PGAM1, PHB, PIK3CA, PIK3CB, PIK3CG, PIM1, PKM2, PKMYT1, PLK2, PPARD, PPARG, PPIH, PPP1CA, PPP2R5A, PRDX2, PRDX4, PRKAR1A, PRKCBP1, PRNP, PRSS15, PSMA1, PTCH, PTEN, PTGS1, PTMA, PTN, PTPRN, RAB5A, RAC1, RAD50, RAF1, RALBP1, RAP1A, RARA, RARB, RASGRF1, RB1, RBBP4, RBL2, REA, REL, RELA, RELB, RET, RFC2, RGS19, RHOA, RHOB, RHOC, RHOD, RIPK1, RPN2, RPS6 KB1, RRM1, SARS, SELENBP1, SEMA3C, SEMA4D, SEPP1, SERPINH1, SFN, SFPQ, SFRS7, SHB, SHH, SIAH2, SIVA, SIVA TP53, SKI, SKIL, SLC16A1, SLC1A4, SLC20A1, SMO, sphingomyelin phosphodiesterase 1 (SMPD1), SNAI2, SND1, SNRPB2, SOCS1, SOCS3, SOD1, SORT1, SPINT2, SPRY2, SRC, SRPX, STAT1, STAT2, STAT3, STAT5B, STC1, TAF1, TBL3, TBRG4, TCF1, TCF7L2, TFAP2C, TFDP1, TFDP2, TGFA, TGFB1, TGFB1, TGFBR2, TGFBR3, THBS1, TIE, TIMP1, TIMP3, TJP1, TK1, TLE1, TNF, TNFRSF10A, TNFRSF10B, TNFRSF1A, TNFRSF1B, TNFRSF6, TNFSF7, TNK1, TOB1, TP53, TP53BP2, TP5313, TP73, TPBG, TPT1, TRADD, TRAM1, TRRAP, TSG101, TUFM, TXNRD1, TYRO3, UBC, UBE2L6, UCHL1, USP7, VDAC1, VEGF, VHL, VIL2, WEE1, WNT1, WNT2, WNT2B, WNT3, WNT5A, WT1, XRCC1, YES1, YWHAB, YWHAZ, ZAP70, and ZNF9.

A rAAV vector may comprise as a transgene, a nucleic acid encoding a protein or functional RNA that modulates apoptosis. The following is a non-limiting list of genes associated with apoptosis and nucleic acids encoding the products of these genes and their homologues and encoding small interfering nucleic acids (e.g., shRNAs, miRNAs) that inhibit the expression of these genes and their homologues are useful as transgenes in certain embodiments of the invention: RPS27A, ABL1, AKT1, APAF1, BAD, BAG1, BAG3, BAG4, BAK1, BAX, BCL10, BCL2, BCL2A1, BCL2L1, BCL2L10, BCL2L11, BCL2L12, BCL2L13, BCL2L2, BCLAF1, BFAR, BID, BIK, NAIP, BIRC2, BIRC3, XIAP, BIRC5, BIRC6, BIRC7, BIRC8, BNIP1, BNIP2, BNIP3, BNIP3L, BOK, BRAF, CARD10, CARD11, NLRC4, CARD14, NOD2, NOD1, CARD6, CARDS, CARDS, CASP1, CASP10, CASP14, CASP2, CASP3, CASP4, CASP5, CASP6, CASP7, CASP8, CASP9, CFLAR, CIDEA, CIDEB, CRADD, DAPK1, DAPK2, DFFA, DFFB, FADD, GADD45A, GDNF, HRK, IGF1R, LTA, LTBR, MCL1, NOL3, PYCARD, RIPK1, RIPK2, TNF, TNFRSF10A, TNFRSF10B, TNFRSF10C, TNFRSF10D, TNFRSF11B, TNFRSF12A, TNFRSF14, TNFRSF19, TNFRSF1A, TNFRSF1B, TNFRSF21, TNFRSF25, CD40, FAS, TNFRSF6B, CD27, TNFRSF9, TNFSF10, TNFSF14, TNFSF18, CD40LG, FASLG, CD70, TNFSF8, TNFSF9, TP53, TP53BP2, TP73, TP63, TRADD, TRAF1, TRAF2, TRAF3, TRAF4, and TRAF5.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-71, hsa-let-71*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, hsa-miR-136*, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-296-1*, hsa-miR-296-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-5486-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-5481, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*. For example, miRNA targeting chromosome 8 open reading frame 72 (C9orf72) which expresses superoxide dismutase (SOD1), associated with amyotrophic lateral sclerosis (ALS) may be of interest.

A miRNA inhibits the function of the mRNAs it targets and, as a result, inhibits expression of the polypeptides encoded by the mRNAs. Thus, blocking (partially or totally) the activity of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary with the miRNA at all but 1, 2, 3, 4, 5, 6, 7, 8, 9, 10, 11, 12, 13, 14, 15, 16, 17, or 18 bases. A “miRNA Inhibitor” is an agent that blocks miRNA function, expression and/or processing. For instance, these molecules include but are not limited to microRNA specific antisense, microRNA sponges, tough decoy RNAs (TuD RNAs) and microRNA oligonucleotides (double-stranded, hairpin, short oligonucleotides) that inhibit miRNA interaction with a Drosha complex.

Still other useful transgenes may include those encoding immunoglobulins which confer passive immunity to a pathogen. An “immunoglobulin molecule” is a protein containing the immunologically-active portions of an immunoglobulin heavy chain and immunoglobulin light chain covalently coupled together and capable of specifically combining with antigen. Immunoglobulin molecules are of any type (e.g., IgG, IgE, IgM, IgD, IgA and IgY), class (e.g., IgG1, IgG2, IgG3, IgG4, IgA1 and IgA2) or subclass. The terms “antibody” and “immunoglobulin” may be used interchangeably herein.

An “immunoglobulin heavy chain” is a polypeptide that contains at least a portion of the antigen binding domain of an immunoglobulin and at least a portion of a variable region of an immunoglobulin heavy chain or at least a portion of a constant region of an immunoglobulin heavy chain. Thus, the immunoglobulin derived heavy chain has significant regions of amino acid sequence homology with a member of the immunoglobulin gene superfamily. For example, the heavy chain in a Fab fragment is an immunoglobulin-derived heavy chain.

An “immunoglobulin light chain” is a polypeptide that contains at least a portion of the antigen binding domain of an immunoglobulin and at least a portion of the variable region or at least a portion of a constant region of an immunoglobulin light chain. Thus, the immunoglobulin-derived light chain has significant regions of amino acid homology with a member of the immunoglobulin gene superfamily.

An “immunoadhesin” is a chimeric, antibody-like molecule that combines the functional domain of a binding protein, usually a receptor, ligand, or cell-adhesion molecule, with immunoglobulin constant domains, usually including the hinge and Fc regions.

A “fragment antigen-binding” (Fab) fragment” is a region on an antibody that binds to antigens. It is composed of one constant and one variable domain of each of the heavy and the light chain.

The anti-pathogen construct is selected based on the causative agent (pathogen) for the disease against which protection is sought. These pathogens may be of viral, bacterial, or fungal origin, and may be used to prevent infection in humans against human disease, or in non-human mammals or other animals to prevent veterinary disease.

The rAAV may include genes encoding antibodies, and particularly neutralizing antibodies against a viral pathogen. Such anti-viral antibodies may include anti-influenza antibodies directed against one or more of Influenza A, Influenza B, and Influenza C. The type A viruses are the most virulent human pathogens. The serotypes of influenza A which have been associated with pandemics include, H1N1, which caused Spanish Flu in 1918, and Swine Flu in 2009; H2N2, which caused Asian Flu in 1957; H3N2, which caused Hong Kong Flu in 1968; H5N1, which caused Bird Flu in 2004; H7N7; H1N2; H9N2; H7N2; H7N3; and H10N7. Other target pathogenic viruses include, arenaviruses (including funin, machupo, and Lassa), filoviruses (including Marburg and Ebola), hantaviruses, picornoviridae (including rhinoviruses, echovirus), coronaviruses, paramyxovirus, morbillivirus, respiratory synctial virus, togavirus, coxsackievirus, JC virus, parvovirus B19, parainfluenza, adenoviruses, reoviruses, variola (Variola major (Smallpox)) and Vaccinia (Cowpox) from the poxvirus family, and varicella-zoster (pseudorabies). Viral hemorrhagic fevers are caused by members of the arenavirus family (Lassa fever) (which family is also associated with Lymphocytic choriomeningitis (LCM)), filovirus (ebola virus), and hantavirus (puremala). The members of picomavirus (a subfamily of rhinoviruses), are associated with the common cold in humans. The coronavirus family, which includes a number of non-human viruses such as infectious bronchitis virus (poultry), porcine transmissible gastroenteric virus (pig), porcine hemagglutinatin encephalomyelitis virus (pig), feline infectious peritonitis virus (cat), feline enteric coronavirus (cat), canine coronavirus (dog). The human respiratory coronaviruses, have been putatively associated with the common cold, non-A, B or C hepatitis, and sudden acute respiratory syndrome (SARS). The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus (RSV). The parvovirus family includes feline parvovirus (feline enteritis), feline panleucopeniavirus, canine parvovirus, and porcine parvovirus. The adenovirus family includes viruses (EX, AD7, ARD, O.B.) which cause respiratory disease. Thus, in certain embodiments, a rAAV vector as described herein may be engineered to express an anti-ebola antibody, e.g., 2G4, 4G7, 13C6, an anti-influenza antibody, e.g., FI6, CR8033, and anti-RSV antibody, e.g, palivizumab, motavizumab. A neutralizing antibody construct against a bacterial pathogen may also be selected for use in the present invention. In one embodiment, the neutralizing antibody construct is directed against the bacteria itself. In another embodiment, the neutralizing antibody construct is directed against a toxin produced by the bacteria. Examples of airborne bacterial pathogens include, e.g., Neisseria meningitidis (meningitis), Klebsiella pneumonia (pneumonia), Pseudomonas aeruginosa (pneumonia), Pseudomonas pseudomallei (pneumonia), Pseudomonas mallei (pneumonia), Acinetobacter (pneumonia), Moraxella catarrhalis, Moraxella lacunata, Alkaligenes, Cardiobacterium, Haemophilus influenzae (flu), Haemophilus parainfluenzae, Bordetella pertussis (whooping cough), Francisella tularensis (pneumonia/fever), Legionella pneumonia (Legionnaires disease), Chlamydia psittaci (pneumonia), Chlamydia pneumoniae (pneumonia), Mycobacterium tuberculosis (tuberculosis (TB)), Mycobacterium kansasii (TB), Mycobacterium avium (pneumonia), Nocardia asteroides (pneumonia), Bacillus anthracis (anthrax), Staphylococcus aureus (pneumonia), Streptococcus pyogenes (scarlet fever), Streptococcus pneumoniae (pneumonia), Corynebacteria diphtheria (diphtheria), Mycoplasma pneumoniae (pneumonia).

The rAAV may include genes encoding antibodies, and particularly neutralizing antibodies against a bacterial pathogen such as the causative agent of anthrax, a toxin produced by Bacillius anthracis. Neutralizing antibodies against protective agent (PA), one of the three peptides which form the toxoid, have been described. The other two polypeptides consist of lethal factor (LF) and edema factor (EF). Anti-PA neutralizing antibodies have been described as being effective in passively immunization against anthrax. See, e.g., U.S. Pat. No. 7,442,373; R. Sawada-Hirai et al, J Immune Based Ther Vaccines. 2004; 2: 5. (on-line 2004 May 12). Still other anti-anthrax toxin neutralizing antibodies have been described and/or may be generated. Similarly, neutralizing antibodies against other bacteria and/or bacterial toxins may be used to generate an AAV-delivered anti-pathogen construct as described herein.

Antibodies against infectious diseases may be caused by parasites or by fungi, including, e.g., Aspergillus species, Absidia corymbifera, Rhixpus stolonifer, Mucor plumbeaus, Cryptococcus neoformans, Histoplasm capsulatum, Blastomyces dermatitidis, Coccidioides immitis, Penicillium species, Micropolyspora faeni, Thermoactinomyces vulgaris, Alternaria alternate, Cladosporium species, Helminthosporium, and Stachybotrys species.

The rAAV may include genes encoding antibodies, and particularly neutralizing antibodies, against pathogenic factors of diseases such as Alzheimer's disease (AD), Parkinson's disease (PD), GBA-associated-Parkinson's disease (GBA-PD), Rheumatoid arthritis (RA), Irritable bowel syndrome (IBS), chronic obstructive pulmonary disease (COPD), cancers, tumors, systemic sclerosis, asthma and other diseases. Such antibodies may be., without limitation, e.g., alpha-synuclein, anti-vascular endothelial growth factor (VEGF) (anti-VEGF), anti-VEGFA, anti-PD-1, anti-PDL1, anti-CTLA-4, anti-TNF-alpha, anti-IL-17, anti-IL-23, anti-IL-21, anti-IL-6, anti-IL-6 receptor, anti-IL-5, anti-IL-7, anti-Factor XII, anti-IL-2, anti-HIV, anti-IgE, anti-tumour necrosis factor receptor-1 (TNFR1), anti-notch 2/3, anti-notch 1, anti-OX40, anti-erb-b2 receptor tyrosine kinase 3 (ErbB3), anti-ErbB2, anti-beta cell maturation antigen, anti-B lymphocyte stimulator, anti-CD20, anti-HER2, anti-granulocyte macrophage colony-stimulating factor, anti-oncostatin M (OSM), anti-lymphocyte activation gene 3 (LAG3) protein, anti-CCL20, anti-serum amyloid P component (SAP), anti-prolyl hydroxylase inhibitor, anti-CD38, anti-glycoprotein IIb/IIIa, anti-CD52, anti-CD30, anti-IL-ibeta, anti-epidermal growth factor receptor, anti-CD25, anti-RANK ligand, anti-complement system protein C5, anti-CD11a, anti-CD3 receptor, anti-alpha-4 (α4) integrin, anti-RSV F protein, and anti-integrin α₄β₇. Still other pathogens and diseases will be apparent to one of skill in the art. Other suitable antibodies may include those useful for treating Alzheimer's Disease, such as, e.g., anti-beta-amyloid (e.g., crenezumab, solanezumab, aducanumab), anti-beta-amyloid fibril, anti-beta-amyloid plaques, anti-tau, a bapineuzamab, among others. Other suitable antibodies for treating a variety of indications include those described, e.g., in PCT/US2016/058968, filed 27 Oct. 2016, published as WO 2017/075119A1.

Reduction and/or modulation of expression of a gene is particularly desirable for treatment of hyperproliferative conditions characterized by hyperproliferating cells, as are cancers and psoriasis. Target polypeptides include those polypeptides which are produced exclusively or at higher levels in hyperproliferative cells as compared to normal cells. Target antigens include polypeptides encoded by oncogenes such as myb, myc, fyn, and the translocation gene bcr/abl, ras, src, P53, neu, trk and EGRF. In addition to oncogene products as target antigens, target polypeptides for anti-cancer treatments and protective regimens include variable regions of antibodies made by B cell lymphomas and variable regions of T cell receptors of T cell lymphomas which, in some embodiments, are also used as target antigens for autoimmune disease. Other tumor-associated polypeptides can be used as target polypeptides such as polypeptides which are found at higher levels in tumor cells including the polypeptide recognized by monoclonal antibody 17-1A and folate binding polypeptides.

Other suitable therapeutic polypeptides and proteins include those which may be useful for treating individuals suffering from autoimmune diseases and disorders by conferring a broad based protective immune response against targets that are associated with autoimmunity including cell receptors and cells which produce self-directed antibodies. T cell mediated autoimmune diseases include Rheumatoid arthritis (RA), multiple sclerosis (MS), Sjögren's syndrome, sarcoidosis, insulin dependent diabetes mellitus (IDDM), autoimmune thyroiditis, reactive arthritis, ankylosing spondylitis, scleroderma, polymyositis, dermatomyositis, psoriasis, vasculitis, Wegener's granulomatosis, Crohn's disease and ulcerative colitis. Each of these diseases is characterized by T cell receptors (TCRs) that bind to endogenous antigens and initiate the inflammatory cascade associated with autoimmune diseases.

Alternatively, or in addition, the vectors may contain AAV sequences of the invention and a transgene encoding a peptide, polypeptide or protein which induces an immune response to a selected immunogen. For example, immunogens may be selected from a variety of viral families. Example of desirable viral families against which an immune response would be desirable include, the picornavirus family, which includes the genera rhinoviruses, which are responsible for about 50% of cases of the common cold; the genera enteroviruses, which include polioviruses, coxsackieviruses, echoviruses, and human enteroviruses such as hepatitis A virus; and the genera apthoviruses, which are responsible for foot and mouth diseases, primarily in non-human animals. Within the picornavirus family of viruses, target antigens include the VP1, VP2, VP3, VP4, and VPG. Another viral family includes the calcivirus family, which encompasses the Norwalk group of viruses, which are an important causative agent of epidemic gastroenteritis. Still another viral family desirable for use in targeting antigens for inducing immune responses in humans and non-human animals is the togavirus family, which includes the genera alphavirus, which include Sindbis viruses, RossRiver virus, and Venezuelan, Eastern & Western Equine encephalitis, and rubivirus, including Rubella virus. The flaviviridae family includes dengue, yellow fever, Japanese encephalitis, St. Louis encephalitis and tick borne encephalitis viruses. Other target antigens may be generated from the Hepatitis C or the coronavirus family, which includes a number of non-human viruses such as infectious bronchitis virus (poultry), porcine transmissible gastroenteric virus (pig), porcine hemagglutinating encephalomyelitis virus (pig), feline infectious peritonitis virus (cats), feline enteric coronavirus (cat), canine coronavirus (dog), and human respiratory coronaviruses, which may cause the common cold and/or non-A, B or C hepatitis. Within the coronavirus family, target antigens include the E1 (also called M or matrix protein), E2 (also called S or Spike protein), E3 (also called HE or hemagglutin-elterose) glycoprotein (not present in all coronaviruses), or N (nucleocapsid). Still other antigens may be targeted against the rhabdovirus family, which includes the genera vesiculovirus (e.g., Vesicular Stomatitis Virus), and the general lyssavirus (e.g., rabies). Within the rhabdovirus family, suitable antigens may be derived from the G protein or the N protein. The family filoviridae, which includes hemorrhagic fever viruses such as Marburg and Ebola virus may be a suitable source of antigens. The paramyxovirus family includes parainfluenza Virus Type 1, parainfluenza Virus Type 3, bovine parainfluenza Virus Type 3, rubulavirus (mumps virus, parainfluenza Virus Type 2, parainfluenza virus Type 4, Newcastle disease virus (chickens), rinderpest, morbillivirus, which includes measles and canine distemper, and pneumovirus, which includes respiratory syncytial virus. The influenza virus is classified within the family orthomyxovirus and is a suitable source of antigen (e.g., the HA protein, the N1 protein). The bunyavirus family includes the genera bunyavirus (California encephalitis, La Crosse), phlebovirus (Rift Valley Fever), hantavirus (puremala is a hemahagin fever virus), nairovirus (Nairobi sheep disease) and various unassigned bungaviruses. The arenavirus family provides a source of antigens against LCM and Lassa fever virus. The reovirus family includes the genera reovirus, rotavirus (which causes acute gastroenteritis in children), orbiviruses, and cultivirus (Colorado Tick fever, Lebombo (humans), equine encephalosis, blue tongue).

The retrovirus family includes the sub-family oncorivirinal which encompasses such human and veterinary diseases as feline leukemia virus, HTLVI and HTLVII, lentivirinal (which includes human immunodeficiency virus (HIV), simian immunodeficiency virus (SIV), feline immunodeficiency virus (FIV), equine infectious anemia virus, and spumavirinal). Between the HIV and SIV, many suitable antigens have been described and can readily be selected. Examples of suitable HIV and SIV antigens include, without limitation the gag, pol, Vif, Vpx, VPR, Env, Tat and Rev proteins, as well as various fragments thereof. In addition, a variety of modifications to these antigens have been described. Suitable antigens for this purpose are known to those of skill in the art. For example, one may select a sequence encoding the gag, pol, Vif, and Vpr, Env, Tat and Rev, amongst other proteins. See, e.g., the modified gag protein which is described in U.S. Pat. No. 5,972,596. See, also, the HIV and SIV proteins described in D. H. Barouch et al, J. Virol., 75(5):2462-2467 (March 2001), and R. R. Amara, et al, Science, 292:69-74 (6 Apr. 2001). These proteins or subunits thereof may be delivered alone, or in combination via separate vectors or from a single vector.

The papovavirus family includes the sub-family polyomaviruses (BKU and JCU viruses) and the sub-family papillomavirus (associated with cancers or malignant progression of papilloma). The adenovirus family includes viruses (EX, AD7, ARD, O.B.) which cause respiratory disease and/or enteritis. The parvovirus family feline parvovirus (feline enteritis), feline panleucopeniavirus, canine parvovirus, and porcine parvovirus. The herpesvirus family includes the sub-family alphaherpesvirinae, which encompasses the genera simplexvirus (HSVI, HSVII), varicellovirus (pseudorabies, varicella zoster) and the sub-family betaherpesvirinae, which includes the genera cytomegalovirus (HCMV, muromegalovirus) and the sub-family gammaherpesvirinae, which includes the genera lymphocryptovirus, EBV (Burkitts lymphoma), infectious rhinotracheitis, Marek's disease virus, and rhadinovirus. The poxvirus family includes the sub-family chordopoxvirinae, which encompasses the genera orthopoxvirus (Variola (Smallpox) and Vaccinia (Cowpox)), parapoxvirus, avipoxvirus, capripoxvirus, leporipoxvirus, suipoxvirus, and the sub-family entomopoxvirinae. The hepadnavirus family includes the Hepatitis B virus. One unclassified virus which may be suitable source of antigens is the Hepatitis delta virus. Still other viral sources may include avian infectious bursal disease virus and porcine respiratory and reproductive syndrome virus. The alphavirus family includes equine arteritis virus and various Encephalitis viruses.

The rAAV may also deliver a sequence encoding immunogens which are useful to immunize a human or non-human animal against other pathogens including bacteria, fungi, parasitic microorganisms or multicellular parasites which infect human and non-human vertebrates, or from a cancer cell or tumor cell. Examples of bacterial pathogens include pathogenic gram-positive cocci include pneumococci; staphylococci; and streptococci. Pathogenic gram-negative cocci include meningococcus; gonococcus. Pathogenic enteric gram-negative bacilli include enterobacteriaceae; pseudomonas, acinetobacteria and eikenella; melioidosis; salmonella; shigella; haemophilus; moraxella; H. ducreyi (which causes chancroid); brucella; Franisella tularensis (which causes tularemia); yersinia (pasteurella); streptobacillus moniliformis and spirillum; Gram-positive bacilli include Listeria monocytogenes; Erysipelothrix rhusiopathiae; Corynebacterium diphtheria (diphtheria); cholera; B. anthracis (anthrax); donovanosis (granuloma inguinale); and bartonellosis. Diseases caused by pathogenic anaerobic bacteria include tetanus; botulism; other clostridia; tuberculosis; leprosy; and other mycobacteria. Pathogenic spirochetal diseases include syphilis; treponematoses: yaws, pinta and endemic syphilis; and leptospirosis. Other infections caused by higher pathogen bacteria and pathogenic fungi include actinomycosis; nocardiosis; cryptococcosis, blastomycosis, histoplasmosis and coccidioidomycosis; candidiasis, aspergillosis, and mucormycosis; sporotrichosis; paracoccidiodomycosis, petriellidiosis, torulopsosis, mycetoma and chromomycosis; and dermatophytosis. Rickettsial infections include Typhus fever, Rocky Mountain spotted fever, Q fever, and Rickettsialpox. Examples of mycoplasma and chlamydial infections include: Mycoplasma pneumoniae; lymphogranuloma venereum; psittacosis; and perinatal chlamydial infections. Pathogenic eukaryotes encompass pathogenic protozoans and helminths and infections produced thereby include: amebiasis; malaria; leishmaniasis; trypanosomiasis; toxoplasmosis; Pneumocystis carinii; Trichans; Toxoplasma gondii; babesiosis; giardiasis; trichinosis; filariasis; schistosomiasis; nematodes; trematodes or flukes; and cestode (tapeworm) infections.

Many of these organisms and/or toxins produced thereby have been identified by the Centers for Disease Control [(CDC), Department of Health and Human Services, USA], as agents which have potential for use in biological attacks. For example, some of these biological agents, include, Bacillus anthracis (anthrax), Clostridium botulinum and its toxin (botulism), Yersinia pestis (plague), variola major (smallpox), Francisella tularensis (tularemia), and viral hemorrhagic fever, all of which are currently classified as Category A agents; Coxiella burnetti (Q fever); Brucella species (brucellosis), Burkholderia mallei (glanders), Ricinus communis and its toxin (ricin toxin), Clostridium perfringens and its toxin (epsilon toxin), Staphylococcus species and their toxins (enterotoxin B), all of which are currently classified as Category B agents; and Nipan virus and hantaviruses, which are currently classified as Category C agents. In addition, other organisms, which are so classified or differently classified, may be identified and/or used for such a purpose in the future. It will be readily understood that the viral vectors and other constructs described herein are useful to deliver antigens from these organisms, viruses, their toxins or other by-products, which will prevent and/or treat infection or other adverse reactions with these biological agents.

Administration of the vectors of the invention to deliver immunogens against the variable region of the T cells elicit an immune response including CTLs to eliminate those T cells. In rheumatoid arthritis (RA), several specific variable regions of T cell receptors (TCRs) which are involved in the disease have been characterized. These TCRs include V-3, V-14, V-17 and Vα-17. Thus, delivery of a nucleic acid sequence that encodes at least one of these polypeptides will elicit an immune response that will target T cells involved in RA. In multiple sclerosis (MS), several specific variable regions of TCRs which are involved in the disease have been characterized. These TCRs include V-7 and Vα-10. Thus, delivery of a nucleic acid sequence that encodes at least one of these polypeptides will elicit an immune response that will target T cells involved in MS. In scleroderma, several specific variable regions of TCRs which are involved in the disease have been characterized. These TCRs include V-6, V-8, V-14 and Vα-16, Vα-3C, Vα-7, Vα-14, Vα-15, Vα-16, Vα-28 and Vα-12. Thus, delivery of a nucleic acid molecule that encodes at least one of these polypeptides will elicit an immune response that will target T cells involved in scleroderma.

In one embodiment, the transgene is selected to provide optogenetic therapy. In optogenetic therapy, artificial photoreceptors are constructed by gene delivery of light-activated channels or pumps to surviving cell types in the remaining retinal circuit. This is particularly useful for patients who have lost a significant amount of photoreceptor function, but whose bipolar cell circuitry to ganglion cells and optic nerve remains intact. In one embodiment, the heterologous nucleic acid sequence (transgene) is an opsin. The opsin sequence can be derived from any suitable single- or multicellular-organism, including human, algae and bacteria. In one embodiment, the opsin is rhodopsin, photopsin, L/M wavelength (red/green)-opsin, or short wavelength (S) opsin (blue). In another embodiment, the opsin is channelrhodopsin or halorhodopsin.

In another embodiment, the transgene is selected for use in gene augmentation therapy, i.e., to provide replacement copy of a gene that is missing or defective. In this embodiment, the transgene may be readily selected by one of skill in the art to provide the necessary replacement gene. In one embodiment, the missing/defective gene is related to an ocular disorder. In another embodiment, the transgene is NYX, GRM6, TRPM1L or GPR179 and the ocular disorder is Congenital Stationary Night Blindness. See, e.g., Zeitz et al, Am J Hum Genet. 2013 Jan. 10; 92(1):67-75. Epub 2012 Dec. 13 which is incorporated herein by reference. In another embodiment, the transgene is RPGR. In another embodiment, the gene is Rab escort protein 1 (REP-1) encoded by CHM, associated with choroideremia.

In another embodiment, the transgene is selected for use in gene suppression therapy, i.e., expression of one or more native genes is interrupted or suppressed at transcriptional or translational levels. This can be accomplished using short hairpin RNA (shRNA) or other techniques well known in the art. See, e.g., Sun et al, Int J Cancer. 2010 Feb. 1; 126(3):764-74 and O'Reilly M, et al. Am J Hum Genet. 2007 July; 81(1):127-35, which are incorporated herein by reference. In this embodiment, the transgene may be readily selected by one of skill in the art based upon the gene which is desired to be silenced.

In another embodiment, the transgene comprises more than one transgene. This may be accomplished using a single vector carrying two or more heterologous sequences, or using two or more rAAV each carrying one or more heterologous sequences. In one embodiment, the rAAV is used for gene suppression (or knockdown) and gene augmentation co-therapy. In knockdown/augmentation co-therapy, the defective copy of the gene of interest is silenced and a non-mutated copy is supplied. In one embodiment, this is accomplished using two or more co-administered vectors. See, Millington-Ward et al, Molecular Therapy, April 2011, 19(4):642-649 which is incorporated herein by reference. The transgenes may be readily selected by one of skill in the art based on the desired result.

In another embodiment, the transgene is selected for use in gene correction therapy. This may be accomplished using, e.g., a zinc-finger nuclease (ZFN)-induced DNA double-strand break in conjunction with an exogenous DNA donor substrate. See, e.g., Ellis et al, Gene Therapy (epub January 2012) 20:35-42 which is incorporated herein by reference. In one embodiment, the transgene encodes a nuclease selected from a meganuclease, a zinc finger nuclease, a transcription activator-like (TAL) effector nuclease (TALEN), and a clustered, regularly interspaced short palindromic repeat (CRISPR)/endonuclease (Cas9, Cpf1, etc). Examples of suitable meganucleases are described, e.g., in U.S. Pat. Nos. 8,445,251; 9,340,777; 9,434,931; 9,683,257, and WO 2018/195449. Other suitable enzymes include nuclease-inactive S. pyogenes CRISPR/Cas9 that can bind RNA in a nucleic-acid-programmed manner (Nelles et al, Programmable RNA Tracking in Live Cells with CRISPR/Cas9, Cell, 165(2):P488-96 (April 2016)), and base editors (e.g., Levy et al. Cytosine and adenine base editing of the brain, liver, retina, heart and skeletal muscle of mice via adeno-associated viruses, Nature Biomedical Engineering, 4, 97-110 (January 2020)). In certain embodiments, the nuclease is not a zinc finger nuclease. In certain embodiments, the nuclease is not a CRISPR-associated nuclease. In certain embodiments, the nuclease is not a TALEN. In one embodiment, the nuclease is not a meganuclease. In certain embodiments, the nuclease is a member of the LAGLIDADG (SEQ ID NO: 45) family of homing endonucleases. In certain embodiments, the nuclease is a member of the I-CreI family of homing endonucleases which recognizes and cuts a 22 base pair recognition sequence SEQ ID NO: 46—CAAAACGTCGTGAGACAGTTTG. See, e.g., WO 2009/059195. Methods for rationally-designing mono-LAGLIDADG homing endonucleases were described which are capable of comprehensively redesigning ICreI and other homing endonucleases to target widely-divergent DNA sites, including sites in mammalian, yeast, plant, bacterial, and viral genomes (WO 2007/047859).

In certain embodiments, a rAAV-based gene editing nuclease system is provided herein. The gene editing nuclease targets sites in a disease-associated gene, i.e., gene of interest.

In certain embodiments, the AAV-based gene editing nuclease system comprises an rAAV comprising an AAV capsid and enclosed therein a vector genome, wherein the vector genome comprising AAV 5′ inverted terminal repeats (ITR), an expression cassette comprising a nucleic acid sequence encoding a gene editing nuclease which recognizes and cleaves a recognition site in a gene of interest, wherein said gene editing nuclease coding sequence is operably linked to expression control sequences which direct expression thereof in a cell comprising the gene of interest, and an AAV 3′ ITR. In certain embodiments, the rAAV-based gene editing nuclease system is an rAAVhu71/74-based gene editing nuclease system. In certain embodiments, the rAAV-based gene editing nuclease system is an rAAVhu79-based gene editing nuclease system. In certain embodiments, the rAAV-based gene editing nuclease system is an rAAVhu80-based gene editing nuclease system. In certain embodiments, the rAAV-based gene editing nuclease system is an rAAVhu83-based gene editing nuclease system. In certain embodiments, the rAAV-based gene editing nuclease system is an rAAVhu74/71-based gene editing nuclease system. In certain embodiments, the rAAV-based gene editing nuclease system is an rAAVhu77-based gene editing nuclease system. In certain embodiments, the rAAV-based gene editing nuclease system is an rAAVhu78/88-based gene editing nuclease system. In certain embodiments, the rAAV-based gene editing nuclease system is an rAAVhu70-based gene editing nuclease system. In certain embodiments, the rAAV-based gene editing nuclease system is an rAAVhu72-based gene editing nuclease system. In certain embodiments, the rAAV-based gene editing nuclease system is an rAAVhu75-based gene editing nuclease system. In certain embodiments, the rAAV-based gene editing nuclease system is an rAAVhu76-based gene editing nuclease system. In certain embodiments, the rAAV-based gene editing nuclease system is an rAAVhu81-based gene editing nuclease system. In certain embodiments, the rAAV-based gene editing nuclease system is an rAAVhu82-based gene editing nuclease system. In certain embodiments, the rAAV-based gene editing nuclease system is an rAAVhu84-based gene editing nuclease system. In certain embodiments, the rAAV-based gene editing nuclease system is an rAAVhu86-based gene editing nuclease system. In certain embodiments, the rAAV-based gene editing nuclease system is an rAAVhu87-based gene editing nuclease system. In certain embodiments, the rAAV-based gene editing nuclease system is an rAAVhu88/78-based gene editing nuclease system. In certain embodiments, the rAAV-based gene editing nuclease system is an rAAVhu69-based gene editing nuclease system. In certain embodiments, the rAAV-based gene editing nuclease system is an rAAVrh75-based gene editing nuclease system. In certain embodiments, the rAAV-based gene editing nuclease system is an rAAVrh76-based gene editing nuclease system. In certain embodiments, the rAAV-based gene editing nuclease system is an rAAVrh77-based gene editing nuclease system. In certain embodiments, the rAAV-based gene editing nuclease system is an rAAVrh78-based gene editing nuclease system. In certain embodiments, the rAAV-based gene editing nuclease system is an rAAVrh79-based gene editing nuclease system. In certain embodiments, the rAAV-based gene editing nuclease system is an rAAVrh81-based gene editing nuclease system. In certain embodiments, the rAAV-based gene editing nuclease system is an rAAVrh89-based gene editing nuclease system. In certain embodiments, the rAAV-based gene editing nuclease system is an rAAVrh82-based gene editing nuclease system. In certain embodiments, the rAAV-based gene editing nuclease system is an rAAVrh83-based gene editing nuclease system. In certain embodiments, the rAAV-based gene editing nuclease system is an rAAVrh84-based gene editing nuclease system. In certain embodiments, the rAAV-based gene editing nuclease system is an rAAVrh85-based gene editing nuclease system. In certain embodiments, the rAAV-based gene editing nuclease system is an rAAVrh87-based gene editing nuclease system. In certain embodiments, the rAAV-based gene editing nuclease system is an rAAVhu73-based gene editing nuclease system.

Provided herein also is a method of treatment using an rAAV-based gene editing nuclease system.

In some embodiments, the rAAV-based gene editing meganuclease system is used for treating diseases, disorders, syndrome and/or conditions. In some embodiments, the gene editing nuclease is targeted to a gene of interest, wherein the gene of interest has one or more genetic mutation, deletion, insertion, and/or a defect which is associated with and/or implicated in a disease, disorder, syndrome and/or conditions. In some embodiments, the disorder is selected but not limited to cardiovascular, hepatic, endocrine or metabolic, musculoskeletal, neurological, and/or renal disorders.

In certain embodiments, the indicated cardiovascular diseases, disorders, syndrome and/or conditions include, but not limited to, cardiovascular disease (associated lysophosphatidic acid, lipoprotein (a), or angiopoietin-like 3 (ANGPTL3), or apolipoprotein C-III (APOC3) encoding genes), block coagulation, thrombosis, end stage renal disease, clotting disorders (associated with Factor XI (F11) encoding gene), hypertension (angiotensinogen (AGT) encoding gene), and heart failure (angiotensinogen (AGT) encoding gene).

In certain embodiments, the indicated hepatic diseases, disorders, syndrome and/or conditions include, but not limited to, idiopathic pulmonary fibrosis (associated with SERPINH1/Hsp47 gene), liver disease (associated with hydroxysteroid 17-beta dehydrogenase 13 (HSD17B13) encoding gene, non-alcoholic steatohepatitis (NASH) (associated with diacylglycerol O-acyltransferase-2 (DGAT2), hydroxysteroid 17-Beta Dehydrogenase 13 (HSD17B13), or patatin-like phospholipase domain-containing 3 (PNPLA3) encoding genes), and alcohol use disorder (associated with aldehyde dehydrogenase 2 (ALDH2) encoding gene).

In certain embodiments, the indicated musculoskeletal diseases, disorders, syndrome and/or conditions include, but not limited to, muscular dystrophy (associated with dystrophin, or integrin alpha(4) (VLA-4) (CD49D) encoding genes), Duchene muscular dystrophy (DMD) (associated with dystrophin (DMD) gene), centronuclear myopathy (associated with dynamin 2 (DNM2) encoding gene), and myotonic dystrophy (DM1) (associated with myotonic dystrophy protein kinase (DMPK) encoding gene).

In certain embodiments, the indicated endocrine or metabolic diseases, disorders, syndrome and/or conditions include, but not limited to, hypertriglyceridemia (associated with apolipoprotein C-III (APOC3), or angiopoietin-like 3 (ANGPTL3) encoding genes), lipodystrophy, hyperlipidemia (associated with apolipoprotein C-III (APOC3) encoding gene), hypercholesterolemia (associated with apolipoprotein B-100 (APOB-100), proprotein convertase subtilisin kexin type 9 (PCSK9)), or amyloidosis (associated with transthyretin (TTR) encoding gene), porphyria (associated with aminolevulinate synthase-1 (ALAS-1) encoding gene), neuropathy (associated with transthyretin (TTR) encoding gene), primary hyperoxaluria type 1 (associated with glycolate oxidase encoding gene), diabetes (associated with Glucagon receptor (GCGR) encoding gene), acromegaly (growth hormone receptor (GHR) encoding gene), alpha-1 antitrypsin deficiency (AATD) (associated with alpha-1 antitrypsin (AAT) encoding gene), propionic acidemia (propionyl-CoA carboxylase (PCCA/PCCB) encoding gene), glycogen storage disease type III (GDSIII) (associated with glycogen debranching enzyme (GSDIII) encoding gene), cardiometabolic disease (associated with asialoglycoprotein (ASGPR), hydroxyacid Oxidase 1 (HAO1), or alpha-1-antitrypsin (SERPINA1) encoding genes), methylmalonic acidemia (MMA) (associated with methylmalonyl CoA mutase (MMUT), cob(I)alamin adenosyltransferase (MMAA or MMAB), methylmalonyl-CoA epimerase (MCEE), LMBR1 domain containing 1 (LMBRD1), or ATP-binding cassette subfamily D member 4 (ABCD4) encoding genes), glycogen storage disease type 1a (associated with Glucose-6-phosphatase catalytic subunit-related protein (G6PC) encoding gene), and phenylketonuria (PKU) (associated with phenylalanine hydroxylase (PAH) encoding gene).

In certain embodiments, the indicated neurological diseases, disorders, syndrome and/or conditions include, but not limited to, spinal muscular atrophy (SMA) (associated with survival motor neuron protein (SMN2) gene), amyotrophic lateral sclerosis (ALS) (superoxide dismutase type 1 (SOD1), FUS RNA binding protein (FUS), microRNA-155, chromosome 9 open reading frame 72 (C9orf72), or ataxin-2 (ATXN2) genes), Huntington disease (associated with huntingtin (HTT) gene), hATTR polyneuropathy (associated with transthyretin (TTR) gene), Alzheimer's disease (associated with MAP-tau (MAPT) gene), Multiple System Atrophy (associated with alpha-synuclein (SNCA)), Parkinson's disease (associated with alpha-synuclein (SNCA), leucine rich repeat kinase 2 (LRRK2) genes), centronuclear myopathy (associated with dynamin 2 (DNM2) gene), Angelman syndrome (associated with ubiquitin protein ligase E3A (UBE3A) gene), epilepsy (associated with glycogen synthase 1 (GYS1) gene), Dravet Syndrome (associated with sodium voltage-gated channel alpha subunit 1 (SNC1A) gene), Leukodystrophy (associated with glial fibrillary acidic protein (GFAP) gene), prion disease (associated with prion protein (PRNP) gene), and Hereditary cerebral hemorrhage with amyloidosis-Dutch type (HCHWA-D) (associated with amyloid beta precursor protein (APP) gene).

In certain embodiments, the indicated renal diseases, disorders, syndrome and/or conditions include, but not limited to, Glomerulonephritis (IgA Nephropathy) (associated with complement factor B encoding gene), Alport syndrome (associated with proteins in the PPARα signaling pathway), and neuropathy (associated with apolipoprotein L1 (APOL1) encoding gene) or an APOL1-associated chronic kidney disease.

In certain embodiments, the gene editing nuclease is targeted to the gene of interest, wherein the gene of interest includes but not limited to lysophosphatidic acid encoding gene, lipoprotein (a) encoding gene, ANGPTL3, APOC3, F11, AGT, SERPINH1/Hsp47, HSD17B13, DGAT2, PNPLA3, ALDH2, DMD, VLA-4, DNM2DM1, DMPK, APOC3, ANGPTL3, APOB-100, PCSK9, TTR, ALAS-1, glycolate oxidase encoding gene, GCGR, GHR, AATD, AAT, PCCA, PCCB, GDSIII, ASGPR, HAO1, SERPINA1, MMA, MMUT, MMAA, MMAB, MCEE, LMBRD1, ABCD4, G6PC, PAH, SMN2, SOD1, FUS, C9orf72, ATXN2, HTT, MAPT, SNCA, LRRK2, UBE3A, GYS1, SNC1A, GFAP, PRNP, APP, complement factor B encoding gene, APOL1, AAS1, SLC25A13 genes.

Suitable gene editing targets include, e.g., liver-expressed genes such as, without limitation, proprotein convertase subtilisin/kexin type 9 (PCSK9) (cholesterol related disorders), transthyretin (TTR) (transthyretin amyloidosis), HAO, apolipoprotein C-III (APOC3), Factor VIII, Factor IX, low density lipoprotein receptor (LDLr), lipoprotein lipase (LPL) (Lipoprotein Lipase Deficiency), lecithin-cholesterol acyltransferase (LCAT), ornithine transcarbamylase (OTC), camosinase (CN1), sphingomyelin phosphodiesterase (SMPD1) (Niemann-Pick disease), hypoxanthine-guanine phosphoribosyltransferase (HGPRT), branched-chain alpha-keto acid dehydrogenase complex (BCKDC) (maple syrup urine disease), erythropoietin (EPO), Carbamyl Phosphate Synthetase (CPS1), N-Acetylglutamate Synthetase (NAGS), Argininosuccinic Acid Synthetase (Citrullinemia), Argininosuccinate Lyase (ASL) (Argininosuccinic Aciduria), and Arginase (AG).

Other gene editing targets may include, e.g., hydroxymethylbilane synthase (HMBS), carbamoyl synthetase I, ornithine transcarbamylase (OTC), arginosuccinate synthetase, alpha 1 anti-trypsin (A1AT), aaporginosuccinate lyase (ASL) for treatment of argunosuccinate lyase deficiency, arginase, fumarylacetate hydrolase, phenylalanine hydroxylase, alpha-1 antitrypsin, rhesus alpha-fetoprotein (AFP), rhesus chorionic gonadotrophin (CG), glucose-6-phosphatase, porphobilinogen deaminase, cystathione beta-synthase, branched chain ketoacid decarboxylase, albumin, isovaleryl-coA dehydrogenase, propionyl CoA carboxylase, methyl malonyl CoA mutase (MUT), glutaryl CoA dehydrogenase, insulin, beta-glucosidase, pyruvate carboxylate, hepatic phosphorylase, phosphorylase kinase, glycine decarboxylase, H-protein, T-protein, a cystic fibrosis transmembrane regulator (CFTR) sequence, and a dystrophin gene product [e.g., a mini- or micro-dystrophin]. Still other useful gene products include enzymes such as may be useful in enzyme replacement therapy, which is useful in a variety of conditions resulting from deficient activity of enzyme. For example, enzymes that contain mannose-6-phosphate may be utilized in therapies for lysosomal storage diseases (e.g., a suitable gene includes that encoding β-glucuronidase (GUSB)). In another example, the gene product is ubiquitin protein ligase. glucose-6-phosphatase, associated with glycogen storage disease or deficiency type 1A (GSD1), phosphoenolpyruvate-carboxykinase (PEPCK), associated with PEPCK deficiency; cyclin-dependent kinase-like 5 (CDKL5), also known as serine/threonine kinase 9 (STK9) associated with seizures and severe neurodevelopmental impairment; galactose-1 phosphate uridyl transferase, associated with galactosemia; phenylalanine hydroxylase (PAH), associated with phenylketonuria (PKU); gene products associated with Primary Hyperoxaluria Type 1 including Hydroxyacid Oxidase 1 (GO/HAO1) and AGXT, branched chain alpha-ketoacid dehydrogenase, including BCKDH, BCKDH-E2, BAKDH-E1a, and BAKDH-E1b, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; ornithine transcarbamylase (OTC), associated with omithine transcarbamylase deficiency; argininosuccinic acid synthetase (ASS1), associated with citrullinemia; lecithin-cholesterol acyltransferase (LCAT) deficiency; amethylmalonic acidemia (MMA); NPC1 associated with Niemann-Pick disease, type C1); propionic academia (PA); TTR associated with Transthyretin (TTR)-related Hereditary Amyloidosis; low density lipoprotein receptor (LDLR) protein, associated with familial hypercholesterolemia (FH), LDLR variant, such as those described in WO 2015/164778; PCSK9; ApoE and ApoC proteins, associated with dementia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotimidase, associated with biotimidase deficiency; alpha-galactosidase A (a-Gal A) associated with Fabry disease); beta-galactosidase (GLB1) associated with GM1 gangliosidosis; ATP7B associated with Wilson's Disease; beta-glucocerebrosidase, associated with Gaucher disease type 2 and 3; peroxisome membrane protein 70 kDa, associated with Zellweger syndrome; arylsulfatase A (ARSA) associated with metachromatic leukodystrophy, galactocerebrosidase (GALC) enzyme associated with Krabbe disease, alpha-glucosidase (GAA) associated with Pompe disease; sphingomyelinase (SMPD1) gene associated with Nieman Pick disease type A; argininosuccsinate synthase associated with adult onset type II citrullinemia (CTLN2); carbamoyl-phosphate synthase 1 (CPS1) associated with urea cycle disorders; survival motor neuron (SMN) protein, associated with spinal muscular atrophy; ceramidase associated with Farber lipogranulomatosis; b-hexosaminidase associated with GM2 gangliosidosis and Tay-Sachs and Sandhoff diseases; aspartylglucosaminidase associated with aspartyl-glucosaminuria; a-fucosidase associated with fucosidosis; a-mannosidase associated with alpha-mannosidosis; porphobilinogen deaminase, associated with acute intermittent porphyria (AIP); alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarco(endo)plasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin or GLP-1 for the treatment of diabetes.

In one embodiment, the capsids described herein are useful in the CRISPR-Cas dual vector system described in US Published Patent Application 2018/0110877, filed Apr. 26, 2018, each of which is incorporated herein by reference. The capsids are also useful for delivery homing endonucleases or other meganucleases.

In another embodiment, the transgenes useful herein include reporter sequences, which upon expression produce a detectable signal. Such reporter sequences include, without limitation, DNA sequences encoding P-lactamase, β-galactosidase (LacZ), alkaline phosphatase, thymidine kinase, green fluorescent protein (GFP), red fluorescent protein (RFP), chloramphenicol acetyltransferase (CAT), luciferase, membrane bound proteins including, for example, CD2, CD4, CD8, the influenza hemagglutinin protein, and others well known in the art, to which high affinity antibodies directed thereto exist or can be produced by conventional means, and fusion proteins comprising a membrane bound protein appropriately fused to an antigen tag domain from, among others, hemagglutinin or Myc.

In certain embodiments, in addition to the transgene coding sequence, another non-AAV coding sequence may be included, e.g., a peptide, polypeptide, protein, functional RNA molecule (e.g., miRNA, miRNA inhibitor) or other gene product, of interest. Useful gene products may include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors which are subsequently processed into a miRNA duplex, and further into a “mature” single stranded miRNA molecule. This mature miRNA guides a multiprotein complex, miRISC, which identifies target site, e.g., in the 3′ UTR regions, of target mRNAs based upon their complementarity to the mature miRNA.

These above coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, where the marker sequence is the LacZ gene, the presence of the vector carrying the signal is detected by assays for beta-galactosidase activity. Where the transgene is green fluorescent protein or luciferase, the vector carrying the signal may be measured visually by color or light production in a luminometer.

Desirably, the transgene encodes a product which is useful in biology and medicine, such as proteins, peptides, RNA, enzymes, or catalytic RNAs. Desirable RNA molecules include shRNA, tRNA, dsRNA, ribosomal RNA, catalytic RNAs, and antisense RNAs. One example of a useful RNA sequence is a sequence which extinguishes expression of a targeted nucleic acid sequence in a target cell.

Regulatory sequences include conventional control elements which are operably linked to the transgene in a manner which permits its transcription, translation and/or expression in a cell transfected with the vector or infected with the virus produced as described herein. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters, are known in the art and may be utilized.

The regulatory sequences useful in the constructs provided herein may also contain an intron, desirably located between the promoter/enhancer sequence and the gene. One desirable intron sequence is derived from SV-40, and is a 100 bp mini-intron splice donor/splice acceptor referred to as SD-SA. Another suitable sequence includes the woodchuck hepatitis virus post-transcriptional element. (See, e.g., L. Wang and I. Verma, 1999 Proc. Natl. Acad. Sci., USA, 96:3906-3910). PolyA signals may be derived from many suitable species, including, without limitation SV-40, human and bovine.

Another regulatory component of the rAAV useful in the methods described herein is an internal ribosome entry site (IRES). An IRES sequence, or other suitable systems, may be used to produce more than one polypeptide from a single gene transcript. An IRES (or other suitable sequence) is used to produce a protein that contains more than one polypeptide chain or to express two different proteins from or within the same cell. An exemplary IRES is the poliovirus internal ribosome entry sequence, which supports transgene expression in photoreceptors, RPE and ganglion cells. Preferably, the IRES is located 3′ to the transgene in the rAAV vector.

In certain embodiments, the vector genome comprises a promoter (or a functional fragment of a promoter). The selection of the promoter to be employed in the rAAV may be made from among a wide number of constitutive or inducible promoters that can express the selected transgene in the desired target cell. In one embodiment, the target cell is an ocular cell. The promoter may be derived from any species, including human. Desirably, in one embodiment, the promoter is “cell specific”. The term “cell-specific” means that the particular promoter selected for the recombinant vector can direct expression of the selected transgene in a particular cell tissue. In one embodiment, the promoter is specific for expression of the transgene in muscle cells. In another embodiment, the promoter is specific for expression in lung. In another embodiment, the promoter is specific for expression of the transgene in liver cells. In another embodiment, the promoter is specific for expression of the transgene in airway epithelium. In another embodiment, the promoter is specific for expression of the transgene in neurons. In another embodiment, the promoter is specific for expression of the transgene in heart.

The vector genome typically contains a promoter sequence as part of the expression control sequences, e.g., located between the selected 5′ ITR sequence and the immunoglobulin construct coding sequence. In one embodiment, expression in liver is desirable. Thus, in one embodiment, a liver-specific promoter is used. Examples of liver-specific promoters may include, e.g., thyroid hormone-binding globulin (TBG), albumin, Miyatake et al., (1997) J. Virol., 71:5124 32; hepatitis B virus core promoter, Sandig et al., (1996) Gene Ther., 3:1002 9; or human alpha 1-antitrypsin, phosphoenolpyruvate carboxykinase (PECK), or alpha fetoprotein (AFP), Arbuthnot et al., (1996) Hum. Gene Ther., 7:1503 14). Tissue specific promoters, constitutive promoters, regulatable promoters [see, e.g., WO 2011/126808 and WO 2013/04943], or a promoter responsive to physiologic cues may be used may be utilized in the vectors described herein. In another embodiment, expression in muscle is desirable. Thus, in one embodiment, a muscle-specific promoter is used. In one embodiment, the promoter is an MCK based promoter, such as the dMCK (509-bp) or tMCK (720-bp) promoters (see, e.g., Wang et al, Gene Ther. 2008 November; 15(22):1489-99. doi: 10.1038/gt.2008.104. Epub 2008 Jun. 19, which is incorporated herein by reference). Another useful promoter is the SPc5-12 promoter (see Rasowo et al, European Scientific Journal June 2014 edition vol. 10, No. 18, which is incorporated herein by reference). In certain embodiments, a promoter specific for the eye or a subpart thereof (e.g., retina) may be selected.

In one embodiment, the promoter is a CMV promoter. In another embodiment, the promoter is a TBG promoter. In another embodiment, a CB7 promoter is used. CB7 is a chicken β-actin promoter with cytomegalovirus enhancer elements. Alternatively, other liver-specific promoters may be used [see, e.g., The Liver Specific Gene Promoter Database, Cold Spring Harbor, rulai.schl.edu/LSPD, alpha 1 anti-trypsin (A1AT); human albumin Miyatake et al., J. Virol., 71:5124 32 (1997), humAlb; and hepatitis B virus core promoter, Sandig et al., Gene Ther., 3:1002 9 (1996)]. TTR minimal enhancer/promoter, alpha-antitrypsin promoter, LSP (845 nt)25 (requires intron-less scAAV).

The promoter(s) can be selected from different sources, e.g., human cytomegalovirus (CMV) immediate-early enhancer/promoter, the SV40 early enhancer/promoter, the JC polymovirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, CBA, matrix metalloprotein promoter (MPP), and the chicken beta-actin promoter.

The vector genome may contain at least one enhancer, i.e., CMV enhancer. Still other enhancer elements may include, e.g., an apolipoprotein enhancer, a zebrafish enhancer, a GFAP enhancer element, and brain specific enhancers such as described in WO 2013/1555222, woodchuck post hepatitis post-transcriptional regulatory element. Additionally, or alternatively, other, e.g., the hybrid human cytomegalovirus (HCMV)-immediate early (IE)-PDGR promoter or other promoter-enhancer elements may be selected. Other enhancer sequences useful herein include the IRBP enhancer (Nicoud 2007, J Gene Med. 2007 December; 9(12):1015-23), immediate early cytomegalovirus enhancer, one derived from an immunoglobulin gene or SV40 enhancer, the cis-acting element identified in the mouse proximal promoter, etc.

In addition to a promoter, a vector genome may contain other appropriate transcription initiation, termination, enhancer sequences, efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A variety of suitable polyA are known. In one example, the polyA is rabbit beta globin, such as the 127 bp rabbit beta-globin polyadenylation signal (GenBank #V00882.1). In other embodiments, an SV40 polyA signal is selected. Still other suitable polyA sequences may be selected. In certain embodiments, an intron is included. One suitable intron is a chicken beta-actin intron. In one embodiment, the intron is 875 bp (GenBank #X00182.1). In another embodiment, a chimeric intron available from Promega is used. However, other suitable introns may be selected. In one embodiment, spacers are included such that the vector genome is approximately the same size as the native AAV vector genome (e.g., between 4.1 and 5.2 kb). In one embodiment, spacers are included such that the vector genome is approximately 4.7 kb. See, Wu et al, Effect of Genome Size on AAV Vector Packaging, Mol Ther. 2010 January; 18(1): 80-86, which is incorporated herein by reference.

In certain embodiments, the vector genome further comprises dorsal root ganglion (drg)-specific miRNA detargeting sequences operably linked to the transgene coding sequence. In certain embodiments, the tandem miRNA target sequences are continuous or are separated by a spacer of 1 to 10 nucleic acids, wherein said spacer is not an miRNA target sequence. In certain embodiments, there are at least two drg-specific miRNA sequences located at 3′ to a functional transgene coding sequence. In certain embodiments, the start of the first of the at least two drg-specific miRNA tandem repeats is within 20 nucleotides from the 3′ end of the transgene coding sequence. In certain embodiments, the start of the first of the at least two drg-specific miRNA tandem repeats is at least 100 nucleotides from the 3′ end of the functional transgene coding sequence. In certain embodiments, the miRNA tandem repeats comprise 200 to 1200 nucleotides in length. In certain embodiments, there are at least two drg-specific miRNA target sequences located at 5′ to the functional transgene coding sequence. In certain embodiments, at least two drg-specific miRNA target sequences are located in both 5′ and 3′ to the functional transgene coding sequence. In certain embodiments, the miRNA target sequence for the at least first and/or at least second miRNA target sequence for the expression cassette mRNA or DNA positive strand is selected from (i) AGTGAATTCTACCAGTGCCATA (SEQ ID NO: 78); (ii) AGCAAAAATGTGCTAGTGCCAAA (SEQ ID NO: 79), (iii) AGTGTGAGTTCTACCATTGCCAAA (SEQ ID NO: 80); or (iv) AGGGATTCCTGGGAAAACTGGAC (SEQ ID NO: 81). In certain embodiments, the miRNA target sequence for the at least first and/or at least second miRNA target sequence for the expression cassette mRNA or DNA positive strand is AGTGAATTCTACCAGTGCCATA (SEQ ID NO: 78). In certain embodiments, the miRNA target sequence for the at least first and/or at least second miRNA target sequence for the expression cassette mRNA or DNA positive strand is AGTGAATTCTACCAGTGCCATA (SEQ ID NO: 78). In certain embodiments, two or more consecutive miRNA target sequences are continuous and not separated by a spacer. In certain embodiments, two or more of the miRNA target sequences are separated by a spacer and each spacer is independently selected from one or more of (A) GGAT; (B) CACGTG; or (C) GCATGC. In certain embodiments, the spacer located between the miRNA target sequences may be located 3′ to the first miRNA target sequence and/or 5′ to the last miRNA target sequence. In certain embodiments, the spacers between the miRNA target sequences are the same. See International Patent Application No. PCT/US19/67872, filed Dec. 20, 2019, U.S. Provisional Patent Application No. 63/023,594, filed May 12, 2020, U.S. Provisional Patent Application No. 63/038,488, filed Jun. 12, 2020, U.S. Provisional Patent Application No. 63/043,562, filed Jun. 24, 2020, and U.S. Provisional Patent Application No. 63/079,299, filed Sep. 16, 2020, all of which are incorporated by reference in their entireties.

Selection of these and other common vector and regulatory elements are conventional and many such sequences are available. See, e.g., Sambrook et al, and references cited therein at, for example, pages 3.18-3.26 and 16.17-16.27 and Ausubel et al., Current Protocols in Molecular Biology, John Wiley & Sons, New York, 1989. Of course, not all vectors and expression control sequences will function equally well to express all of the transgenes as described herein. However, one of skill in the art may make a selection among these, and other, expression control sequences without departing from the scope of this invention.

In another embodiment, a method of generating a recombinant adeno-associated virus is provided. A suitable recombinant adeno-associated virus (AAV) is generated by culturing a host cell which contains a nucleic acid sequence encoding an AAV capsid protein as described herein, or fragment thereof; a functional rep gene; a minigene composed of, at a minimum, AAV inverted terminal repeats (ITRs) and a heterologous nucleic acid sequence encoding a desirable transgene; and sufficient helper functions to permit packaging of the minigene into the AAV capsid protein. The components required to be cultured in the host cell to package an AAV minigene in an AAV capsid may be provided to the host cell in trans. Alternatively, any one or more of the required components (e.g., minigene, rep sequences, cap sequences, and/or helper functions) may be provided by a stable host cell which has been engineered to contain one or more of the required components using methods known to those of skill in the art.

Also provided herein are host cells transfected with an AAV as described herein. Most suitably, such a stable host cell will contain the required component(s) under the control of an inducible promoter. However, the required component(s) may be under the control of a constitutive promoter. Examples of suitable inducible and constitutive promoters are provided herein, in the discussion below of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contains the rep and/or cap proteins under the control of inducible promoters. Still other stable host cells may be generated by one of skill in the art. In another embodiment, the host cell comprises a nucleic acid molecule (e.g., a plasmid) as described herein.

The minigene, rep sequences, cap sequences, and helper functions required for producing the rAAV described herein may be delivered to the packaging host cell in the form of any genetic element which transfers the sequences carried thereon. The selected genetic element may be delivered by any suitable method, including those described herein. The methods used to construct any embodiment of this invention are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, NY. Similarly, methods of generating rAAV virions are well known and the selection of a suitable method is not a limitation on the present invention. See, e.g., K. Fisher et al, 1993 J Virol., 70:520-532 and U.S. Pat. No. 5,478,745, among others. These publications are incorporated by reference herein.

Also provided herein, are plasmids for use in producing the vectors described herein. Such plasmids include a nucleic acid sequence encoding at least one of the vp1, vp2, and vp3 of AAVhu71/74 (SEQ ID NO: 4), AAVhu79 (SEQ ID NO: 6), AAVhu80 (SEQ ID NO: 8), AAVhu83 (SEQ ID NO: 10), AAVhu74/71 (SEQ ID NO: 12), AAVhu77 (SEQ ID NO: 14), AAVhu78/88 (SEQ ID NO: 16), AAVhu70 (SEQ ID NO: 18), AAVhu72 (SEQ ID NO: 20), AAVhu75 (SEQ ID NO: 22), AAVhu76 (SEQ ID NO: 24), AAVhu81 (SEQ ID NO: 26), AAVhu82 (SEQ ID NO: 28), AAVhu84 (SEQ ID NO: 30), AAVhu86 (SEQ ID NO: 32), AAVhu87 (SEQ ID NO: 34), AAVhu88/78 (SEQ ID NO: 36), AAVhu69 (SEQ ID NO: 38), AAVrh75 (SEQ ID NO: 40), AAVrh76 (SEQ ID NO: 42), AAVrh77 (SEQ ID NO: 44), AAVrh78 (SEQ ID NO: 46), AAVrh79 (SEQ ID NO: 48), AAVrh81 (SEQ ID NO: 50), AAVrh89 (SEQ ID NO: 52), AAVrh82 (SEQ ID NO: 54), AAVrh83 (SEQ ID NO: 56), AAVrh84 (SEQ ID NO: 58), AAVrh85 (SEQ ID NO: 60), AAVrh87 (SEQ ID NO: 62), or AAVhu73 (SEQ ID NO: 74). In certain embodiments, provided are plasmids having the a vp1, vp2, and/or vp3 sequence of AAVhu71/74 (SEQ ID NO: 3), AAVhu79 (SEQ ID NO: 5), AAVhu80 (SEQ ID NO: 7), AAVhu83 (SEQ ID NO: 9), AAVhu74/71 (SEQ ID NO: 11), AAVhu77 (SEQ ID NO: 13), AAVhu78/88 (SEQ ID NO: 15), AAVhu70 (SEQ ID NO: 17), AAVhu72 (SEQ ID NO: 19), AAVhu75 (SEQ ID NO: 21), AAVhu76 (SEQ ID NO: 23), AAVhu81 (SEQ ID NO: 25), AAVhu82 (SEQ ID NO: 27), AAVhu84 (SEQ ID NO: 29), AAVhu86 (SEQ ID NO: 31), AAVhu87 (SEQ ID NO: 33), AAVhu88/78 (SEQ ID NO: 35), AAVhu69 (SEQ ID NO: 37), AAVrh75 (SEQ ID NO: 39), AAVrh76 (SEQ ID NO: 41), AAVrh77 (SEQ ID NO: 43), AAVrh78 (SEQ ID NO: 45), AAVrh79 (SEQ ID NO: 47), AAVrh81 (SEQ ID NO: 49), AAVrh89 (SEQ ID NO: 51), AAVrh82 (SEQ ID NO: 53), AAVrh83 (SEQ ID NO: 55), AAVrh84 (SEQ ID NO: 57), AAVrh85 (SEQ ID NO: 59), AAVrh87 (SEQ ID NO: 61), or AAVhu73 (SEQ ID NO: 73), or a sequence sharing at least 95%, at least 96%, at least 97%, at least 98%, or at least 99% identity with any of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, or 61. In further embodiments, the plasmids include a non-AAV sequence. Cultured host cells containing the plasmids described herein are also provided.

In certain embodiments, the plasmids generated are an AAV cis-plasmid encoding the AAV genome and the gene of interest, an AAV trans-plasmid containing AAV rep and the novel hu68 cap gene, and a helper plasmid. These plasmids may be used in any suitable ratio, e.g., about 1 to about 1 to about 1, based on the total weight of the genetic elements. In other embodiments, the pRepCap to AAV cis-plasmid ratio of about 1:1 by weight of each coding sequence and the pHelper is about 2 times the weight. In other embodiments, the ratio may be about 3 to 1 helper: 10 to 1 pRepCap: 1 to 0.10 rAAV plasmid, by weight. Other suitable ratios may be selected. In certain embodiments, the host cell may be stably transformed with one or more of these elements. For example, the host cell may contain a stable nucleic acid molecule comprising the AAVhu68M191 vp1 coding sequence operably linked to regulatory sequences, a nucleic acid molecule encoding the rep coding sequences and/or one or more nucleic acid molecules encoding helper functions (e.g., adenovirus Ela, or the like). In such embodiments, the various genetic elements may be used in any suitable ratio, e.g., about 1 to about 1 to about 1, based on the total weight of the genetic elements. In certain embodiments, the pRep DNA to Cap DNA to the AAV molecule (e.g., plasmid carrying the vector genome to be packaged) ratio of about 1 to about 1 to about 1 (1:1:1) by weight. In certain embodiments, certain host cells contain some helper elements (e.g., Ad E2a and/or AdE2b) provided in trans and others in cis (e.g., Ad E1a and/or E1b). The helper sequences may be present in about 2 times the amount of the other genetic elements. Still other ratios may be determined.

The vector generation process can include method steps such as initiation of cell culture, passage of cells, seeding of cells, transfection of cells with the plasmid DNA, post-transfection medium exchange to serum free medium, and the harvest of vector-containing cells and culture media. The harvested vector-containing cells and culture media are referred to herein as crude cell harvest. In yet another system, the gene therapy vectors are introduced into insect cells by infection with baculovirus-based vectors. For reviews on these production systems, see generally, e.g., Clement and Grieger, Mol Ther Methods Clin Dev, 2016: 3: 16002, published online 2016 Mar. 16. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

The crude cell harvest may thereafter be subject method steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector.

A variety of AAV purification methods are known in the art. See, e.g., WO 2017/160360 entitled “Scalable Purification Method for AAV9”, which is incorporated by reference herein, and describes methods generally useful for Clade F capsids. A two-step affinity chromatography purification followed by anion exchange resin chromatography are used to purify the vector drug product and to remove empty capsids. The crude cell harvest may be subject steps such as concentration of the vector harvest, diafiltration of the vector harvest, microfluidization of the vector harvest, nuclease digestion of the vector harvest, filtration of microfluidized intermediate, crude purification by chromatography, crude purification by ultracentrifugation, buffer exchange by tangential flow filtration, and/or formulation and filtration to prepare bulk vector. An affinity chromatography purification followed anion exchange resin chromatography are used to purify the vector drug product and to remove empty capsids. In one example, for the Affinity Chromatography step, the diafiltered product may be applied to a Capture Select™ Poros-AAV2/9 affinity resin (Life Technologies) that efficiently captures the AAV2/9 serotype. Under these ionic conditions, a significant percentage of residual cellular DNA and proteins flow through the column, while AAV particles are efficiently captured. See, also, WO2021/158915; WO2019/241535; and WO 2021/165537. Alternatively, other purification methods may be selected.

Methods for characterization or quantification of rAAV are available to one of skill in the art. For example, to calculate empty and full particle content, VP3 band volumes for a selected sample (e.g., in examples herein an iodixanol gradient-purified preparation where # of GC=# of particles) are plotted against GC particles loaded. The resulting linear equation (y=mx+c) is used to calculate the number of particles in the band volumes of the test article peaks. The number of particles (pt) per 20 μL loaded is then multiplied by 50 to give particles (pt)/mL. Pt/mL divided by GC/mL gives the ratio of particles to genome copies (pt/GC). Pt/mL-GC/mL gives empty pt/mL. Empty pt/mL divided by pt/mL and x 100 gives the percentage of empty particles.

In certain embodiments, the yield of packaged AAV vector genome copies (VG or GC) may be assessed through use of a bioactivity assay for the encoded transgene. For example, after production, culture supernatants may be collected and spun down to remove cell debris. The yields may be measured by a bioactivity assay using equal volume of the supernatant from a test sample as compared to a control (reference standard) to transduce a selected target cell and to evaluate bioactivity of the encoded protein. Other suitable methods for assessing yield may be selected, including, for example, nanoparticle tracking [Povlich, S. F., et al. (2016) Particle Titer Determination and Characterization of rAAV Molecules Using Nanoparticle Tracking Analysis. Molecular Therapy: AAV Vectors II, 24(S1), S122], enzyme linked immunosorbent assay (ELISA) [Grimm, D., et al (1999). Titration of AAV-2 particles via a novel capsid ELISA: packaging of genomes can limit production of recombinant AAV-2. Gene therapy, 6(7), 1322-1330. doi.org/10.1038/sj.gt.3300946]; digital droplet (dd) polymerase chain reaction (PCR)Methods for determining single-stranded and self-complementary AAV vector genome titers by digital droplet (dd) polymerase chain reaction (PCR) have been described. See, e.g., M. Lock et al, Hum Gene Ther Methods. 2014 April; 25(2):115-25. doi: 10.1089/hgtb.2013.131. Epub 2014 Feb. 14]. Another suitable method is qPCR. An optimized-PCR method may be used which utilizes a broad spectrum serine protease, e.g., proteinase K (such as is commercially available from Qiagen). More particularly, the optimized qPCR genome titer assay is similar to a standard assay, except that after the DNase I digestion, samples are diluted with proteinase K buffer and treated with proteinase K followed by heat inactivation. Suitably samples are diluted with proteinase K buffer in an amount equal to the sample size. The proteinase K buffer may be concentrated to 2 fold or higher. Typically, proteinase K treatment is about 0.2 mg/mL, but may be varied from 0.1 mg/mL to about 1 mg/mL. The treatment step is generally conducted at about 55° C. for about 15 minutes, but may be performed at a lower temperature (e.g., about 37° C. to about 50° C.) over a longer time period (e.g., about 20 minutes to about 30 minutes), or a higher temperature (e.g., up to about 60° C.) for a shorter time period (e.g., about 5 to 10 minutes). Similarly, heat inactivation is generally at about 95° C. for about 15 minutes, but the temperature may be lowered (e.g., about 70 to about 90° C.) and the time extended (e.g., about 20 minutes to about 30 minutes). Samples are then diluted (e.g., 1000 fold) and subjected to TaqMan analysis as described in the standard assay. Yet another method is the quantitative DNA dot blot [Wu, Z., et al, (2008). Optimization of self-complementary AAV vectors for liver-directed expression results in sustained correction of hemophilia B at low vector dose. Molecular therapy: the journal of the American Society of Gene Therapy, 16(2), 280-289. doi.org/10.1038/sj.mt.6300355]. Still other methods may be selected.

Methods for assaying for empty capsids and AAV vector particles with packaged genomes have been known in the art. See, e.g., Grimm et al., Gene Therapy (1999) 6:1322-1330; Sommer et al., Molec. Ther. (2003) 7:122-128. To test for denatured capsid, the methods include subjecting the treated AAV stock to SDS-polyacrylamide gel electrophoresis, consisting of any gel capable of separating the three capsid proteins, for example, a gradient gel containing 3-8% Tris-acetate in the buffer, then running the gel until sample material is separated, and blotting the gel onto nylon or nitrocellulose membranes, preferably nylon. Anti-AAV capsid antibodies are then used as the primary antibodies that bind to denatured capsid proteins, preferably an anti-AAV capsid monoclonal antibody, most preferably the B1 anti-AAV-2 monoclonal antibody (Wobus et al., J. Virol. (2000) 74:9281-9293). A secondary antibody is then used, one that binds to the primary antibody and contains a means for detecting binding with the primary antibody, more preferably an anti-IgG antibody containing a detection molecule covalently bound to it, most preferably a sheep anti-mouse IgG antibody covalently linked to horseradish peroxidase. A method for detecting binding is used to semi-quantitatively determine binding between the primary and secondary antibodies, preferably a detection method capable of detecting radioactive isotope emissions, electromagnetic radiation, or colorimetric changes, most preferably a chemiluminescence detection kit. For example, for SDS-PAGE, samples from column fractions can be taken and heated in SDS-PAGE loading buffer containing reducing agent (e.g., DTT), and capsid proteins were resolved on pre-cast gradient polyacrylamide gels (e.g., Novex). Silver staining may be performed using SilverXpress (Invitrogen, CA) according to the manufacturer's instructions or other suitable staining method, i.e., SYPRO ruby or coomassie stains. In one embodiment, the concentration of AAV vector genomes (vg) in column fractions can be measured by quantitative real time PCR (Q-PCR). Samples are diluted and digested with DNase I (or another suitable nuclease) to remove exogenous DNA. After inactivation of the nuclease, the samples are further diluted and amplified using primers and a TaqMan™ fluorogenic probe specific for the DNA sequence between the primers. The number of cycles required to reach a defined level of fluorescence (threshold cycle, Ct) is measured for each sample on an Applied Biosystems Prism 7700 Sequence Detection System. Plasmid DNA containing identical sequences to that contained in the AAV vector is employed to generate a standard curve in the Q-PCR reaction. The cycle threshold (Ct) values obtained from the samples are used to determine vector genome titer by normalizing it to the Ct value of the plasmid standard curve. End-point assays based on the digital PCR can also be used. As used herein, the terms genome copies (GC) and vector genomes (vg) in the context of a dose or dosage (e.g., GC/kg and vg/kg) are meant to be interchangeable.

Methods for determining the ratio among vp1, vp2 and vp3 of capsid protein are also available. See, e.g., Vamseedhar Rayaprolu et al, Comparative Analysis of Adeno-Associated Virus Capsid Stability and Dynamics, J Virol. 2013 December; 87(24): 13150-13160; Buller R M, Rose J A. 1978. Characterization of adenovirus-associated virus-induced polypeptides in KB cells. J. Virol. 25:331-338; and Rose J A, Maizel J V, Inman J K, Shatkin A J. 1971. Structural proteins of adenovirus-associated viruses. J. Virol. 8:766-770.

As used herein, a “stock” of rAAV refers to a population of rAAV. Despite heterogeneity in their capsid proteins due to deamidation, rAAV in a stock are expected to share an identical vector genome. A stock can include rAAV having capsids with, for example, heterogeneous deamidation patterns characteristic of the selected AAV capsid proteins and a selected production system. The stock may be produced from a single production system or pooled from multiple runs of the production system (e.g., different runs of a production system using the same genetic elements for production). A variety of production systems, including but not limited to those described herein, may be selected.

### C. Pharmaceutical Compositions and Administration

In one embodiment, the recombinant AAV containing the desired transgene and promoter for use in the target cells as detailed above is optionally assessed for contamination by conventional methods and then formulated into a pharmaceutical composition intended for administration to a subject in need thereof. Such formulation involves the use of a pharmaceutically and/or physiologically acceptable vehicle or carrier, such as buffered saline or other buffers, e.g., HEPES, to maintain pH at appropriate physiological levels, and, optionally, other medicinal agents, pharmaceutical agents, stabilizing agents, buffers, carriers, adjuvants, diluents, etc. For injection, the carrier will typically be a liquid. Exemplary physiologically acceptable carriers include sterile, pyrogen-free water and sterile, pyrogen-free, phosphate buffered saline. A variety of such known carriers are provided in U.S. Pat. No. 7,629,322, incorporated herein by reference. In one embodiment, the carrier is an isotonic sodium chloride solution. In another embodiment, the carrier is balanced salt solution. In one embodiment, the carrier includes tween. If the virus is to be stored long-term, it may be frozen in the presence of glycerol or Tween20. In another embodiment, the pharmaceutically acceptable carrier comprises a surfactant, such as perfluorooctane (Perfluoron liquid). The vector is formulated in a buffer/carrier suitable for infusion in human subjects. The buffer/carrier should include a component that prevents the rAAV from sticking to the infusion tubing but does not interfere with the rAAV binding activity in vivo.

In certain embodiments of the methods described herein, the pharmaceutical composition described above is administered to the subject intramuscularly (IM). In other embodiments, the pharmaceutical composition is administered by intravenously (IV). In other embodiments, the pharmaceutical composition is administered by intracerebroventricular (ICV) injection. In other embodiments, the pharmaceutical composition is administered by intra-cisterna magna (ICM) injection. Other forms of administration that may be useful in the methods described herein include, but are not limited to, direct delivery to a desired organ (e.g., the eye), including subretinal or intravitreal delivery, oral, inhalation, intranasal, intratracheal, intravenous, intramuscular, subcutaneous, intradermal, and other parental routes of administration. Routes of administration may be combined, if desired.

As used herein, the terms “intrathecal delivery” or “intrathecal administration” refer to a route of administration via an injection into the spinal canal, more specifically into the subarachnoid space so that it reaches the cerebrospinal fluid (CSF). Intrathecal delivery may include lumbar puncture, intraventricular (including intracerebroventricular (ICV)), suboccipital/intracisternal, and/or C1-2 puncture. For example, material may be introduced for diffusion throughout the subarachnoid space by means of lumbar puncture. In another example, injection may be into the cisterna magna.

As used herein, the terms “intracisternal delivery” or “intracisternal administration” refer to a route of administration directly into the cerebrospinal fluid of the cisterna magna cerebellomedularis, more specifically via a suboccipital puncture or by direct injection into the cisterna magna or via permanently positioned tube.

The composition may be delivered in a volume of from about 0.1 μL to about 10 mL, including all numbers within the range, depending on the size of the area to be treated, the viral titer used, the route of administration, and the desired effect of the method. In one embodiment, the volume is about 50 μL. In another embodiment, the volume is about 70 μL. In another embodiment, the volume is about 100 μL. In another embodiment, the volume is about 125 μL. In another embodiment, the volume is about 150 μL. In another embodiment, the volume is about 175 μL. In yet another embodiment, the volume is about 200 μL. In another embodiment, the volume is about 250 μL. In another embodiment, the volume is about 300 μL. In another embodiment, the volume is about 450 μL. In another embodiment, the volume is about 500 μL. In another embodiment, the volume is about 600 μL. In another embodiment, the volume is about 750 μL. In another embodiment, the volume is about 850 μL. In another embodiment, the volume is about 1000 μL. In another embodiment, the volume is about 1.5 mL. In another embodiment, the volume is about 2 mL. In another embodiment, the volume is about 2.5 mL. In another embodiment, the volume is about 3 mL. In another embodiment, the volume is about 3.5 mL. In another embodiment, the volume is about 4 mL. In another embodiment, the volume is about 5 mL. In another embodiment, the volume is about 5.5 mL. In another embodiment, the volume is about 6 mL. In another embodiment, the volume is about 6.5 mL. In another embodiment, the volume is about 7 mL. In another embodiment, the volume is about 8 mL. In another embodiment, the volume is about 8.5 mL. In another embodiment, the volume is about 9 mL. In another embodiment, the volume is about 9.5 mL. In another embodiment, the volume is about 10 mL.

An effective concentration of a recombinant adeno-associated virus carrying a nucleic acid sequence encoding the desired transgene under the control of the regulatory sequences desirably ranges from about 10⁷ and 10¹⁴ vector genomes per milliliter (vg/mL) (also called genome copies/mL (GC/mL)). In one embodiment, the rAAV vector genomes are measured by real-time PCR. In another embodiment, the rAAV vector genomes are measured by digital PCR. See, Lock et al, Absolute determination of single-stranded and self-complementary adeno-associated viral vector genome titers by droplet digital PCR, Hum Gene Ther Methods. 2014 April; 25(2):115-25. doi: 10.1089/hgtb.2013.131. Epub 2014 Feb. 14, which are incorporated herein by reference. In another embodiment, the rAAV infectious units are measured as described in S. K. McLaughlin et al, 1988 J. Virol., 62:1963, which is incorporated herein by reference.

Preferably, the concentration is from about 1.5×10⁹ vg/mL to about 1.5×10¹³ vg/mL, and more preferably from about 1.5×10⁹ vg/mL to about 1.5×10¹¹ vg/mL. In one embodiment, the effective concentration is about 1.4×10⁸ vg/mL. In one embodiment, the effective concentration is about 3.5×10¹⁰ vg/mL. In another embodiment, the effective concentration is about 5.6×10¹¹ vg/mL. In another embodiment, the effective concentration is about 5.3×10¹² vg/mL. In yet another embodiment, the effective concentration is about 1.5×10¹² vg/mL. In another embodiment, the effective concentration is about 1.5×10¹³ vg/mL. All ranges described herein are inclusive of the endpoints.

In one embodiment, the dosage is from about 1.5×10⁹ vg/kg of body weight to about 1.5×10¹³ vg/kg, and more preferably from about 1.5×10⁹ vg/kg to about 1.5×10¹¹ vg/kg. In one embodiment, the dosage is about 1.4×10⁸ vg/kg. In one embodiment, the dosage is about 3.5×10¹⁰ vg/kg. In another embodiment, the dosage is about 5.6×10¹¹ vg/kg. In another embodiment, the dosage is about 5.3×10¹² vg/kg. In yet another embodiment, the dosage is about 1.5×10¹² vg/kg. In another embodiment, the dosage is about 1.5×10¹³ vg/kg. In another embodiment, the dosage is about 3.0×10¹³ vg/kg. In another embodiment, the dosage is about 1.0×10¹⁴ vg/kg. All ranges described herein are inclusive of the endpoints.

In one embodiment, the effective dosage (total genome copies delivered) is from about 10⁷ to 10¹³ vector genomes. In one embodiment, the total dosage is about 10⁸ genome copies. In one embodiment, the total dosage is about 10⁹ genome copies. In one embodiment, the total dosage is about 10¹⁰ genome copies. In one embodiment, the total dosage is about 10¹¹ genome copies. In one embodiment, the total dosage is about 10¹² genome copies. In one embodiment, the total dosage is about 10¹³ genome copies. In one embodiment, the total dosage is about 10¹⁴ genome copies. In one embodiment, the total dosage is about 10¹⁵ genome copies.

It is desirable that the lowest effective concentration of virus be utilized in order to reduce the risk of undesirable effects, such as toxicity. Still other dosages and administration volumes in these ranges may be selected by the attending physician, taking into account the physical state of the subject, preferably human, being treated, the age of the subject, the particular disorder and the degree to which the disorder, if progressive, has developed. Intravenous delivery, for example may require doses on the order of 1.5×10¹³ vg/kg.

### D. Methods

In another aspect, a method of transducing a target cell or tissue is provided. In one embodiment, the method includes administering an rAAV as described herein.

In one embodiment, the dosage of an rAAV is about 1×10⁹ GC to about 1×10¹⁵ genome copies (GC) per dose (to treat an average subject of 70 kg in body weight), and preferably 1.0×10¹² GC to 2.0×10¹⁵ GC for a human patient. In another embodiment, the dose is less than about 1×10¹⁴ GC/kg body weight of the subject. In certain embodiments, the dose administered to a patient is at least about 1.0×10⁹ GC/kg, about 1.5×10⁹ GC/kg, about 2.0×10⁹ GC/g, about 2.5×10⁹ GC/kg, about 3.0×10⁹ GC/kg, about 3.5×10⁹ GC/kg, about 4.0×10⁹ GC/kg, about 4.5×10⁹ GC/kg, about 5.0×10⁹ GC/kg, about 5.5×10⁹ GC/kg, about 6.0×10⁹ GC/kg, about 6.5×10⁹ GC/kg, about 7.0×10⁹ GC/kg, about 7.5×10⁹ GC/kg, about 8.0×10⁹ GC/kg, about 8.5×10⁹ GC/kg, about 9.0×10⁹ GC/kg, about 9.5×10⁹ GC/kg, about 1.0×10¹⁰ GC/kg, about 1.5×10¹⁰ GC/kg, about 2.0×10¹⁰ GC/kg, about 2.5×10¹⁰ GC/kg, about 3.0×10¹⁰ GC/kg, about 3.5×10¹⁰ GC/kg, about 4.0×10¹⁰ GC/kg, about 4.5×10¹⁰ GC/kg, about 5.0×10¹⁰ GC/kg, about 5.5×10¹⁰ GC/kg, about 6.0×10¹⁰ GC/kg, about 6.5×10¹⁰ GC/kg, about 7.0×10¹⁰ GC/kg, about 7.5×10¹⁰ GC/kg, about 8.0×10¹⁰ GC/kg, about 8.5×10¹⁰ GC/kg, about 9.0×10¹⁰ GC/kg, about 9.5×10¹⁰ GC/kg, about 1.0×10¹¹ GC/kg, about 1.5×10¹¹ GC/kg, about 2.0×10¹¹ GC/kg, about 2.5×10¹¹ GC/kg, about 3.0×10¹¹ GC/kg, about 3.5×10¹¹ GC/kg, about 4.0×10¹¹ GC/kg, about 4.5×10¹¹ GC/kg, about 5.0×10¹¹ GC/kg, about 5.5×10¹¹ GC/kg, about 6.0×10¹¹ GC/kg, about 6.5×10¹¹ GC/kg, about 7.0×10¹¹ GC/kg, about 7.5×10¹¹ GC/kg, about 8.0×10¹¹ GC/kg, about 8.5×10¹¹ GC/kg, about 9.0×10¹¹ GC/kg, about 9.5×10¹¹ GC/kg, about 1.0×10¹² GC/kg, about 1.5×10¹² GC/kg, about 2.0×10¹² GC/kg, about 2.5×10¹² GC/kg, about 3.0×10¹² GC/kg, about 3.5×10¹² GC/kg, about 4.0×10¹² GC/kg, about 4.5×10¹² GC/kg, about 5.0×10¹² GC/kg, about 5.5×10¹² GC/kg, about 6.0×10¹² GC/kg, about 6.5×10¹² GC/kg, about 7.0×10¹² GC/kg, about 7.5×10¹² GC/kg, about 8.0×10¹² GC/kg, about 8.5×10¹² GC/kg, about 9.0×10¹² GC/kg, about 9.5×10¹² GC/kg, about 1.0×10¹³ GC/kg, about 1.5×10¹³ GC/kg, about 2.0×10¹³ GC/kg, about 2.5×10¹³ GC/kg, about 3.0×10¹³ GC/kg, about 3.5×10¹³ GC/kg, about 4.0×10¹³ GC/kg, about 4.5×10¹³ GC/kg, about 5.0×10¹³ GC/kg, about 5.5×10¹³ GC/kg, about 6.0×10¹³ GC/kg, about 6.5×10¹³ GC/kg, about 7.0×10¹³ GC/kg, about 7.5×10¹³ GC/kg, about 8.0×10¹³ GC/kg, about 8.5×10¹³ GC/kg, about 9.0×10¹³ GC/kg, about 9.5×10¹³ GC/kg, or about 1.0×10¹⁴ GC/kg body weight or the subject.

In one embodiment, the method further comprises administering an immunosuppressive co-therapy to the subject. Such immunosuppressive co-therapy may be started prior to delivery of an rAAV or a composition as disclosed, e.g., if undesirably high neutralizing antibody levels to the AAV capsid are detected. In certain embodiments, co-therapy may also be started prior to delivery of the rAAV as a precautionary measure. In certain embodiments, immunosuppressive co-therapy is started following delivery of the rAAV, e.g., if an undesirable immune response is observed following treatment.

Immunosuppressants for such co-therapy include, but are not limited to, a glucocorticoid, steroids, antimetabolites, T-cell inhibitors, a macrolide (e.g., a rapamycin or rapalog), and cytostatic agents including an alkylating agent, an anti-metabolite, a cytotoxic antibiotic, an antibody, or an agent active on immunophilin. The immune suppressant may include prednelisone, a nitrogen mustard, nitrosourea, platinum compound, methotrexate, azathioprine, mercaptopurine, fluorouracil, dactinomycin, an anthracycline, mitomycin C, bleomycin, mithramycin, IL-2 receptor- (CD25-) or CD3-directed antibodies, anti-IL-2 antibodies, ciclosporin, tacrolimus, sirolimus, IFN-β, IFN-γ, an opioid, or TNF-α (tumor necrosis factor-alpha) binding agent. In certain embodiments, the immunosuppressive therapy may be started 0, 1, 2, 7, or more days prior to the rAAV administration, or 0, 1, 2, 3, 7, or more days post the rAAV administration. Such therapy may involve a single drug (e.g., prednelisone) or co-administration of two or more drugs, the (e.g., prednisolone, micophenolate mofetil (MMF) and/or sirolimus (i.e., rapamycin)) on the same day. One or more of these drugs may be continued after gene therapy administration, at the same dose or an adjusted dose. Such therapy may be for about 1 week (7 days), two weeks, three weeks, about 60 days, or longer, as needed. In certain embodiments, a tacrolimus-free regimen is selected.

Further embodiments are listed below as 1 through 12.

1. A recombinant adeno-associated virus (rAAV) comprising a capsid and a vector genome comprising an AAV 5′ inverted terminal repeat (ITR), an expression cassette comprising a nucleic acid sequence encoding a gene product operably linked to expression control sequences, and an AAV 3′ ITR, wherein the capsid is:
(a) an AAVrh75 capsid consisting of (i) a capsid produced from a nucleic acid sequence encoding SEQ ID NO: 40 or a sequence at least 99% identical thereto having an Asn (N) amino acid residue at position 24 based on the numbering of SEQ ID NO: 40; (ii) a capsid produced from a nucleic acid sequence of SEQ ID NO: 39 of a sequence or a sequence at least 95% identical thereto encoding SEQ ID NO: 40; or (iii) a capsid which is heterogeneous mixture of AAVrh75 vp1, vp2 and vp3 proteins which are 95% to 100% deamidated in at least position N57, N262, N384, and/or N512 of SEQ ID NO: 40, and optionally deamidated in other positions;
(b) an AAVhu71/74 capsid consisting of (i) a capsid produced from a nucleic acid sequence encoding SEQ ID NO: 3; (ii) a capsid produced from a nucleic acid sequence of SEQ ID NO: 3 of a sequence or a sequence at least 95% identical thereto encoding SEQ ID NO: 4; or (iii) a capsid which is a heterogeneous mixture of AAVrh71/74 vp1, vp2, and vp3 proteins which are 95% to 100% deamidated in at least 4 positions of SEQ ID NO: 4, and optionally deamidated in other positions;
(c) an AAVhu79 capsid consisting of (i) a capsid produced from a nucleic acid sequence encoding SEQ ID NO: 6; (ii) a capsid produced from a nucleic acid sequence of SEQ ID NO: 5 of a sequence or a sequence at least 95% identical thereto encoding SEQ ID NO: 6; or (iii) a capsid which is a heterogeneous mixture of AAVhu79 vp1, vp2, and vp3 proteins which are 95% to 100% deamidated in at least four positions of SEQ ID NO: 6, and optionally deamidated in other positions;
(d) an AAVhu80 capsid consisting of (i) a capsid produced from a nucleic acid sequence encoding SEQ ID NO: 8; (ii) a capsid produced from a nucleic acid sequence of SEQ ID NO: 7 of a sequence or a sequence at least 95% identical thereto encoding SEQ ID NO: 8; or (iii) a capsid which is a heterogeneous mixture of AAVhu80 vp1, vp2, and vp3 proteins which are 95% to 100% deamidated in at least four positions of SEQ ID NO: 8, and optionally deamidated in other positions;
(e) an AAVhu83 capsid consisting of (i) a capsid produced from a nucleic acid sequence encoding SEQ ID NO: 10; (ii) a capsid produced from a nucleic acid sequence of SEQ ID NO: 9 of a sequence or a sequence at least 95% identical thereto encoding SEQ ID NO: 10; or (iii) a capsid which is a heterogeneous mixture of AAVhu83 vp1, vp2, and vp3 proteins which are 95% to 100% deamidated in at least four positions of SEQ ID NO: 10, and optionally deamidated in other positions;
(f) an AAVhu74/71 capsid consisting of (i) a capsid produced from a nucleic acid sequence encoding SEQ ID NO: 12; (ii) a capsid produced from a nucleic acid sequence of SEQ ID NO: 11 of a sequence or a sequence at least 95% identical thereto encoding SEQ ID NO: 12; or (iii) a capsid which is a heterogeneous mixture of AAVhu74/71 vp1, vp2, and vp3 proteins which are 95% to 100% deamidated in at least four positions of SEQ ID NO: 12, and optionally deamidated in other positions;
(g) an AAVhu77 capsid, consisting of (i) a capsid produced from a nucleic acid sequence encoding SEQ ID NO: 14; (ii) a capsid produced from a nucleic acid sequence of SEQ ID NO: 13 of a sequence or a sequence at least 95% identical thereto encoding SEQ ID NO: 14; or (iii) a capsid which is a heterogeneous mixture of AAVhu77 vp1, vp2, and vp3 proteins which are 95% to 100% deamidated in at least four positions of SEQ ID NO: 14, and optionally deamidated in other positions;
(h) an AAVhu78/88 capsid consisting of (i) a capsid produced from a nucleic acid sequence encoding SEQ ID NO: 16; (ii) a capsid produced from a nucleic acid sequence of SEQ ID NO: 15 of a sequence or a sequence at least 95% identical thereto encoding SEQ ID NO: 16; or (iii) a capsid which is a heterogeneous mixture of AAVhu78/88 vp1, vp2, and vp3 proteins which are 95% to 100% deamidated in at least four positions of SEQ ID NO: 16, and optionally deamidated in other positions;
(i) an AAVhu70 capsid consisting of (i) a capsid produced from a nucleic acid sequence encoding SEQ ID NO: 18; (ii) a capsid produced from a nucleic acid sequence of SEQ ID NO: 17 of a sequence or a sequence at least 95% identical thereto encoding SEQ ID NO: 18; or (iii) a capsid which is a heterogeneous mixture of AAVhu70 vp1, vp2, and vp3 proteins which are 95% to 100% deamidated in at least four positions of SEQ ID NO: 18, and optionally deamidated in other positions;
(j) an AAVhu72 capsid consisting of (i) a capsid produced from a nucleic acid sequence encoding SEQ ID NO: 20; (ii) a capsid produced from a nucleic acid sequence of SEQ ID NO: 19 of a sequence or a sequence at least 95% identical thereto encoding SEQ ID NO: 20; or (iii) a capsid which is a heterogeneous mixture of AAVhu72 vp1, vp2, and vp3 proteins which are 95% to 100% deamidated in at least four positions of SEQ ID NO: 20, and optionally deamidated in other positions;
(k) an AAVhu75 capsid consisting of (i) a capsid produced from a nucleic acid sequence encoding SEQ ID NO: 22; (ii) a capsid produced from a nucleic acid sequence of SEQ ID NO: 21 of a sequence or a sequence at least 95% identical thereto encoding SEQ ID NO: 22; or (iii) a capsid which is a heterogeneous mixture of AAVhu75 vp1, vp2, and vp3 proteins which are 95% to 100% deamidated in at least four positions of SEQ ID NO: 22, and optionally deamidated in other positions;
(l) an AAVhu76 capsid consisting of (i) a capsid produced from a nucleic acid sequence encoding SEQ ID NO: 24; (ii) a capsid produced from a nucleic acid sequence of SEQ ID NO: 23 of a sequence or a sequence at least 95% identical thereto encoding SEQ ID NO: 24; or (iii) a capsid which is a heterogeneous mixture of AAVhu76 vp1, vp2, and vp3 proteins which are 95% to 100% deamidated in at least four positions of SEQ ID NO: 24, and optionally deamidated in other positions;
(m) an AAVhu81 capsid consisting of (i) a capsid produced from a nucleic acid sequence encoding SEQ ID NO: 26; (ii) a capsid produced from a nucleic acid sequence of SEQ ID NO: 25 of a sequence or a sequence at least 95% identical thereto encoding SEQ ID NO: 26; or (iii) a capsid which is a heterogeneous mixture of AAVhu81 vp1, vp2, and vp3 proteins which are 95% to 100% deamidated in at least four positions of SEQ ID NO: 26, and optionally deamidated in other positions;
(n) an AAVhu82 capsid consisting of (i) a capsid produced from a nucleic acid sequence encoding SEQ ID NO: 28; (ii) a capsid produced from a nucleic acid sequence of SEQ ID NO: 27 of a sequence or a sequence at least 95% identical thereto encoding SEQ ID NO: 28; or (iii) a capsid which is a heterogeneous mixture of AAVhu82 vp1, vp2, and vp3 proteins which are 95% to 100% deamidated in at least four positions of SEQ ID NO: 28, and optionally deamidated in other positions;
(o) an AAVhu84 capsid consisting of (i) a capsid produced from a nucleic acid sequence encoding SEQ ID NO: 30; (ii) a capsid produced from a nucleic acid sequence of SEQ ID NO: 29 of a sequence or a sequence at least 95% identical thereto encoding SEQ ID NO: 30; or (iii) a capsid which is a heterogeneous mixture of AAVhu84 vp1, vp2, and vp3 proteins which are 95% to 100% deamidated in at least four positions of SEQ ID NO: 30, and optionally deamidated in other positions;
(p) an AAVhu86 capsid consisting of (i) a capsid produced from a nucleic acid sequence encoding SEQ ID NO: 32; (ii) a capsid produced from a nucleic acid sequence of SEQ ID NO: 31 of a sequence or a sequence at least 95% identical thereto encoding SEQ ID NO: 32; or (iii) a capsid which is a heterogeneous mixture of AAVhu86 vp1, vp2, and vp3 proteins which are 95% to 100% deamidated in at least four positions of SEQ ID NO: 32, and optionally deamidated in other positions;
(q) an AAVhu87 capsid consisting of (i) a capsid produced from a nucleic acid sequence encoding SEQ ID NO: 34; (ii) a capsid produced from a nucleic acid sequence of SEQ ID NO: 33 of a sequence or a sequence at least 95% identical thereto encoding SEQ ID NO: 34; or (iii) a capsid which is a heterogeneous mixture of AAVhu87 vp1, vp2, and vp3 proteins which are 95% to 100% deamidated in at least four positions of SEQ ID NO: 34, and optionally deamidated in other positions;
(r) an AAVhu88/78 capsid consisting of (i) a capsid produced from a nucleic acid sequence encoding SEQ ID NO: 36; (ii) a capsid produced from a nucleic acid sequence of SEQ ID NO: 35 of a sequence or a sequence at least 95% identical thereto encoding SEQ ID NO: 36; or (iii) a capsid which is a heterogeneous mixture of AAVhu88/78 vp1, vp2, and vp3 proteins which are 95% to 100% deamidated in at least four positions of SEQ ID NO: 36, and optionally deamidated in other positions;
(s) an AAVhu69 capsid consisting of (i) a capsid produced from a nucleic acid sequence encoding SEQ ID NO: 38; (ii) a capsid produced from a nucleic acid sequence of SEQ ID NO: 37 of a sequence or a sequence at least 95% identical thereto encoding SEQ ID NO: 38; or (iii) a capsid which is a heterogeneous mixture of AAVhu69 vp1, vp2, and vp3 proteins which are 95% to 100% deamidated in at least four positions of SEQ ID NO: 38, and optionally deamidated in other positions;
(t) an AAVrh76 capsid consisting of (i) a capsid produced from a nucleic acid sequence encoding SEQ ID NO: 42; (ii) a capsid produced from a nucleic acid sequence of SEQ ID NO: 41 of a sequence or a sequence at least 95% identical thereto encoding SEQ ID NO: 42; or (iii) a capsid which is a heterogeneous mixture of AAVhu69 vp1, vp2, and vp3 proteins which are 95% to 100% deamidated in at least four positions of SEQ ID NO: 42, and optionally deamidated in other positions;
(u) an AAVrh77 capsid consisting of (i) a capsid produced from a nucleic acid sequence encoding SEQ ID NO: 44; (ii) a capsid produced from a nucleic acid sequence of SEQ ID NO: 43 of a sequence or a sequence at least 95% identical thereto encoding SEQ ID NO: 44; or (iii) a capsid which is a heterogeneous mixture of AAVrh71 vp1, vp2, and vp3 proteins which are 95% to 100% deamidated in at least four positions of SEQ ID NO: 44, and optionally deamidated in other positions;
(v) an AAVrh78 capsid consisting of (i) a capsid produced from a nucleic acid sequence encoding SEQ ID NO: 46; (ii) a capsid produced from a nucleic acid sequence of SEQ ID NO: 45 of a sequence or a sequence at least 95% identical thereto encoding SEQ ID NO: 46; or (iii) a capsid which is a heterogeneous mixture of AAVrh78 vp1, vp2, and vp3 proteins which are 95% to 100% deamidated in at least four positions of SEQ ID NO: 46, and optionally deamidated in other positions;
(w) an AAVrh81 capsid consisting of (i) a capsid produced from a nucleic acid sequence encoding SEQ ID NO: 50; (ii) a capsid produced from a nucleic acid sequence of SEQ ID NO: 49 of a sequence or a sequence at least 95% identical thereto encoding SEQ ID NO: 50; or (iii) a capsid which is a heterogeneous mixture of AAVrh81 vp1, vp2, and vp3 proteins which are 95% to 100% deamidated in at least four positions of SEQ ID NO: 50, and optionally deamidated in other positions;
(x) an AAVrh89 capsid consisting of (i) a capsid produced from a nucleic acid sequence encoding SEQ ID NO: 52; (ii) a capsid produced from a nucleic acid sequence of SEQ ID NO: 51 of a sequence or a sequence at least 95% identical thereto encoding SEQ ID NO: 52; or (iii) a capsid which is a heterogeneous mixture of AAVrh89 vp1, vp2, and vp3 proteins which are 95% to 100% deamidated in at least four positions of SEQ ID NO: 52, and optionally deamidated in other positions;
(y) an AAVrh82 capsid consisting of (i) a capsid produced from a nucleic acid sequence encoding SEQ ID NO: 54; (ii) a capsid produced from a nucleic acid sequence of SEQ ID NO: 53 of a sequence or a sequence at least 95% identical thereto encoding SEQ ID NO: 54; or (iii) a capsid which is a heterogeneous mixture of AAVrh82 vp1, vp2, and vp3 proteins which are 95% to 100% deamidated in at least four positions of SEQ ID NO: 54, and optionally deamidated in other positions;
(z) an AAVrh83 capsid consisting of (i) a capsid produced from a nucleic acid sequence encoding SEQ ID NO: 56; (ii) a capsid produced from a nucleic acid sequence of SEQ ID NO: 55 of a sequence or a sequence at least 95% identical thereto encoding SEQ ID NO: 56; or (iii) a capsid which is a heterogeneous mixture of AAVrh83 vp1, vp2, and vp3 proteins which are 95% to 100% deamidated in at least four positions of SEQ ID NO: 56, and optionally deamidated in other positions;
(aa) an AAVrh84 capsid consisting of (i) a capsid produced from a nucleic acid sequence encoding SEQ ID NO: 58; (ii) a capsid produced from a nucleic acid sequence of SEQ ID NO: 57 of a sequence or a sequence at least 95% identical thereto encoding SEQ ID NO: 58; or (iii) a capsid which is a heterogeneous mixture of AAVrh84 vp1, vp2, and vp3 proteins which are 95% to 100% deamidated in at least four positions of SEQ ID NO: 58, and optionally deamidated in other positions;
(bb) an AAVrh85 capsid consisting of (i) a capsid produced from a nucleic acid sequence encoding SEQ ID NO: 60; (ii) a capsid produced from a nucleic acid sequence of SEQ ID NO: 59 of a sequence or a sequence at least 95% identical thereto encoding SEQ ID NO: 60; or (iii) a capsid which is a heterogeneous mixture of AAVrh85 vp1, vp2, and vp3 proteins which are 95% to 100% deamidated in at least four positions of SEQ ID NO: 60, and optionally deamidated in other positions;
(cc) an AAVrh87 capsid consisting of (i) a capsid produced from a nucleic acid sequence encoding SEQ ID NO: 62; (ii) a capsid produced from a nucleic acid sequence of SEQ ID NO: 61 of a sequence or a sequence at least 95% identical thereto encoding SEQ ID NO: 62; or (iii) a capsid which is a heterogeneous mixture of AAVrh87 vp1, vp2, and vp3 proteins which are 95% to 100% deamidated in at least four positions of SEQ ID NO: 62, and optionally deamidated in other positions; or
(dd) an AAVhu73 capsid consisting of (i) a capsid produced from a nucleic acid sequence encoding SEQ ID NO: 74; (ii) a capsid produced from a nucleic acid sequence of SEQ ID NO: 73 of a sequence or a sequence at least 95% identical thereto encoding SEQ ID NO: 74; or (iii) a capsid which is a heterogeneous mixture of AAVrh73 vp1, vp2, and vp3 proteins which are 95% to 100% deamidated in at least four positions of SEQ ID NO: 74, and optionally deamidated in other positions.

2. The rAAV according to embodiment 1, wherein the gene product is useful in treating a disorder or disease of the liver, and wherein the capsid is an AAVrh75, AAVrh79, AAVrh83, or AAVrh84 capsid.

3. The rAAV according to embodiment 1, wherein the gene product is a gene editing nuclease.

4. The rAAV according to claim 1, wherein the gene encodes a gene editing nucleasefor.

5. The rAAV according to any one of embodiments 1 to 4, wherein the expression cassette comprises a tissue-specific promoter.

6. A host cell containing the rAAV according to any one of embodiments 1 to 5.

7. A pharmaceutical composition comprising the rAAV according to any one of embodiments 1 to 5, and a physiologically compatible carrier, buffer, adjuvant, and/or diluent.

8. A method of delivering a transgene to a cell, said method comprising the step of contacting the cell with the rAAV according to any one of embodiments 1 to 5, wherein said rAAV comprises the transgene.

9. A method of generating a recombinant adeno-associated virus (rAAV) comprising an AAV capsid, the method comprising culturing a host cell containing: (a) a molecule encoding an AAV vp1, vp2, and/or vp3 capsid protein of AAVrh75 (SEQ ID NO: 40), AAVhu71/74 (SEQ ID NO: 4), AAVhu79 (SEQ ID NO: 6), AAVhu80 (SEQ ID NO: 8), AAVhu83 (SEQ ID NO: 10), AAVhu74/71 (SEQ ID NO: 12), AAVhu77 (SEQ ID NO: 14), AAVhu78/88 (SEQ ID NO: 16), AAVhu70 (SEQ ID NO: 18), AAVhu72 (SEQ ID NO: 20), AAVhu75 (SEQ ID NO: 22), AAVhu76 (SEQ ID NO: 24), AAVhu81 (SEQ ID NO: 26), AAVhu82 (SEQ ID NO: 28), AAVhu84 (SEQ ID NO: 30), AAVhu86 (SEQ ID NO: 32), AAVhu87 (SEQ ID NO: 34), AAVhu88/78 (SEQ ID NO: 36), AAVhu69 (SEQ ID NO: 38), AAVrh76 (SEQ ID NO: 42), AAVrh77 (SEQ ID NO: 44), AAVrh78 (SEQ ID NO: 46), AAVrh81 (SEQ ID NO: 50), AAVrh89 (SEQ ID NO: 52), AAVrh82 (SEQ ID NO: 54), AAVrh83 (SEQ ID NO: 56), AAVrh84 (SEQ ID NO: 58), AAVrh85 (SEQ ID NO: 60), AAVrh87 (SEQ ID NO: 62), or AAVhu73 (SEQ ID NO: 74), or an AAV vp1, vp2, and/or vp3 capsid protein sharing at least 99% identity with any of SEQ ID NOs: 40, 4, 6, 8, 10, 12, 14, 16, 18, 20, 22, 24, 26, 28, 30, 32, 34, 36, 38, 42, 44, 46, 50, 52, 54, 56, 58, 60, 62, or 74, (b) a functional rep gene; (c) a vector genome comprising AAV inverted terminal repeats (ITRs) and a transgene; and (d) sufficient helper functions to permit packaging of the vector genome into the AAV capsid protein.

10. A plasmid comprising a vp1, vp2, and/or vp3 sequence of AAVrh75 (SEQ ID NO: 39), AAVhu71/74 (SEQ ID NO: 3), AAVhu79 (SEQ ID NO: 5), AAVhu80 (SEQ ID NO: 7), AAVhu83 (SEQ ID NO: 9), AAVhu74/71 (SEQ ID NO: 11), AAVhu77 (SEQ ID NO: 13), AAVhu78/88 (SEQ ID NO: 15), AAVhu70 (SEQ ID NO: 17), AAVhu72 (SEQ ID NO: 19), AAVhu75 (SEQ ID NO: 21), AAVhu76 (SEQ ID NO: 23), AAVhu81 (SEQ ID NO: 25), AAVhu82 (SEQ ID NO: 27), AAVhu84 (SEQ ID NO: 29), AAVhu86 (SEQ ID NO: 31), AAVhu87 (SEQ ID NO: 33), AAVhu88/78 (SEQ ID NO: 35), AAVhu69 (SEQ ID NO: 37), AAVrh76 (SEQ ID NO: 41), AAVrh77 (SEQ ID NO: 43), AAVrh78 (SEQ ID NO: 45), AAVrh81 (SEQ ID NO: 49), AAVrh89 (SEQ ID NO: 51), AAVrh82 (SEQ ID NO: 53), AAVrh83 (SEQ ID NO: 55), AAVrh84 (SEQ ID NO: 57), AAVrh85 (SEQ ID NO: 59), AAVrh87 (SEQ ID NO: 61), or AAVhu73 (SEQ ID NO: 73), or vp1, vp2, and/or vp3 sequence sharing at least 95% identity with any of SEQ ID NO: 39, 3, 5, 7, 9, 11, 13, 15, 17, 19, 21, 23, 25, 27, 29, 31, 33, 35, 37, 41, 43, 45, 49, 51, 53, 55, 57, 59, 61, or 73.

11. A cultured host cell containing the plasmid according to embodiment 10.

The following examples are illustrative of certain embodiments of the invention and are not a limitation thereon.

### EXAMPLES

Adeno-associated viruses (AAV) are advantageous as gene-transfer vectors due to their favorable biological and safety characteristics, with discovering novel AAV variants being key to improving this treatment platform. To date, researchers have isolated over 200 AAVs from natural sources using polymerase chain reaction (PCR)-based methods. We compared two modern DNA polymerases and their utility for isolating and amplifying the AAV genome. Compared to the HotStar polymerase, the higher-fidelity Q5 Hot Start High-Fidelity DNA Polymerase provided more precise and accurate amplification of the input AAV sequences. The lower-fidelity HotStar DNA polymerase introduced mutations during the isolation and amplification processes, thus generating multiple mutant capsids with variable bioactivity compared to the input AAV gene. The Q5 polymerase enabled the successful discovery of novel AAV capsid sequences from human and nonhuman primate tissue sources. Novel AAV sequences from these sources showed evidence of positive selection. This study highlights the importance of using the highest fidelity DNA polymerases available to accurately isolate and characterize AAV genomes from natural sources to ultimately develop more effective gene therapy vectors.

Adeno-associated viruses (AAVs) are safe and effective vehicles used for gene transfer for several clinical indications. AAV-mediated gene therapy drugs have been approved by the FDA for the treatment of Spinal Muscular Atrophy and Leber Congenital Amaurosis. These approved gene therapy products, as well as many others currently under development, utilize AAV capsids isolated from natural sources as the delivery vehicle 4. The AAV genome consists of two major open reading frames (ORFs), Rep and Cap, which encode sequences for the translation of multiple protein products. The Cap ORF translation occurs from multiple start sites to produce the three AAV structural proteins, VP1, VP2, and VP3. These structural protein subunits are assembled into icosahedral virions 5 which carry a genetic payload to their target. The sequence and structural diversity of AAV capsid genes contribute to variability in viral tropism, antigenicity, and packaging efficiency that is observed between viral clades. Discovering novel capsids with an array of tissue tropisms are necessary to advance the efficacy and utility of gene therapy.

AAV Cap sequences have been isolated from natural sources using a variety of techniques that have emerged and evolved over time, although the most common approach involves PCR amplification. Firstly, extracted viral DNA can be directly sequenced; this method was used to identify AAV2, which was found to be propagated with helper Adenovirus in cell culture. Secondly, extracted viral DNA can be extracted, cloned into a plasmid backbone, and sequenced (AAV1, AAV3, AAV3B, AAV6, and AAV5). Thirdly, it is possible to extract viral genomes via PCR and clone the amplicons into plasmids before Sanger sequencing. Many AAVs from primate, bovine, porcine, rodent, and others have been isolated using this method. Next-generation sequencing (NGS) analyses of mammalian genomic DNA have detected fragments of endogenous AAV genomic elements. More recently, metagenomic virome sequencing studies, which use shotgun-NGS to simultaneously sequence thousands of DNA molecules in complex samples, have identified many novel AAV sequences.

The use of PCR for AAV amplification provides a straightforward and effective means to discover novel AAV capsid sequences. However, it is important to utilize PCR enzymes with high-fidelity replication capabilities to amplify the viral sequences as accurately as possible. Enzymes with high misincorporation and template-switching rates can significantly confound sequencing data and interfere with novel AAV capsid discovery. Indeed, the artificial variability introduced by low-fidelity polymerases while amplifying capsid sequences can impair the study of AAV biology and diversity due to the amplification error skewing the ‘true’ genetic variation in a sample.

We aimed to compare multiple AAV PCR methods to screen tissue samples for AAV natural isolate genomes to expand the breadth of capsid sequences available for characterization as potential gene delivery vectors. Discovering more capsids increases the chance of successfully identifying those that can transfer therapeutic transgenes to a range of tissues at high efficiency, have reduced immunogenicity at high doses, and have less prevalent neutralizing antibody profiles in the human population than existing AAV capsids. Given that DNA polymerase technology has undergone significant development since the last wave of AAV discovery almost 20 years ago, we compared two modern DNA polymerases and amplification methods to isolate AAV sequences. We found that the Q5 Hot Start High-Fidelity DNA Polymerase produced PCR products from the input templates at higher accuracy compared to the lower-fidelity HotStar DNA polymerase. Using the Q5 DNA polymerase, we also studied the genetic diversity of the newly isolated AAV capsid sequences by performing phylogenetic analyses. Furthermore, we found that the novel AAV natural isolates showed evidence of evolution by positive selection.

### Example 1: Materials and Methods

DNA Extraction from Nonhuman Primate and Human Tissue

Nonhuman primate (Macaca mulatta) tissue samples were collected postmortem from the Gene Therapy Program at the University of Pennsylvania's Perelman School of Medicine. Human tissue samples (including aortic valve, bone marrow, brain, breast, cervix, colon, heart, intestine, kidney, liver, lung, lymph node, ovary, pancreas, pericardium, skeleton muscle, and spleen) were obtained. Genomic DNA was extracted using the QIAamp DNA Mini Kit (QIAGEN Inc., Germantown, MD).

### Conventional AAV Isolation

To amplify 3.1 kb AAV genome sequences from host genomic DNA, we utilized the Q5 Hot Start High-Fidelity DNA polymerase, using working conditions determined by the manufacturer (New England Biolabs, Ipswich, MA). We used the previously described AV1NS forward primer and the AV2CAS reverse primer to isolate AAV genomes; we replaced the degenerate base Y in AV1NS with a T (AV1NS 5′-GCTGCGTCAACTGGACCAATGAGAAC-3′; SEQ ID NO: 63) and AV2CAS (5′-CGCAGAGACCAAAGTTCAACTGAAACGA-3′; SEQ ID NO: 64) (Gao G P et al. PNAS USA. 2002; 99:11854-59) because T is the primary nucleotide that is represented in the AAV sequence phylogeny across many clades of AAV. Each primer was used at a 0.5 μM final concentration, as described in the Q5 protocol (New England Biolabs, Ipswich, MA). The following thermal cycling conditions were applied: 98° C. for 30 s; 98° C. for 10 s, 59° C. for 10 s, 72° C. for 93 s, 50 cycles; and a 72° C. extension for 120 s. PCR products were TOPO-cloned (Thermo Fisher Scientific, Waltham, MA) and Sanger-sequenced (GENEWIZ, South Plainfield, NJ). For most PCR products, we sequenced at least three clones.

### AAV Isolation by Single Genome Amplification

Genomic DNA from a human heart tissue sample that was previously found to be AAV-positive by conventional AAV isolation PCR was subjected to AAV-SGA. AAV-containing genomic DNA was endpoint-diluted in 20 ng/μL sheared-salmon sperm DNA (Ambion, Inc, Austin, TX) by serial dilutions. Material from each serial dilution was used as the template for 96 PCR reactions using the AV1NS and AV2CAS primers (Mueller C et al. Curr Protoc Microbiol 2012; Chapter 14:Unit14D11). We utilized Q5 Hot Start High-Fidelity DNA polymerase (New England Biolabs, Ipswich, MA) to amplify AAV DNA using the following cycling conditions: 98° C. for 30 s; 98° C. for 10 s, 59° C. for 10 s, 72° C. for 93 s, 50 cycles; and a 72° C. extension for 120 s. For a Poisson distribution, the DNA dilution that yields PCR products in no more than 30% of wells contains one amplifiable AAV DNA template per positive PCR in more than 80% of cases (Salazar-Gonzalez J F et al. Journal of Virology 2008; 82:3952-70). AAV DNA amplicons from positive PCR reactions were purified using Agencourt Ampure XP Beads (Beckman Coulter, Brea, CA), libraries were constructed using the NEBNext® Ultra™ II DNA Library Prep Kit for Illumina® (NEB, Ipswich, MA), and sequenced using the Illumina MiSeq 2×250 (Illumina, San Diego, CA) paired-end sequencing platform, and the resulting reads were assembled de novo using the SPAdes assembler (cab.spbu.ru/software/spades/).

### Sequence Analysis

We aligned AAV sequences using the AlignX component of Vector NTI Advance® 11.5.4 (Thermo Fisher Scientific, Waltham, MA) or Geneious Prime version 2019.2 (geneious.com). A GenBank sequence comparison was performed on the NCBI BLAST server (blast.ncbi.nlm.nih.gov/Blast.cgi).

### Polymerase Fidelity Comparison

The pAAV2/9 trans plasmid was used as the template. To make sure the template was pure, we first re-transformed the plasmid into Stable Competent E. coli cells (Thermo Fisher, Waltham, MA), and sequenced two, single colony clones via NGS (Illumina, San Diego, CA) as described previously (Saveliev A et al. Human Gene Therapy Methods 2018; 29:201-11). To ensure complete sequence identity to the input pAAV2/9 trans plasmid, we used one of the two sequenced plasmids as the template for subsequent experiments. In this comparative study, the Hot Star HiFidelity polymerase (“HiFi”) (QIAGEN Inc., Germantown, MD) was the lower-fidelity polymerase whereas the Q5 Hot Start High-Fidelity DNA polymerase (Q5) (New England Biolabs, Ipswich, MA) was the higher-fidelity polymerase. For “HiFi Circular,” the pAAV2/9 trans plasmid was diluted and used as the PCR template. For “HiFi Linear” and “Q5 Linear,” the pAAV2/9 trans plasmid was linearized with the restriction enzyme PvuII (New England Biolabs, Ipswich, MA) and then diluted for use as the template. For all first-round PCRs, we utilized five copies of the template in a 25-μL reaction. In the second round, we used 1 μL of the first-round PCR product as the template in a 50-μL reaction. PCR conditions were based on the manufacturer's guidelines.

For all “HiFi” experiments, we employed the HotStar HiFidelity polymerase (QIAGEN Inc., Germantown, MD). AV1NS' and AV2CAS primers were used in accordance with the manufacturer's protocol. We applied the following thermal cycling conditions for the first-round PCR: 95° C. for 300 s; 94° C. for 15 s, 63° C. for 60 s, 68° C. for 371 s, 40 cycles; and a 72° C. extension for 600 s. For the second round of PCR, we used the primers McapF3SpeI (5′-ATCGATACTAGTCCATCGACGTCAGACGCGGAAG-3′; SEQ ID NO: 65) and McapR1NotI (5′-ATCGATGCGGCCGCAGTTCAACTGAAACGAATTAAACGGT-3′; SEQ ID NO: 66) to perform a nested reaction. McapF3SpeI and McapR1NotI′ were described in a previous publication on an AAV PCR technique (Smith U et al. Molecular Therapy 2014; 22:1625-1634). McapR1NotI′ is a modified version of the primer McapR1NotI from the aforementioned publication; we modified McapR1NotI to correct for two base pairs near its 3′ end that do not align with any reported AAV sequences, including the isolates reported in the previous publication. 1 μL of the first-round PCR product was used as the template in the second, nested, round of PCR. The following thermal cycling conditions were used for the second round of PCR: 95° C. for 300 s; 94° C. for 15 s, 63° C. for 60 s, 68° C. for 315 s, 40 cycles; and a 72° C. extension for 600 s.

For the first round of the “Q5” reaction, we used the Q5 Hot Start High-Fidelity DNA polymerase master mix (New England Biolabs, Ipswich, MA). We used AV1NS' and AV2CAS primers in each reaction in accordance with the manufacturer's protocol. The thermal cycling conditions were as follows: 98° C. for 30 s; 98° C. for 10 s, 59° C. for 30 s, 72° C. for 186 s, 40 cycles; and a 72° C. extension for 120 s. For the second round of “Q5” reactions, we utilized the primers McapF3SpeI and McapR1NotI′. 1 μL of the first-round “Q5” PCR product was used as the template in the second, nested, round of PCR in each 50-μL reaction. The thermal cycling conditions were as follows: 98° C. for 30 s; 98° C. for 10 s, 66° C. for 30 s, 72° C. for 164 s, 40 cycles; and a 72° C. extension for 120 s. The PCR products were then TOPO-cloned and sequenced.

Vector Production, Quantitative PCR (qPCR) Titration, and Huh7 Transduction Assay

For AAV vector production in six-well plates, we adapted a previously described 1-cell-stack-scale HEK293 triple-transfection protocol based on the reduced culture areas, with a few modifications: 1) the plasmid ratio used was 2:1:0.1 (helper plasmid containing the required Adenovirus helper genes: trans plasmid containing AAV2 Rep and AAV capsid genes: cis plasmid containing the CB7 promoter, Firefly luciferase gene, and the rabbit beta globin polyadenylation sequence transgene (i.e., CB7.ffluciferase.rBG), by weight), and 2) at harvest, no other treatment was performed beyond freezing/thawing (Lock M et al. Human Gene Therapy 2010; 21:1259-1271). We measured the vector production titer by qPCR using primers and probe against the vector poly A sequence.

### AAV VP1 Sequence Evolution Analysis

Geneious version 2019.2 (geneious.com) was applied to construct the DNA sequence alignment and used the Geneious alignment algorithm. We used the branch-site unrestricted statistical test for episodic diversification (BUSTED) and mixed-effects model of evolution (MEME) programs to perform positive-selection hypothesis testing statistical analyses on AAV VP1 DNA sequences. The Fixed Effects Likelihood (FEL) test was used to perform negative-selection hypothesis testing. These programs ran on the HyPhy server at datamonkey.org. For the human and rhesus macaque AAV natural isolates, we used BUSTED and FEL to compare each new isolate's phylogenetic branch to the branch that ended in its closest BLASTn hit. For the AAVHSC and AAV HiFi PCR mutant variants, we tested all branches of the phylogeny as a whole to determine whether positive selection occurred at any possible site over the entire tree due to the inherent sequence similarity of these populations (Smith L J et al. Molecular Therapy 2014; 22:1625-34). BUSTED and FEL utilize the likelihood ratio test to determine significance i.e., whether or not there is evidence for positive or negative selection across a gene. For MEME analysis, we evaluated each phylogeny (human, rhesus, HSC, and HiFi) for the presence of positive episodic or pervasive selection. MEME uses the likelihood ratio test to determine significance. Results that produced p<0.05 were considered to be significant. AAVrh81 was removed from the rhesus phylogeny for analysis due to its significant sequence divergence from the remainder of the group.

We constructed all phylogenetic trees using the MAFFT version 7 server (mafft.cbrc.jp/alignment/server/) using the neighbor-joining method. Trees were bootstrapped 100 times and formatted using FigTree (tree.bio.ed.ac.uk/software/figtree/).

### Statistics

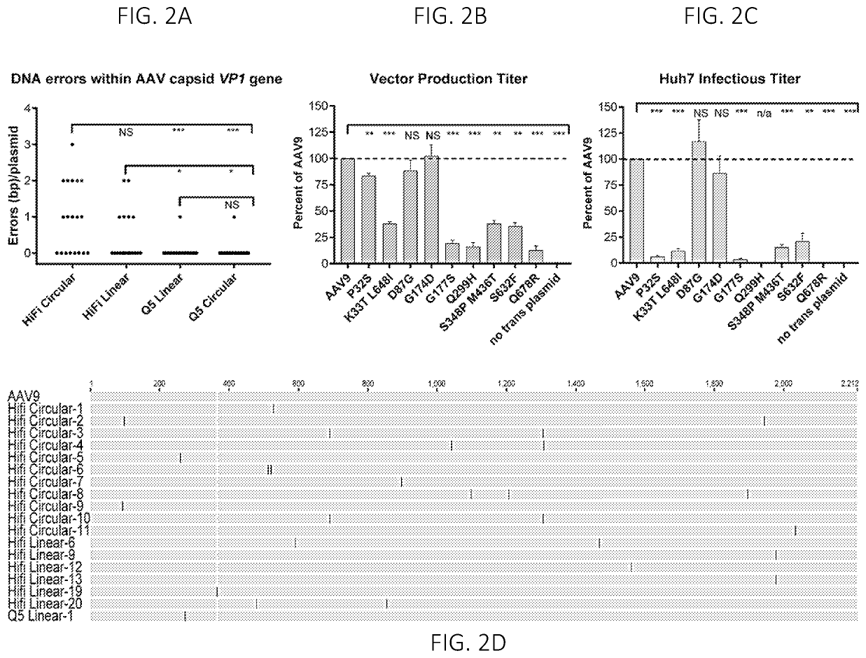

For FIG. 2A, we performed pairwise comparison between each group using the Wilcoxon rank-sum test using the “wilcox.test” function within the R Program (version 3.5.0; cran.r-project.org). For FIG. 2B and FIG. 2C, the Student's t-test was used to compare each mutant to AAV9 using the “t.test” function within the R Program (version 4.0.0; cran.r-project.org). Statistical significance was assessed at the 0.05 level.

### Example 2: A Lower-Fidelity DNA Polymerase Produced More Random Mismatch Errors

We first evaluated the impact of polymerase fidelity on AAV isolation to test the assertion that lower-fidelity DNA polymerases would produce amplicons with a higher frequency of PCR error. We used a pure, NGS-verified, AAV9 trans plasmid (i.e., pAAV2/9) containing the AAV2 Rep gene and the AAV9 Cap gene as the PCR template in reactions containing DNA polymerases with varying levels of replication fidelity. We applied a high-fidelity polymerase, the Q5 Hot Start High-Fidelity DNA polymerase (Q5), and a relatively lower-fidelity polymerase, the HotStar HiFidelity (HiFi) polymerase, due to their varying levels of known polymerase fidelity. Employing the same protocol used to isolate AAV natural isolates AAVHSC1-17 with the HiFi polymerase (Smith L J et al. Molecular Therapy 2014; 22:16-1634), we found that plasmids cloned and sequenced from the HiFi polymerase PCR products contained 30%-60% more occurrences of random errors across the VP1 region compared to those generated using the high-fidelity Q5 DNA polymerase: eleven out of nineteen and six out of twenty total sequenced PCR product clones from the HiFi Circular and Linear groups, respectively, contained at least one mismatch. In contrast, only one out of 20 and 24 sequenced PCR product clones had a mismatch in the Q5 linear and circular groups, respectively (FIG. 2A, FIG. 2D, and Table 1).

We next aimed to determine whether the AAV9 PCR isolate capsid sequences generated from the HiFi polymerase experiments were functional. We cloned the isolates into pAAV2/9 trans plasmids containing the AAV2 Rep gene such that each plasmid contained a mutant AAV9 VP1 Cap gene, with these mutant trans plasmids then producing AAV vectors containing the firefly luciferase transgene (i.e., CB7.ffluciferase.rBG). Two of the mutant capsids produced vector titers at levels similar to those of wild-type AAV9 (D87G, and G174D). The remainder of the mutants showed reduced vector production capacity compared to AAV9 (FIG. 2B). P32S had a titer that was 17% lower than AAV9 while G177S, Q299H, and Q678R showed an 80%-90% reduction in production titer. S632F, K33T L6481, and S348P M436T showed a 60%-65% reduction compared to AAV9. The mutants' Huh7 infectious titers (FIG. 2C) show a pattern similar to their vector production titers, with a few exceptions—for example, the mutant P32S has a production titer of ˜83% of AAV9, but its Huh7 infectious titer is only ˜6% of AAV9, implying the mutation P32S may impair the capsid's Huh7 transduction, which warrants further investigation. Together, these results indicate that the lower-fidelity HiFi DNA polymerase produces mutants with variable functional properties in an unpredictable manner that can impair the discovery and characterization of novel isolates.

TABLE 1
Listed clones with PCR polymerase-mediated DNA mutations and their
associated amino acid changes. Mutation DNA and protein numbering
based on AAV9 VP1 sequence. AAV9 VP nucleic acid sequence (SEQ
ID NO: 67). AAV9 VP1 amino acid sequence (SEQ ID NO: 68).

| | Number | | | | | | |
|---|---|---|---|---|---|---|---|
| | of PCR | | | | | | |
| | mutations | | | | | | |
| Clone name | in VP1 | DNA mutation | | | Protein change | | |
| HiFi Circular-8 | 3 | g1098a | g1206a | c1895t | silent | silent | S632F |
| HiFi Circular-2 | 2 | a98c | c1942a | | K33T | L648I | |
| HiFi Circular-3 | 2 | c690t | a1305g | | silent | silent | |
| HiFi Circular-4 | 2 | t1042c | t1307c | | S348P | M436T | |
| HiFi Circular-6 | 2 | c513t | g521a | | silent | G174D | |
| HiFi Circular-10 | 2 | c690t | a1305g | | silent | silent | |
| HiFi Linear-6 | 2 | g592c | c1467a | | V198L | silent | |
| HiFi Linear-20 | 2 | g479a | c855t | | G160D | silent | |
| HiFi Circular-1 | 1 | g529a | | | G177S | | |
| HiFi Circular-5 | 1 | a260g | | | D87G | | |
| HiFi Circular-7 | 1 | g897t | | | Q299H | | |
| HiFi Circular-9 | 1 | c94t | | | P32S | | |
| HiFi Circular-11 | 1 | a2033g | | | Q678R | | |
| HiFi Linear-9 | 1 | a1977g | | | silent | | |
| HiFi Linear-12 | 1 | t1560c | | | silent | | |
| HiFi Linear-13 | 1 | a1977g | | | silent | | |
| HiFi Linear-19 | 1 | 368 insertion | | | frameshift | | |
| Q5 Linear-1 | 1 | a275g | | | K92R | | |
| Q5 Circular-1 | 1 | c287a | | | A96D | | |

### Example 3: Novel AAV Sequences from Multiple Clades were Isolated from Nonhuman Primate and Human Tissues Using a High-Fidelity PCR Polymerase

The advancement of gene therapy requires the identification of novel AAV capsids. The majority of currently used AAV natural variants have been derived from primate tissue. Using our validated high-fidelity Q5 PCR-based technique, we investigated whether new capsid sequences can be isolated from a panel of primate tissue samples. We used primers that bind to conserved regions of the capsid sequence to amplify a 3.1-kb AAV amplicon in order to detect and amplify the AAV genomes present in 50 nonhuman primate intestinal tissue samples. In this manner, we discovered 12 AAV natural isolate sequences. Most of these isolates belonged to clades D or E or the primate outgroup clade containing AAVrh32.33 (Table 2).

TABLE 2
Novel AAV natural isolates recovered from nonhuman
primate intestinal tissue samples and sequence
similarity to closest known AAVs.

| | | | Closest sequence hit in GenBank | |
|---|---|---|---|---|
| Source | Isolate | | Number of base differences (identities) | |
| ID | name | Clade | DNA | Protein |
| NHP1 | AAVrh75 | E | 170 (AAVrh8, | 2 (AAVrh8, |
| | | | 2041/2211) | 734/736) |
| | AAVrh76 | D | 87 (AAVrh48, | 5 (AAVrh48, |
| | | | 2127/2214) | 732/737) |
| | AAVrh77 | AAVrh32/ | 30 (AAV11, | 2 (AAV11, |
| | | rh33 like | 2172/2202) | 731/733) |
| NHP2 | AAVrh78 | AAVrh32/ | 94 (AAV11, | 5 (AAV11, |
| | | rh33 like | 2108/2202) | 728/733) |
| | AAVrh79 | E | 67 (AAVrh40, | 2 (AAVhu37, |
| | | | 2150/2217) | 736/738) |
| | AAVrh81^{a} | B | | 121 (AAVhuT70, |
| | | | | 622/743) |
| | AAVrh89 | D | 165 (AAVrh35, | 34 (AAVrh22, |
| | | | 2029/2194) | 694/728) |
| NHP3 | AAVrh82 | AAVrh32/ | 11 (AAVrh32, | 1 (AAVrh32, |
| | | rh33 like | 2191/2202) | 732/733) |
| NHP4 | AAVrh83 | E | 57 (AAVrh46, | 20 (AAVrh46, |
| | | | 2154/2211) | 718/738) |
| | AAVrh84 | E | 100 (AAVrh46, | 35 (AAVrh46, |
| | | | 2114/2214) | 703/738) |
| | AAVrh85 | D | 62 (AAV7, | 9 (AAV7, |
| | | | 2152/2214) | 728/737) |
| | AAVrh87 | D | 94 (AAV7, | 22 (AAV7, |
| | | | 2121/2215) | 715/737) |

^{a}The DNA sequence of AAVrh81 was substantially different from that of all AAVs in the GenBank database; hence, the DNA difference value is not included in this table.

We also screened genomic DNA from 271 human tissue samples using the Q5 polymerase and obtained 22 new AAV natural isolate capsid sequences including clade F member AAVhu68 (SEQ ID NO: 1). Those new AAV sequences were isolated from heart, intestine, kidney, liver, lung, and spleen. Overall, 8% of the human samples were positive for AAV. Most of the novel human isolates could be classified as clade B and C viruses or were similar to AAV2 and AAV2-AAV3 hybrids (Table 3). Three human-derived natural isolates exhibited novel DNA sequences despite having the same protein sequences as previously reported GenBank entries (i.e., AAVhu32, AAV9, and CHC367_AAV).

TABLE 3
Novel AAV natural isolates recovered from human tissue
samples and sequence similarity to closest known AAVs.

| | | | Closest sequence hit | |
|---|---|---|---|---|
| Tissue | | | Number of differences (identities) | |
| type | Isolate name | Clade | DNA | Protein |
| Heart | hu32^{b} | F | 2 (AAVhu32 2209/2211) | 0 (AAVhu32 736/736) |
| 22% (5/23) | AAVhu68 | F | 20 (AAV9 2191/2211) | 2 (AAV9 734/736) |
| | AAVhu71.74^{a} | C | 27 (CHC2107_AAV 2181/2208) | 1 (CHC367_AAV 734/735) |
| | AAVhu79 | B | 41 (CHC473_AAV 2167/2208) | 7 (AAVhuT40 728/735) |
| | AAVhu80 | B | 32 (AAVhu13 2176/2208) | 2 (CHC371_AAV 733/735) |
| Intestine | AAVhu83 | B | 33 (AAVhu29 2175/2208) | 3 (AAVhu29 732/735) |
| 25% (5/20) | AAV9^{b} | F | 10 (AAV9 2201/2211) | 0 (AAV9 736/736) |
| | AAVhu74.71^{a} | C | 23 (CHC976_AAV 2185/2208) | 1 (CHC367_AAV 734/735) |
| | AAVhu77 | C | 25 (CHC367_AAV 2183/2208) | 0 (CHC367_AAV 735/735) |
| | AAVhu78.88^{a} | C | 68 (CHC3142_AAV 2140/2208) | 9 (CHC3142_AAV 726/735) |
| Kidney 5% (1/20) | AAVhu70 | C | 33 (CHC685_AAV 2175/2208) | 3 (AAVhu60 732/735) |
| Liver 17% (9/54) | AAVhu72 | B | 36 (AAVhu13 2172/2208) | 2 (CHC2206_AAV 733/735) |
| | AAVhu75 | B | 36 (CHC473_AAV 2172/2208) | 2 (CHC1919_AAV 733/735) |
| | AAVhu76 | C | 2 (AAVhu55 2203/2205) | 2 (AAVhu55 732/734) |
| | AAVhu81 | B | 42 (CHC2087_AAV 2166/2208) | 6 (CHC371_AAV 729/735) |
| | AAVhu82 | B | 26 (AAVhuT70 2182/2208) | 2 (AAVhuT70 733/735) |
| | AAVhu84 | C | 29 (AAVhu25 2179/2208) | 2 (AAVhu60 733/735) |
| | AAVhu86 | B | 45 (CHC387_AAV 2163/2208) | 8 (CHC877_AAV 727/735) |
| | AAVhu87 | C | 52 (CHC1158_AAV 2156/2208) | 4 (Human/China/Shanghai/FX3- |
| | | | | 1613263/AAV 730/734) |
| | AAVhu88.78^{a} | C | 65 (CHC3142_AAV 2145/2210) | 9 (CHC3142_AAV 726/735) |
| Lung 3% (1/33) | AAVhu73 | C | 34 (CHC976_AAV 2174/2208) | 2 (AAVhu7 733/735) |
| Spleen 3% (1/34) | AAVhu69 | C | 6 (AAVhu18 2202/2208) | 3 (AAVhu18 732/735) |

^{a}The protein sequences of AAVhu71/AAVhu74 and AAVhu78/AAVhu88 are identical AAVhu71 = AAVhu74, AAVhu78 = AAVhu88), while their DNA sequences are different.
^{b}Recovered clones have the same amino acid sequence as previously reported AAVs, but exhibit variation in their DNA sequences.

### Example 4: AAV Single Genome Amplification (AAV-SGA) Identifies Natural Isolate AAVhu68 Capsid Sequences with High Precision and Accuracy

Single Genome Amplification (SGA) can accurately amplify individual virus sequences from a mixed sample. Based on previous reports by Salazar et al. and others for the amplification and study of HIV genome dynamics in infected patients (Salazar-Gonzalez J F et al. Journal of Virology 2008; 82:3952-70; Simmonds P et al. Journal of Virology 1990; 64:5840-50), we adapted SGA (FIG. 1) to accurately isolate AAV sequences from mammalian tissue samples using the aforementioned high-fidelity Q5 polymerase (data not shown). In this technique, endpoint-diluted genomic DNA acts as the PCR template and contains only one amplifiable AAV genome in each amplicon-positive PCR. This method prevents sequence ambiguity caused by DNA polymerase-induced mutations due to the method's replicative nature. This technique also mitigates possible DNA polymerase template-switching issues that can occur in DNA mixtures (thus leading lead to the recovery of artificially recombined amplicons) because only one AAV genome is amplified in each reaction.

We sought to verify the sequence of previously isolated AAVhu68 by performing AAV-SGA on the same tissue sample from which it originated, as described in Table 2. This technique, combined with the use of the high-fidelity Q5 polymerase, allowed us to confirm the identity of this sequence with high precision and accuracy. Our results show that all of the single-AAV genomes recovered from this sample had 99.94%-100% capsid-sequence identity to the previous, conventional Q5 PCR-isolated AAVhu68 sequence. Of the 61 single-AAV genome-derived amplicons recovered from this sample, only seven amplicons had 1- to 2 nucleotide mismatches from the original sequence. The vast majority (54/61) of amplicons had 100% DNA-sequence identity to the previously isolated AAVhu68 capsid sequence, indicating that sequence data generated using the Q5 polymerase can be interpreted with a high degree of confidence.

### Example 5: AAV Natural Isolate Capsid Protein Sequences Show Evidence of Positive Selection

Using the Q5 polymerase AAV-isolation strategy, we were able to investigate the evolutionary properties of AAV genomes with minimal influence from PCR-mediated errors. We observed that several recovered AAV natural isolate capsid sequences had greater numbers of DNA differences than corresponding protein sequence changes when compared with their closest, previously reported AAV sequence according to the GenBank sequence database.

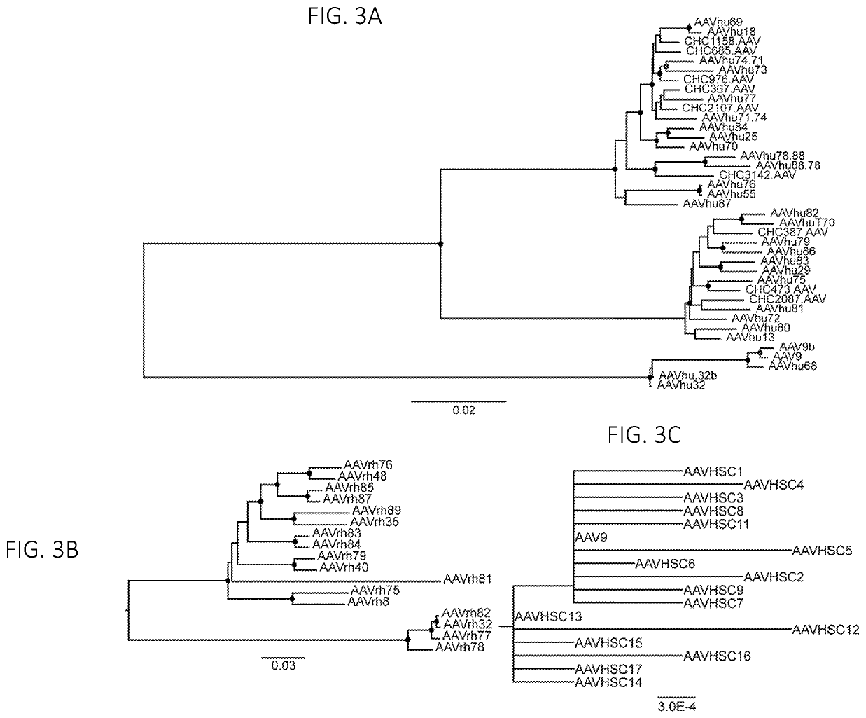

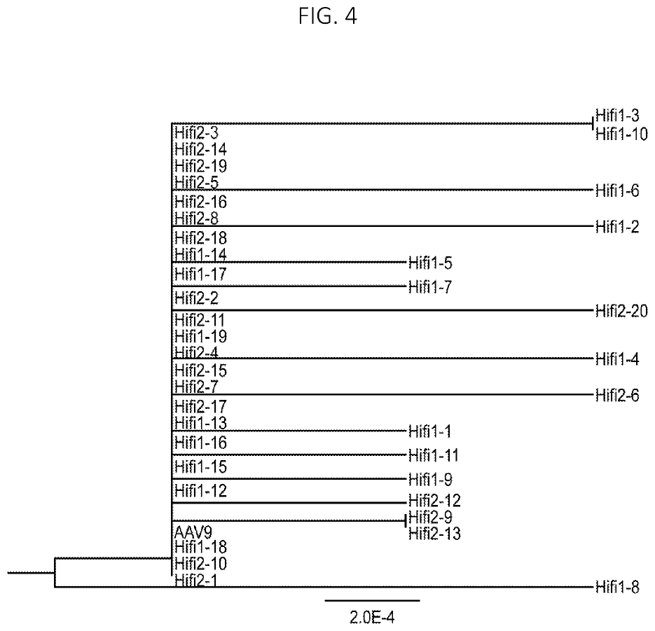

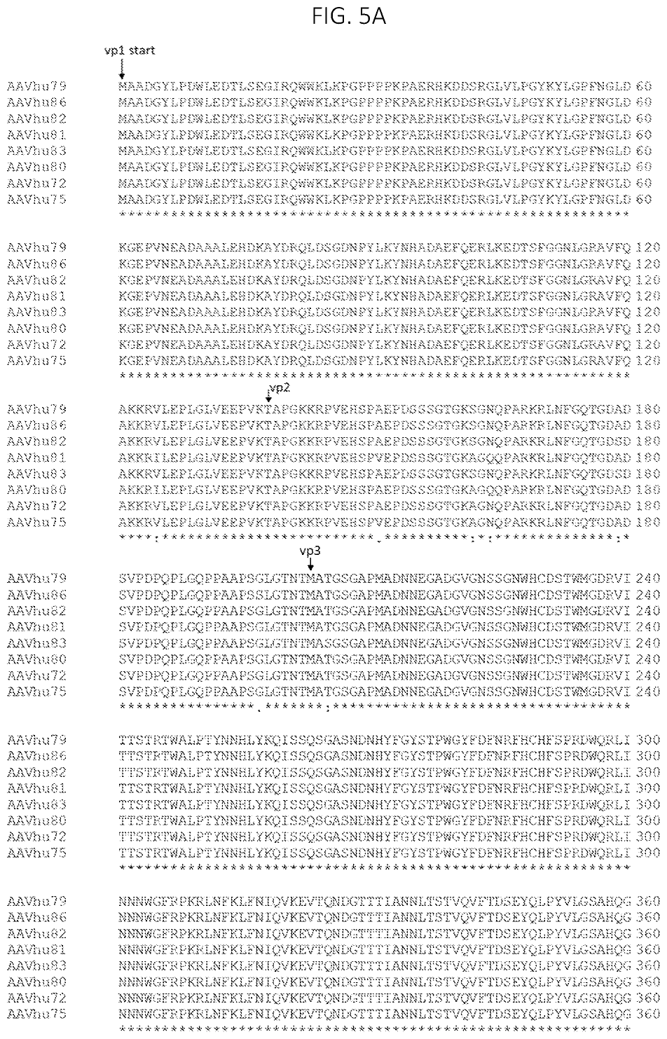

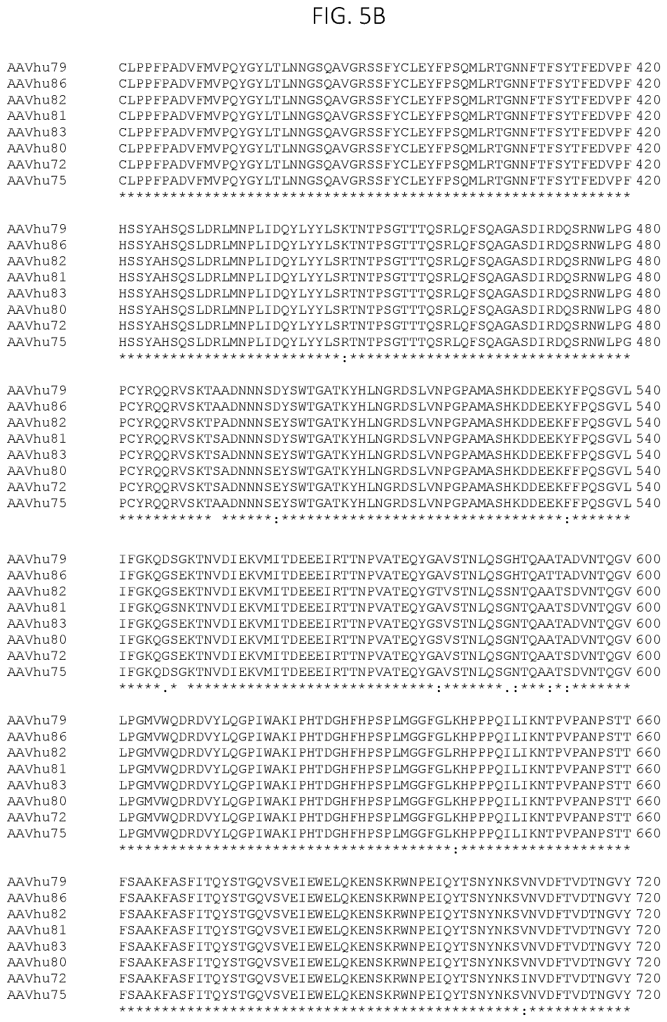

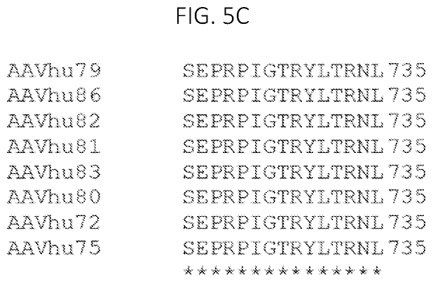

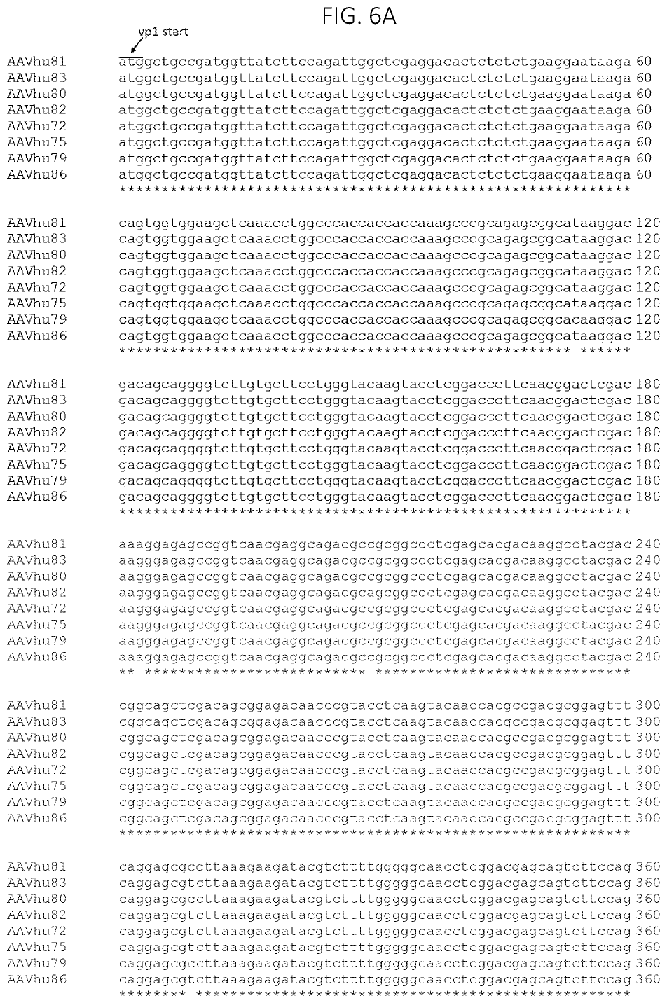

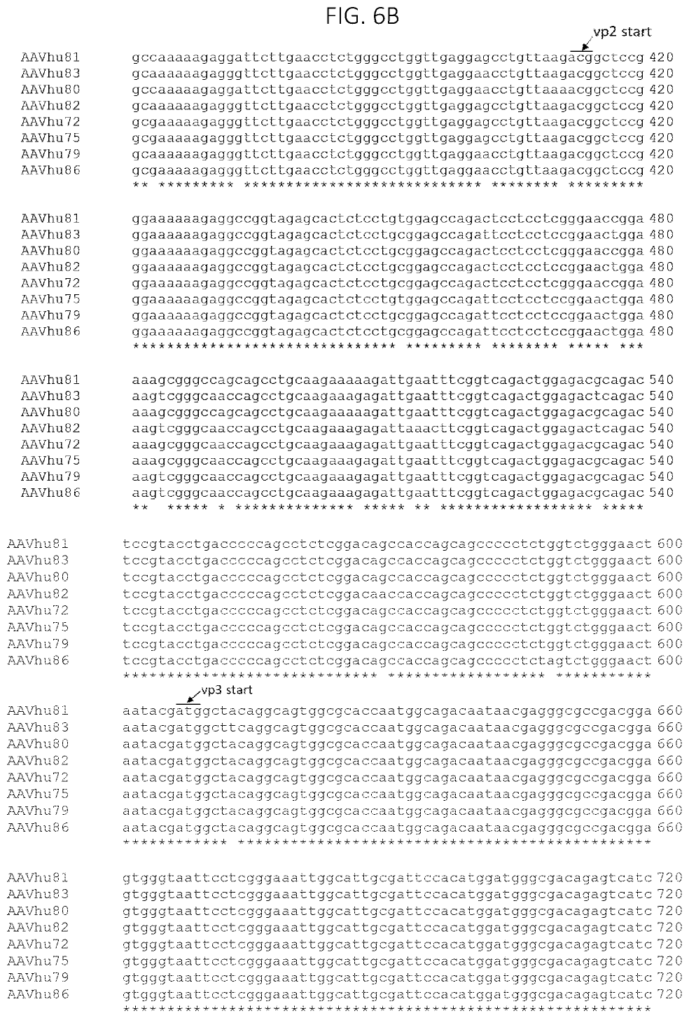

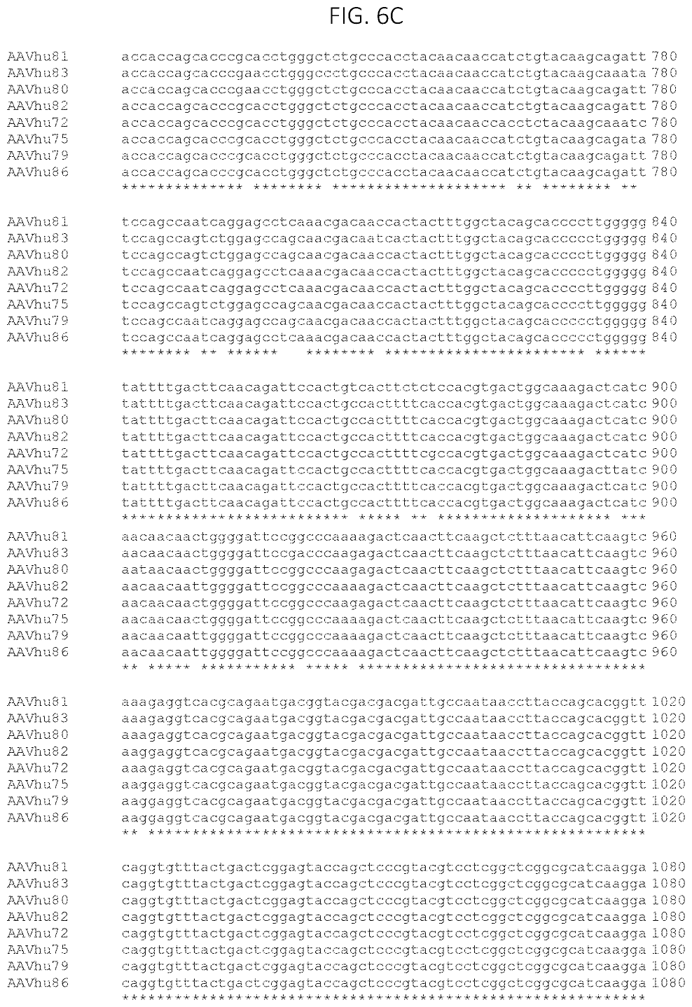

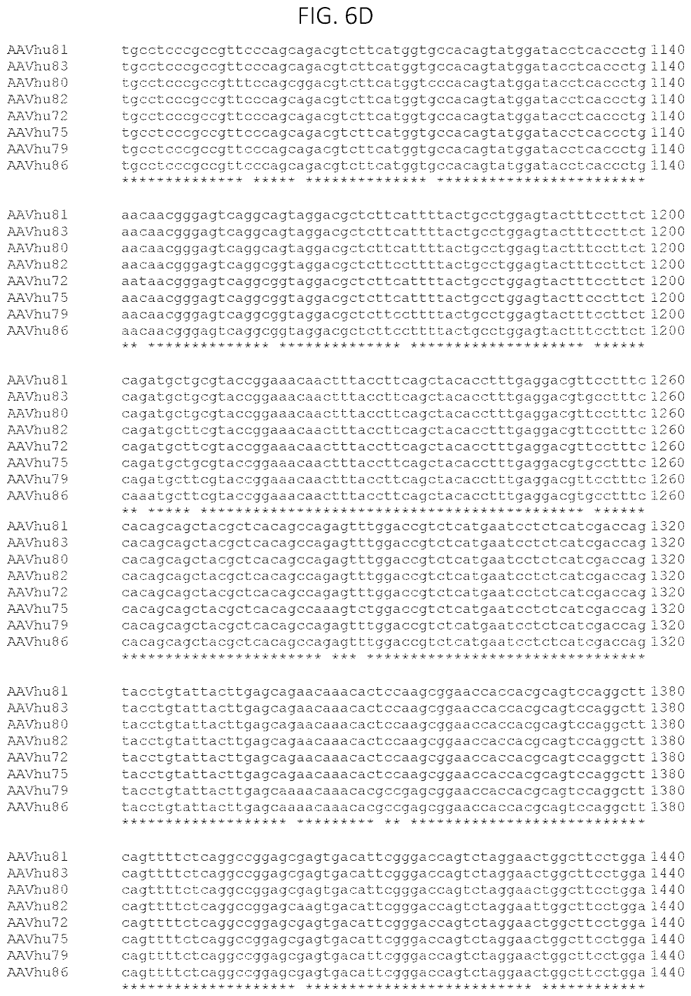

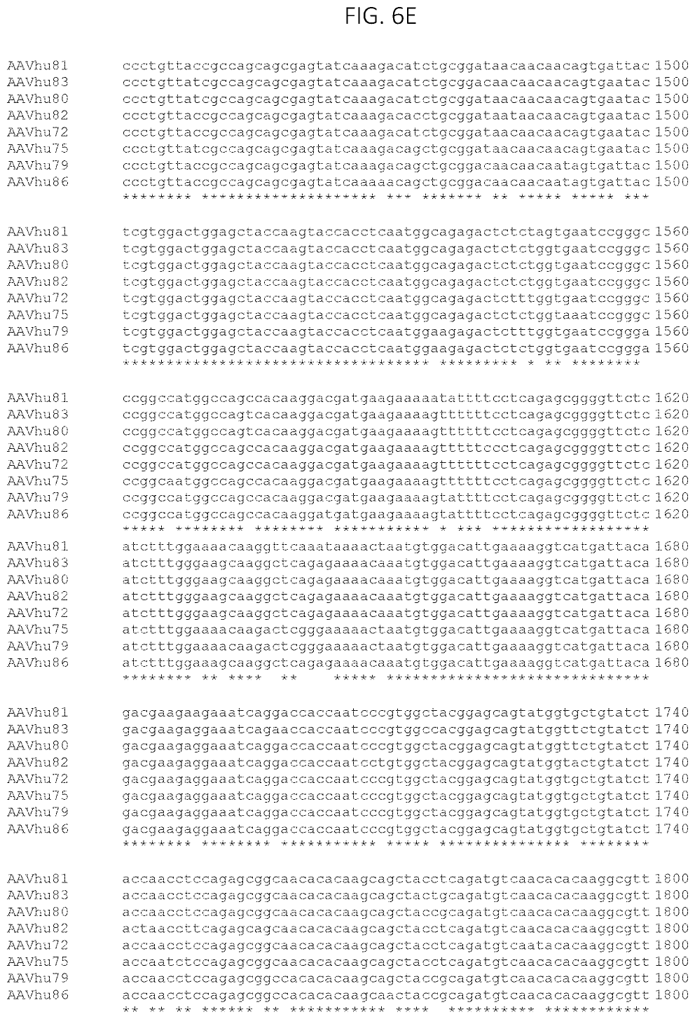

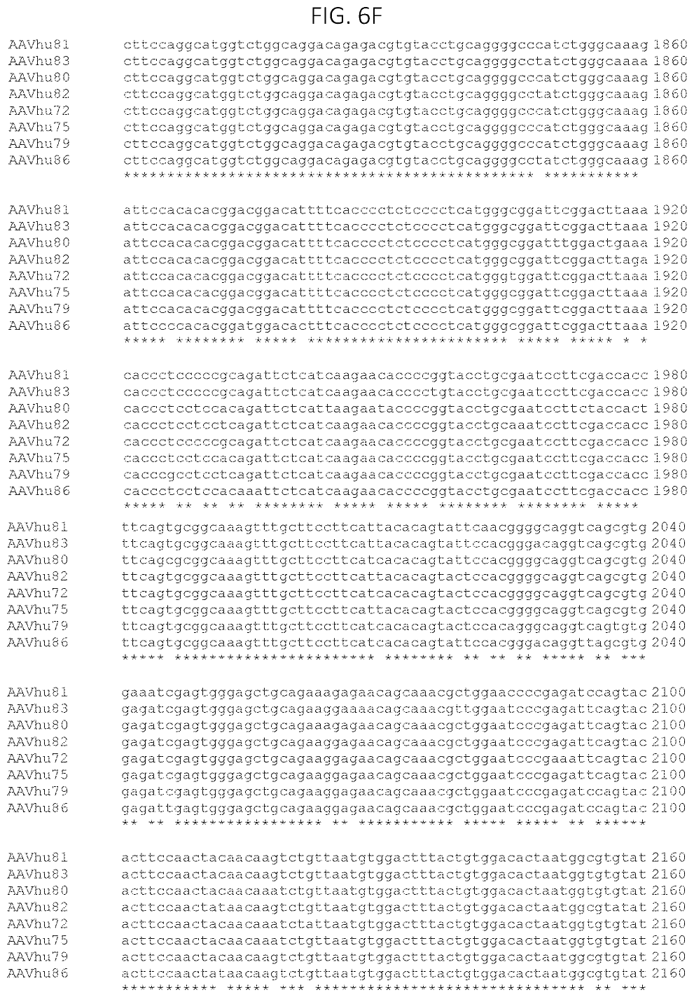

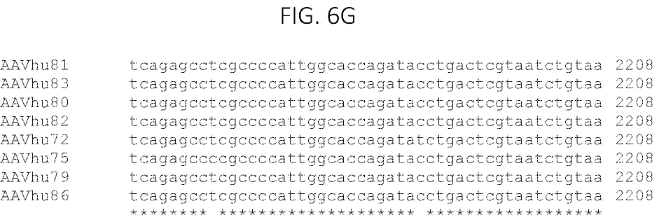

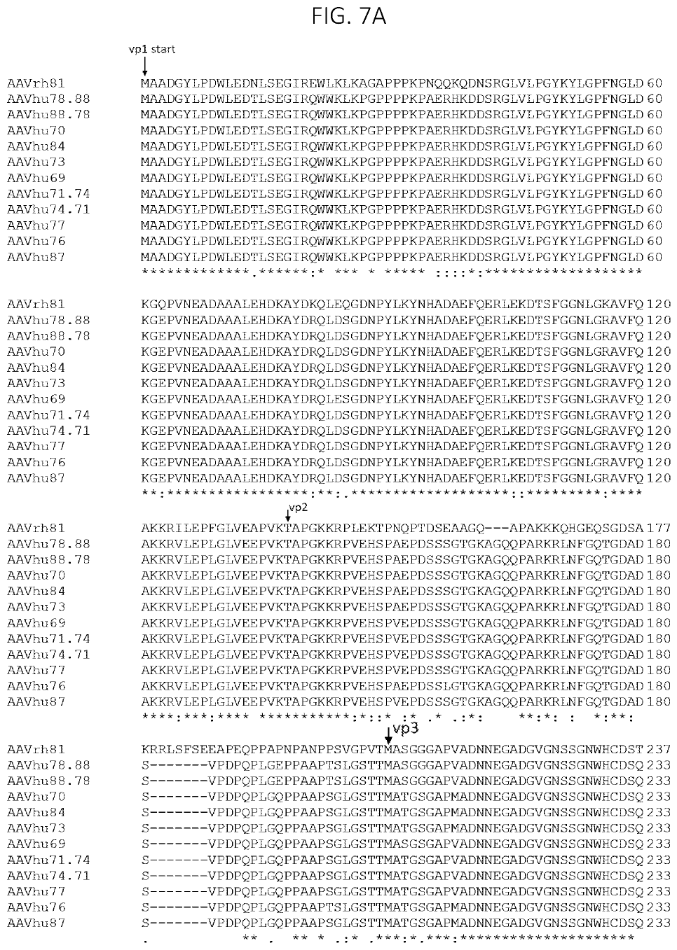

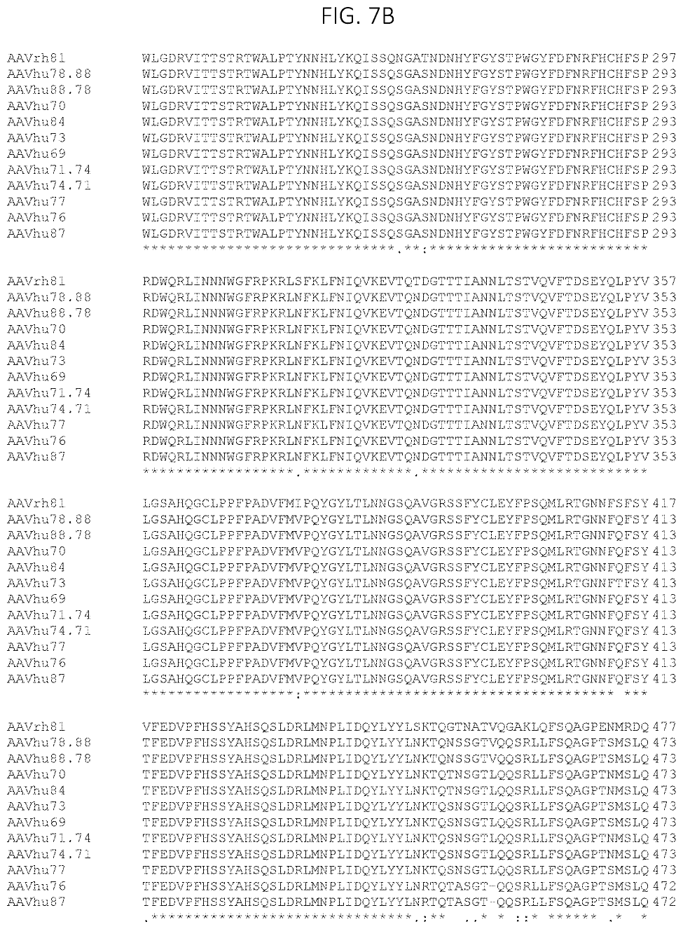

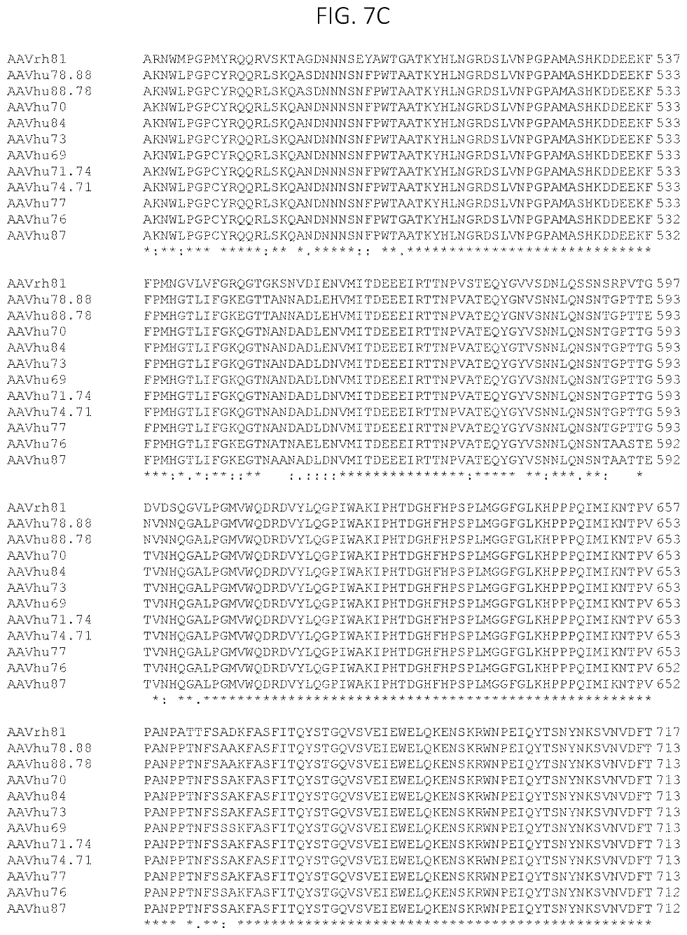

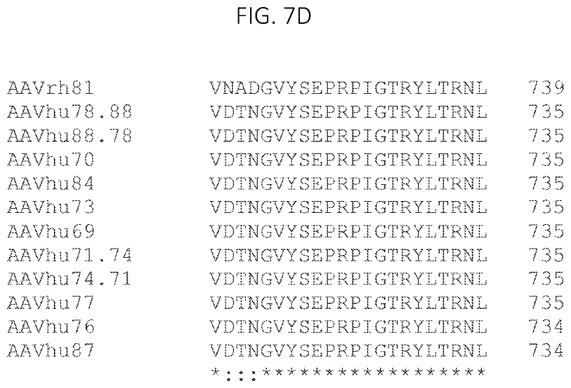

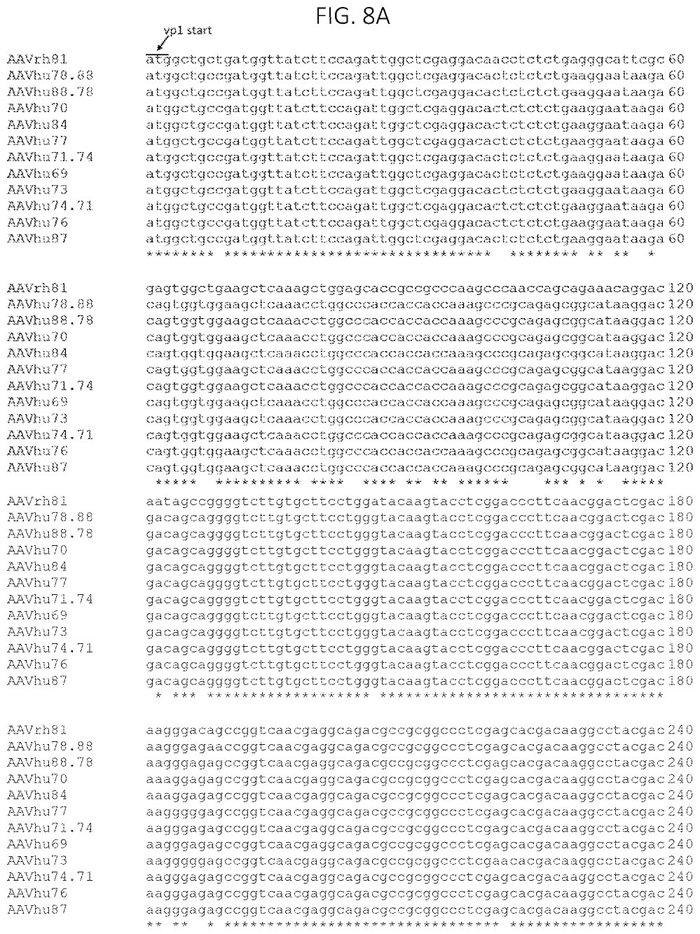

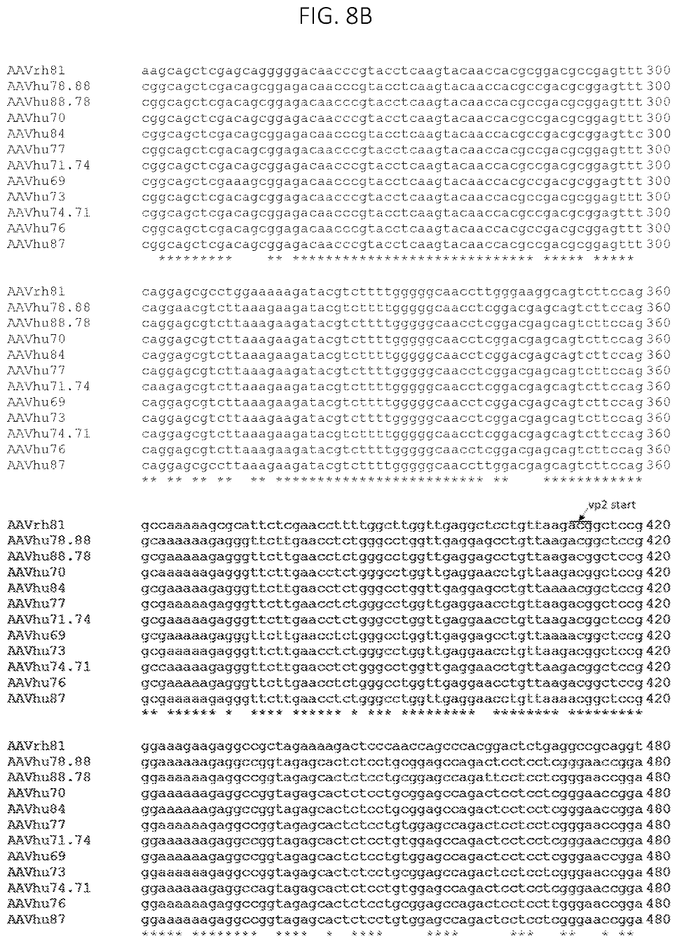

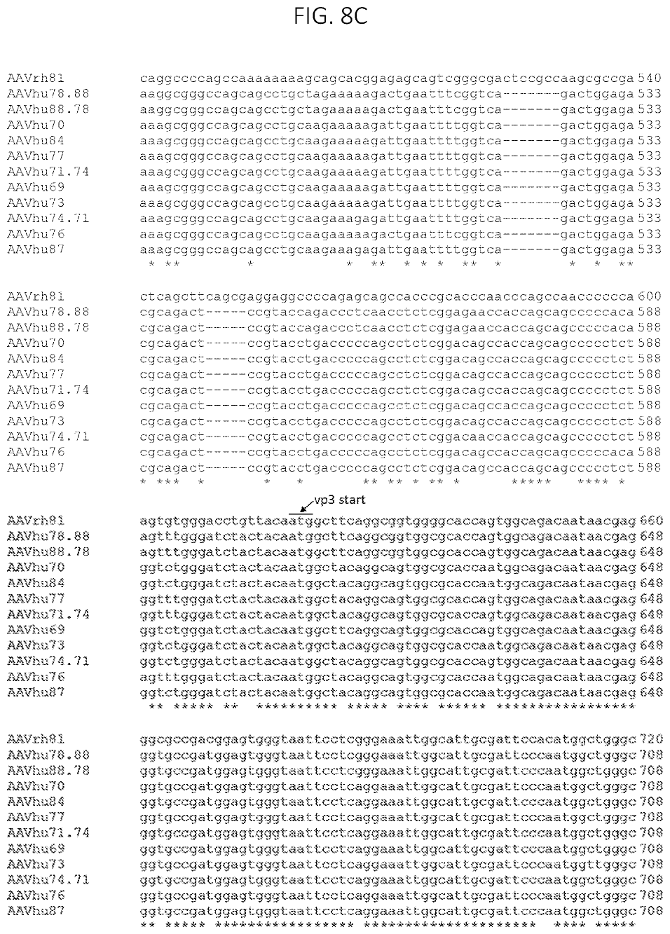

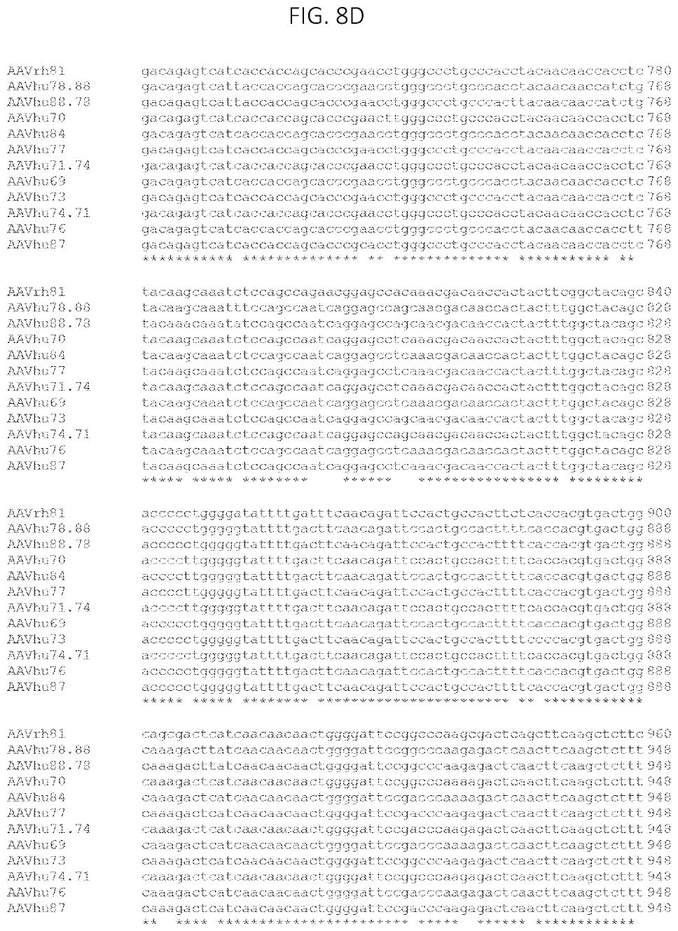

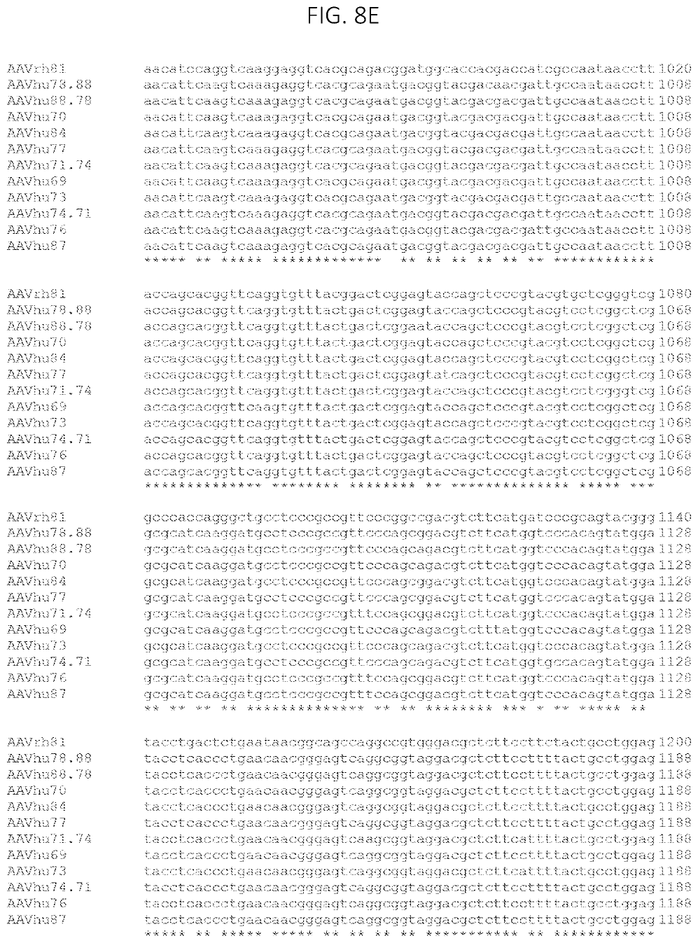

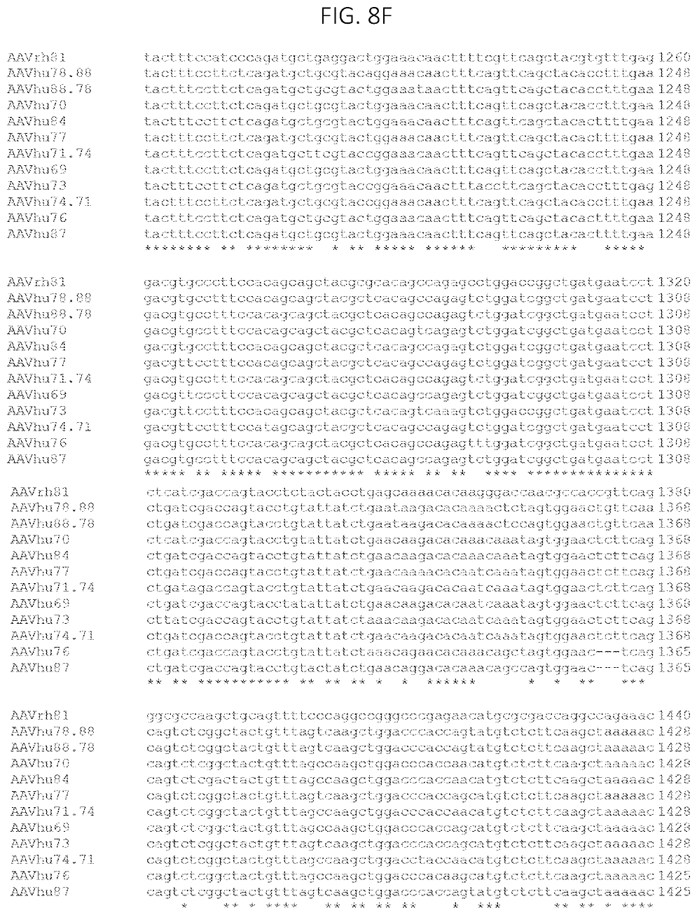

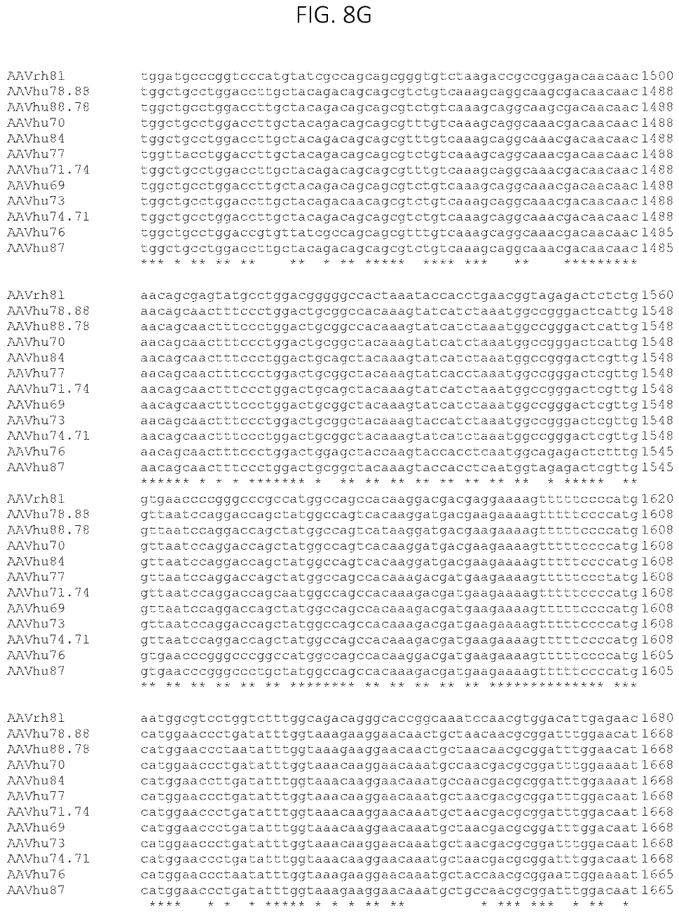

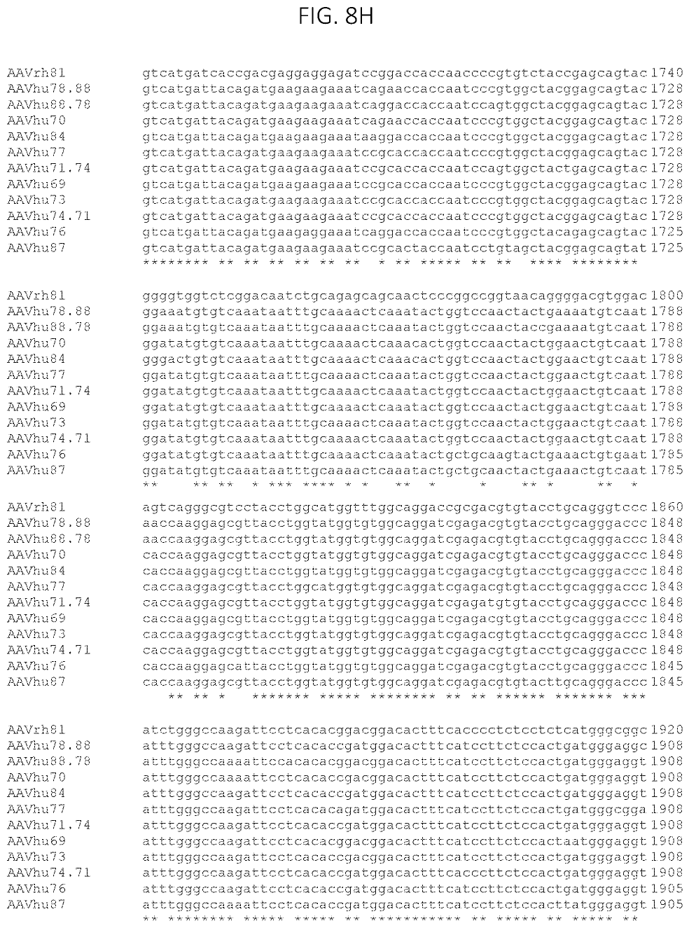

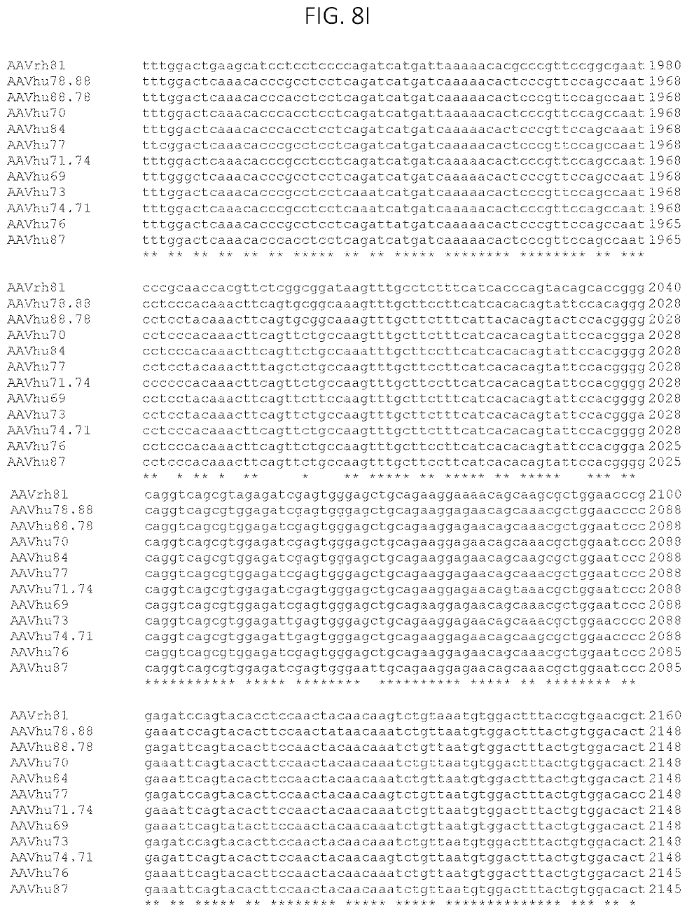

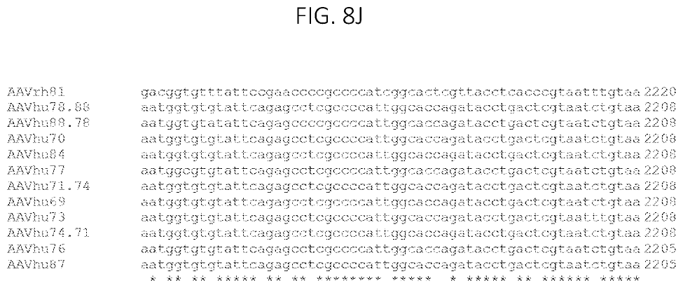

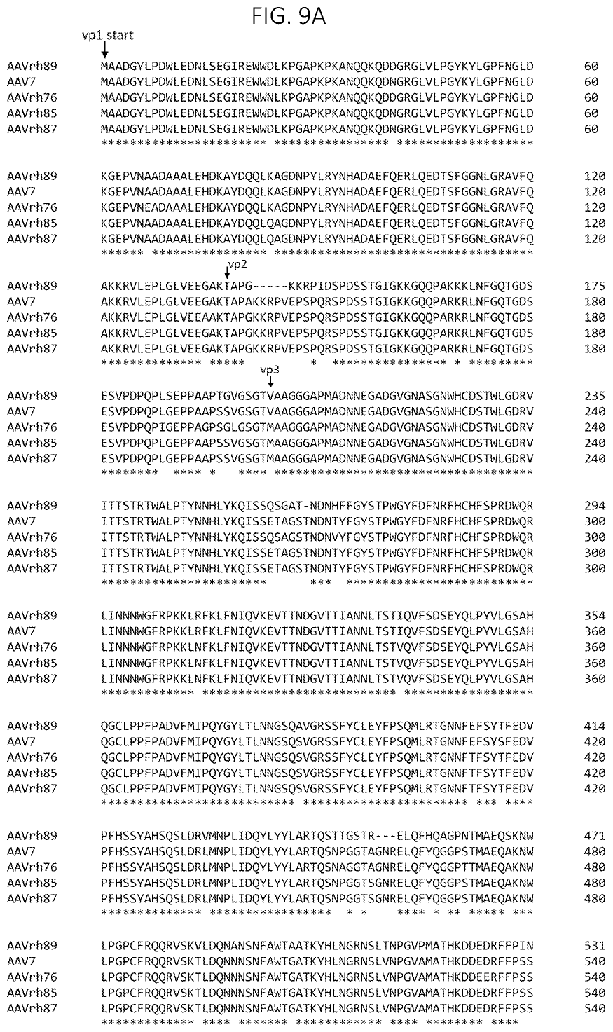

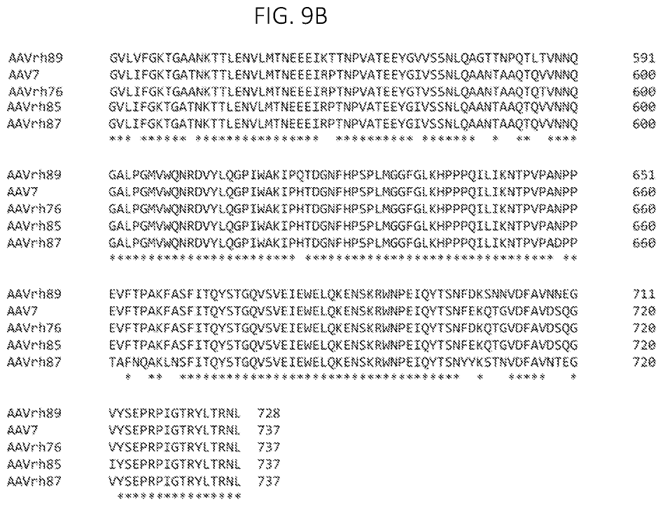

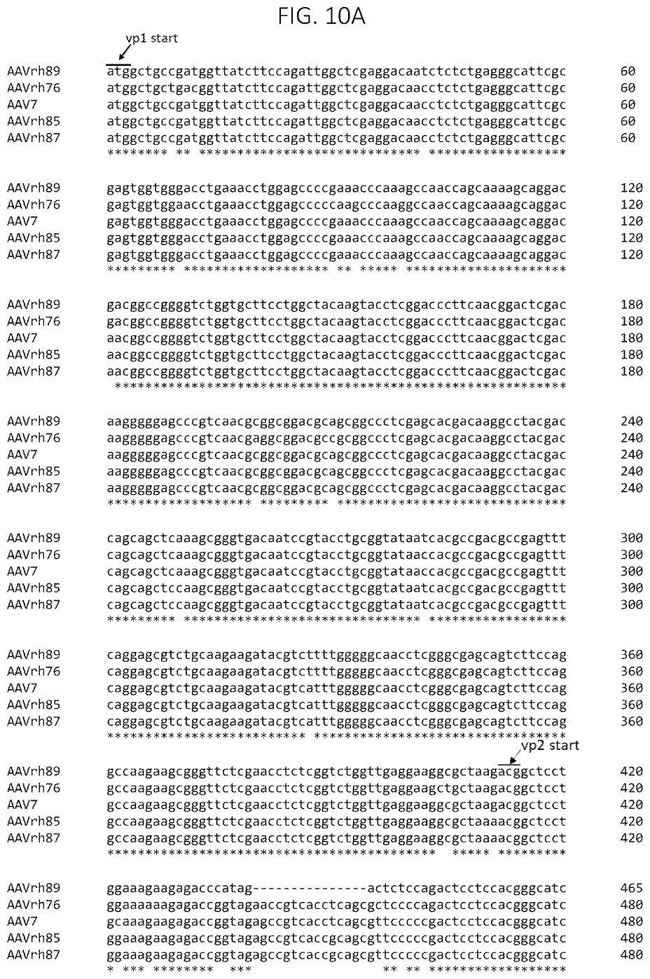

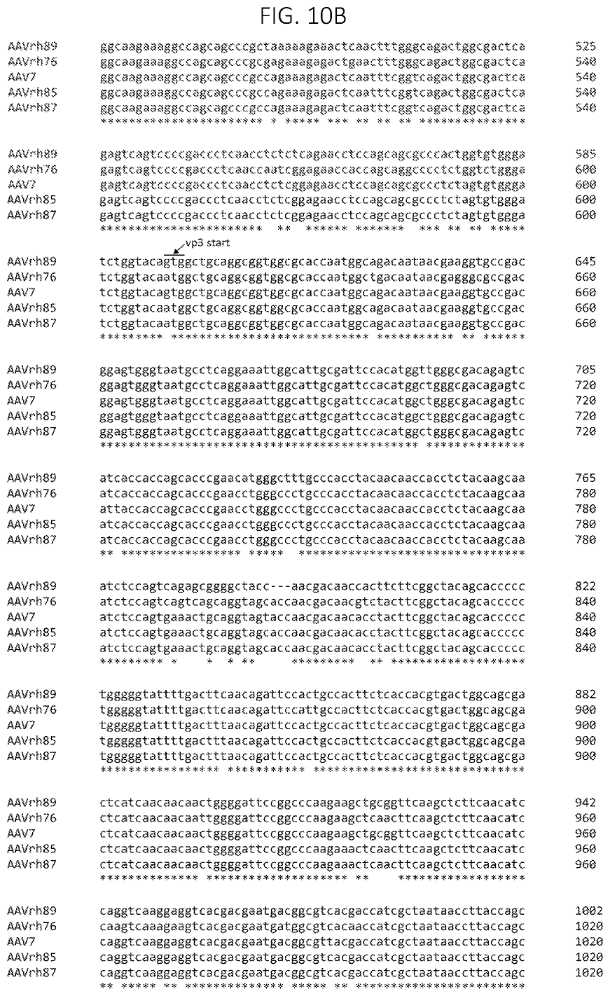

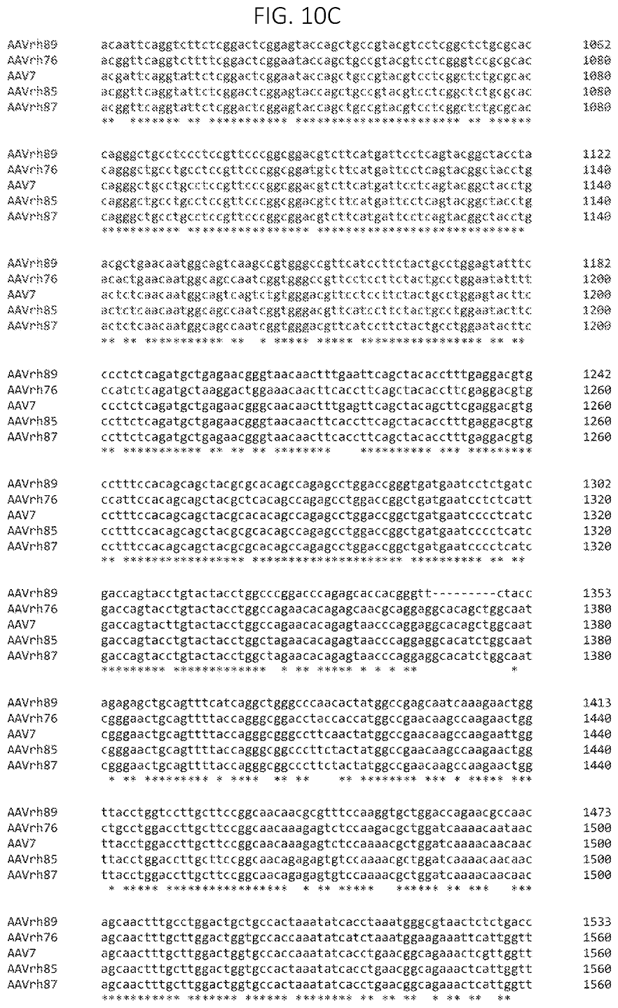

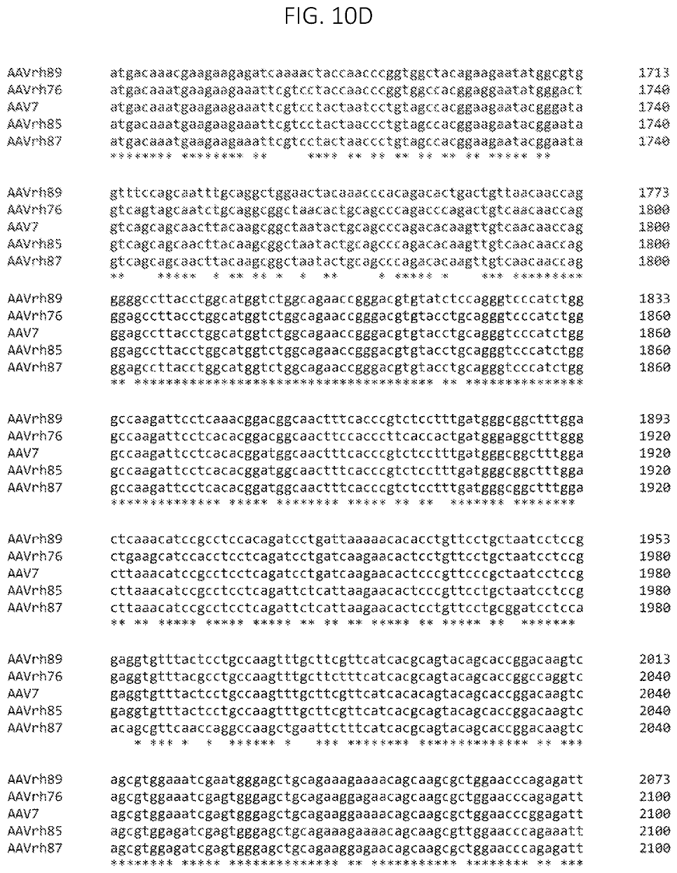

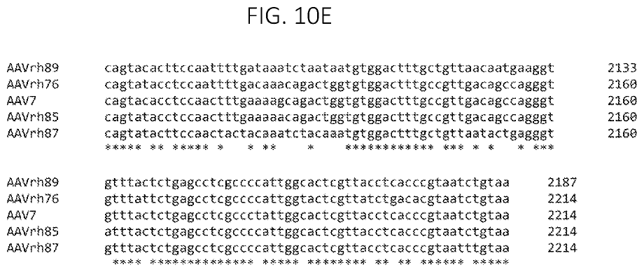

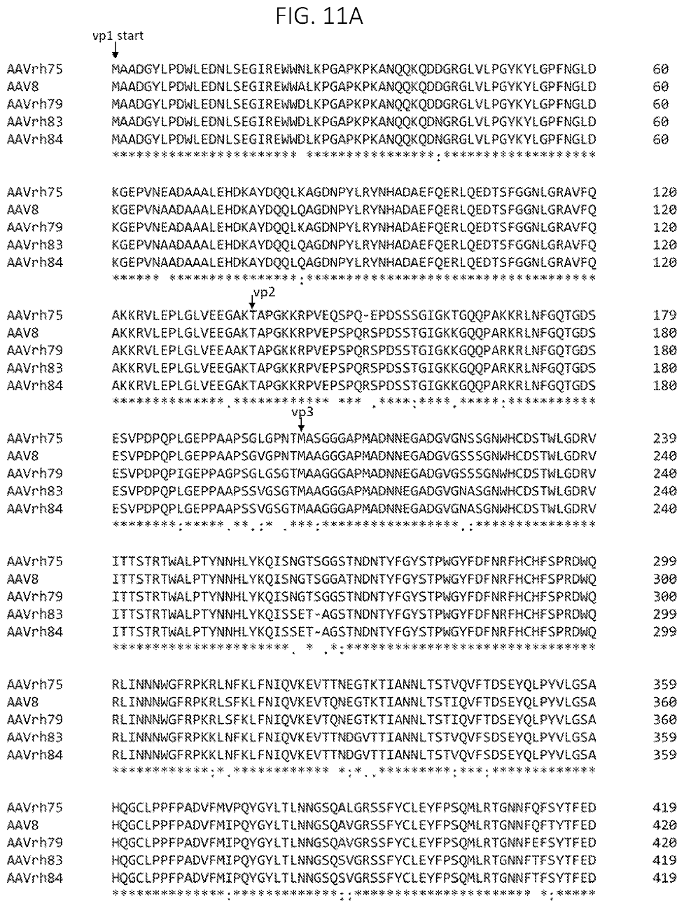

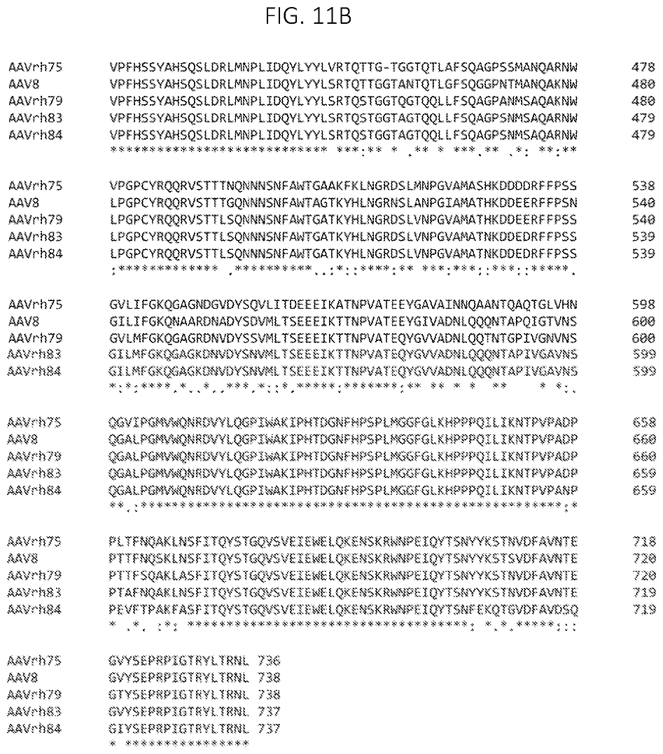

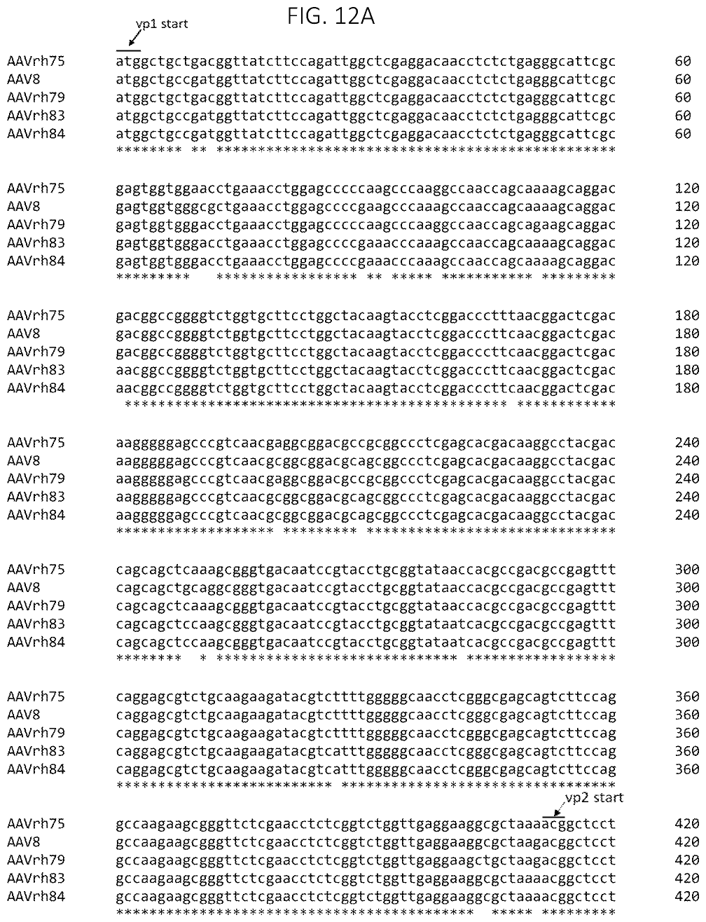

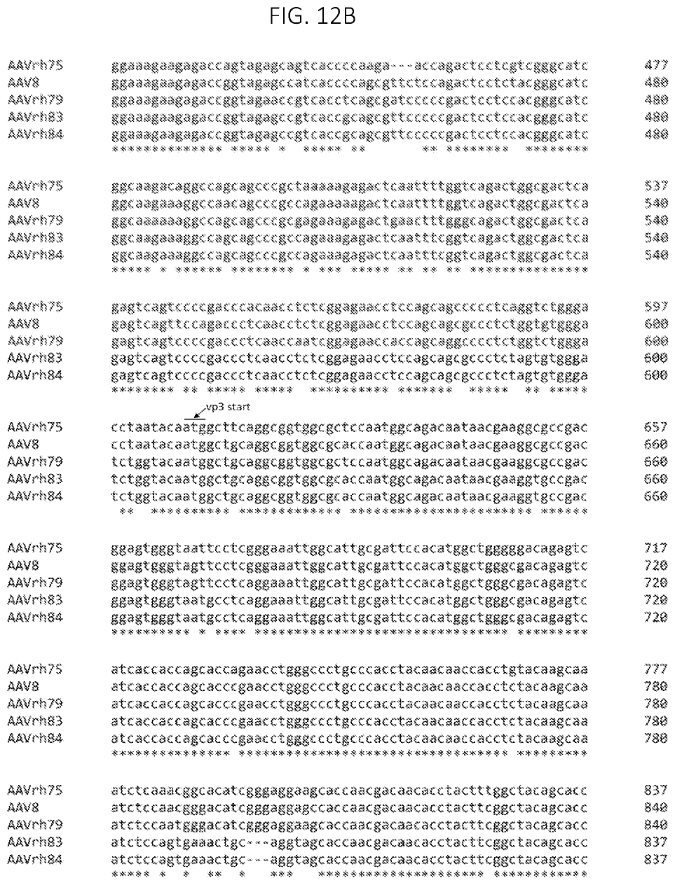

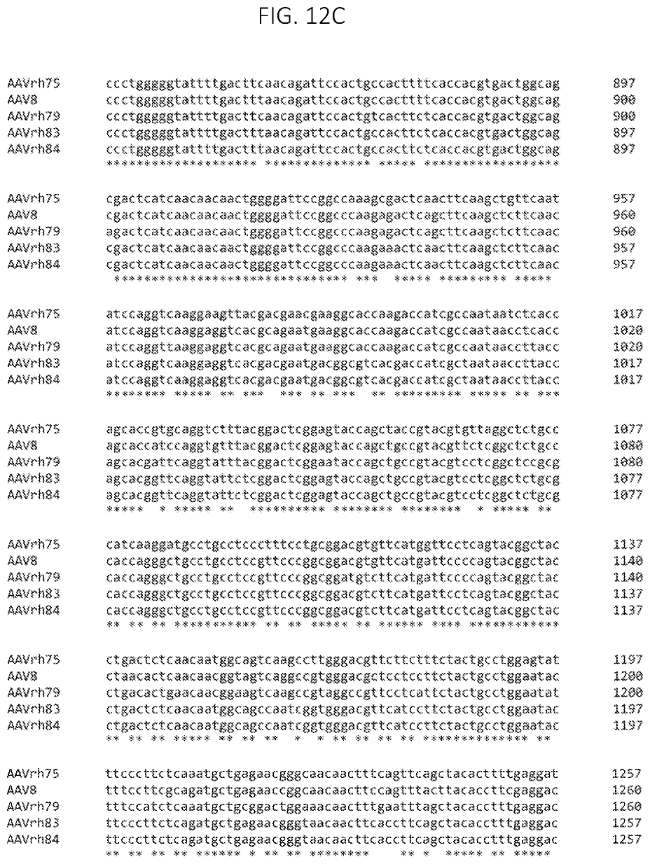

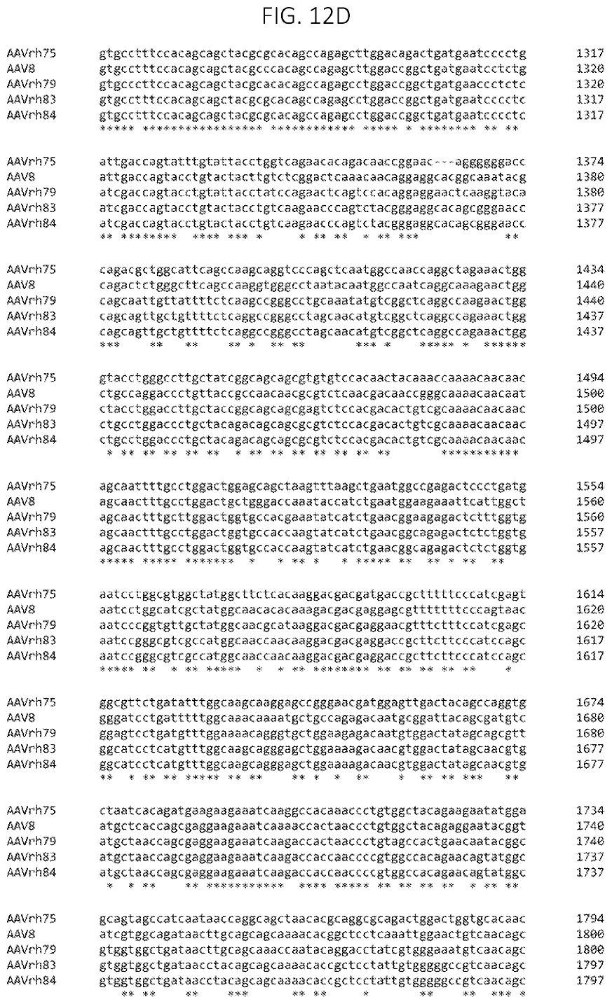

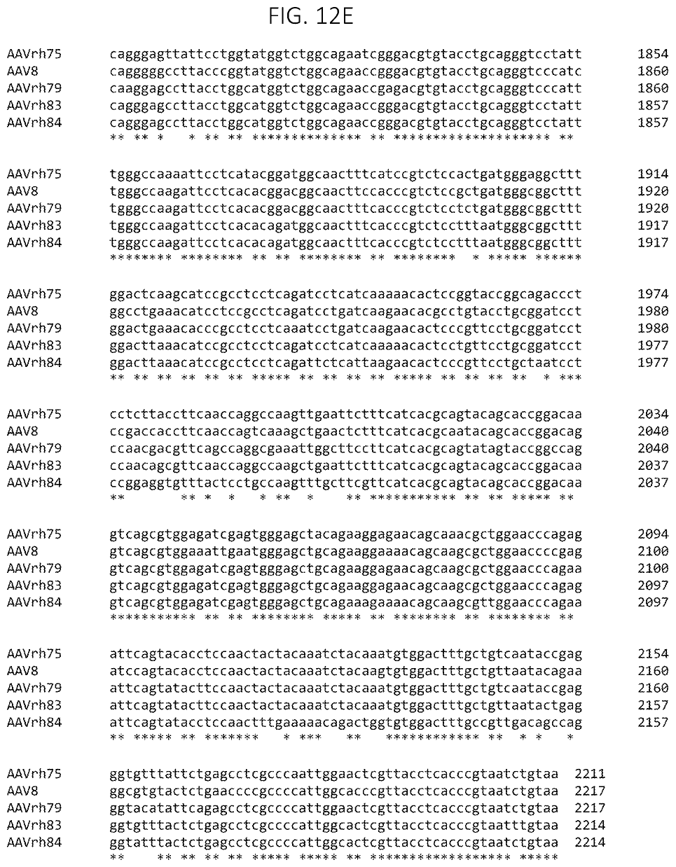

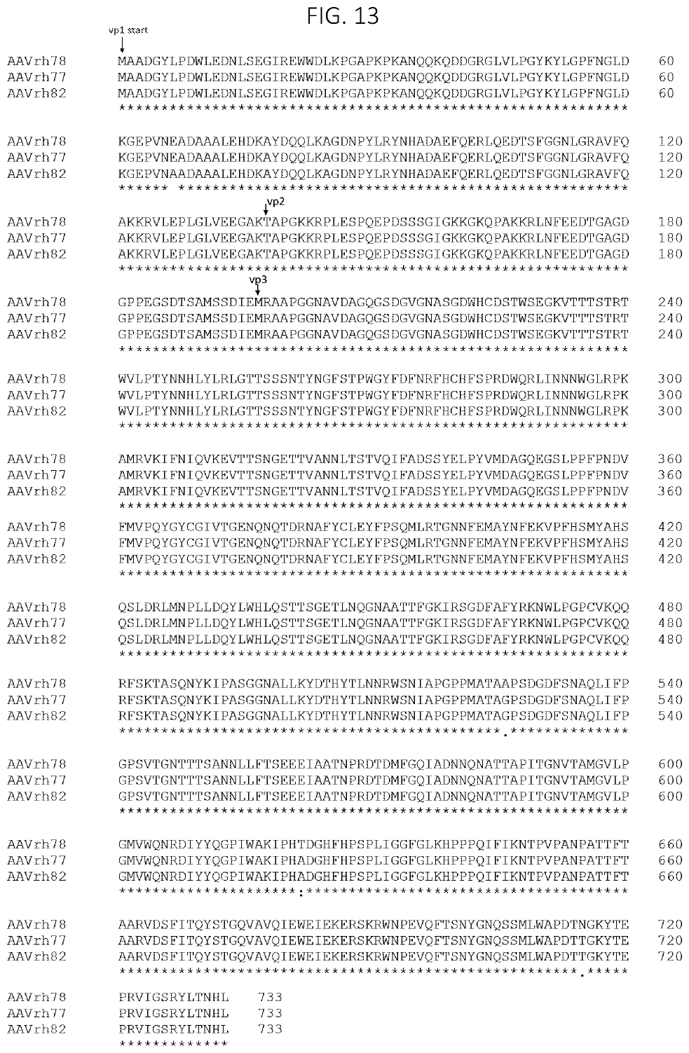

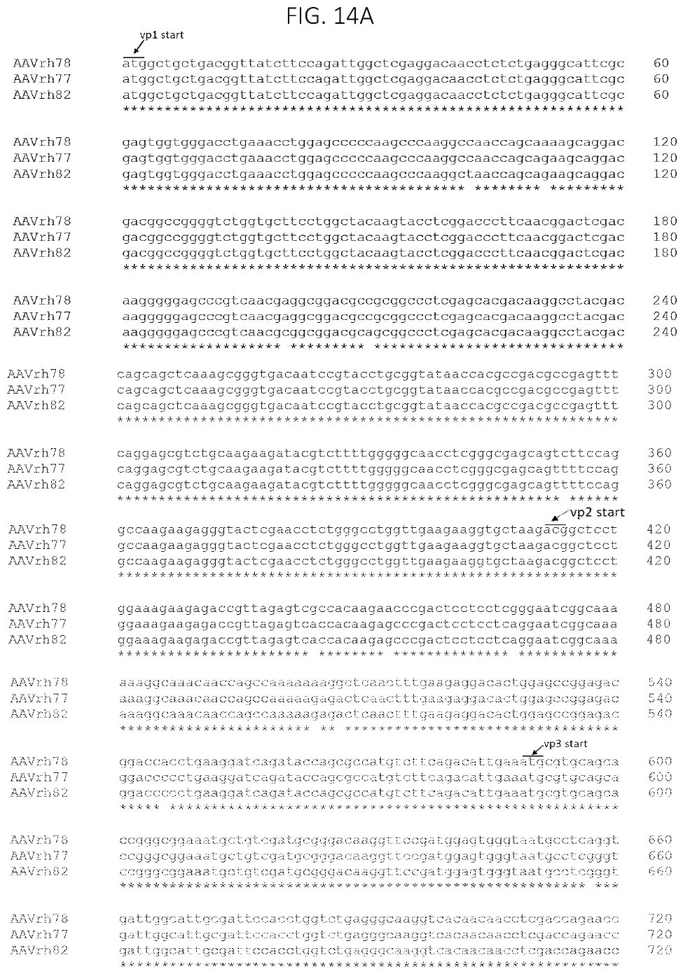

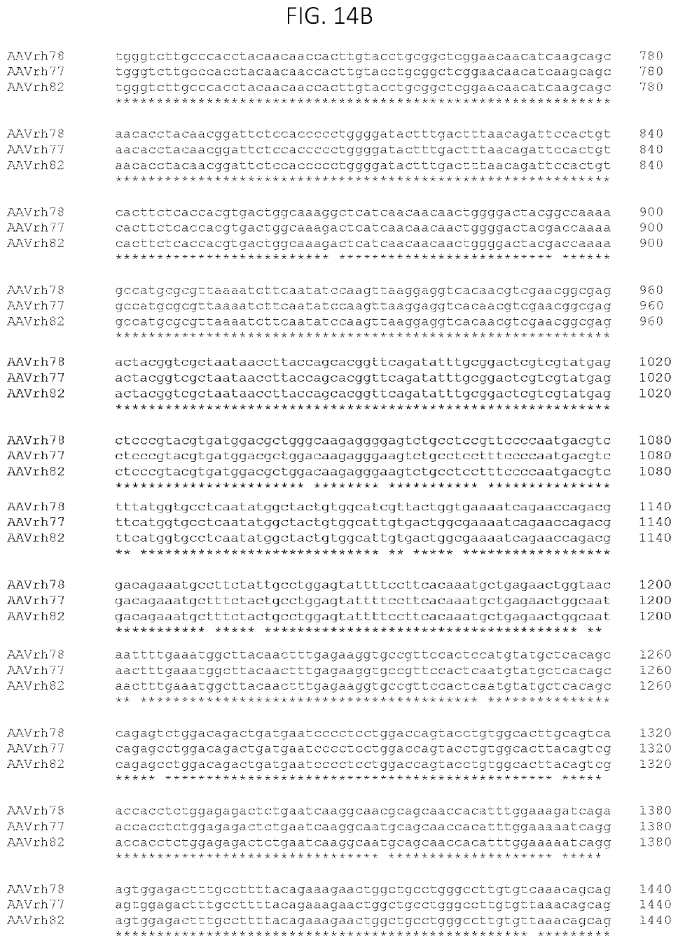

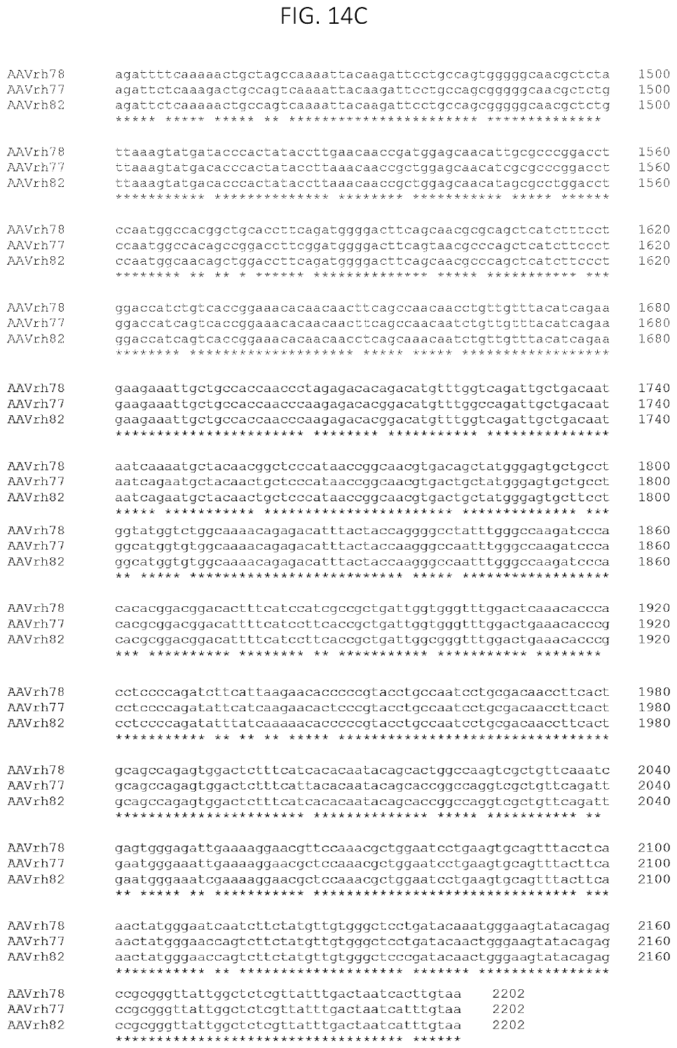

If the virus experiences selective pressure in favor of a particular genetic mutation, we would expect the nonsynonymous mutation rate (dN) to be higher than the synonymous mutation rate (dS) in that region. The contrary is true for deleterious mutations within a sequence. To evaluate the evolutionary stability of the AAV sequences isolated from primate tissues, we performed statistical analyses to determine whether there was evidence of positive, diversifying selection across the entire VP1 genes of our novel AAV when compared to their closest natural isolate sequence. We used the branch-site unrestricted statistical test for episodic diversification (BUSTED) due to its ease of use for evolutionary analyses on small sets of similar sequences (Murrell B et al. Molecular Biology and Evolution 2015; 32:1365-71). BUSTED determines whether the dN/dS rates over the entire gene of interest-across different groups of branches within a phylogenetic tree—are suggestive of positive selection. We detected statistical significance (p<0.05) at several branch points, indicating that at least one site in the VP1 gene experienced diversifying selection between test branches in the phylogeny (FIG. 3A-FIG. 3C, FIG. 4, and Table 4).

TABLE 4
BUSTED analysis of novel AAV VP1 genes to
closest natural isolate sequence. p-values

| | | | Gene-wide test |
|---|---|---|---|
| | | | for positive |
| VP1 branches compared | | | selection |
| Novel Isolate | Closest DNA hit | Closest Protein hit | (p-value)^{a} |
| hu32^{b} | AAVhu32 | AAVhu32 | 0.5 |
| AAVhu68 | AAV9 | AAV9 | 0.014 |
| AAVhu71.74 | CHC2107_AAV | CHC367_AAV | 0.5 |
| AAVhu80 | AAVhu13 | CHC371_AAV | 0.424 |
| AAVhu83 | AAVhu29 | AAVhu29 | 0.352 |
| AAV9^{b} | AAV9 | AAV9 | 0.5 |
| AAVhu74.71 | CHC976_AAV | CHC367_AAV | 0.5 |
| AAVhu77 | CHC367_AAV | CHC367_AAV | 0.5 |
| AAVhu78.88 | AAVhu88.78 | CHC3142_AAV | 0.5 |
| AAVhu88.78 | AAVhu78.88 | CHC3142_AAV | |
| AAVhu70 | AAVhu84 | AAVhu60 | 0.5 |
| AAVhu72 | AAVhu13 | CHC2206_AAV | 0.393 |
| AAVhu75 | CHC473_AAV | CHC1919_AAV | 0.267 |
| AAVhu76 | AAVhu55 | AAVhu55 | 0.286 |
| AAVhu81 | CHC2087_AAV | CHC371_AAV | 0.127 |
| AAVhu82 | AAVhuT70 | AAVhuT70 | 0.5 |
| AAVhu84 | AAVhu25 | AAVhu60 | 0.5 |
| AAVhu79 | AAVhu86 | AAVhuT40 | 0.002 |
| AAVhu86 | AAVhu79 | CHC877_AAV | |
| AAVhu87 | CHC1158_AAV | Human/China/Shanghai/FX3- | 0.5 |
| | | 1613263/AAV | |
| AAVhu73 | CHC976_AAV | AAVhu7 | 0.002 |
| AAVhu69 | AAVhu18 | AAVhu18 | 0.441 |
| AAVrh75 | AAVrh8 | AAVrh8 | 0.13 |
| AAVrh76 | AAVrh48 | AAVrh48 | 0.436 |
| AAVrh77 | AAVrh82 | AAV11 | 0.5 |
| AAVrh78 | AAVrh77 | AAV11 | 0.5 |
| AAVrh79 | AAVrh40 | AAVhu37 | 0.5 |
| AAVrh81 | | AAVhuT70 | |
| AAVrh89 | AAVrh35 | AAVrh22 | 0.001 |
| AAVrh82 | AAVrh32 | AAV11 | 0.5 |
| AAVrh83 | AAVrh84 | AAVrh46 | <0.001 |
| AAVrh84 | AAVrh83 | AAVrh46 | |
| AAVrh85 | AAVrh87 | AAV7 | <0.001 |
| AAVrh87 | AAVrh85 | AAV7 | |
| AAVHSCs | AAVHSCs | | 1.000 |
| AAVHiFi PCR | AAVHiFi PCR | | 1.000 |
| mutants | mutants | | |

^{a}Statistical significance determined by BUSTED, Likelihood ratio test

In 3/20 cases, our human-derived AAV natural isolates were positive for diversifying selection from their closest natural isolate clade member (FIG. 3A, Table 4). In 3/9 instances of rhesus isolates, diversifying selection was apparent in at least one region across the capsid sequence (FIG. 3B, Table 4). In contrast, BUSTED analysis did not show evidence of positive, diversifying selection when we compared test branches across the entire phylogeny of sequences from a group of previously published AAV natural isolates derived from human hematopoietic stem cells (HSCs) (FIG. 3C, Table 4). Similarly, the HiFi PCR mutant AAV VP 1 genes did not show evidence of positive selection (Table 1, Table 4, and FIG. 4).

In addition to performing gene-wide tests for positive selection, we assessed whether individual sites within VP1 genes for each phylogeny showed evidence of positive or negative selection. To analyze each group of AAV sequences for the presence of positively selected evolutionary hotspots, we used the mixed-effects model of evolution (MEME) program due to its ability to detect episodic and pervasive selection.

MEME detected thirteen sites that displayed evidence of positive diversifying selection in the VP1 genes of the AAVs isolated from human samples (Table 5). Four of these sites are located in the hypervariable regions (HVRs) of the capsid gene (i.e., surface-exposed capsid regions that display significant sequence diversity). Six sites are located in the internal VP1 unique region (VP1u). Additionally, we found 19 sites of significance in the capsid sequence dataset in samples from rhesus macaques (Table 5). Among these 19 sites, are located in HVR regions, while one was located in VP1u. Both sets of sequences also showed evidence of positive selection in areas between the HVRs, which comprise the non-surface-exposed regions of the capsid structure (Table 5). MEME was unable to detect any sites that were subject to positive selection in either the AAVHSC sequences or the HiFi PCR mutant-capsid sequences.

We also used the Fixed Effects Likelihood (FEL) program (Kosakovsky Pond S L et al. Molecular Biological Evolution 2005; 22:1208-22) to detect sites across branch pairs in the novel human and nonhuman primate AAV phylogenies that had undergone negative selection (Table 6). Sites within 15 out of 29 novel AAV natural isolate sequences compared to their closest known AAV relatives showed evidence of negative purifying selection. In contrast, neither the AAVHSC variants nor the HiFi PCR mutants contained any sites across the entire phylogeny that showed evidence for evolution by negative selection.

TABLE 5
MEME analysis of novel AAV VP1 phylogenies.
All sites with p < 0.05 shown.

| AAV | | MEME | | |
|---|---|---|---|---|
| Sequence | | p- | | |
| Source | Site | value^{a} | AAV Cap Location | |
| Human | 16 | <0.01 | VP1u | AAV9 S16 |
| | 24 | <0.01 | VP1u | AAV9 A24 |
| | 29 | 0.01 | VP1u | AAV9 A29 |
| | 35 | <0.01 | VP1u | AAV9 N35 |
| | 42 | 0.01 | VP1u | AAV9 A42 |
| | 164 | <0.01 | VP1u | AAV9 A164 |
| | 205 | 0.01 | VP3 start | AAV9 S205 |
| | 233 | 0.03 | Between VP3 start and | AAV9 Q233 |
| | | | HVR I | |
| | 269 | <0.01 | HVR I | AAV9 S269 |
| | 412 | <0.01 | Between HVR III and | AAV9 Q412 |
| | | | IV | |
| | 580 | 0.02 | HVR XIII | AAV9 Q579 |
| | 591 | 0.03 | HVR XIII | AAV9 Q590 |
| | 723 | <0.01 | HVR IX | AAV9 S722 |
| Rhesus | 193 | <0.01 | VP1u | AAVrh8 G189 |
| macaque | 269 | 0.01 | HVR I | AAVrh8 S265 |
| | 277 | <0.01 | Between HVR I and II | AAVrh8 T273 |
| | 318 | 0.02 | Between HVR I and II | AAVrh8 N314 |
| | 331 | 0.01 | HVR II | AAVrh8 T327 |
| | 418 | 0.01 | Between HVR III and | AAVrh8 Q412 |
| | | | IV | |
| | 461 | 0.03 | HVR IV | AAVrh8 G454 |
| | 484 | 0.01 | HVR IV | AAVrh8 A472 |
| | 506 | 0.04 | HVR V | AAVrh8 N494 |
| | 573 | <0.01 | HVR VII | AAVrh8 S556 |
| | 604 | 0.03 | HVR VIII | AAVrh8 A587 |
| | 677 | <0.01 | Between HVR VIII and | AAVrh8 L660 |
| | | | IX | |
| | 678 | 0.02 | Between HVR VIII and | AAVrh8 T661 |
| | | | IX | |
| | 681 | <0.01 | Between HVR VIII and | AAVrh8 Q664 |
| | | | IX | |
| | 685 | <0.01 | Between HVR VIII and | AAVrh8 N668 |
| | | | IX | |
| | 723 | 0.03 | HVR IX | AAVrh8 Y706 |
| | 725 | <0.01 | HVR IX | AAVrh8 S708 |
| | 727 | <0.01 | HVR IX | AAVrh8 N710 |
| | 739 | 0.03 | Between HVR IX and C | AAVrh8 S722 |
| | | | terminus | |

^{a}Statistical significance determined by MEME, Likelihood ratio test

TABLE 6
Fixed Effects Likelihood analysis of novel AAV
VP1 genes to closest natural isolate sequence.

| | | | Number of sites of |
|---|---|---|---|
| | | | negative selection, |
| Novel Isolate | Closest DNA hit | Closest Protein hit | * p < 0.05 |
| hu32^{b} | AAVhu32 | AAVhu32 | 0 |
| AAVhu68 | AAV9 | AAV9 | 0 |
| AAVhu71.74 | CHC2107_AAV | CHC367_AAV | 4 |
| AAVhu80 | AAVhu13 | CHC371_AAV | 1 |
| AAVhu83 | AAVhu29 | AAVhu29 | 1 |
| AAV9^{b} | AAV9 | AAV9 | 0 |
| AAVhu74.71 | CHC976_AAV | CHC367_AAV | 0 |
| AAVhu77 | CHC367_AAV | CHC367_AAV | 0 |
| AAVhu78.88 | AAVhu88.78 | CHC3142_AAV | 4 |
| AAVhu88.78 | AAVhu78.88 | CHC3142_AAV | |
| AAVhu70 | AAVhu84 | AAVhu60 | 0 |
| AAVhu72 | AAVhu13 | CHC2206_AAV | 0 |
| AAVhu75 | CHC473_AAV | CHC1919_AAV | 3 |
| AAVhu76 | AAVhu55 | AAVhu55 | 0 |
| AAVhu81 | CHC2087_AAV | CHC371_AAV | 4 |
| AAVhu82 | AAVhuT70 | AAVhuT70 | 0 |
| AAVhu84 | AAVhu25 | AAVhu60 | 0 |
| AAVhu79 | AAVhu86 | AAVhuT40 | 1 |
| AAVhu86 | AAVhu79 | CHC877_AAV | |
| AAVhu87 | CHC1158_AAV | Human/China/Shanghai/FX3- | 2 |
| | | 1613263/AAV | |
| AAVhu73 | CHC976_AAV | AAVhu7 | 0 |
| AAVhu69 | AAVhu18 | AAVhu18 | 0 |
| AAVrh75 | AAVrh8 | AAVrh8 | 82 |
| AAVrh76 | AAVrh48 | AAVrh48 | 23 |
| AAVrh77 | AAVrh82 | AAV11 | 0 |
| AAVrh78 | AAVrh77 | AAV11 | 10 |
| AAVrh79 | AAVrh40 | AAVhu37 | 9 |
| AAVrh81 | | AAVhuT70 | |
| AAVrh89 | AAVrh35 | AAVrh22 | 43 |
| AAVrh82 | AAVrh32 | AAV11 | 0 |
| AAVrh83 | AAVrh84 | AAVrh46 | 1 |
| AAVrh84 | AAVrh83 | AAVrh46 | |
| AAVrh85 | AAVrh87 | AAV7 | 1 |
| AAVrh87 | AAVrh85 | AAV7 | |
| AAVHSCs | AAVHSCs | | 0 |
| AAVHiFi PCR | AAVHiFi PCR | | 0 |
| mutants | mutants | | |

* Likelihood Ratio Test

AAV sequence isolation techniques have greatly evolved since the discovery of AAVs in 1965. In this study, we compared the DNA-replication fidelity of two DNA polymerases in terms of AAV isolation: HotStar HiFidelity polymerase and Q5 Hot Start High-Fidelity polymerase. We found that using the HiFi polymerase and a protocol with a high number of PCR cycles—a method previously used to discover novel AAVs—resulted in a significantly higher rate of random mutations in amplicons generated from template DNA compared to the method utilizing the Q5 polymerase. The mutant-PCR isolates produced vector and transduced Huh7 cells in vitro at variable levels. These experiments highlight the variable and unpredictable impact that low DNA polymerase fidelity can exert on AAV function during capsid-genome isolation.

Tindall et al. were among the first to demonstrate that DNA polymerases can generate mutations in amplified DNA (Tindall K R et al. Biochemistry 1988; 27:6008-6013). Since then, researchers have isolated and engineered a variety of new polymerases to address this issue, including Q5—one of the most accurate polymerases—with a base substitution rate of 5.3×10⁻⁷ bp, which corresponds to an approximately 280-fold higher fidelity compared with Taq polymerase (Potapov V et al. PloS one 2017; 12:e016977). In contrast, the fidelity of the HotStar HiFi polymerase is reported to be only 10-fold higher than that of Taq. We demonstrated that optimal AAV isolation requires using the highest-fidelity DNA polymerases available, in this case Q5.

We also used the Q5 polymerase to perform AAV-SGA to validate the sequence identity of one of the human-derived AAVs isolated in this work, AAVhu68. The replicative nature of this technique, coupled with the high fidelity of the Q5 polymerase, allowed us to precisely and accurately identify the capsid sequence of this isolate. Furthermore, the sequencing data of the resulting amplicons we obtained using the Q5 polymerase-based technique were congruent with the amplicons we obtained via NGS methods, thereby validating the identity of this AAV natural isolate capsid gene. AAV-SGA did recover a small minority of amplicon sequences in which 1-2 nucleotides were mismatched from the AAVhu68 genome, which may be attributed to NGS error, the low error rate of Q5, or DNA damage induced by thermocycling, as characterized by Potapov et al (PloS one 2017; 12:e0169774) These data demonstrate that AAV-SGA is a robust tool for analyzing viral populations with very high precision and accuracy.

By utilizing the high-fidelity Q5-based AAV-isolation method, we found that natural AAV variant capsid protein sequences remain relatively stable, while their DNA sequences can exhibit considerable changes in comparison to their closest relative in GenBank. This finding stands in stark contrast to our HiFi PCR mutant sequences and a subset of AAV sequences identified from human HSCs (AAVHSCs), in which many more amino acid changes correlated with DNA-sequence alterations. In any viral population, one would expect host-mediated evolutionary pressure from the immune system or factors that mediate tissue tropism to promote positive, diversifying selection in relation to processes involving host-capsid interactions such as cellular adhesion, entry, and viral trafficking. However, these selection pressures are absent in an in vitro replication environment, such as that used when generating PCR mutants.

We used the BUSTED program to determine whether the overall AAV capsid sequence was subjected to positive selection in its recent evolutionary lineage. Our results showed evidence of diversifying selection, even for cases exhibiting high DNA sequence variation yet high amino acid sequence homology between two isolates. Conversely, BUSTED analysis gave no evidence of diversifying selection for the few instances in which DNA sequence variation between multiple AAVs resulted in amino acid changes (i.e., AAVHSCs and AAV HiFi PCR mutants). An unexpected finding was that a population of AAVs recovered from natural sources, such as human HSCs, showed no evidence of evolutionary pressure-mediated changes despite having a high nonsynonymous mutation rate.

We used MEME to elucidate patterns of site-specific evolution in the novel AAV natural variants (Murrell B et al. PLoS Genetics 2012; 8:e1002764). The majority of sites exhibiting evidence of evolution mapped to the AAV HVRs; surface-exposed HVRs mediate interactions with host factors such as antibodies and cell-surface receptors. Additionally, a few of the sites were positioned prior to the start of VP3 in the VP1u region that interacts with host-cell intracellular trafficking machinery. The evolutionary pressure exhibited at these sites could provide a good indication of which capsid regions are amenable to modification from a vector-engineering standpoint. In contrast, neither the AAVHSC isolates nor the HiFi PCR mutants contained any sites that displayed significant selective pressure, further confirming that polymerase-introduced errors can significantly influence AAV sequence analysis, discovery, and function. While high-fidelity DNA polymerases are necessary for optimal PCR-based AAV isolation and characterization from natural sources, error-prone polymerases can expand and diversify the library of candidate AAVs by introducing random mutations into a given AAV capsid backbone.

These results highlight the need for accurate AAV-isolation methods to reach valid conclusions about AAV evolution, genetics, and biological functions arising from genome variation. Our findings indicate that not all “high-fidelity” DNA polymerases are created equal and that one must use caution when analyzing AAV sequences generated with a lower-fidelity polymerase. Utilizing methods such as SGA in conjunction with high-fidelity polymerases enables the accurate isolation of natural AAV populations that may contain the next candidate gene therapy vector.

The novel AAV natural isolates recovered from human tissue samples non-human primate tissue samples and sequences thereof are summarized in Table 7 and Table 8 below.

TABLE 7
Novel AAV natural isolates recovered from
human tissue samples and sequences thereof.

SEQ ID NOS

| Isolate name | DNA | Protein |
|---|---|---|
| hu32 | 77 | 70 |
| hu68 | 1 | 2 |
| hu71/74 | 3 | 4 |
| hu79 | 5 | 6 |
| hu80 | 7 | 8 |
| hu83 | 9 | 10 |
| AAV9 | 76 | 68 |
| hu74/71 | 11 | 12 |
| hu77 | 13 | 14 |
| hu78/88 | 15 | 16 |
| hu70 | 17 | 18 |
| hu72 | 19 | 20 |
| hu75 | 21 | 22 |
| hu76 | 23 | 24 |
| hu81 | 25 | 26 |
| hu82 | 27 | 28 |
| hu84 | 29 | 30 |
| hu86 | 31 | 32 |
| hu87 | 33 | 34 |
| hu88/78 | 35 | 36 |
| hu73 | 73 | 74 |
| hu69 | 37 | 38 |

TABLE 8
Novel AAV natural isolates recovered from nonhuman primate
intestinal tissue samples and sequences thereof.

SEQ ID NOS

| Isolate name | DNA | Protein |
|---|---|---|
| rh75 | 39 | 40 |
| rh76 | 41 | 42 |
| rh77 | 43 | 44 |
| rh78 | 45 | 46 |
| rh79 | 47 | 48 |
| rh81 | 49 | 50 |
| rh89 | 51 | 52 |
| rh82 | 53 | 54 |
| rh83 | 55 | 56 |
| rh84 | 57 | 58 |
| rh85 | 59 | 60 |
| rh87 | 61 | 62 |

### Example 6: Evaluation of Production Yields and Transduction Levels for Recombinant AAV Vectors with Novel Capsids

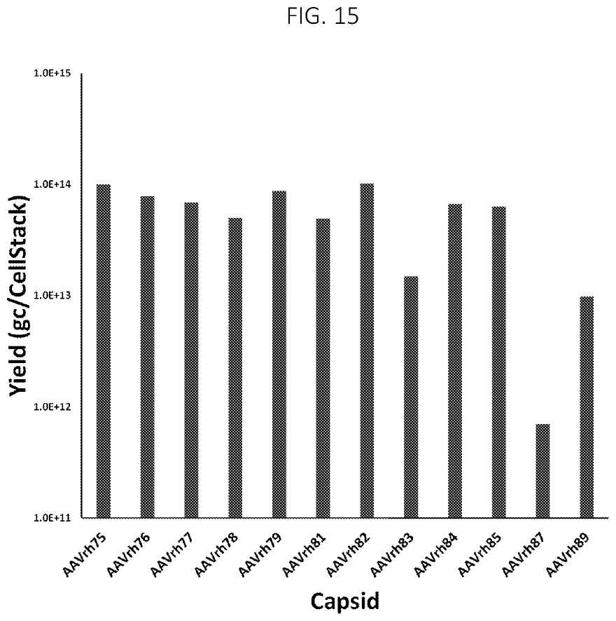

For CellSTACK® scale production, rAAV vectors were produced and purified using the protocol described by Lock et al. (Human Gene Therapy 21:1259-1271, October 2010). The titers of the purified products were measured by Droplet Digital PCR described by Lock et al. (Human Gene Therapy 25:115-25, April 2014). The three plasmids used in the triple-transfection part of the protocol were: adenovirus helper plasmid pAdAF6, a trans plasmid carrying AAV2 rep gene and the capsid gene of a novel AAV isolate, and a cis plasmid carrying a transgene cassette flanked by AAV2 5′ and 3′ ITRs. The cis plasmid included an expression cassette having TBG promoter and eGFP transgene. Yields for the recombinant vectors having AAVrh75, AAVrh76, AAVrh77, AAVrh78, AAAVrh79, AAVrh81, AAVrh82, AAVrh83, AAVrh84, AAVrh87, AAVrh89 capsids are shown in FIG. 15.

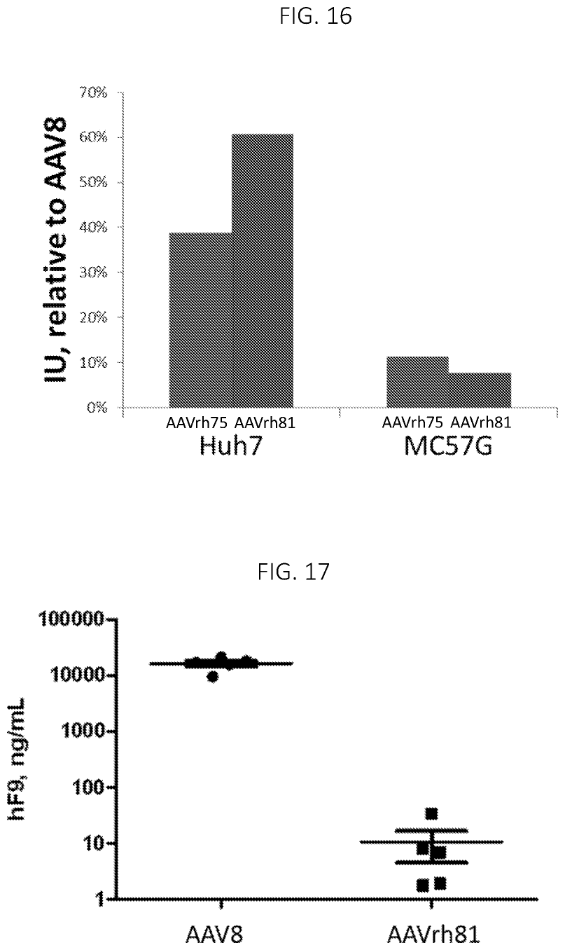

For 12-well plate scale production, the protocol was adapted from the CellSTACK® protocol mentioned above without the purification step, mainly by reducing the materials used proportionally to cell culture areas. The trans plasmids used here included AAVrh75 and AAVrh81 capsid genes. The cis plasmid used here included a CB7 promoter and firefly luciferase gene. After production, culture supernatants were collected and spun down to remove cell debris. The yields were then measured by a bioactivity assay where an equal volume of the supernatants was used to transduce Huh7 and MC57G cells, and luciferase activity was measured with a luminometer (BioTek). FIG. 16 shows infectious titers relative to a comparable AAV8 vector. The AAVrh81 vector had higher levels of infectivity than the AAVrh75 vector in the human cell line Huh7, but exhibited lower levels of infectivity in the mouse cell line MC57G.

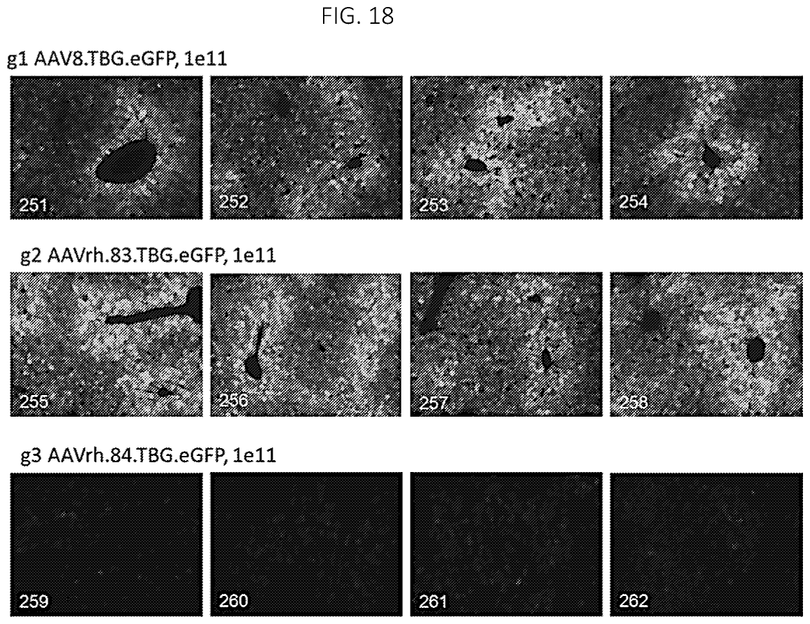

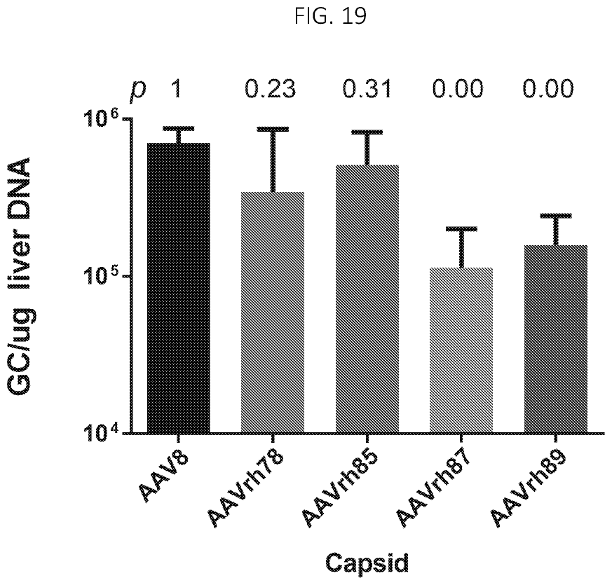

In addition, delivery of transgenes was evaluated in vivo. Mice were injected intravenously with rAAV having an AAV8 or AAVrh81 capsid and a vector genome containing a liver-specific promoter (LSP) promoter and human factor IX transgene. On day 28, plasma was collected to measure factor IX levels. Expression of human factor IX following AAVrh81 vector delivery was much lower than for AAV8 (FIG. 17). In further studies, rAAV vectors having AAVrh78, AAVrh83, AAVrh84, AAVrh85, AAVrh87, AAVrh89, or AAV8 capsids and a vector genome with a TBG promoter and eGFP transgene were administered intravenously at 1×10¹¹ GC/mouse. Livers were harvested on day 14 to evaluate GFP expression. Transduction was comparable to AAV8 for AAVrh83, while levels were GFP were very low following delivery of the AAVrh84 vector (FIG. 18). Genomic DNA was extracted from liver to measure vector genome copies qPCR. Liver transduction levels for AAVrh78, AAVrh85, AAVrh87, and AAVrh89 were about 49%, 72%, 16%, and 22% of levels detected with AAV8, respectively (FIG. 19).

(Sequence Listing Free Text)
The following information is provided for sequences containing free text
under numeric identifier <223>.

| SEQ ID NO: | |
|---|---|
| (containing free text) | Free text under <223> |
| 1 | <223> adeno-associated virus hu68 |
| | <221> misc_feature |
| | <222> (1) . . . (2208) |
| | <223> vp1 |
| | <221> misc_feature |
| | <222> (412) . . . (2208) |
| | <223> vp2 |
| | <221> misc_feature |
| | <222> (604) . . . (2208) |
| | <223> vp3 |
| 2 | <223> adeno-associated virus hu68 |
| | <221> MISC_FEATURE |
| | <222> (1) . . . (736) |
| | <223> vp1 |
| | <221> MISC_FEATURE |
| | <222> (138) . . . (736) |
| | <223> vp2 |
| | <221> MISC_FEATURE |
| | <222> (202) . . . (736) |
| | <223> vp3 |
| 3 | <223> adeno-associated virus hu71/74 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 4 | <223> adeno-associated virus hu71/74 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 5 | <223> adeno-associated virus hu79 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 6 | <223> adeno-associated virus hu79 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 7 | <223> adeno-associated virus hu80 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 8 | <223> adeno-associated virus hu80 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 9 | <223> adeno-associated virus hu83 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 10 | <223> adeno-associated virus hu83 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 11 | <223> adeno-associated virus hu74/71 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 12 | <223> adeno-associated virus hu74/71 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 13 | <223> adeno-associated virus hu77 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 14 | <223> adeno-associated virus hu77 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 15 | <223> adeno-associated virus hu78/88 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 16 | <223> adeno-associated virus hu78/88 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 17 | <223> adeno-associated virus hu70 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 18 | <223> adeno-associated virus hu70 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 19 | <223> adeno-associated virus hu72 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 20 | <223> adeno-associated virus hu72 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 21 | <223> adeno-associated virus hu75 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 22 | <223> adeno-associated virus hu75 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 23 | <223> adeno-associated virus hu76 |
| | <221> misc_feature |
| | <222> (1) . . . (2202) |
| | <223> vp1 |
| | <221> misc_feature |
| | <222> (412) . . . (2202) |
| | <223> vp2 |
| | <221> misc_feature |
| | <222> (607) . . . (2202) |
| | <223> vp3 |
| 24 | <223> adeno-associated virus hu76 |
| | <221> MISC_FEATURE |
| | <222> (1) . . . (734) |
| | <223> vp1 |
| | <221> MISC_FEATURE |
| | <222> (138) . . . (734) |
| | <223> vp2 |
| | <221> MISC_FEATURE |
| | <222> (203) . . . (734) |
| | <223> vp3 |
| 25 | <223> adeno-associated virus hu81 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 26 | <223> adeno-associated virus hu81 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 27 | <223> adeno-associated virus hu82 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 28 | <223> adeno-associated virus hu82 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 29 | <223> adeno-associated virus hu84 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 30 | <223> adeno-associated virus hu84 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 31 | <223> adeno-associated virus hu86 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 32 | <223> adeno-associated virus hu86 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 33 | <223> adeno-associated virus hu87 |
| | <221> misc_feature |
| | <222> (1) . . . (2202) |
| | <223> vp1 |
| | <221> misc_feature |
| | <222> (412) . . . (2202) |
| | <223> vp2 |
| | <221> misc_feature |
| | <222> (607) . . . (2202) |
| | <223> vp3 |
| 34 | <223> adeno-associated virus hu87 |
| | <221> MISC_FEATURE |
| | <222> (1) . . . (734) |
| | <223> vp1 |
| | <221> MISC_FEATURE |
| | <222> (138) . . . (734) |
| | <223> vp2 |
| | <221> MISC_FEATURE |
| | <222> (203) . . . (734) |
| | <223> vp3 |
| 35 | <223> adeno-associated virus hu88/78 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 36 | <223> adeno-associated virus hu88/78 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 37 | <223> adeno-associated virus hu69 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 38 | <223> adeno-associated virus hu69 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 39 | <223> adeno-associated virus rh75 |
| | <221> misc_feature |
| | <222> (1) . . . (2208) |
| | <223> vp1 |
| | <221> misc_feature |
| | <222> (412) . . . (2208) |
| | <223> vp2 |
| | <221> misc_feature |
| | <222> (607) . . . (2208) |
| | <223> vp3 |
| 40 | <223> adeno-associated virus rh75 |
| | <221> MISC_FEATURE |
| | <222> (1) . . . (736) |
| | <223> vp1 |
| | <221> MISC_FEATURE |
| | <222> (138) . . . (736) |
| | <223> vp2 |
| | <221> MISC_FEATURE |
| | <222> (203) . . . (736) |
| | <223> vp3 |
| 41 | <223> adeno-associated virus rh76 |
| | <221> misc_feature |
| | <222> (1) . . . (2211) |
| | <223> vp1 |
| | <221> misc_feature |
| | <222> (412) . . . (2211) |
| | <223> vp2 |
| | <221> misc_feature |
| | <222> (610) . . . (2211) |
| | <223> vp3 |
| 42 | <223> adeno-associated virus rh76 |
| | <221> MISC_FEATURE |
| | <222> (1) . . . (737) |
| | <223> vp1 |
| | <221> MISC_FEATURE |
| | <222> (138) . . . (737) |
| | <223> vp2 |
| | <221> MISC_FEATURE |
| | <222> (204) . . . (737) |
| | <223> vp3 |
| 43 | <223> adeno-associated virus rh77 |
| | <221> misc_feature |
| | <222> (1) . . . (2199) |
| | <223> vp1 |
| | <221> misc_feature |
| | <222> (412) . . . (2199) |
| | <223> vp2 |
| | <221> misc_feature |
| | <222> (589) . . . (2199) |
| | <223> vp3 |
| 44 | <223> adeno-associated virus rh77 |
| | <221> MISC_FEATURE |
| | <222> (1) . . . (733) |
| | <223> vp1 |
| | <221> MISC_FEATURE |
| | <222> (138) . . . (733) |
| | <223> vp2 |
| | <221> MISC_FEATURE |
| | <222> (197) . . . (733) |
| | <223> vp3 |
| 45 | <223> adeno-associated virus rh78 |
| | <221> misc_feature |
| | <222> (1) . . . (2199) |
| | <223> vp1 |
| | <221> misc_feature |
| | <222> (412) . . . (2199) |
| | <223> vp2 |
| | <221> misc_feature |
| | <222> (589) . . . (2199) |
| | <223> vp3 |
| 46 | <223> adeno-associated virus rh78 |
| | <221> MISC_FEATURE |
| | <222> (1) . . . (733) |
| | <223> vp1 |
| | <221> MISC_FEATURE |
| | <222> (138) . . . (733) |
| | <223> vp2 |
| | <221> MISC_FEATURE |
| | <222> (197) . . . (733) |
| | <223> vp3 |
| 47 | <223> adeno-associated virus rh79 |
| | <221> misc_feature |
| | <222> (1) . . . (2214) |
| | <223> vp1 |
| | <221> misc_feature |
| | <222> (412) . . . (2214) |
| | <223> vp2 |
| | <221> misc_feature |
| | <222> (610) . . . (2214) |
| | <223> vp3 |
| 48 | <223> adeno-associated virus rh79 |
| | <221> MISC_FEATURE |
| | <222> (1) . . . (738) |
| | <223> vp1 |
| | <221> MISC_FEATURE |
| | <222> (138) . . . (738) |
| | <223> vp2 |
| | <221> MISC_FEATURE |
| | <222> (204) . . . (738) |
| | <223> vp3 |
| 49 | <223> adeno-associated virus rh81 |
| | <221> misc_feature |
| | <222> (1) . . . (2217) |
| | <223> vp1 |
| | <221> misc_feature |
| | <222> (412) . . . (2217) |
| | <223> vp2 |
| | <221> misc_feature |
| | <222> (619) . . . (2217) |
| | <223> vp3 |
| 50 | <223> adeno-associated virus rh81 |
| | <221> MISC_FEATURE |
| | <222> (1) . . . (739) |
| | <223> vp1 |
| | <221> MISC_FEATURE |
| | <222> (138) . . . (739) |
| | <223> vp2 |
| | <221> MISC_FEATURE |
| | <222> (207) . . . (739) |
| | <223> vp3 |
| 51 | <223> adeno-associated virus rh89 |
| | <221> misc_feature |
| | <222> (1) . . . (2184) |
| | <223> vp1 |
| | <221> misc_feature |
| | <222> (412) . . . (2184) |
| | <223> vp2 |
| | <221> misc_feature |
| | <222> (595) . . . (2184) |
| | <223> vp3 |
| 52 | <223> adeno-associated virus rh89 |
| | <221> MISC_FEATURE |
| | <222> (1) . . . (728) |
| | <223> vp1 |
| | <221> MISC_FEATURE |
| | <222> (138) . . . (728) |
| | <223> vp2 |
| | <221> MISC_FEATURE |
| | <222> (199) . . . (728) |
| | <223> vp3 |
| 53 | <223> adeno-associated virus rh82 |
| | <221> misc_feature |
| | <222> (1) . . . (2199) |
| | <223> vp1 |
| | <221> misc_feature |
| | <222> (412) . . . (2199) |
| | <223> vp2 |
| | <221> misc_feature |
| | <222> (589) . . . (2199) |
| | <223> vp3 |
| 54 | <223> adeno-associated virus rh82 |
| | <221> MISC_FEATURE |
| | <222> (1) . . . (733) |
| | <223> vp1 |
| | <221> MISC_FEATURE |
| | <222> (138) . . . (733) |
| | <223> vp2 |
| | <221> MISC_FEATURE |
| | <222> (197) . . . (733) |
| | <223> vp3 |
| 55 | <223> adeno-associated virus rh83 |
| | <221> misc_feature |
| | <222> (1) . . . (2211) |
| | <223> vp1 |
| | <221> misc_feature |
| | <222> (412) . . . (2211) |
| | <223> vp2 |
| | <221> misc_feature |
| | <222> (610) . . . (2211) |
| | <223> vp3 |
| 56 | <223> adeno-associated virus rh83 |
| | <221> MISC_FEATURE |
| | <222> (1) . . . (737) |
| | <223> vp1 |
| | <221> MISC_FEATURE |
| | <222> (138) . . . (737) |
| | <223> vp2 |
| | <221> MISC_FEATURE |
| | <222> (204) . . . (737) |
| | <223> vp3 |
| 57 | <223> adeno-associated virus rh84 |
| | <221> misc_feature |
| | <222> (1) . . . (2211) |
| | <223> vp1 |
| | <221> misc_feature |
| | <222> (412) . . . (2211) |
| | <223> vp2 |
| | <221> misc_feature |
| | <222> (610) . . . (2211) |
| | <223> vp3 |
| 58 | <223> adeno-associated virus rh84 |
| | <221> MISC_FEATURE |
| | <222> (1) . . . (737) |
| | <223> vp1 |
| | <221> MISC_FEATURE |
| | <222> (138) . . . (737) |
| | <223> vp2 |
| | <221> MISC_FEATURE |
| | <222> (204) . . . (737) |
| | <223> vp3 |
| 59 | <223> adeno-associated virus rh85 |
| | <221> misc_feature |
| | <222> (1) . . . (2211) |
| | <223> vp1 |
| | <221> misc_feature |
| | <222> (412) . . . (2211) |
| | <223> vp2 |
| | <221> misc_feature |
| | <222> (610) . . . (2211) |
| | <223> vp3 |
| 60 | <223> adeno-associated virus rh85 |
| | <221> MISC_FEATURE |
| | <222> (1) . . . (737) |
| | <223> vp1 |
| | <221> MISC_FEATURE |
| | <222> (138) . . . (737) |
| | <223> vp2 |
| | <221> MISC_FEATURE |
| | <222> (204) . . . (737) |
| | <223> vp3 |
| 61 | <223> adeno-associated virus rh87 |
| | <221> misc_feature |
| | <222> (1) . . . (2211) |
| | <223> vp1 |
| | <221> misc_feature |
| | <222> (412) . . . (2211) |
| | <223> vp2 |
| | <221> misc_feature |
| | <222> (610) . . . (2211) |
| | <223> vp3 |
| 62 | <223> adeno-associated virus rh87 |
| | <221> MISC_FEATURE |
| | <222> (1) . . . (737) |
| | <223> vp1 |
| | <221> MISC_FEATURE |
| | <222> (138) . . . (737) |
| | <223> vp2 |
| | <221> MISC_FEATURE |
| | <222> (204) . . . (737) |
| | <223> vp3 |
| 63 | <223> primer sequence |
| 64 | <223> primer sequence |
| 65 | <223> primer sequence |
| 66 | <223> primer sequence |
| 67 | <223> adeno-associated virus 9 |
| | <221> misc_feature |
| | <222> (1) . . . (2208) |
| | <223> vp1 |
| | <221> misc_feature |
| | <222> (412) . . . (2208) |
| | <223> vp2 |
| | <221> misc_feature |
| | <222> (604) . . . (2208) |
| | <223> vp3 |
| 68 | <223> adeno-associated virus 9 |
| | <221> MISC_FEATURE |
| | <222> (1) . . . (736) |
| | <223> vp1 |
| | <221> MISC_FEATURE |
| | <222> (138) . . . (736) |
| | <223> vp2 |
| | <221> MISC_FEATURE |
| | <222> (202) . . . (736) |
| | <223> vp3 |
| 69 | <223> adeno-associated virus hu32 |
| | <221> misc_feature |
| | <222> (1) . . . (2208) |
| | <223> vp1 |
| | <221> misc_feature |
| | <222> (412) . . . (2208) |
| | <223> vp2 |
| | <221> misc_feature |
| | <222> (604) . . . (2208) |
| | <223> vp3 |
| 70 | <223> adeno-associated virus hu32 |
| | <221> MISC_FEATURE |
| | <222> (1) . . . (736) |
| | <223> vp1 |
| | <221> MISC_FEATURE |
| | <222> (138) . . . (736) |
| | <223> vp2 |
| | <221> MISC_FEATURE |
| | <222> (202) . . . (736) |
| | <223> vp3 |
| 71 | <223> adeno-associated virus rh8 |
| 72 | <223> adeno-associated virus rh8 |
| 73 | <223> adeno-associated virus hu73 |
| | <221> misc_feature |
| | <222> (1) . . . (2205) |
| | <223> vp1 |
| | <221> misc_feature |
| | <222> (412) . . . (2205) |
| | <223> vp2 |
| | <221> misc_feature |
| | <222> (607) . . . (2205) |
| | <223> vp3 |
| 74 | <223> adeno-associated virus hu73 |
| | <221> MISC_FEATURE |
| | <222> (1) . . . (735) |
| | <223> vp1 |
| | <221> MISC_FEATURE |
| | <222> (138) . . . (735) |
| | <223> vp2 |
| | <221> MISC_FEATURE |
| | <222> (203) . . . (735) |
| | <223> vp3 |
| 75 | <223> adeno-associated virus rh.32.33 |
| 76 | <223> adeno-associated virus 9 isolated |
| | nucleic acid sequence |
| | <221> misc_feature |
| | <222> (1) . . . (2208) |
| | <223> vp1 |
| | <221> misc_feature |
| | <222> (412) . . . (2208) |
| | <223> vp2 |
| | <221> misc_feature |
| | <222> (604) . . . (2208) |
| | <223> vp3 |
| 77 | <223> adeno-associated virus hu32 isolated |
| | nucleic acid sequence |
| | <221> misc_feature |
| | <222> (1) . . . (2208) |
| | <223> vp1 |
| | <221> misc_feature |
| | <222> (412) . . . (2208) |
| | <223> vp2 |
| | <221> misc_feature |
| | <222> (604) . . . (2208) |
| | <223> vp3 |
| 78 | <223> synthetic construct |
| 79 | <223> synthetic construct |
| 80 | <223> synthetic construct |
| 81 | <223> synthetic construct |

All patents, patent publications, and other publications listed in this specification are incorporated herein by reference. U.S. Provisional Patent Application No. 63/107,030, filed Oct. 29, 2020, and U.S. Provisional Patent Application No. 63/214,530, filed Jun. 24, 2021, are incorporated herein by reference. The appended Sequence Listing labeled “21-9492.PCT_ST25” is incorporated herein by reference. While the invention has been described with reference to a particularly preferred embodiment, it will be appreciated that modifications can be made without departing from the spirit of the invention. Such modifications are intended to fall within the scope of the appended claims.

1. A recombinant adeno-associated virus (rAAV) comprising a capsid and a vector genome comprising an AAV 5′ inverted terminal repeat (ITR), an expression cassette comprising a nucleic acid sequence encoding a gene product operably linked to expression control sequences, and an AAV 3′ ITR, wherein the capsid is:
(a) an AAVrh75 capsid consisting of (i) a capsid produced from a nucleic acid sequence encoding SEQ ID NO: 40 or a sequence at least 99% identical thereto having an Asn (N) amino acid residue at position 24 based on the numbering of SEQ ID NO: 40; (ii) a capsid produced from a nucleic acid sequence of SEQ ID NO: 39 of a sequence or a sequence at least 95% identical thereto encoding SEQ ID NO: 40; or (iii) a capsid which is heterogeneous mixture of AAVrh75 vp1, vp2 and vp3 proteins which are 95% to 100% deamidated in at least position N57, N262, N384, and/or N512 of SEQ ID NO: 40, and optionally deamidated in other positions;
(b) an AAVhu71/74 capsid consisting of (i) a capsid produced from a nucleic acid sequence encoding SEQ ID NO: 4; (ii) a capsid produced from a nucleic acid sequence of SEQ ID NO: 3 of a sequence or a sequence at least 95% identical thereto encoding SEQ ID NO: 4; or (iii) a capsid which is a heterogeneous mixture of AAVrh71/74 vp1, vp2, and vp3 proteins which are 95% to 100% deamidated in at least 4 positions of SEQ ID NO: 4, and optionally deamidated in other positions;
(c) an AAVhu79 capsid consisting of (i) a capsid produced from a nucleic acid sequence encoding SEQ ID NO: 6; (ii) a capsid produced from a nucleic acid sequence of SEQ ID NO: 5 of a sequence or a sequence at least 95% identical thereto encoding SEQ ID NO: 6; or (iii) a capsid which is a heterogeneous mixture of AAVhu79 vp1, vp2, and vp3 proteins which are 95% to 100% deamidated in at least four positions of SEQ ID NO: 6, and optionally deamidated in other positions;
(d) an AAVhu80 capsid consisting of (i) a capsid produced from a nucleic acid sequence encoding SEQ ID NO: 8; (ii) a capsid produced from a nucleic acid sequence of SEQ ID NO: 7 of a sequence or a sequence at least 95% identical thereto encoding SEQ ID NO: 8; or (iii) a capsid which is a heterogeneous mixture of AAVhu80 vp1, vp2, and vp3 proteins which are 95% to 100% deamidated in at least four positions of SEQ ID NO: 8, and optionally deamidated in other positions;
(e) an AAVhu83 capsid consisting of (i) a capsid produced from a nucleic acid sequence encoding SEQ ID NO: 10; (ii) a capsid produced from a nucleic acid sequence of SEQ ID NO: 9 of a sequence or a sequence at least 95% identical thereto encoding SEQ ID NO: 10; or (iii) a capsid which is a heterogeneous mixture of AAVhu83 vp1, vp2, and vp3 proteins which are 95% to 100% deamidated in at least four positions of SEQ ID NO: 10, and optionally deamidated in other positions;
(f) an AAVhu74/71 capsid consisting of (i) a capsid produced from a nucleic acid sequence encoding SEQ ID NO: 12; (ii) a capsid produced from a nucleic acid sequence of SEQ ID NO: 11 of a sequence or a sequence at least 95% identical thereto encoding SEQ ID NO: 12; or (iii) a capsid which is a heterogeneous mixture of AAVhu74/71 vp1, vp2, and vp3 proteins which are 95% to 100% deamidated in at least four positions of SEQ ID NO: 12, and optionally deamidated in other positions;
(g) an AAVhu77 capsid, consisting of (i) a capsid produced from a nucleic acid sequence encoding SEQ ID NO: 14; (ii) a capsid produced from a nucleic acid sequence of SEQ ID NO: 13 of a sequence or a sequence at least 95% identical thereto encoding SEQ ID NO: 14; or (iii) a capsid which is a heterogeneous mixture of AAVhu77 vp1, vp2, and vp3 proteins which are 95% to 100% deamidated in at least four positions of SEQ ID NO: 14, and optionally deamidated in other positions;
(h) an AAVhu78/88 capsid consisting of (i) a capsid produced from a nucleic acid sequence encoding SEQ ID NO: 16; (ii) a capsid produced from a nucleic acid sequence of SEQ ID NO: 15 of a sequence or a sequence at least 95% identical thereto encoding SEQ ID NO: 16; or (iii) a capsid which is a heterogeneous mixture of AAVhu78/88 vp1, vp2, and vp3 proteins which are 95% to 100% deamidated in at least four positions of SEQ ID NO: 16, and optionally deamidated in other positions;
(i) an AAVhu70 capsid consisting of (i) a capsid produced from a nucleic acid sequence encoding SEQ ID NO: 18; (ii) a capsid produced from a nucleic acid sequence of SEQ ID NO: 17 of a sequence or a sequence at least 95% identical thereto encoding SEQ ID NO: 18; or (iii) a capsid which is a heterogeneous mixture of AAVhu70 vp1, vp2, and vp3 proteins which are 95% to 100% deamidated in at least four positions of SEQ ID NO: 18, and optionally deamidated in other positions;
(j) an AAVhu72 capsid consisting of (i) a capsid produced from a nucleic acid sequence encoding SEQ ID NO: 20; (ii) a capsid produced from a nucleic acid sequence of SEQ ID NO: 19 of a sequence or a sequence at least 95% identical thereto encoding SEQ ID NO: 20; or (iii) a capsid which is a heterogeneous mixture of AAVhu72 vp1, vp2, and vp3 proteins which are 95% to 100% deamidated in at least four positions of SEQ ID NO: 20, and optionally deamidated in other positions;
(k) an AAVhu75 capsid consisting of (i) a capsid produced from a nucleic acid sequence encoding SEQ ID NO: 22; (ii) a capsid produced from a nucleic acid sequence of SEQ ID NO: 21 of a sequence or a sequence at least 95% identical thereto encoding SEQ ID NO: 22; or (iii) a capsid which is a heterogeneous mixture of AAVhu75 vp1, vp2, and vp3 proteins which are 95% to 100% deamidated in at least four positions of SEQ ID NO: 22, and optionally deamidated in other positions;
(l) an AAVhu76 capsid consisting of (i) a capsid produced from a nucleic acid sequence encoding SEQ ID NO: 24; (ii) a capsid produced from a nucleic acid sequence of SEQ ID NO: 23 of a sequence or a sequence at least 95% identical thereto encoding SEQ ID NO: 24; or (iii) a capsid which is a heterogeneous mixture of AAVhu76 vp1, vp2, and vp3 proteins which are 95% to 100% deamidated in at least four positions of SEQ ID NO: 24, and optionally deamidated in other positions;
(m) an AAVhu81 capsid consisting of (i) a capsid produced from a nucleic acid sequence encoding SEQ ID NO: 26; (ii) a capsid produced from a nucleic acid sequence of SEQ ID NO: 25 of a sequence or a sequence at least 95% identical thereto encoding SEQ ID NO: 26; or (iii) a capsid which is a heterogeneous mixture of AAVhu81 vp1, vp2, and vp3 proteins which are 95% to 100% deamidated in at least four positions of SEQ ID NO: 26, and optionally deamidated in other positions;
(n) an AAVhu82 capsid consisting of (i) a capsid produced from a nucleic acid sequence encoding SEQ ID NO: 28; (ii) a capsid produced from a nucleic acid sequence of SEQ ID NO: 27 of a sequence or a sequence at least 95% identical thereto encoding SEQ ID NO: 28; or (iii) a capsid which is a heterogeneous mixture of AAVhu82 vp1, vp2, and vp3 proteins which are 95% to 100% deamidated in at least four positions of SEQ ID NO: 28, and optionally deamidated in other positions;
(o) an AAVhu84 capsid consisting of (i) a capsid produced from a nucleic acid sequence encoding SEQ ID NO: 30; (ii) a capsid produced from a nucleic acid sequence of SEQ ID NO: 29 of a sequence or a sequence at least 95% identical thereto encoding SEQ ID NO: 30; or (iii) a capsid which is a heterogeneous mixture of AAVhu84 vp1, vp2, and vp3 proteins which are 95% to 100% deamidated in at least four positions of SEQ ID NO: 30, and optionally deamidated in other positions;
(p) an AAVhu86 capsid consisting of (i) a capsid produced from a nucleic acid sequence encoding SEQ ID NO: 32; (ii) a capsid produced from a nucleic acid sequence of SEQ ID NO: 31 of a sequence or a sequence at least 95% identical thereto encoding SEQ ID NO: 32; or (iii) a capsid which is a heterogeneous mixture of AAVhu86 vp1, vp2, and vp3 proteins which are 95% to 100% deamidated in at least four positions of SEQ ID NO: 32, and optionally deamidated in other positions;
(q) an AAVhu87 capsid consisting of (i) a capsid produced from a nucleic acid sequence encoding SEQ ID NO: 34; (ii) a capsid produced from a nucleic acid sequence of SEQ ID NO: 33 of a sequence or a sequence at least 95% identical thereto encoding SEQ ID NO: 34; or (iii) a capsid which is a heterogeneous mixture of AAVhu87 vp1, vp2, and vp3 proteins which are 95% to 100% deamidated in at least four positions of SEQ ID NO: 34, and optionally deamidated in other positions;
(r) an AAVhu88/78 capsid consisting of (i) a capsid produced from a nucleic acid sequence encoding SEQ ID NO: 36; (ii) a capsid produced from a nucleic acid sequence of SEQ ID NO: 35 of a sequence or a sequence at least 95% identical thereto encoding SEQ ID NO: 36; or (iii) a capsid which is a heterogeneous mixture of AAVhu88/78 vp1, vp2, and vp3 proteins which are 95% to 100% deamidated in at least four positions of SEQ ID NO: 36, and optionally deamidated in other positions;
(s) an AAVhu69 capsid consisting of (i) a capsid produced from a nucleic acid sequence encoding SEQ ID NO: 38; (ii) a capsid produced from a nucleic acid sequence of SEQ ID NO: 37 of a sequence or a sequence at least 95% identical thereto encoding SEQ ID NO: 38; or (iii) a capsid which is a heterogeneous mixture of AAVhu69 vp1, vp2, and vp3 proteins which are 95% to 100% deamidated in at least four positions of SEQ ID NO: 38, and optionally deamidated in other positions;
(t) an AAVrh76 capsid consisting of (i) a capsid produced from a nucleic acid sequence encoding SEQ ID NO: 42; (ii) a capsid produced from a nucleic acid sequence of SEQ ID NO: 41 of a sequence or a sequence at least 95% identical thereto encoding SEQ ID NO: 42; or (iii) a capsid which is a heterogeneous mixture of AAVhu69 vp1, vp2, and vp3 proteins which are 95% to 100% deamidated in at least four positions of SEQ ID NO: 42, and optionally deamidated in other positions;
(u) an AAVrh77 capsid consisting of (i) a capsid produced from a nucleic acid sequence encoding SEQ ID NO: 44; (ii) a capsid produced from a nucleic acid sequence of SEQ ID NO: 43 of a sequence or a sequence at least 95% identical thereto encoding SEQ ID NO: 44; or (iii) a capsid which is a heterogeneous mixture of AAVrh71 vp1, vp2, and vp3 proteins which are 95% to 100% deamidated in at least four positions of SEQ ID NO: 44, and optionally deamidated in other positions;
(v) an AAVrh78 capsid consisting of (i) a capsid produced from a nucleic acid sequence encoding SEQ ID NO: 46; (ii) a capsid produced from a nucleic acid sequence of SEQ ID NO: 45 of a sequence or a sequence at least 95% identical thereto encoding SEQ ID NO: 46; or (iii) a capsid which is a heterogeneous mixture of AAVrh78 vp1, vp2, and vp3 proteins which are 95% to 100% deamidated in at least four positions of SEQ ID NO: 46, and optionally deamidated in other positions;
(w) an AAVrh81 capsid consisting of (i) a capsid produced from a nucleic acid sequence encoding SEQ ID NO: 50; (ii) a capsid produced from a nucleic acid sequence of SEQ ID NO: 49 of a sequence or a sequence at least 95% identical thereto encoding SEQ ID NO: 50; or (iii) a capsid which is a heterogeneous mixture of AAVrh81 vp1, vp2, and vp3 proteins which are 95% to 100% deamidated in at least four positions of SEQ ID NO: 50, and optionally deamidated in other positions;
(x) an AAVrh89 capsid consisting of (i) a capsid produced from a nucleic acid sequence encoding SEQ ID NO: 52; (ii) a capsid produced from a nucleic acid sequence of SEQ ID NO: 51 of a sequence or a sequence at least 95% identical thereto encoding SEQ ID NO: 52; or (iii) a capsid which is a heterogeneous mixture of AAVrh89 vp1, vp2, and vp3 proteins which are 95% to 100% deamidated in at least four positions of SEQ ID NO: 52, and optionally deamidated in other positions;
(y) an AAVrh82 capsid consisting of (i) a capsid produced from a nucleic acid sequence encoding SEQ ID NO: 54; (ii) a capsid produced from a nucleic acid sequence of SEQ ID NO: 53 of a sequence or a sequence at least 95% identical thereto encoding SEQ ID NO: 54; or (iii) a capsid which is a heterogeneous mixture of AAVrh82 vp1, vp2, and vp3 proteins which are 95% to 100% deamidated in at least four positions of SEQ ID NO: 54, and optionally deamidated in other positions;
(z) an AAVrh83 capsid consisting of (i) a capsid produced from a nucleic acid sequence encoding SEQ ID NO: 56; (ii) a capsid produced from a nucleic acid sequence of SEQ ID NO: 55 of a sequence or a sequence at least 95% identical thereto encoding SEQ ID NO: 56; or (iii) a capsid which is a heterogeneous mixture of AAVrh83 vp1, vp2, and vp3 proteins which are 95% to 100% deamidated in at least four positions of SEQ ID NO: 56, and optionally deamidated in other positions;
(aa) an AAVrh84 capsid consisting of (i) a capsid produced from a nucleic acid sequence encoding SEQ ID NO: 58; (ii) a capsid produced from a nucleic acid sequence of SEQ ID NO: 57 of a sequence or a sequence at least 95% identical thereto encoding SEQ ID NO: 58; or (iii) a capsid which is a heterogeneous mixture of AAVrh84 vp1, vp2, and vp3 proteins which are 95% to 100% deamidated in at least four positions of SEQ ID NO: 58, and optionally deamidated in other positions;
(bb) an AAVrh85 capsid consisting of (i) a capsid produced from a nucleic acid sequence encoding SEQ ID NO: 60; (ii) a capsid produced from a nucleic acid sequence of SEQ ID NO: 59 of a sequence or a sequence at least 95% identical thereto encoding SEQ ID NO: 60; or (iii) a capsid which is a heterogeneous mixture of AAVrh85 vp1, vp2, and vp3 proteins which are 95% to 100% deamidated in at least four positions of SEQ ID NO: 60, and optionally deamidated in other positions;
(cc) an AAVrh87 capsid consisting of (i) a capsid produced from a nucleic acid sequence encoding SEQ ID NO: 62; (ii) a capsid produced from a nucleic acid sequence of SEQ ID NO: 61 of a sequence or a sequence at least 95% identical thereto encoding SEQ ID NO: 62; or (iii) a capsid which is a heterogeneous mixture of AAVrh87 vp1, vp2, and vp3 proteins which are 95% to 100% deamidated in at least four positions of SEQ ID NO: 62, and optionally deamidated in other positions; or
(dd) an AAVhu73 capsid consisting of (i) a capsid produced from a nucleic acid sequence encoding SEQ ID NO: 74; (ii) a capsid produced from a nucleic acid sequence of SEQ ID NO: 73 of a sequence or a sequence at least 95% identical thereto encoding SEQ ID NO: 74; or (iii) a capsid which is a heterogeneous mixture of AAVrh73 vp1, vp2, and vp3 proteins which are 95% to 100% deamidated in at least four positions of SEQ ID NO: 74, and optionally deamidated in other positions.

2. The rAAV according to claim 1, wherein the gene product is useful in treating a disorder or disease of the liver, and wherein the capsid is an AAVrh75, AAVrh79, AAVrh83, or AAVrh84 capsid.

3. The rAAV according to claim 1, wherein the gene product is a gene editing nuclease.

4. (canceled)

5. The rAAV according to claim 1, wherein the expression cassette comprises a tissue-specific promoter.

6. A host cell containing the rAAV according to claim 1.

7. A pharmaceutical composition comprising the rAAV according to claim 1, and a physiologically compatible carrier, buffer, adjuvant, and/or diluent.

8. A method of delivering a transgene to a cell, said method comprising the step of contacting the cell with the rAAV according to claim 1, wherein said rAAV comprises the transgene.

9. A method of generating a recombinant adeno-associated virus (rAAV) comprising an AAV capsid, the method comprising culturing a host cell containing: (a) a molecule encoding an AAV vp1, vp2, and/or vp3 capsid protein of AAVrh75 (SEQ ID NO: 40), AAVhu71/74 (SEQ ID NO: 4), AAVhu79 (SEQ ID NO: 6), AAVhu80 (SEQ ID NO: 8), AAVhu83 (SEQ ID NO: 10), AAVhu74/71 (SEQ ID NO: 12), AAVhu77 (SEQ ID NO: 14), AAVhu78/88 (SEQ ID NO: 16), AAVhu70 (SEQ ID NO: 18), AAVhu72 (SEQ ID NO: 20), AAVhu75 (SEQ ID NO: 22), AAVhu76 (SEQ ID NO: 24), AAVhu81 (SEQ ID NO: 26), AAVhu82 (SEQ ID NO: 28), AAVhu84 (SEQ ID NO: 30), AAVhu86 (SEQ ID NO: 32), AAVhu87 (SEQ ID NO: 34), AAVhu88/78 (SEQ ID NO: 36), AAVhu69 (SEQ ID NO: 38), AAVrh76 (SEQ ID NO: 42), AAVrh77 (SEQ ID NO: 44), AAVrh78 (SEQ ID NO: 46), AAVrh81 (SEQ ID NO: 50), AAVrh89 (SEQ ID NO: 52), AAVrh82 (SEQ ID NO: 54), AAVrh83 (SEQ ID NO: 56), AAVrh84 (SEQ ID NO: 58), AAVrh85 (SEQ ID NO: 60), AAVrh87 (SEQ ID NO: 62), or AAVhu73 (SEQ ID NO: 74), or an AAV vp1, vp2, and/or vp3 capsid protein sharing at least 99% identity with any of SEQ ID NOs: 40, 4, 6, 8, 10, 12, 14, 16, 18, 20, 22, 24, 26, 28, 30, 32, 34, 36, 38, 42, 44, 46, 50, 52, 54, 56, 58, 60, 62, or 74, (b) a functional rep gene; (c) a vector genome comprising AAV inverted terminal repeats (ITRs) and a transgene; and (d) sufficient helper functions to permit packaging of the vector genome into the AAV capsid protein.

10. A plasmid comprising a vp1, vp2, and/or vp3 sequence of AAVrh75 (SEQ ID NO: 39), AAVhu71/74 (SEQ ID NO: 3), AAVhu79 (SEQ ID NO: 5), AAVhu80 (SEQ ID NO: 7), AAVhu83 (SEQ ID NO: 9), AAVhu74/71 (SEQ ID NO: 11), AAVhu77 (SEQ ID NO: 13), AAVhu78/88 (SEQ ID NO: 15), AAVhu70 (SEQ ID NO: 17), AAVhu72 (SEQ ID NO: 19), AAVhu75 (SEQ ID NO: 21), AAVhu76 (SEQ ID NO: 23), AAVhu81 (SEQ ID NO: 25), AAVhu82 (SEQ ID NO: 27), AAVhu84 (SEQ ID NO: 29), AAVhu86 (SEQ ID NO: 31), AAVhu87 (SEQ ID NO: 33), AAVhu88/78 (SEQ ID NO: 35), AAVhu69 (SEQ ID NO: 37), AAVrh76 (SEQ ID NO: 41), AAVrh77 (SEQ ID NO: 43), AAVrh78 (SEQ ID NO: 45), AAVrh81 (SEQ ID NO: 49), AAVrh89 (SEQ ID NO: 51), AAVrh82 (SEQ ID NO: 53), AAVrh83 (SEQ ID NO: 55), AAVrh84 (SEQ ID NO: 57), AAVrh85 (SEQ ID NO: 59), AAVrh87 (SEQ ID NO: 61), or AAVhu73 (SEQ ID NO: 73), or vp1, vp2, and/or vp3 sequence sharing at least 95% identity with any of SEQ ID NO: 39, 3, 5, 7, 9, 11, 13, 15, 17, 19, 21, 23, 25, 27, 29, 31, 33, 35, 37, 41, 43, 45, 49, 51, 53, 55, 57, 59, 61, or 73.

11. A cultured host cell containing the plasmid according to claim 10.
